SEQUENCE LISTING (64 sequences)

SEQ ID NO: 1 - protein, 404 aa, Homo sapiens
  MISC_FEATURE: Genbank Accession No. NP_001127
MAAGTAVGAWVLVLSLWGAVVGAQNITARIGEPLVLKCKGAPKKPPQRLEWKLNTGRTEAWKVLSPQGGGPWDSVARVLPNGSLFLPAVGIQDEGIFRCQAMNRNGKETKSNYRVRVYQIPGKPEIVDSASELTAGVPNKVGTCVSEGSYPAGTLSWHLDGKPLVPNEKGVSVKEQTRRHPETGLFTLQSELMVTPARGGDPRPTFSCSFSPGLPRHRALRTAPIQPRVWEPVPLEEVQLVVEPEGGAVAPGGTVTLTCEVPAQPSPQIHWMKDGVPLPLPPSPVLILPEIGPQDQGTYSCVATHSSHGPQESRAVSISIIEPGEEGPTAGSVGGSGLGTLALALGILGGLGTAALLIGVILWQRRQRRGEERKAPENQEEEEERAELNQSEEPEAGESSTGGP

SEQ ID NO: 2 - DNA, 1414 bp, Homo sapiens
  misc_feature: Genbank Accession No. NM_001136
gccaggaccctggaaggaagcaggatggcagccggaacagcagttggagcctgggtgctggtcctcagtctgtggggggcagtagtaggtgctcaaaacatcacagcccggattggcgagccactggtgctgaagtgtaagggggcccccaagaaaccaccccagcggctggaatggaaactgaacacaggccggacagaagcttggaaggtcctgtctccccagggaggaggcccctgggacagtgtggctcgtgtccttcccaacggctccctcttccttccggctgtcgggatccaggatgaggggattttccggtgccaggcaatgaacaggaatggaaaggagaccaagtccaactaccgagtccgtgtctaccagattcctgggaagccagaaattgtagattctgcctctgaactcacggctggtgttcccaataaggtggggacatgtgtgtcagagggaagctaccctgcagggactcttagctggcacttggatgggaagcccctggtgcctaatgagaagggagtatctgtgaaggaacagaccaggagacaccctgagacagggctcttcacactgcagtcggagctaatggtgaccccagcccggggaggagatccccgtcccaccttctcctgtagcttcagcccaggccttccccgacaccgggccttgcgcacagcccccatccagccccgtgtctgggagcctgtgcctctggaggaggtccaattggtggtggagccagaaggtggagcagtagctcctggtggaaccgtaaccctgacctgtgaagtccctgcccagccctctcctcaaatccactggatgaaggatggtgtgcccttgccccttccccccagccctgtgctgatcctccctgagatagggcctcaggaccagggaacctacagctgtgtggccacccattccagccacgggccccaggaaagccgtgctgtcagcatcagcatcatcgaaccaggcgaggaggggccaactgcaggctctgtgggaggatcagggctgggaactctagccctggccctggggatcctgggaggcctggggacagccgccctgctcattggggtcatcttgtggcaaaggcggcaacgccgaggagaggagaggaaggccccagaaaaccaggaggaagaggaggagcgtgcagaactgaatcagtcggaggaacctgaggcaggcgagagtagtactggagggccttgaggggcccacagacagatcccatccatcagctcccttttctttttcccttgaactgttctggcctcagaccaactctctcctgtataatctctctcctgtataaccccaccttgccaagctttcttctacaaccagagccccccacaatgatgattaaacacctgacacatcttga

SEQ ID NO: 3 - protein, 403 aa, Murine
  MISC_FEATURE: Genbank Accession No. NP_031451
MPAGTAARAWVLVLALWGAVAGGQNITARIGEPLVLSCKGAPKKPPQQLEWKLNTGRTEAWKVLSPQGGPWDSVAQILPNGSLLLPATGIVDEGTFRCRATNRRGKEVKSNYRVRVYQIPGKPEIVDPASELTASVPNKVGTCVSEGSYPAGTLSWHLDGKLLIPDGKETLVKEETRRHPETGLFTLRSELTVIPTQGGTTHPTFSCSFSLGLPRRRPLNTAPIQLRVREPGPPEGIQLLVEPEGGIVAPGGTVTLTCAISAQPPPQVHWIKDGAPLPLAPSPVLLLPEVGHADEGTYSCVATHPSHGPQESPPVSIRVTETGDEGPAEGSVGESGLGTLALALGILGGLGVVALLVGAILWRKRQPRREERKAPESQEDEEERAELNQSEEAEMPENGAGGP

SEQ ID NO: 4 - DNA, 1348 bp, Murine
  misc_feature: Genbank Accession No. NM_007425.1
gcaccatgccagcggggacagcagctagagcctgggtgctggttcttgctctatggggagctgtagctggtggtcagaacatcacagcccggattggagagccacttgtgctaagctgtaagggggcccctaagaagccgccccagcagctagaatggaaactgaacacaggaagaactgaagcttggaaggtcctctctccccagggaggcccctgggacagcgtggctcaaatcctccccaatggttccctcctccttccagccactggaattgtcgatgaggggacgttccggtgtcgggcaactaacaggcgagggaaggaggtcaagtccaactaccgagtccgagtctaccagattcctgggaagccagaaattgtggatcctgcctctgaactcacagccagtgtccctaataaggtggggacatgtgtgtctgagggaagctaccctgcagggacccttagctggcacttagatgggaaacttctgattcccgatggcaaagaaacactcgtgaaggaagagaccaggagacaccctgagacgggactctttacactgcggtcagagctgacagtgatccccacccaaggaggaaccacccatcctaccttctcctgcagtttcagcctgggccttccccggcgcagacccctgaacacagcccctatccaactccgagtcagggagcctgggcctccagagggcattcagctgttggttgagcctgaaggtggaatagtcgctcctggtgggactgtgaccttgacctgtgccatctctgcccagccccctcctcaggtccactggataaaggatggtgcacccttgcccctggctcccagccctgtgctgctcctccctgaggtggggcacgcggatgagggcacctatagctgcgtggccacccaccctagccacggacctcaggaaagccctcctgtcagcatcagggtcacagaaaccggcgatgaggggccagctgaaggctctgtgggtgagtctgggctgggtacgctagccctggccttggggatcctgggaggcctgggagtagtagccctgctcgtcggggctatcctgtggcgaaaacgacaacccaggcgtgaggagaggaaggccccggaaagccaggaggatgaggaggaacgtgcagagctgaatcagtcagaggaagcggagatgccagagaatggtgccgggggaccgtaagagcacccagatcgagcctgtgtgatggccctagagcagctcccccacattccatcccaattcctccttgaggcacttccttctccaaccagagcccacatgatccatgctgagtaaacatttgatacggc

SEQ ID NO: 5 - protein, 8 aa, Artificial
  Streptavidin tag sequence
WSHPQFEK

SEQ ID NO: 6 - DNA, 1250 bp, Baboon
  CDS (20)..(1231)
gaccctggaaggaagcaggatggcagccggagcagcagttggagcctgggtgctggtcctcagtctgtggggggcagtagtaggtgctcaaaacatcacagcccggatcggtgagccactggtgctgaagtgtaagggggcccccaagaaaccaccccagcagctggaatggaaactgaatacaggccggacagaagcttggaaggtcctatctccccagggaggcccctgggatagtgtggctcgtgtccttcccaacggctccctcttccttccggctgtcgggatccaggatgaggggattttccggtgccaggcaatgaacaggaatggaaaggagaccaagtccaactaccgagtccgtgtctaccagattcctgggaagccggaaattatagattctgcctctgaactcacggctggtgttcccaataaggtggggacatgtgtgtcagagggaagctaccctgcagggactcttagctggcacttggatgggaagcccctggtgcctaatgagaagggagtatctgtgaaggaagagaccaggagacaccctgagacggggctcttcacactgcagtcggagctaatggtgaccccagcccggggaggaaatccccatcccaccttctcctgtagcttcagcccaggccttccccgacgccgggccttgcacacagcccctatccagccccgtgtctgggagcctgtgcctctggaggaggtccaattggtggtagagccagaaggtggagcagtagctcctggtggaaccgtaaccctgacctgtgaagtccctgcccagccctctcctcagatccactggatgaaggatggtgtgcccttacccctttcccccagccctgtgctgatcctccctgagatagggcctcaggaccagggaacctacaggtgtgtggccacccatcccagccacgggccccaggaaagccgtgctgtcagcatcagcatcatcgaaccaggcgaggaggggccaactgcaggctctgtgggaggatcagggccaggaactctagccctggccctggggatcctgggaggcctggggacagccgctctgctcattggggtcatcttgtggcaaaggcggcgacgccaacgagaggagaggaaggcctcagaaaaccaggaggaagaggaggagcgtgcagagctgaatcagtcggaggaacctgaggcaggcgagagtggtactggagggccttgaggggcccacagacagatcc

SEQ ID NO: 7 - protein, 403 aa, Baboon
MAAGAAVGAWVLVLSLWGAVVGAQNITARIGEPLVLKCKGAPKKPPQQLEWKLNTGRTEAWKVLSPQGGPWDSVARVLPNGSLFLPAVGIQDEGIFRCQAMNRNGKETKSNYRVRVYQIPGKPEIIDSASELTAGVPNKVGTCVSEGSYPAGTLSWHLDGKPLVPNEKGVSVKEETRRHPETGLFTLQSELMVTPARGGNPHPTFSCSFSPGLPRRRALHTAPIQPRVWEPVPLEEVQLVVEPEGGAVAPGGTVTLTCEVPAQPSPQIHWMKDGVPLPLSPSPVLILPEIGPQDQGTYRCVATHPSHGPQESRAVSISIIEPGEEGPTAGSVGGSGPGTLALALGILGGLGTAALLIGVILWQRRRRQREERKASENQEEEEERAELNQSEEPEAGESGTGGP

SEQ ID NO: 8 - DNA, 1250 bp, Cynomologus Monkey
  CDS (20)..(1231)
gaccctggaaggaagcaggatggcagccggagcagcagttggagcctgggtgctggtcctcagtctgtggggggcagtagtaggtgctcaaaacatcacagcccggatcggtgagccactggtgctgaagtgtaagggggcccccaagaaaccaccccagcagctggaatggaaactgaatacaggccggacagaagcttggaaggtcctatctccccagggaggcccctgggatagtgtggctcgtgtccttcccaacggctccctcttccttccggctgtcgggatccaggatgaggggattttccggtgccaggcaatgaacaggaatggaaaggagaccaagtccaactaccgagtccgtgtctaccagattcctgggaagccggaaattatagattctgcctctgaactcacggctggtgttcccaataaggtggggacatgtgtgtcagagggaagctaccctgcagggactcttagctggcacttggatgggaagcccctggtgcctaatgagaagggagtatctgtgaaggaagagaccaggagacaccctgagacggggctcttcacactgcagtcggagctaatggtgaccccagcccggggaggaaatccccatcccaccttctcctgtagcttcagcccaggccttccccgacgccgggccttgcacacagcccctatccagccccgtgtctgggagcctgtgcctctggaggaggtccaattggtggtagagccagaaggtggagcagtagctcctggtggaaccgtaaccctgacctgtgaagtccctgcccagccctctcctcaaatccactggatgaaggatggtgtgcccttacccctttcccccagccctgtgctgatcctccctgagatagggcctcaggaccagggaacctacaggtgtgtggccacccatcccagccacgggccccaggaaagccgtgctgtcagcatcagcatcatcgaaccaggcgaggaggggccaactgcaggctctgtgggaggatcagggccaggaactctagccctggccctggggatcctgggaggcctggggacagccgctctgctcattggggtcatcttgtggcaaaggcggcgacgccaaggagaggagaggaaggcctcagaaaaccaggaggaagaggaggagcgtgcagagctgaatcagtcggaggaacctgaggcaggcgagagtggtactggagggccttgaggggcccacagacagatcc

SEQ ID NO: 9 - protein, 403 aa, Cynomologus Monkey
MAAGAAVGAWVLVLSLWGAVVGAQNITARIGEPLVLKCKGAPKKPPQQLEWKLNTGRTEAWKVLSPQGGPWDSVARVLPNGSLFLPAVGIQDEGIFRCQAMNRNGKETKSNYRVRVYQIPGKPEIIDSASELTAGVPNKVGTCVSEGSYPAGTLSWHLDGKPLVPNEKGVSVKEETRRHPETGLFTLQSELMVTPARGGNPHPTFSCSFSPGLPRRRALHTAPIQPRVWEPVPLEEVQLVVEPEGGAVAPGGTVTLTCEVPAQPSPQIHWMKDGVPLPLSPSPVLILPEIGPQDQGTYRCVATHPSHGPQESRAVSISIIEPGEEGPTAGSVGGSGPGTLALALGILGGLGTAALLIGVILWQRRRRQGEERKASENQEEEEERAELNQSEEPEAGESGTGGP

SEQ ID NO: 10 - DNA, 1220 bp, Rabbit
  CDS (18)..(1196)
actagactagtcggaccatggcagcaggggcagcggccggagcctgggtgctggtcttcagtctgtggggggcagcagtaggtggtcagaacatcacagcccggattggggagccgctggtgctgaagtgtaagggggcccccaagaagccaccccagcagctggaatggaaactgaacacaggcaggacagaagcttggaaagtcctttctccccagggaggctcatgggacagtgtggcccgtgtcctccccaatggctccctcctccttccggctgttgggatccaggatgaggggactttccggtgccggacaacaaacaggaatggaaaggagaccaagtccaattaccgagtccgggtctaccagattcctgggaagccagagatcctggatcctgcctctgaactcacggccggtatccccagtaaggtggggacatgtgtgtctgatgggggatatcctctggggactctcagctggcacatggatgggaaactcctggtacctgacgggaagggagtgtctgtgaaggagcagaccaggaggcaccctgacacggggctcttcaccctgcagtcagagctgatggtgactccagccgggggaggagggcctccccccaccttctcctgtagcttcagccccggcctgccccgccgccgggcctcatacacagcccccatccagcccagtgtctgggagcctgggcccctggaggttcgcttgctggtggagccagaaggtggagcagtagctcctggtgagactgtgaccctgacctgcgaagctcctgcccagccccctcctcaaatccactggatgaaggatggtatgtccctacccctgccccccagccccgtcctgctcctccctgaggtggggcctcaagatgaggggacttacagctgcgtggccacccatcccaaccgtgggccccaggaaagccttcccatcagcatcagtgtcggctctgagggtggctcaggcctggggactctagctctggccctggggatcctgggaggcctgggaacagctgccctgcttgtcggagtcatcctgtggcgaaggcggaaacgccaaggagagcagaggaaagtccctgaaaaccaggaggacgaggaggaacgcacagaactgcatcagtcggaggctcgggaggcgatggagagcggtacaggagagccctgaatagtttagcggccgcattcttat

SEQ ID NO: 11 - protein, 392 aa, Rabbit
MAAGAAAGAWVLVFSLWGAAVGGQNITARIGEPLVLKCKGAPKKPPQQLEWKLNTGRTEAWKVLSPQGGSWDSVARVLPNGSLLLPAVGIQDEGTFRCRTTNRNGKETKSNYRVRVYQIPGKPEILDPASELTAGIPSKVGTCVSDGGYPLGTLSWHMDGKLLVPDGKGVSVKEQTRRHPDTGLFTLQSELMVTPAGGGGPPPTFSCSFSPGLPRRRASYTAPIQPSVWEPGPLEVRLLVEPEGGAVAPGETVTLTCEAPAQPPPQIHWMKDGMSLPLPPSPVLLLPEVGPQDEGTYSCVATHPNRGPQESLPISISVGSEGGSGLGTLALALGILGGLGTAALLVGVILWRRRKRQGEQRKVPENQEDEEERTELHQSEAREAMESGTGEP

SEQ ID NO: 12 - DNA, 1247 bp, Rabbit
  CDS (18)..(1223)
actagactagtcggaccatggcagcaggggcagcggccggagcctgggtgctggtcttcagtctgtggggggcagcagtaggtggtcagaacatcacagcccggattggggagccgctggtgctgaagtgtaagggggcccccaagaagccaccccagcagctggaatggaaactgaacacaggcagaacagaagcttggaaagtcctttctccccagggaggctcatgggacagtgtggcccatgtcctccccaatggctccctcctccttccggctgttgggatccaggatgaggggactttccggtgccggacaacaaacaggaatggaaaggagaccaagtccaattaccgagtccgggtctaccagattcctgggaagccagagatcctggatcctgcctctgaactcacggccggtatccccagtaaggtggggacatgtgtgtctgatgggggatatcctctggggactctcagctggcacatggatgggaaactcctggtacctgacgggaagggagtgtctgtgaaggagcagaccaggaggcatcctgacacggggctcttcaccctgcagtcagagctgatggtgactccagctgggggaggagggcctccccccaccttctcctgtagcttcagccccggcctaccccgccgccgggcctcatacacagcccccatccagcccagtgtctgggagcctgggcccctggaggttcgcttgctggtggagccagaaggtggagcagtagctcctggtgagactgtgaccctgacctgtgaagctcctgcccagccccctcctcaaatccactggatgaaggatggtatgtccctacccctgccccccagccccgtcctgctcctccctgaggtggggcctcaagatgaggggacttacagctgcgtggccacccatcccaaccgtgggccccaggaaagccttcccatcagcatcagtgtcgagacaggcgaggatgggccgactgcaggctctgagggtggctcaggcctggggactctagctctggccctggggatcctgggaggcctgggaacagctgccctgcttgtcggagtcatcctgtggcgaaggcggaaacgccaaggagagcagaggaaagtccccgaaaaccaggaggacgaggaggaacgcacagaactgcatcagtcggaggctcgggaggcgatggagagcggtacaggagagccctgaatagtttagcggccgcattcttat

SEQ ID NO: 13 - protein, 401 aa, Rabbit
MAAGAAAGAWVLVFSLWGAAVGGQNITARIGEPLVLKCKGAPKKPPQQLEWKLNTGRTEAWKVLSPQGGSWDSVAHVLPNGSLLLPAVGIQDEGTFRCRTTNRNGKETKSNYRVRVYQIPGKPEILDPASELTAGIPSKVGTCVSDGGYPLGTLSWHMDGKLLVPDGKGVSVKEQTRRHPDTGLFTLQSELMVTPAGGGGPPPTFSCSFSPGLPRRRASYTAPIQPSVWEPGPLEVRLLVEPEGGAVAPGETVTLTCEAPAQPPPQIHWMKDGMSLPLPPSPVLLLPEVGPQDEGTYSCVATHPNRGPQESLPISISVETGEDGPTAGSEGGSGLGTLALALGILGGLGTAALLVGVILWRRRKRQGEQRKVPENQEDEEERTELHQSEAREAMESGTGEP

SEQ ID NO: 14 - protein, 402 aa, Rattus norvegicus
  MISC_FEATURE: Genbank No. NP_445788
MPTGTVARAWVLVLALWGAVAGGQNITARIGEPLMLSCKGAPKKPTQKLEWKLNTGRTEAWKVLSPQGDPWDSVARILPNGSLLLPAIGIVDEGTFRCRATNRLGKEVKSNYRVRVYQIPGKPEIVNPASELTANVPNKVGTCVSEGSYPAGTLSWHLDGKPLIPDGKGTVVKEETRRHPETGLFTLRSELTVTPAQGGTTPTYSCSFSLGLPRRRPLNTAPIQPRVREPLPPEGIQLLVEPEGGTVAPGGTVTLTCAISAQPPPQIHWIKDGTPLPLAPSPVLLLPEVGHEDEGIYSCVATHPSHGPQESPPVNIRVTETGDEGQAAGSVDGSGLGTLALALGILGGLGIAALLIGAILWRKRQPRLEERKAPESQEDEEERAELNQSEEAEMPENGAGGP

SEQ ID NO: 15 - DNA, 5301 bp, baboon
ttttttaaaaactactatttgaaaattggagggggaagagtgggaagggagttattgccaaatatgttaaatatgggttggggtgcttgtatatgtatcttcctcaatttccccagaaatgaggtatctttttgtcacaccaaaatcaagtggtagggagagggaggaggttgcaaaaagccaagtgtgggggaaaagtaaaatcaacactgtcccatcctcagccctaaaccctaccatctgatcccctcagacattctcaggattttgcaagactgtcagagtggggaacccctcccattaaagatctgggcaggactggggacaggttggaagtgtgatgggtgggggggtgggaggcatgggctgggggcagttctctcctcacttgtaaacttgtgtagtttcacagaaaaaaaacaaaatgcagttttaaataaagaagtttctttttttcctgggtttagttgagaatttttttcaaaaaacatgagaaaccccagaaaaaaaaatatatattttctttcacgaagctccaaacaggtttctctcctgtcccccagccttgccttcacgatgcagtcccaattgcacccttgcaaacaacagtctggcctcaacgctattgatgcaaccttgcccagtcaacatggggctccagtgggtcaccaggcagccctgatggactgatggaataaataggatagggggctctgagggaatgagaccctagagggtacactccccatcccccagggaagtgactgtgtccagaggctggtagtgcccaggggtggggtgataattatttctctagtacctgaaggactcttgtcccaaaggcatgaattcctagcattccctgtgacaagatgactgaaagatgggggctggagagagggtacaggccccacctagggcggaggccacagcgcggagaggggcagacagagctatgagcctggaaggaagcaggatggcagccggagcagcagttggagcctgggtgctggtcctcagtctgtggggtgagccactccccccacccactgacccttcctacagaaagcacttgaaccccataccccaattgccctagaacttttcccagaacccagggaactgcttttcaaggtcccgcacgcaccctgtccaaattttgttagccctcattaccttcctgcccctctaccacgatgctgtctcccaggggcagtagtaggtgctcaaaacatcacagcccggatcggtgagccactggtgctgaagtgtaagggggcccccaagaaaccaccccagcagctggaatggaaactggtaagtggggatcctgttgcagcttcccaacttccagggagaccagcaatgattcggatccccatcactctgcctcacagtactttcccaaaggccttgcactgtttaggccctgcttctctgcttctagaatacaggccggacagaagcttggaaggtcctatctccccagggaggcccctgggatagtgtggctcgtgtccttcccaacggctccctcttccttccggctgtcgggatccaggatgaggggattttccggtgccaggcaatgaacaggaatggaaaggagaccaagtccaactaccgagtccgtgtctaccgtaagaattccagggccttctccaaggccccctctcttatctcagaaaaagccttcaaccctagccttggcccatgagggcctctgacttccactggccccatttccacacacagagtttgagaaccttcacaattacagcctctgattggatttttccttcttcagagattcctgggaagccggaaattatagattctgcctctgaactcacggctggtgttcccaacaaggtagtgaaagaaaggagaagtagaaaatggtcctgtgaacaggaggcgagtgtgtgtgggtgtgtggcatctctcattttcaaaggattctgaggtcaccactctttccccaggtggggacatgtgtgtcagagggaagctaccctgcagggactcttagctggcacttggatgggaagcccctggtgcctaatgagaagggtgagtccgaaggtgcccgccaagctgccttctccctgatctcactcccacacccaccctgggataatttgtcttatcctcctaccataggagtatctgtgaaggaagagaccaggagacaccctgagacggggctcttcacactgcagtcggagctaatggtgaccccagcccggggaggaaatccccatcccaccttctcctgtagcttcagcccaggccttccccgacgccgggccttgcacacagcccctatccagccccgtgtctggggtgagcagaggtggggagggctccaagctcatgtgagtgcattctggaagtcggacccttagggaaagagggagtcaagcccatggccactgggatcactcacaagtgtaactctccacctcataacccttccaactcccagagcctgtgcctctggaggaggtccaattggtggtagagccagaaggtggagcagtagctcctggtggaaccgtaaccctgacctgtgaagtccctgcccagccctctcctcagatccactggatgaaggatgtgagtgacctggagagaggggctgggaggtagggtgaaccataactagcaacagggagggcagagggctaacgagggaaaggcaggctaggagctgaggaggaagagagggtatttgaagatgtggagacaaaaagataagagttttgaaatagtctcctctccccttcccccaccagggtgtgcccttacccctttcccccagccctgtgctgatcctccctgagatagggcctcaggaccagggaacctacaggtgtgtggccacccatcccagccacgggccccaggaaagccgtgctgtcagcatcagcatcatcggtgagacctctctccaagccctacagaccctggggctagggtgcaggatagcacaggctctaatttcctgccccattctggccttacccccaagagccagcccacctctccctccgtgcacccacacccaaacctcccctgccccactcaaattctgccaagagagcagccaagcctctcccttcttccctctgaactaaaaaaagggaaagacggctgggcgcagtggctcacgcctgtaatcccaacactttgggaggctgaggccggtggatcacctgaggttgggagttcaagaccagtctgaccaacatggaggaaccccatttctactaaaaatacaaaattagccagttgtggtggcacgtgcctgtaatcccagctacttgggaggctgagacaggaaaatcacttgaacccgggaggcgtaagttgcggtgagccaagatcctgccattgcatgccagcctgggcaacaagagcgaaactccatctcaaaaaaaaaaaaaaaagaaagggaaagactccactggagctcccactaaataaccctctctcaacccgaagtcttcctttctgactggatccaactttgtcttccagaaccaggcgaggaggggccaactgcaggtgaggggttcgagaaagtcagggaagcagaagatagcccccaacacatgtgactgcggggatggtcaacaagaaaggaatggtggccgggcgcggtggctcaagcctgtaatcccagcactttgggaggccgagatgggcggatcacgaggtcaggagatcgagaccatcctggctaacacagtaaaaccccatctctaccaaaaaaaaatacaaaaaactagccgggcgacgtggcgggcgcctgtagtcccagctactcgggaggctgaggcaggagaatggtgtaaacccgggaggcggagcttgcagtgagctgagatccggccactgcactccagcctgggcaacagagccagactccatctcaaaaaaaaaaaaaaagaaagaaaggaatggtgagtggtgggggctgtgctctcaattttccctgtctccctacaggctctgtgggaggatcagggccaggaactctagccctggccctggggatcctgggaggcctggggacagccgctctgctcattggggtcatcttgtggcaaaggcggcgacgccaacgagaggagaggtgagtggagaaagccagactcctcagacctagggcttccaggcagcaagtgaagagggatggggggtggaacgacaacgtgcagcattctccacaatcttcctcctcaggaaggcctcagaaaaccaggaggaagaggaggagcgtgcagagctgaatcagtcggaggaacctgaggcaggcgagagtggtactggagggccttgaggggcccacagagagatcccatccatcagctcccttttcttcttcccttgaactgttctggccccagaccaactctctcctgtataaccccaccttgccaaactttcttctacaaccagagccccccacaatgatgagtaaacacctgacacatcttgctcttgtgtgtctgtgtatatgtgtgtgagacacaacctcacccctacacccttgagggccctgaaggaaagggactcacccccacactgcaccaaacatctactcaagttggggagaagatgcttctgtcaagagagggagggaggaaggtggggggcaaacttgggaagagatcccatcaatacatttcaccttttttattgaatttgtattaaaggaggtagtaagggggaggaagcacttaagagtcagaacccatattagactctggggagtgaaaaattaaattaaatcaataagatgggagtgggggaagagtcagagggagctttgcccccctttcaagatcaaatcaagaaatcagggaaagcaaagacttagaagagaagaaagacattctctcaatccatcctccttccccagggcagagaattataatgttactgagtgagcttctgagcagaaggctctcccatctatgcacagacttcactcctcctccccaagctttcctggagaatgtccagggctggccttagccaacagaaatagagaggtcaagggggtccatgagtaaggaagggtcagcagggacccccaatactgattctcctctggctggaggtgggcaggaaggagacatagctcaaatactgagcagccaaaaaaagaagaagatggcgagaaacaggaagagggaatcctgccagctggaggccgggtgaccctgtcccagatccacacctgtgggagagaggaaagctgtggaagcatatgcttctaggctgggaggg

SEQ ID NO: 16 - protein, 119 aa, Rat
  MISC_FEATURE: XT-M4 VH
  MISC_FEATURE: Variable Heavy Region
EVQLVESGGGLVQPGRSLKLSCVVSGFTFNNYWMTWIRQTPGKGLEWVASINNSGDNTYYPDSVKDRFTISRDNAKSTLYLQMNSLRSEDTATYYCTRGGDITTGFDYWGQGVMVTVSS

SEQ ID NO: 17 - protein, 107 aa, Rat
  MISC_FEATURE: XT-M4 VL
  MISC_FEATURE: Variable Light Region
DIQMTQSPSSMSVSLGDTITITCRASQDVGIYVNWFQQKPGKSPRRMIYRATNLADGVPSRFSGSRSGSIYSLTISSLESEDVADYHCLEFDEHPLTFGSGTKVEIK

SEQ ID NO: 18 - protein, 119 aa, Murine
  MISC_FEATURE: XT-H1 VH
  MISC_FEATURE: Variable Heavy Region
QVQLKESGPGLVAPSQSLSITCTISGFSITSYGVHWVRQPPGKGLEWLVVIWSDGRTTYNSTLKSRLSISKDNSKSQVFLKMNSLQTDDTAMYYCVRHGGYFPYAMDYWGQGTSVTVSS

SEQ ID NO: 19 - protein, 106 aa, Murine
  MISC_FEATURE: XT-H1 VL
  MISC_FEATURE: Variable Light Region
DIQMTQSPSSLSTSLGGKVTITCKASQDINKFIAWYQHTPGKGPRLFIYYTSTLQPGIPSRFSGNGSGRDYSFSISNLEPEDIATYYCLQYDNMYTFGGGTKLEIK

SEQ ID NO: 20 - protein, 119 aa, Murine
  MISC_FEATURE: XT-H5 VH
  MISC_FEATURE: Variable Heavy Region
EVLLQQSGPELVKPGASVKISCKASGYTFTDYNMDWVKQSHGKSLEWIGDINPDNGGTIYKQKFKGKATLTVDKSSSTAYMELRSLTSEDTAVYYCTTHDHYYYALDYWGQGTSVTVSS

SEQ ID NO: 21 - protein, 117 aa, Murine
  MISC_FEATURE: XT-H2_VH
  MISC_FEATURE: Variable Heavy Region
QVQLQQSGAELAKPGASVKMSCKASGYTFTTYWMHWVKQRPGQGLYWIGYINPSTGYTEYNQKFKDKATLTADKSSSTAYMQLSSLTSEDSAVYYCARWAGYTIDYWGQGTSVTVSS

SEQ ID NO: 22 - protein, 112 aa, Murine
  MISC_FEATURE: XT-H2-VL
  MISC_FEATURE: Variable Light Region
DVVLTQTPLSLPVNIGDQASISCKSTKSLLNSDGFTYLDWYLQKPGQSPQLLIYLVSDRFSGVPDRFSGSGSGTDFTLTISRVEAEDLGVYYCFQSNSLPLTFGSGTKLEIK

SEQ ID NO: 23 - protein, 111 aa, Murine
  MISC_FEATURE: XT-H5 VL
  MISC_FEATURE: Variable Light Region
DIVLTQSPASLAVSLGQRATISCRASESVDNYGISFMNWFQQKPGQPPRLLIYTVSNQGSGVPARFSGSGSGTDFSLNIHPMEEDDTAMYFCQQSKEVPWTFGGGTKLEIK

SEQ ID NO: 24 - protein, 116 aa, Murine
  MISC_FEATURE: XT-H3 VH
  MISC_FEATURE: Variable Heavy Region
QVQLQQPGSELVRPGASVKLSCKTSGYTFTNYWMHWVKQRHGQGLEWIGNLYPGSGRTNYDEKFKSKVTLTEDTSSSTAYMHLSNLTSEDSAVYYCTSLRRGFVYWGQGTLVTVSA

SEQ ID NO: 25 - protein, 107 aa, Murine
  MISC_FEATURE: XT-H3 VL
  MISC_FEATURE: Variable Light Region
NIVMTQSPEYMSMSFGERVTLTCKASENVGSYVSWYQQKSEQSPKLLIYGASNRYTGVPDRFTGSGSATDFTLTITSVQAEDLADYHCGQTYTYPYTFGGGTKLEIK

SEQ ID NO: 26 - protein, 116 aa, Murine
  MISC_FEATURE: XT-H7 VH
  MISC_FEATURE: Variable Heavy Region
QVQLKESGPGLVAPSQSLSITCTVSGFSLTNYGVHWVRQPPGKGLEWLGVMWAGGSTTYNSALMSRLSISKDNSKSQVFLKMNSLQTDDTAMYYCARYGNYAMDYWGQGTSVTVSS

SEQ ID NO: 27 - protein, 106 aa, Murine
  MISC_FEATURE: XT-H7 VL
  MISC_FEATURE: Variable Light Region
DIQMTQSPSSLSASLGGKVTITCKASQDIYKYIAWYQHKPGKGPRLLIHYTSTLQPGIPSRFSGSGSGRDYSFSISNLEPEDIATYYCLRYDNLYTFGGGTKLEIK

SEQ ID NO: 28 - protein, 117 aa, Artificial
  Humanized XT-H2_hVH_V2.0
  MISC_FEATURE: Amino acid sequences of humanized heavy chain variable regions
QVQLVQSGAEVKKPGASVKVSCKASGYTFTTYWMHWVRQRPGQGLYWIGYINPSTGYTEYNQKFKDRVTMTADTSISTAYMELSRLRSDDTAVYYCARWAGYTIDYWGQGTLVTVSS

SEQ ID NO: 29 - protein, 117 aa, Artificial
  Humanized XT-H2_hVH_V2.7
  MISC_FEATURE: Amino acid sequences of humanized heavy chain variable regions
QVQLVQSGAEVKKPGASVKVSCKASGYTFTTYWMHWVRQAPGQGLEWMGYINPSTGYTEYNQKFKDRVTMTRDKSISTAYMELSRLRSDDTAVYYCARWAGYTIDYWGQGTLVTVSS

SEQ ID NO: 30 - protein, 117 aa, Artificial
  XT-H2_hVH_V4.0
  MISC_FEATURE: Amino acid sequences of humanized heavy chain variable regions
EVQLVESGGGLVQPGGSLRLSCAASGYTFTTYWMHWVRQAPGKGLEWVAYINPSTGYTEYNQKFKDRFTISRDNAKNSLYLQMNSLRAEDTAVYYCARWAGYTIDYWGQGTLVTVSS

SEQ ID NO: 31 - protein, 120 aa, Artificial
  Humanized XT-H2_hVH_V4.1
  MISC_FEATURE: Amino acid sequences of humanized heavy chain variable regions
EVQLVESGGGLVQPGGSLRLSCAASGYTFTTYWMHWMSWVRQAPGKGLYWVAYINPSTGYTEYNQKFKDRFTISADKAKSSLYLQMNSLRAEDTAVYYCARWAGYTIDYWGQGTLVTVSS

SEQ ID NO: 32 - protein, 112 aa, Artificial
  Humanized XT-H2_hVL_V2.0
  MISC_FEATURE: Amino acid sequence of humanized light chain variable regions
DVVLTQTPLSLSVTPGQPASISCKSTKSLLNSDGFTYLDWYLQKPGQSPQLLIYLVSDRFSGVPDRFSGSGSGTDFTLKISRVEAEDVGVYYCFQSNSLPLTFGGGTKVEIK

SEQ ID NO: 33 - protein, 112 aa, Artificial
  Humanized XT-H2_hVL_V3.0
  MISC_FEATURE: Amino acid sequence of humanized light chain variable regions
DIVMTQSPDSLAVSLGERATINCKSTKSLLNSDGFTYLDWYQQKPGQPPKLLIYLVSDRFSGVPDRFSGSGSGTDFTLTISSLQAEDVAVYYCFQSNSLPLTFGGGTKVEIK

SEQ ID NO: 34 - protein, 112 aa, Artificial
  Humanized XT-H2_hVL_V4.0
  MISC_FEATURE: Amino acid sequence of humanized light chain variable regions
DIQMTQSPSSLSASVGDRVTITCKSTKSLLNSDGFTYLDWYQQKPGKAPKLLIYLVSDRFSGVPSRFSGSGSGTDFTLTISSLQPEDFATYYCFQSNSLPLTFGGGTKVEIK

SEQ ID NO: 35 - protein, 112 aa, Artificial
  Humanized XT-H2_hVL_V4.1
  MISC_FEATURE: Amino acid sequence of humanized light chain variable regions
DVQMTQSPSSLSASVGDRVTITCKSTKSLLNSDGFTYLDWYQQKPGKAPKLLIYLVSDRFSGVPSRFSGSGSGTDFTLTISSLQPEDFATYYCFQSNSLPLTFGGGTKVEIK

SEQ ID NO: 36 - protein, 119 aa, Artificial
  Humanized XT-M4_hVH_V1.0
  MISC_FEATURE: Amino acid sequences of humanized heavy chain variable regions
EVQLVESGGGLVQPGGSLRLSCAASGFTFNNYWMTWVRQAPGKGLEWVASINNSGDNTYYPDSVKDRFTISRDNAKNSLYLQMNSLRSEDTAVYYCARGGDITTGFDYWGQGTLVTVSS

SEQ ID NO: 37 - protein, 119 aa, Artificial
  Humanized XT-M4_hVH_V1.1
  MISC_FEATURE: Amino acid sequences of humanized heavy chain variable regions
EVQLVESGGGLVQPGGSLRLSCAASGFTFNNYWMTWVRQAPGKGLEWVASIDNSGDNTYYPDSVKDRFTISRDNAKNSLYLQMNSLRSEDTAVYYCARGGDITTGFDYWGQGTLVTVSS

SEQ ID NO: 38 - protein, 119 aa, Artificial
  Humanized XT-M4_hVH_V2.0
  MISC_FEATURE: Amino acid sequences of humanized heavy chain variable regions
EVQLVESGGGLVQPGGSLRLSCAASGFTFNNYWMTWVRQAPGKGLEWVASIDNSGDNTYYPDSVKDRFTISRDNAKNSLYLQMNSLRAEDTAVYYCARGGDITTGFDYWGQGTLVTVSS

SEQ ID NO: 39 - protein, 107 aa, Artificial
  Humanized XT-M4_hVL_V2.4 \ (G66R)
  MISC_FEATURE: Amino acid sequences of humanized light chain variable regions
DIQMTQSPSSLSASVGDRVTITCRASQDVGIYVNWYQQKPGKAPKLLIYRATNLADGVPSRFSGSRSGTDFTLTISSLQPEDFATYYCLEFDEHPLTFGGGTKVEIK

SEQ ID NO: 40 - protein, 107 aa, Artificial
  Humanized XT-M4_hVL_V2.5 \ (D70I)
  MISC_FEATURE: Amino acid sequences of humanized light chain variable regions
DIQMTQSPSSLSASVGDRVTITCRASQDVGIYVNWYQQKPGKAPKLLIYRATNLADGVPSRFSGSGSGTIFTLTISSLQPEDFATYYCLEFDEHPLTFGGGTKVEIK

SEQ ID NO: 41 - protein, 107 aa, Artificial
  Humanized XT-M4_hVL_V2.6 \ (T69S)
  MISC_FEATURE: Amino acid sequences of humanized light chain variable regions
DIQMTQSPSSLSASVGDRVTITCRASQDVGIYVNWYQQKPGKAPKLLIYRATNLADGVPSRFSGSGSGSDFTLTISSLQPEDFATYYCLEFDEHPLTFGGGTKVEIK

SEQ ID NO: 42 - protein, 107 aa, Artificial
  Humanized XT-M4_hVL_V2.7 \ (L46R)
  MISC_FEATURE: Amino acid sequences of humanized light chain variable regions
DIQMTQSPSSLSASVGDRVTITCRASQDVGIYVNWYQQKPGKAPKRLIYRATNLADGVPSRFSGSGSGTDFTLTISSLQPEDFATYYCLEFDEHPLTFGGGTKVEIK

SEQ ID NO: 43 - protein, 107 aa, Artificial
  Humanized XT-M4_hVL_V2.8
  MISC_FEATURE: Amino acid sequences of humanized light chain variable regions
DIQMTQSPSSLSASVGDRVTITCRASQDVGIYVNWYQQKPGKAPKLMIYRATNLADGVPSRFSGSGSGTDFTLTISSLQPEDFATYYCLEFDEHPLTFGGGTKVEIK

SEQ ID NO: 44 - protein, 107 aa, Artificial
  Humanized XT-M4_hVL_V2.9 \ (F71Y)
  MISC_FEATURE: Amino acid sequences of humanized light chain variable regions
DIQMTQSPSSLSASVGDRVTITCRASQDVGIYVNWYQQKPGKAPKLLIYRATNLADGVPSRFSGSGSGTDYTLTISSLQPEDFATYYCLEFDEHPLTFGGGTKVEIK

SEQ ID NO: 45 - protein, 107 aa, Artificial
  Humanized XT-M4_hVL_V2.10
  MISC_FEATURE: Amino acid sequences of humanized light chain variable regions
DIQMTQSPSSLSASVGDRVTITCRASQDVGIYVNWFQQKPGKAPKRLIYRATNLADGVPSRFSGSGSGTDFTLTISSLQPEDFATYYCLEFDEHPLTFGGGTKVEIK

SEQ ID NO: 46 - protein, 107 aa, Artificial
  Humanized XT-M4_hVL_V2.11
  MISC_FEATURE: Amino acid sequences of humanized light chain variable regions
DIQMTQSPSSLSASVGDRVTITCRASQDVGIYVNWFQQKPGKAPRRLIYRATNLADGVPSRFSGSRSGTDFTLTISSLQPEDFATYYCLEFDEHPLTFGGGTKVEIK

SEQ ID NO: 47 - protein, 107 aa, Artificial
  Humanized XT-M4_hVL_V2.12
  MISC_FEATURE: Amino acid sequences of humanized light chain variable regions
DIQMTQSPSSLSASVGDRVTITCRASQDVGIYVNWYQQKPGKAPRRLIYRATNLADGVPSRFSGSRSGTDFTLTISSLQPEDFATYYCLEFDEHPLTFGGGTKVEIK

SEQ ID NO: 48 - protein, 107 aa, Artificial
  Humanized XT-M4_hVL_V2.13
  MISC_FEATURE: Amino acid sequences of humanized light chain variable regions
DIQMTQSPSSLSASVGDRVTITCRASQDVGIYVNWYQQKPGKAPKRLIYRATNLADGVPSRFSGSRSGTDFTLTISSLQPEDFATYYCLEFDEHPLTFGGGTKVEIK

SEQ ID NO: 49 - protein, 107 aa, Artificial
  Humanized XT-M4_hVL_V2.14
  MISC_FEATURE: Amino acid sequences of humanized light chain variable regions
DIQMTQSPSSLSASVGDRVTITCRASQDVGIYVNWFQQKPGKAPKRLIYRATNLADGVPSRFSGSRSGTDFTLTISSLQPEDFATYYCLEFDEHPLTFGGGTKVEIK

SEQ ID NO: 50 - protein, 16 aa, Artificial
  Linker
DGGGSGGGGSGGGGSS

SEQ ID NO: 51 - DNA, 783 bp, Artificial
  XT.H2_VL-VH (direct) ScFv sequence
gtggcccaggcggccggcgcgcactccgacatccagatgacccagtccccctcctccctgtccgcctccgtgggcgaccgggtgaccatcacctgcaagtccaccaagtccctgctgaactccgacggcttcacctacctggactggtatcagcagaagcctggcaaggcccctaagctgctgatctacctggtgtccgaccggttctccggcgtgccttcccggttcagcggctccggctccggcaccgacttcaccctgaccatctcctccctccagcctgaggacttcgccacctactactgcttccagtccaactccctgcctctgacctttggcggcggaacaaaggtggaaatcaaagatggcggtggatcgggcggtggtggatctggaggaggtggaagctctgaggtgcagctcgtggagtctggcggcggactggtgcagcctggcggctccctgcggctgtcctgcgccgcctccggctacaccttcaccacctactggatgcactgggtgcggcaggcccctggcaagggcctggagtgggtcgcctacatcaacccttccaccggctataccgagtacaaccagaagttcaaggaccggttcaccatctcccgggacaacgccaagaactccctgtacctccagatgaactccctgcgggccgaggacaccgccgtgtactactgcgccagatgggctggctacaccatcgactactggggccagggcaccctggtgaccgtgtcctcatctgatcaggcctcaggggcc

SEQ ID NO: 52 - DNA, 783 bp, Artificial
  XT.H2_VH-VL (direct) ScFv sequence
gtggcccaggcggccggcgcgcactccgaggtgcagctcgtggagtctggcggcggactggtgcagcctggcggctccctgcggctgtcctgcgccgcctccggctacaccttcaccacctactggatgcactgggtgcggcaggcccctggcaagggcctggagtgggtcgcctacatcaacccttccaccggctataccgagtacaaccagaagttcaaggaccggttcaccatctcccgggacaacgccaagaactccctgtacctccagatgaactccctgcgggccgaggacaccgccgtgtactactgcgccagatgggctggctacaccatcgactactggggccagggcaccctggtgaccgtgtcctcagatggcggtggatcgggcggtggtggatctggaggaggtggaagctctgacatccagatgacccagtccccctcctccctgtccgcctccgtgggcgaccgggtgaccatcacctgcaagtccaccaagtccctgctgaactccgacggcttcacctacctggactggtatcagcagaagcctggcaaggcccctaagctgctgatctacctggtgtccgaccggttctccggcgtgccttcccggttcagcggctccggctccggcaccgacttcaccctgaccatctcctccctccagcctgaggacttcgccacctactactgcttccagtccaactccctgcctctgacctttggcggcggaacaaaggtggaaatcaaatctgatcaggcctcaggggcc

SEQ ID NO: 53 - DNA, 774 bp, Artificial
  XT.M4_VH_VL ScFv sequence
gtggcccaggcggccggcgcgcactccgaggtgcagctggtggagtctggcggcggactggtgcagcctggcggctctctgagactgtcttgtgccgcctccggcttcaccttcaacaactactggatgacctgggtgaggcaggcccctggcaagggcctggagtgggtggcctccatcgacaactccggcgacaacacctactaccccgactccgtgaaggaccggttcaccatctccagggacaacgccaagaactccctgtacctccagatgaactccctgagggccgaggataccgccgtgtactactgtgccagaggcggcgatatcaccaccggcttcgactactggggccagggcaccctggtgaccgtgtcctctgatggcggtggatcgggcggtggtggatctggaggaggtggaagctctgacatccagatgacccagtccccctcttctctgtctgcctctgtgggcgacagagtgaccatcacctgtcgggcctctcaggatgtgggcatctacgtgaactggtttcagcagaagcctggcaaggctcccaggcgcctgatctaccgggccaccaacctggccgatggcgtgccttccagattctccggctctcgctctggcaccgatttcaccctgaccatctcctccctccagcctgaggatttcgccacctactactgcctggagttcgacgagcaccctctgacctttggcggcggaacaaaggtggagatcaagtctgatcaggcctcaggggcc

SEQ ID NO: 54 - DNA, 774 bp, Artificial
  XT.M4_VL_VH  ScFv sequence
gtggcccaggcggccggcgcgcactccgacatccagatgacccagtccccctcttctctgtctgcctctgtgggcgacagagtgaccatcacctgtcgggcctctcaggatgtgggcatctacgtgaactggtttcagcagaagcctggcaaggctcccaggcgcctgatctaccgggccaccaacctggccgatggcgtgccttccagattctccggctctcgctctggcaccgatttcaccctgaccatctcctccctccagcctgaggatttcgccacctactactgcctggagttcgacgagcaccctctgacctttggcggcggaacaaaggtggagatcaaggatggcggtggatcgggcggtggtggatctggaggaggtggaagctctgaggtgcagctggtggagtctggcggcggactggtgcagcctggcggctctctgagactgtcttgtgccgcctccggcttcaccttcaacaactactggatgacctgggtgaggcaggcccctggcaagggcctggagtgggtggcctccatcgacaactccggcgacaacacctactaccccgactccgtgaaggaccggttcaccatctccagggacaacgccaagaactccctgtacctccagatgaactccctgagggccgaggataccgccgtgtactactgtgccagaggcggcgatatcaccaccggcttcgactactggggccagggcaccctggtgaccgtgtcctcttctgatcaggcctcaggggcc

SEQ ID NO: 55 - protein, 41 aa, Artificial
  C terminal end of M4 ScFv - VL/VH format
LRAEDTAVYYCARGGDITTGFDYWGQGTLVTVSSSDQASGA

SEQ ID NO: 56 - DNA, 123 bp, Artificial
  C terminal end of M4 ScFv VL/VH format
ctgagggccgaggataccgccgtgtactactgtgccagaggcggcgatatcaccaccggcttcgactactggggccagggcaccctggtgaccgtgtcctcttctgatcaggcctcaggggcc

SEQ ID NO: 57 - DNA, 86 bp, Artificial
  Spiking oligonucleotide for VH3-CDR3 mutagenesis of M4 VL/VH
  misc_feature (61)..(70): N is A, T, C or G
caggttgtcggcccctgaggcctgatcagaggacacggtcaccagggtgccctggccccannnnnnnnnntctggcacagtagtac

SEQ ID NO: 58 - DNA, 53 bp, Artificial
  Spiking oligonucleotide for VH3-CDR3 mutagenesis of M4 VL/VH
  misc_feature (28)..(37): N is A, T, C or G
caggttgtccagggtgccctggccccannnnnnnnnntctggcacagtagtac

SEQ ID NO: 59 - DNA, 22 bp, Artificial
  Artificial Sequence
gaccctggaaggaagcaggatg

SEQ ID NO: 60 - DNA, 27 bp, Artificial
  Artificial Sequence
ggatctgtctgtgggcccctcaaggcc

SEQ ID NO: 61 - DNA, 41 bp, Artificial
  Artificial Sequence
actagactagtcggaccatggcagcaggggcagcggccgga

SEQ ID NO: 62 - DNA, 48 bp, Artificial
  Artificial Sequence
ataagaatgcggccgctaaactattcagggctctcctgtaccgctctc

SEQ ID NO: 63 - protein, 476 aa, Artificial
  anti-RAGE scFv-Fc fusion protein
  MISC_FEATURE: huXT-M4 V2.11 scFv-Fc with wt human IgG1 Fc VL-linker-VH
DIQMTQSPSSLSASVGDRVTITCRASQDVGIYVNWFQQKPGKAPRRLIYRATNLADGVPSRFSGSRSGTDFTLTISSLQPEDFATYYCLEFDEHPLTFGGGTKVEIKDGGGSGGGGSGGGGSSEVQLVESGGGLVQPGGSLRLSCAASGFTFNNYWMTWVRQAPGKGLEWVASIDNSGDNTYYPDSVKDRFTISRDNAKNSLYLQMNSLRAEDTAVYYCARGGDITTGFDYWGQGTLVTVSSDQEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 64 - DNA, 1428 bp, Artificial
  Encodes anti-RAGE scFv-Fc fusion protein
  misc_feature: huXT-M4 VL V2.11-VH V2.0 wt-Fc sequence
gacatccagatgacccagtccccctcttctctgtctgcctctgtgggcgacagagtgaccatcacctgtcgggcctctcaggatgtgggcatctacgtgaactggtttcagcagaagcctggcaaggctcccaggcgcctgatctaccgggccaccaacctggccgatggcgtgccttccagattctccggctctcgctctggcaccgatttcaccctgaccatctcctccctccagcctgaggatttcgccacctactactgcctggagttcgacgagcaccctctgacctttggcggcggaacaaaggtggagatcaaggatggcggtggatcgggcggtggtggatctggaggaggtgggagctctgaggtgcagctggtggagtctggcggcggactggtgcagcctggcggctctctgagactgtcttgtgccgcctccggcttcaccttcaacaactactggatgacctgggtgaggcaggcccctggcaagggcctggagtgggtggcctccatcgacaactccggcgacaacacctactaccccgactccgtgaaggaccggttcaccatctccagggacaacgccaagaactccctgtacctccagatgaactccctgagggccgaggataccgccgtgtactactgtgccagaggcggcgatatcaccaccggcttcgactactggggccagggcaccctggtgaccgtgtcctctgatcaggagcccaaatcttctgacaaaactcacacatgtccaccgtgcccagcacctgaactcctgggtggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatccaagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

(19) United States
(12) Patent Application Publication
(10) Pub. No.: US 2007/0286858 A1
(43) Pub. Date: Dec. 13, 2007

(54) Methods and Compositions for Antagonism of RAGE

(75) Inventors: Brian Clancy, Ashland, MA (US); Debra Pittman, Windham, NH (US); Xiang-Yang Tan, Reading, MA (US); Lioudmila Tchistiakova, Andover, MA (US); Kodangattil R. Sreekumar, Plainsboro, NJ (US); Janet Elizabeth Paulsen, Londonderry, NH (US); Angela Widom, Acton, MA (US); Nicole Piche-Nicholas, Waltham, MA (US); Ying Sun, Maynard, MA (US)

Correspondence Address: PILLSBURY WINTHROP SHAW PITTMAN, LLP, P.O. BOX 10500, MCLEAN, VA, 22102, US

(73) Assignee: Wyeth, Madison, NJ (US)

(21) Appl. No.: 11/689,480

(22) Filed: Mar. 21, 2007

Related U.S. Application Data
(60) Provisional application No. 60/784,575, filed on Mar. 21, 2006.
(60) Provisional application No. 60/895,303, filed on Mar. 16, 2007.

(51) Int. Cl. A61K 39/395; A61P 19/02; A61P 7/12; C07H 21/04; C07K 16/28

(52) U.S. Cl. 424/133.1; 424/130.1; 530/387.1; 530/387.3; 530/387.9; 536/23.1

(57) ABSTRACT

Antibodies that bind specifically to receptor for advanced glycation end products (RAGE) and RAGE-binding fragments thereof are disclosed. Also disclosed are pharmaceutical compositions comprising such anti-RAGE antibodies and RAGE-binding antibody fragments thereof, and their use for treatment of RAGE related diseases.

### CROSS-REFERENCES TO RELATED APPLICATIONS

Priority is claimed under 35 U.S.C. §119(e) of U.S. Provisional Patent Application No. 60/895,303, filed Mar. 16, 2007, and U.S. Provisional Patent Application No. 60/784,575, filed Mar. 21, 2006, the contents of both of which are incorporated herein by reference in their entirety.

### FIELD OF THE INVENTION

The present invention generally relates to antibodies and fragments thereof that bind specifically to a receptor for advanced glycation endproducts (RAGE), to methods in which such antibodies and fragments thereof are administered to human patients and non-human mammals to treat or prevent RAGE-related diseases and disorders.

### BACKGROUND OF THE INVENTION

The receptor for advanced glycation endproducts (RAGE) is a multi-ligand cell surface member of the immunoglobulin super-family. RAGE consists of an extracellular domain, a single membrane-spanning domain, and a cytosolic tail. The extracellular domain of the receptor consists of one V-type immunoglobulin domain followed by two C-type immunoglobulin domains. RAGE also exists in a soluble form (sRAGE). RAGE is expressed by many cell types, e.g., endothelial and smooth muscle cells, macrophages and lymphocytes, in many different tissues, including lung, heart, kidney, skeletal muscle and brain. Expression is increased in chronic inflammatory states such as rheumatoid arthritis and diabetic nephropathy. Although its physiologic function is unclear, it is involved in the inflammatory response and may have a role in diverse developmental processes, including myoblast differentiation and neural development.

RAGE is an unusual pattern-recognition receptor that binds several different classes of endogenous molecules leading to various cellular responses, including cytokine secretion, increased cellular oxidant stress, neurite outgrowth and cell migration. The ligands of RAGE include advanced glycation end products (AGE's), which form in prolonged hyperglycemic states. However, AGE's may be only incidental, pathogenic ligands. In addition to AGES, known ligands of RAGE include proteins having β-sheet fibrils that are characteristic of amyloid deposits and pro-inflammatory mediators, including Sloo/calgranulins (e.g., S100A12, S100B, S100A8-A9), serum amyloid (SAA) (fibrillar form), beta-Amyloid protein (Aβ), and high mobility group box-1 chromosomal protein 1 (HMGB1, also known as amphoterin). HMGB-1 has been shown to be a late mediator of lethality in two models of murine sepsis, and interaction between RAGE and ligands such as HMGB1 is believed to play an important role in the pathogenesis of sepsis and other inflammatory diseases.

A number of significant human disorders are associated with an increased production of ligands for RAGE or with increased production of RAGE itself. Consistently effective therapeutics are not available for many of these disorders. These disorders include, for example, many chronic inflammatory diseases, including rheumatoid and psoriatic arthritis and intestinal bowel disease, cancers, diabetes and diabetic nephropathy, amyloidoses, cardiovascular diseases and sepsis. It would be beneficial to have safe and effective treatments for such RAGE-related disorders.

Sepsis is a systemic inflammatory response (SIRS) to infection, and remains a profound outcome in even previously normal patients. Sepsis is defined by the presence of at least 2 of the 4 clinical signs: hypo- or hyperthermia, tachycardia, tachypnea, hyperventilation, or abnormal leukogram. Sepsis with one organ dysfunction/failure is defined as severe sepsis, and severe sepsis with intractable hypotension is septic shock. Additional types of sepsis include septicemia and neonatal sepsis. More than 2 million cases of sepsis occur each year in the U.S., Europe, and Japan, with estimated annual costs of $17 billion and mortality rates ranging from 20-50%. In patients surviving sepsis, the intensive care unit (ICU) stay is extended on average by 65% compared to ICU patients not experiencing sepsis.

Despite recent market entries and continually improving hospital care, sepsis remains a significant unmet medical need. Treatment of septic patients is time and resource intensive. Newer agents, including the introduction of XIGRIS®, have a modest effect on outcomes. The syndrome continues to exhibit a 20-50% mortality rate. Safe and well-tolerated therapeutic agents that could reduce the progression from early sepsis to severe sepsis or septic shock, and thereby improve survival, could provide a break-through in sepsis therapy.

### SUMMARY OF THE INVENTION

The present invention provides new immunological reagents, in particular, therapeutic antibody reagents that bind to RAGE, for the prevention and treatment of RAGE-related diseases and disorders, e.g., sepsis, diabetes and diabetes-associated pathologies, cardiovascular diseases and cancer.

Representative antibodies of the invention include antibodies that specifically bind RAGE (i.e., anti-RAGE antibodies), which compete for binding to RAGE with an XT-H1, XT-H2, XT-H3, XT-H5, XT-H7, or XT-M4 antibody, or which bind to an epitope of RAGE bound by an XT-H1, XT-H2, XT-H3, XT-H5, XT-H7, or XT-M4 antibody. Additional representative anti-RAGE antibodies of the invention may comprise one or more complementarity determining regions (CDRs) of a light chain or heavy chain of an antibody selected from the group consisting of XT-H1, XT-H2, XT-H3, XT-H5, XT-H7, and XT-M4. Still further provided are RAGE-binding fragments of the foregoing antibodies. The anti-RAGE antibodies of the invention may block the binding of a RAGE body partner.

For example, an anti-RAGE antibody of the invention may comprise (a) a light chain variable region of XT-H1_VL (SEQ ID NO: 19), XT-H2_VL (SEQ ID NO: 22), XT-H3_VL (SEQ ID NO: 25), XT-H5_VL (SEQ ID NO: 23), XT-H7_VL (SEQ ID NO: 27), or XT-M4_VL (SEQ ID NO: 17); (b) a light chain variable region having an amino acid sequence that is at least 90% identical to an amino acid sequence of SEQ ID NO: 19, SEQ ID NO: 22, SEQ ID NO: 25, SEQ ID NO: 23, SEQ ID NO: 27, or SEQ ID NO: 17; or (c) a RAGE-binding fragment of an antibody according to (a) or (b). As another example, an anti-RAGE antibody of the invention may comprise (a) a heavy chain variable region of IXT-H1_VH (SEQ ID NO: 18), XT-H2_VH (SEQ ID NO: 21), XT-H3_VH (SEQ ID NO: 24), XT-H5_VH (SEQ ID NO: 20), XT-H7_VH (SEQ ID NO: 26), or XT-M4_VH (SEQ ID NO: 16); (b) a heavy chain variable region having an amino acid sequence that is at least 90% identical to an amino acid sequence of SEQ ID NO: 18, SEQ ID NO: 21, SEQ ID NO: 24, SEQ ID NO: 20, SEQ ID NO: 26, or SEQ ID NO: 16; or (c) a RAGE-binding fragment of an antibody according to (a) or (b).

The present invention further provides anti-RAGE antibodies having any one of the above-noted light chain variable regions and any one of the above-noted heavy chain variable regions. For example, an anti-RAGE antibody of the invention may be a chimeric antibody, or a RAGE-binding fragment thereof, having a light chain variable region amino acid sequence that is at least 90% identical to the XT-M4 light chain variable region amino acid sequence (SEQ ID NO: 17), a heavy chain variable region amino acid sequence that is at least 90% identical to the XT-M4 heavy chain variable region amino acid sequence (SEQ ID NO: 16), and constant regions derived from human constant regions, such as an antibody having a light chain variable region having the amino acid sequence of the XT-M4 light chain variable region (SEQ ID NO: 17), a heavy chain variable region having the amino acid sequence of the XT-M4 heavy chain variable region sequence (SEQ ID NO: 16), a human kappa light chain constant region, and a human IgG1 heavy chain constant region.

Additional representative anti-RAGE antibodies of the invention include humanized antibodies, for example, and antibody having a humanized light chain variable region that is at least 90% identical to an amino acid sequence XT-H2_hVL_V2.0 (SEQ ID NO:32), XT-H2_hVL_V3.0 (SEQ ID NO: 33), XT-H2_hVL_V4.0 (SEQ ID NO: 34), XT-H2_hVL_V4.1 (SEQ ID NO: 35), XT-M4_hVL_V2.4 (SEQ ID NO:39), XT-M4_hVL_V2.5 (SEQ ID NO: 40), XT-M4_hVL_V2.6 (SEQ ID NO: 41), XT-M4_hVL_V2.7 (SEQ ID NO: 42), XT-M4_hVL_V2.8 (SEQ ID NO: 43), XT-M4_hVL_V2.9 (SEQ ID NO: 44), XT-M4_hVL_V2.10 (SEQ ID NO: 45), XT-M4_hVL_V2.11 (SEQ ID NO: 46), XT-M4_hVL_V2.12 (SEQ ID NO: 47), XT-M4_hVL_V2.13 (SEQ ID NO: 48), or XT-M4_hVL_V2.14 (SEQ ID NO: 49). As another example, a humanized anti-RAGE antibody may comprise a humanized heavy chain variable region that is at least 90% identical to an amino acid sequence of XT-H2_hVH_V2.0 (SEQ ID NO: 28), XT-H2_hVH_V2.7 (SEQ ID NO: 29), XT-H2_hVH_V4 (SEQ ID NO: 30), XT-H2_hVH_V4.1 (SEQ ID NO: 31), XT-M4_hVH_V1.0 (SEQ ID NO: 36), XT-M4_hVH_V1.1 (SEQ ID NO: 37), or XT-M4_hVH_V2.0 (SEQ ID NO: 38). Humanized antibodies can be semi-human (i.e., wherein only one of the light chain and heavy chain variable regions is humanized), or fully humanized (i.e., wherein both light chain and heavy chain variable regions are humanized). Additional representative humanized anti-RAGE antibodies disclosed herein include a humanized XT-M4 antibody and a humanized XT-H2 antibody.

Still further provided are anti-RAGE antibodies having CDRs with at least 8 of the following characteristics: (a) amino acid sequence Y-X-M (Y32; X33; M34) in VH CDR1, where X is preferentially W or N; (b) amino acid sequence I-N-X-S (I51; N52; X53 and S54) in VH CDR2, where X is P or N; (c) amino acid at position 58 in CDR2 of VH is Threonine; (d) amino acid at position 60 in CDR2 of VH is Tyrosine; (e) amino acid at position 103 in CDR3 of VH is Threonine; (f) one or more Tyrosine residues in CDR3 of VH; (g) positively charged residue (Arg or Lys) at position 24 in CDR1 of VL; (h) hydrophilic residue (Thr or Ser) at position 26 in CDR1 of VL; (i) small residue Ser or Ala at the position 25 in CDR1 of VL; (j) negatively charged residue (Asp or Glu) at position 33 in CDR1 of VL; (k) aromatic residue (Phe or Tyr or Trp) at position 37 in CDR1 of VL; (I) hydrophilic residue (Ser or Thr) at position 57 in CDR2 of VL; (m) P-X-T sequence at the end of CDR3 of VL where X could be hydrophobic residue Leu or Trp; wherein amino acid position is as shown in the light and heavy chain amino acid sequences in SEQ ID NO:22 and SEQ ID NO:16, respectively.

Also provided are isolated nucleic acids encoding any of the disclosed anti-RAGE antibodies or antibody variable regions, and isolated nucleic acids that specifically hybridize to a nucleic acid having a nucleotide sequence that is the complement of a nucleotide sequence encoding any of the disclosed anti-RAGE antibodies or antibody variable regions under stringent hybridization conditions.

Isolated nucleic acids of the invention further include (a) nucleic acids encoding a RAGE protein of baboon, monkey or rabbit having an amino acid sequence selected from the group consisting of SEQ ID NO: 7, SEQ ID NO: 9, SEQ ID NO: 11, and SEQ ID NO: 13; nucleic acids that specifically hybridize to the complement of (a); and (c) nucleic acids having a nucleotide sequence that is 95% identical to a nucleotide sequence encoding RAGE of baboon, monkey or rabbit selected from the group consisting of SEQ ID NO: 6, SEQ ID NO: 8, SEQ ID NO: 10, and SEQ ID NO: 12, when the query coverage is 100%.

The invention also includes methods for preventing or treating RAGE-related disease or disorder of a subject having such a disease or disorder, that comprises administering to the subject a therapeutically effective amount of an anti-RAGE antibody or a RAGE-binding fragment thereof of the invention.

The invention includes a method for preventing or treating a RAGE-related disease or disorder is selected from the group consisting of sepsis, septic shock, including conditions such as community-acquired pneumonia, which result in sepsis or septic shock, listeriosis, inflammatory diseases, cancers, arthritis, Crohn's disease, chronic acute inflammatory diseases, cardiovascular diseases, erectile dysfunction, diabetes, complications of diabetes, vasculitis, nephropathies, retinopathies, and neuropathies. Such a method of the invention can comprise administering a composition comprising an anti-RAGE antibody or RAGE-binding fragment thereof of the invention in combination with one or more agents useful in the treatment of the RAGE-related disease or disorder that is to be treated. Such agents of the invention include antibiotics, anti-inflammatory agents, antioxidants, β-blockers, antiplatelet agents, ACE inhibitors, lipid-lowering agents, anti-angiogenic agents, and chemotherapeutics.

The invention provides a method for treating sepsis, septic shock, or listeriosis (e.g., systemic listeriosis) in a human subject comprising administering to the subject a therapeutically effective amount of a chimeric anti-RAGE antibody, or a RAGE-binding fragment thereof that comprises a light chain variable region having the amino acid sequence of the XT-M4 light chain variable region (SEQ ID NO: 17), a heavy chain variable region having the amino acid sequence of the XT-M4 heavy chain variable region sequence (SEQ ID NO: 16), a human kappa light chain constant region, and a human IgG1 heavy chain constant region.

### BRIEF DESCRIPTION OF THE DRAWINGS

FIGS. 1A-1C show aligned amino acid sequences of RAGE of mouse, rat, rabbit (2 isoforms), baboon, cynomolgus monkey, and human (SEQ ID NOs: 3, 14, 11, 13, 7, 9, 1).

FIG. 2 is a graph of data from direct binding ELISA that demonstrate binding of XT-H2 to hRAGE with EC50 of 90 pM and binding of XT-M4 to hRAGE-Fc with EC50 of 300 pM.

FIG. 3 is a graph of data from direct binding ELISA analysis that demonstrate binding of antibodies XT-M4 and XT-H2 to the hRAGE V-domain-Fc of with EC50 of 100 pM.

FIG. 4 is graph of data from ligand competition ELISA binding assays showing the ability of XT-H2 and XT-M4 to block the binding of HMG1 to hRAGE-Fc.

FIG. 5 is a graph of data from antibody competition ELISA binding assays showing that XT-H2 and XT-M4 share a similar epitope and bind to overlapping sites on human RAGE.

FIG. 6 shows aligned amino acid sequences of the heavy chain variable regions of murine anti-RAGE antibodies XT-H1, XT-H2, XT-H3, XT-H5 and XT-H7, and of rat anti-RAGE antibody XT-M4 (SEQ ID NOs: 18, 21, 24, 20, 26, 16).

FIG. 7 shows aligned amino acid sequences of the light chain variable regions of murine anti-RAGE antibodies XT-H1, XT-H2, XT-H3, XT-H5 and XT-H7, and of rat anti-RAGE antibody XT-M4 (SEQ ID NOs: 19, 22, 25, 23, 27, 17).

FIG. 8 shows the nucleotide sequence of cDNA encoding baboon RAGE (SEQ ID NO: 6).

FIG. 9 shows the nucleotide sequence of cDNA encoding cynomolgus monkey RAGE (SEQ ID NO: 8).

FIG. 10 shows the nucleotide sequence of cDNA encoding rabbit RAGE isoform 1 (SEQ ID NO: 10).

FIG. 11 shows the nucleotide sequence of cDNA encoding rabbit RAGE isoform 2 (SEQ ID NO: 12).

FIGS. 12A-12E show the nucleotide sequence of cloned baboon genomic DNA encoding baboon RAGE (clone 18.2) (SEQ ID NO: 15).

FIG. 13 presents four graphs showing the abilities of chimeric XT-M4 antibody and rat antibody XT-M4 to block the binding of RAGE ligands HMGB1, amyloid β 1-42 peptide, S100-A, and S100-B to hRAGE-Fc, as determined by competition ELISA binding assay.

FIG. 14 presents graphs showing the ability of chimeric XT-M4 to compete for binding to hRAGE-Fc with antibodies XT-M4 and XT-H2, as determined by antibody competition ELISA binding assay.

FIG. 15 depicts IHC-staining of lung tissues of cynomologus monkey, rabbit, and baboon, showing that the XT-M4 binds to endogenous cell-surface RAGE in these tissues. Control samples are CHO cells expressing hRAGE contacted by XT-M4, NGBCHO cells that do not express RAGE, and CHO cells expressing hRAGE contacted by a control IgG antibody.

FIG. 16 shows that the rat antibody XT-M4 binds to RAGE in normal human lung and lung of a human with chronic obstructive pulmonary disease (COPD).

FIG. 17 shows amino acid sequences of humanized murine XT-H2 HV region.

FIG. 18 shows amino acid sequences of humanized murine XT-H2 HL region.

FIG. 19 shows amino acid sequences of humanized rat XT-M4 HV region.

FIGS. 20A-20B show amino acid sequences of humanized rat XT-H2 HL region.

FIG. 21 depicts expression vectors used to produce humanized light and heavy chain polypeptides.

FIG. 22 shows ED50 values for the binding of humanized XT-H2 antibodies to human RAGE-Fc as determined by competition ELISA.

FIG. 23 shows kinetic rate constants (kₐ and k_{d}) and association and dissociation constants (Kₐ and K_{d}) for binding of XT-M4 and humanized antibodies XT-M4-V10, XT-M4-V11, and XT-M4-V14 to hRAGE-SA, as determined by BIACORE™ binding assay.

FIG. 24 shows kinetic rate constants (kₐ and k_{d}) and association and dissociation constants (Kₐ and K_{d}) for binding of XT-M4 and humanized antibodies XT-M4-V10, XT-M4-V11, and XT-M4-V14 to mRAGE-SA, as determined by BIACORE™ binding assay.

FIG. 25 shows the nucleotide sequence of a murine XT-H2 VL-VH ScFv construct (SEQ ID NO:51).

FIG. 26 shows the nucleotide sequence of a murine XT-H2 VH-VL ScFv construct (SEQ ID NO: 52).

FIG. 27 shows the nucleotide sequence of a rat XT-M4 VL-VH ScFv construct (SEQ ID NO: 54).

FIG. 28 shows the nucleotide sequence of a rat XT-M4 VH-VL ScFv construct (SEQ ID NO: 53).

FIG. 29 is a graph of ELISA data showing binding to human RAGE-Fc by ScFv constructs of the XT-H2 and XT-M4 anti-RAGE antibodies in either the VL/VH or VHNL configuration.

FIG. 30 is a graph of ELISA data showing binding to human RAGE-Fc and BSA by ScFv constructs of the XT-H2 and XT-M4 anti-RAGE antibodies in the VLNH or VHNL configuration expressed as soluble protein in Escherichia coli. ActRIIb is a non-binding protein expressed from the same vector as a negative control.

FIG. 31 schematically represents the use of PCR to introduce spiked mutations into a CDR of XT-M4.

FIG. 32 shows the nucleotide sequence of the C terminal end of the XT-M4 VL-VH ScFv construct (SEQ ID NO: 56). VH-CDR3 is underlined. Also shown are two spiking oligonucleotides (SEQ ID NOs: 57-58) with a number at each mutation site that identifies the spiking ratio used for mutation at that site. The nucleotide compositions of the spiking ratios corresponding to the numbers are also identified.

FIG. 33 schematically represents the ribosome display vector pWRIL-3. “T7” denotes T7 promotor, “RBS” is the ribosome binding site, “spacer polypeptide” is a spacer polypeptide connecting the folded protein to the ribosome, “Flag-tag” is Flag epitope tag for protein detection.

FIG. 34 schematically represents the phage display vector pWRIL-1.

FIG. 35 schematically represents the combinatorial assembly of VL and VH spiked libraries using the Fab display vector pWRIL-6.

FIG. 36 is a graph of antibody competition ELISA data show increased affinity of the XT-M4 antibody for hRAGE following mutation that removes the glycosylation site at position 52.

FIG. 37 is a survival plot showing a survival advantage following CLP for homozygous and heterozygous RAGE knockout mice and for mice given anti-RAGE antibody compared to wild-type control animals.

FIG. 38 is a graph showing tissue colony counts for enteric bacteria following CLP.

FIG. 39 is a survival plot showing the effects of two different doses of anti-RAGE antibody on the survival of mice following CLP.

FIG. 40 is a survival plot showing the effects of delaying a single dose of anti-RAGE antibody for up to 36 hours following CLP.

FIG. 41 shows levels of L. monocytogenes isolated from liver and spleen of infected homozygous and heterozygous RAGE knockout mice and infected mice given anti-RAGE mAb compared to wild-type control animals.

FIG. 42 is a graph showing serum levels of interferon γ of infected homozygous and heterozygous RAGE knockout mice and infected mice given anti-RAGE antibody compared to wild-type control animals.

FIG. 43 is a survival plot showing a survival advantage following CLP for homozygous and heterozygous RAGE knockout mice compared to wild-type control animals.

FIG. 44 is a survival plot showing a survival advantage following CLP for mice given a single injection of anti-RAGE antibody compared to wild-type control animals.

FIG. 45 is a survival plot showing the effects of delaying a single dose of anti-RAGE antibody for 6 or 12 hours following CLP.

FIG. 46 is a graph showing that mice given anti-RAGE antibody have improved pathology scores compared to control animals.

FIG. 47 is a survival plot showing survival following CLP of mice given anti-RAGE antibody in combination with an antibiotic.

FIG. 48 is a survival plot showing survival following CLP of mice given antibiotic alone.

FIG. 50 is a graph showing L. monocytogenes in liver and spleen of infected homozygous and heterozygous RAGE knockout mice and mice given anti-RAGE antibody.

FIG. 51 is a graph showing serum concentration of chimeric XT-M4 following a single iv administration to mice.

FIG. 52 shows that the chimeric XT-M4 antibody is protective in a CLP model.

FIG. 53 shows that the chimeric XT-M4 antibody is protective in a CLP model up to 24 hours post surgery.

### DETAILED DESCRIPTION OF THE INVENTION

Anti-RAGE Antibodies

The present invention provides antibodies that bind specifically to RAGE, including soluble RAGE and endogenous secretory RAGE, as described herein. Representative anti-RAGE antibodies may comprise at least one of the antibody variable region amino acid sequences shown in SEQ ID NOs: 16-49.

The anti-RAGE antibodies of the invention include antibodies that bind specifically to RAGE and have an amino acid sequence that is identical or substantially identical to any one of SEQ ID NOs: 16-49. An amino acid sequence of an anti-RAGE antibody that is substantially identical is one that has at least 85%, 85%, 87%, 88%, 89%, 90%, 91%, 92%, 93%, 94%, 95%, 96%, 97%, 98%, 99%, 99.5%, or 99.9% identity to any one of SEQ ID NOs: 16-49.

Included in the anti-RAGE antibodies of the invention is an antibody that binds specifically to RAGE, and (a) comprises a light chain variable region selected from the group consisting of SEQ ID NOs: 19, 22, 25, 23, 27 and 17, or (b) comprises a light chain variable region having an amino acid sequence that is at least 90% identical to any one of SEQ ID NOs: 19, 22, 25, 23, 27 and 17, or is a RAGE-binding fragment of an antibody according to (a) or (b).

Also Included in the anti-RAGE antibodies of the invention is an antibody that binds specifically to RAGE, and (a) comprises a heavy chain variable region selected from the group consisting of SEQ ID NOs: 18, 21, 24, 20, 26, and 16, or (b) comprises a heavy chain variable region having an amino acid sequence that is at least 90% identical to any one of SEQ ID NOs: 18, 21, 24, 20, 26, and 16, or is a RAGE-binding fragment of an antibody according to (a) or (b).

Included in the invention is an anti-RAGE antibody that binds specifically to RAGE and:
(a) competes for binding to RAGE with an antibody selected from the group consisting of XT-H1, XT-H2, XT-H3, XT-H5, XT-H7, and XT-M4;
(b) binds to an epitope of RAGE that is bound by an antibody selected from the group consisting of XT-H1, XT-H2, XT-H3, XT-H5, XT-H7, and XT-M4;
(c) comprises one or more complementarity determining regions (CDRs) of a light chain or heavy chain of an antibody selected from the group consisting of XT-H1, XT-H2, XT-H3, XT-H5, XT-H7, and XT-M4; or
(d) is a RAGE-binding fragment of an antibody according to (a), (b) or (c).

The invention includes anti-RAGE antibodies that bind specifically to RAGE-expressing cells in vitro and in vivo, and antibodies that bind to human RAGE with a dissociation constant (Kd) in the range of from at least about 1×10⁻⁷ M to about 1×10⁻¹⁰ M. Also Included are anti-RAGE antibodies of the invention that bind specifically to the V domain of human RAGE, and anti-RAGE antibodies that block the binding of RAGE to a RAGE binding partner (RAGE-BP).

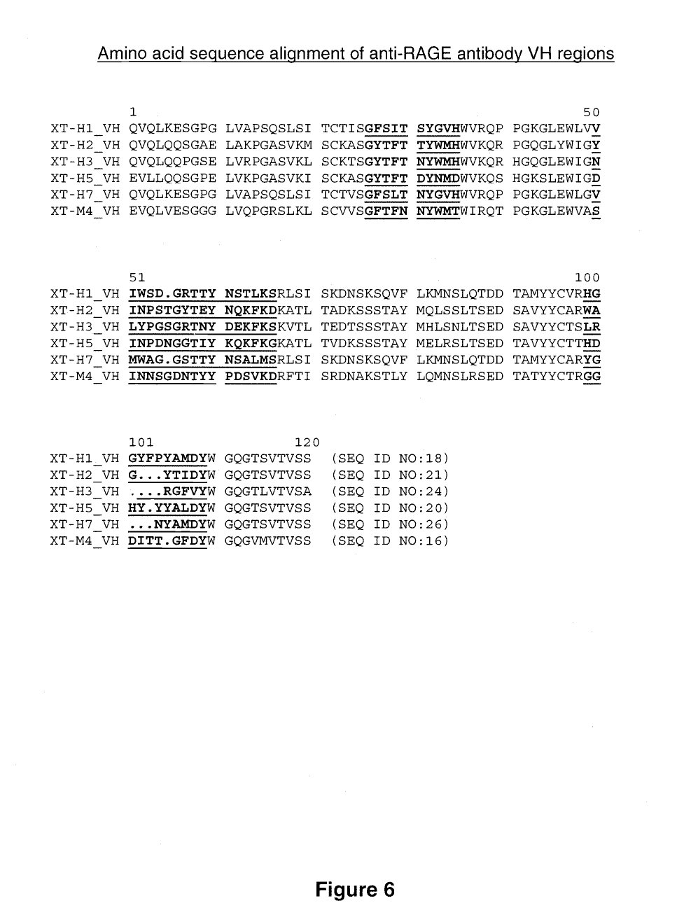

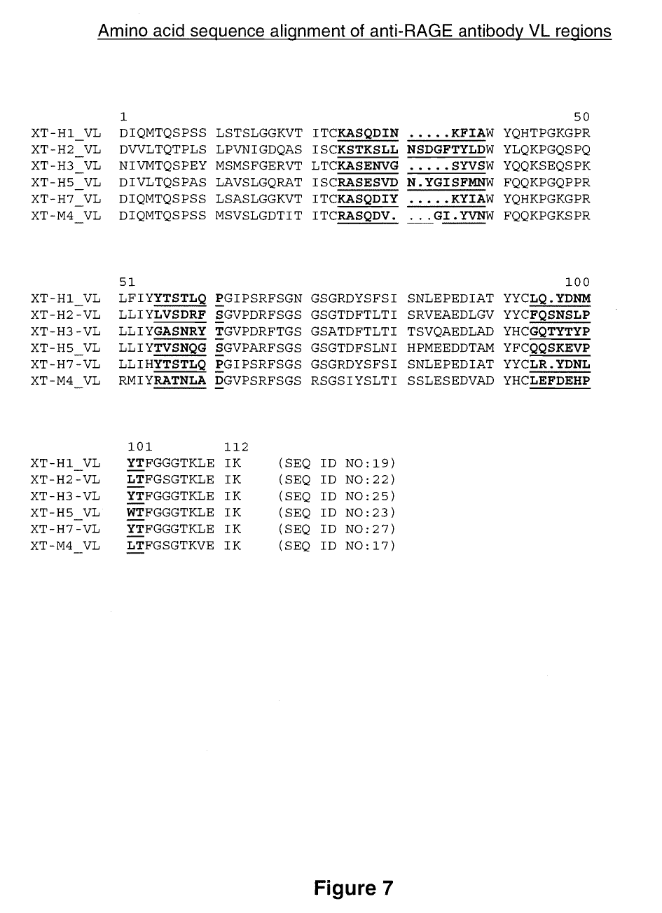

Also included in the invention is an antibody that binds specifically to RAGE and blocks the binding of RAGE to a RAGE-binding partner, e.g. a ligands such as HMGB1, AGE, Aβ, SAA, S100, amphoterin, S100P, S100A (including S100A8 and S100A9), S100A4, CRP, β2-integrin, Mac-1, and p150,95, and has CDRs having 4 or more of the following characteristics (position numbering is with respect to amino acid positions as shown for the VH and VL sequences in FIGS. 6 and 7):
1. Amino acid sequence Y-X-M (Y32; X33; M34) in VH CDR1, where X is preferentially W or N;
2. Amino acid sequence I-N-X-S (I51; N52; X53 and S54) in VH CDR2, where X is P or N;
3. Amino acid at position 58 in CDR2 of VH is Threonine;
4. Amino acid at position 60 in CDR2 of VH is Tyrosine;
5. Amino acid at position 103 in CDR3 of VH is Threonine;
6. One or more Tyrosine residues in CDR3 of VH;
7. Positively charged residue (Arg or Lys) at position 24 in CDR1 of VL;
8. Hydrophilic residue (Thr or Ser) at position 26 in CDR1 of VL;
9. Small residue Ser or Ala at the position 25 in CDR1 of VL;
10. Negatively charged residue (Asp or Glu) at position 33 in CDR1 of VL;
11. Aromatic residue (Phe or Tyr or Trp) at position 37 in CDR1 of VL;
12. Hydrophilic residue (Ser or Thr) at position 57 in CDR2 of VL;
13. P-X-T sequence at the end of CDR3 of VL where X could be hydrophobic residue Leu or Trp.

Anti-RAGE antibodies of the invention include antibodies that bind specifically to the V domain of human RAGE and block the binding of RAGE to its ligands, and have CDRs having 5, 6, 7, 8, 9, 10, 11, 12, or all 13 characteristics.

The anti-RAGE antibodies of the invention include an anti-RAGE antibody as described above, or a RAGE-binding fragment which is selected from the group consisting of a chimeric antibody, a humanized antibody, a single chain antibody, a tetrameric antibody, a tetravalent antibody, a multispecific antibody, a domain-specific antibody, a domain-deleted antibody, a fusion protein, an Fab fragment, an Fab′ fragment, an F(ab′)₂ fragment, an Fv fragment, an ScFv fragment, an Fd fragment, a single domain antibody, a dAb fragment, and an Fc fusion protein (i.e., an antigen binding domain fused to an immunoglobulin constant region). These antibodies can be coupled with a cytotoxic agent, a radiotherapeutic agent, or a detectable label.

For example, an ScFv antibody (SEQ ID NO: 63) comprising the VH and VL domains of the rat XT-M4 antibody has been prepared and shown by cell-based ELISA analysis to have binding affinities for RAGE of baboon, mouse, rabbit, and human comparable to those of the chimeric and wild-type XT-M4 antibodies.

Antibodies of the present invention are further intended to include heteroconjugates, bispecific, single-chain, and chimeric and humanized molecules having affinity for one of the subject polypeptides, conferred by at least one CDR region of the antibody.

Antibodies of the invention that specifically bind to RAGE also include variants of any of the antibodies described herein, which may be readily prepared using known molecular biology and cloning techniques. See, e.g., U.S. Published Patent Application. Nos. 2003/0118592, 2003/0133939, 2004/0058445, 2005/0136049, 2005/0175614, 2005/0180970, 2005/0186216, 2005/0202012, 2005/0202023, 2005/0202028, 2005/0202534, and 2005/0238646, and related patent family members thereof, all of which are hereby incorporated by reference herein in their entireties. For example, a variant antibody of the invention may also comprise a binding domain-immunoglobulin fusion protein that includes a binding domain polypeptide (e.g., scFv) that is fused or otherwise connected to an immunoglobulin hinge or hinge-acting region polypeptide, which in turn is fused or otherwise connected to a region comprising one or more native or engineered constant regions from an immunoglobulin heavy chain, other than CH1, for example, the CH2 and CH3 regions of IgG and IgA, or the CH3 and CH4 regions of IgE (see e.g., U.S. 2005/0136049 by Ledbetter, J. et al., which is incorporated by reference, for a more complete description). The binding domain-immunoglobulin fusion protein can further include a region that includes a native or engineered immunoglobulin heavy chain CH2 constant region polypeptide (or CH3 in the case of a construct derived in whole or in part from IgE) that is fused or otherwise connected to the hinge region polypeptide and a native or engineered immunoglobulin heavy chain CH3 constant region polypeptide (or CH4 in the case of a construct derived in whole or in part from IgE) that is fused or otherwise connected to the CH2 constant region polypeptide (or CH3 in the case of a construct derived in whole or in part from IgE). Typically, such binding domain-immunoglobulin fusion proteins are capable of at least one immunological activity, for example, specific binding to RAGE, inhibition of interaction between RAGE and a RAGE binding partner, induction of antibody dependent cell-mediated cytotoxicity, induction of complement fixation, etc.

Antibodies of the invention may also comprise a label attached thereto and able to be detected, (e.g. the label can be a radioisotope, fluorescent compound, enzyme or enzyme co-factor).

RAGE Polypeptides

The invention also provides isolated RAGE proteins of baboon, cynomologus monkey and rabbit, having the amino acid sequences shown in SEQ ID NOs: 7, 9, 11, or 13, and further includes RAGE proteins having an amino acid sequence that is substantially identical to an amino acid sequences shown in SEQ ID NOs: 7, 9, 11, or 13, in that it is at least 90%, 91%, 92%, 93%, 94%, 95%, 96%, 97%, 98%, 99%, 99.5%, or 99.9% identical in amino acid sequence to any one of SEQ ID NOs: 7, 9, 11, or 13.

Also included in the invention are methods for producing the anti-RAGE antibodies and RAGE-binding fragments thereof of the invention by any means known in the art.

Also Included in the invention is a purified preparation of monoclonal antibody that binds specifically to one or more epitopes of the RAGE amino acid sequence as set forth in any SEQ ID NOs:1, 3, 7, 9, 11, or 13.

Definitions

For convenience, certain terms employed in the specification, examples, and appended claims are collected here. Unless defined otherwise, all technical and scientific terms used herein have the same meaning as commonly understood by one of ordinary skill in the art to which this invention belongs.

The articles “a” and “an” are used herein to refer to one or to more than one (i.e., to at least one) of the grammatical object of the article. By way of example, “an element” means one element or more than one element.

The term “or” is used herein to mean, and is used interchangeably with, the term “and/or,” unless context clearly indicates otherwise.

An “isolated” or “purified” polypeptide or protein, e.g., an “isolated antibody,” is purified to a state beyond that in which it exists in nature. For example, the “isolated” or “purified” polypeptide or protein, e.g., an “isolated antibody,” can be substantially free of cellular material or other contaminating proteins from the cell or tissue source from which the protein is derived, or substantially free from chemical precursors or other chemicals when chemically synthesized. The preparation of antibody protein having less than about 50% of non-antibody protein (also referred to herein as a “contaminating protein”), or of chemical precursors, is considered to be “substantially free.” 40%, 30%, 20%, 10% and more preferably 5% (by dry weight), of non-antibody protein, or of chemical precursors is considered to be substantially free. When the antibody protein or biologically active portion thereof is recombinantly produced, it is also preferably substantially free of culture medium, i.e., culture medium represents less than about 30%, preferably less than about 20%, more preferably less than about 10%, and most preferably less than about 5% of the volume or mass of the protein preparation. Proteins or polypeptides referred to herein as “recombinant” are proteins or polypeptides produced by the expression of recombinant nucleic acids.

The term “antibody” is used interchangeably with the term “immunoglobulin” herein, and includes intact antibodies, fragments of antibodies, e.g., Fab, F(ab′)₂ fragments, and intact antibodies and fragments that have been mutated either in their constant and/or variable region (e.g., mutations to produce chimeric, partially humanized, or fully humanized antibodies, as well as to produce antibodies with a desired trait, e.g., enhanced IL 13 binding and/or reduced FcR binding). The term “fragment” refers to a part or portion of an antibody or antibody chain comprising fewer amino acid residues than an intact or complete antibody or antibody chain. Fragments can be obtained via chemical or enzymatic treatment of an intact or complete antibody or antibody chain. Fragments can also be obtained by recombinant means. Exemplary fragments include Fab, Fab′, F(ab′)₂, Fabc, Fd, dAb, and scFv and/or Fv fragments. The term “antigen-binding fragment” refers to a polypeptide fragment of an immunoglobulin or antibody that binds antigen or competes with intact antibody (i.e., with the intact antibody from which they were derived) for antigen binding (i.e., specific binding). As such these antibodies or fragments thereof are included in the scope of the invention, provided that the antibody or fragment binds specifically to RAGE, and neutralizes or inhibits one or more RAGE-associated activities (e.g., inhibits binding of RAGE binding partners (RAGE-BPs) to RAGE).

The antibody includes a molecular structure comprised of four polypeptide chains, two heavy (H) chains and two light (L) chains inter-connected by disulfide bonds. Each heavy chain is comprised of a heavy chain variable region (abbreviated herein as HCVR or VH) and a heavy chain constant region. The heavy chain constant region is comprised of three domains, CH1, CH2 and CH3. Each light chain is comprised of a light chain variable region (abbreviated herein as LCVR or VL) and a light chain constant region. The light chain constant region is comprised of one domain, CL. The VH and VL regions can be further subdivided into regions of hypervariability, termed complementarity determining regions (CDRs), interspersed with regions that are more conserved, termed framework regions (FR). Each VH and VL is composed of three CDRs and four FRs, arranged from amino-terminus to carboxy-terminus in the following order: FR1, CDR1, FR2, CDR2, FR3, CDR3, FR4.

It is intended that the term “antibody” encompass any Ig class or any Ig subclass (e.g. the IgG₁, IgG₂, IgG₃, and IgG₄ subclassess of IgG) obtained from any source (e.g., humans and non-human primates, and in rodents, lagomorphs, caprines, bovines, equines, ovines, etc.).

The term “Ig class” or “immunoglobulin class”, as used herein, refers to the five classes of immunoglobulin that have been identified in humans and higher mammals, IgG, IgM, IgA, IgD, and IgE. The term “Ig subclass” refers to the two subclasses of IgM (H and L), three subclasses of IgA (IgA1, IgA2, and secretory IgA), and four subclasses of IgG (IgG₁, IgG₂, IgG₃, and IgG₄) that have been identified in humans and higher mammals. The antibodies can exist in monomeric or polymeric form; for example, IgM antibodies exist in pentameric form, and IgA antibodies exist in monomeric, dimeric or multimeric form.

The term “IgG subclass” refers to the four subclasses of immunoglobulin class IgG—IgG₁, IgG₂, IgG₃, and IgG₄ that have been identified in humans and higher mammals by the γ heavy chains of the immunoglobulins, Y₁-Y₄, respectively.

The term “single-chain immunoglobulin” or “single-chain antibody” (used interchangeably herein) refers to a protein having a two-polypeptide chain structure consisting of a heavy and a light chain, said chains being stabilized, for example, by interchain peptide linkers, which has the ability to specifically bind antigen. The term “domain” refers to a globular region of a heavy or light chain polypeptide comprising peptide loops (e.g., comprising 3 to 4 peptide loops) stabilized, for example, by beta.-pleated sheet and/or intrachain disulfide bond. Domains are further referred to herein as “constant” or “variable”, based on the relative lack of sequence variation within the domains of various class members in the case of a “constant” domain, or the significant variation within the domains of various class members in the case of a “variable” domain. Antibody or polypeptide “domains” are often referred to interchangeably in the art as antibody or polypeptide “regions”. The “constant” domains of an antibody light chain are referred to interchangeably as “light chain constant regions”, “light chain constant domains”, “CL” regions or “CL” domains. The “constant” domains of an antibody heavy chain are referred to interchangeably as “heavy chain constant regions”, “heavy chain constant domains”, “CH” regions or “CH” domains). The “variable” domains of an antibody light chain are referred to interchangeably as “light chain variable regions”, “light chain variable domains”, “VL” regions or “VL” domains). The “variable” domains of an antibody heavy chain are referred to interchangeably as “heavy chain constant regions”, “heavy chain constant domains”, “VH” regions or “VH” domains).

The term “region” can also refer to a part or portion of an antibody chain or antibody chain domain (e.g., a part or portion of a heavy or light chain or a part or portion of a constant or variable domain, as defined herein), as well as more discrete parts or portions of said chains or domains. For example, light and heavy chains or light and heavy chain variable domains include “complementarity determining regions” or “CDRs” interspersed among “framework regions” or “FRs”, as defined herein.

The term “conformation” refers to the tertiary structure of a protein or polypeptide (e.g., an antibody, antibody chain, domain or region thereof). For example, the phrase “light (or heavy) chain conformation” refers to the tertiary structure of a light (or heavy) chain variable region, and the phrase “antibody conformation” or “antibody fragment conformation” refers to the tertiary structure of an antibody or fragment thereof.

“Specific binding” of an antibody means that the antibody exhibits appreciable affinity for a particular antigen or epitope and, generally, does not exhibit significant crossreactivity. The term “anti-RAGE antibody” as used herein refers to an antibody that binds specifically to a RAGE. The antibody may exhibit no crossreactivity (e.g., does not crossreact with non-RAGE peptides or with remote epitopes on RAGE. “Appreciable” binding includes binding with an affinity of at least 10⁶, 10⁷, 10⁸, 10⁹ M⁻¹, or 10¹⁰ M⁻¹. Antibodies with affinities greater than 10⁷ M⁻¹ or 10⁸ M⁻¹ typically bind with correspondingly greater specificity. Values intermediate of those set forth herein are also intended to be within the scope of the present invention and antibodies of the invention bind to RAGE with a range of affinities, for example, 10⁶ to 10¹⁰ M⁻¹, or 10⁷ to 10¹⁰ M⁻¹, or 10⁸ to 10¹⁰ M⁻¹. An antibody that “does not exhibit significant crossreactivity” is one that will not appreciably bind to an entity other than its target (e.g., a different epitope or a different molecule). For example, an antibody that specifically binds to RAGE will appreciably bind RAGE but will not significantly react with non-RAGE proteins or peptides. An antibody specific for a particular epitope will, for example, not significantly crossreact with remote epitopes on the same protein or peptide. Specific binding can be determined according to any art-recognized means for determining such binding. Preferably, specific binding is determined according to Scatchard analysis and/or competitive binding assays.

As used herein, the term “affinity” refers to the strength of the binding of a single antigen-combining site with an antigenic determinant. Affinity depends on the closeness of stereochemical fit between antibody combining sites and antigen determinants, on the size of the area of contact between them, on the distribution of charged and hydrophobic groups, etc. Antibody affinity can be measured by equilibrium dialysis or by the kinetic BIACORE™ method. The BIACORE™ method relies on the phenomenon of surface plasmon resonance (SPR), which occurs when surface plasmon waves are excited at a metal/liquid interface. Light is directed at, and reflected from, the side of the surface not in contact with sample, and SPR causes a reduction in the reflected light intensity at a specific combination of angle and wavelength. Bimolecular binding events cause changes in the refractive index at the surface layer, which are detected as changes in the SPR signal.

The dissociation constant, Kd, and the association constant, Ka, are quantitative measures of affinity. At equilibrium, free antigen (Ag) and free antibody (Ab) are in equilibrium with antigen-antibody complex (Ag-Ab), and the rate constants, ka and kd, quantitate the rates of the individual reactions:

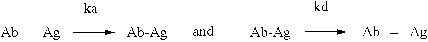

At equilibrium, ka [Ab][Ag]=kd [Ag−Ab]. The dissociation constant, Kd, is given by: Kd=kd/ka=[Ag][Ab]/[Ag−Ab]. Kd has units of concentration, most typically M, mM, μM, nM, pM, etc. When comparing antibody affinities expressed as Kd, having greater affinity for RAGE is indicated by a lower value. The association constant, Ka, is given by: Ka=ka/kd=[Ag−Ab]/[Ag][Ab]. Ka has units of inverse concentration, most typically M⁻¹, mM⁻¹, μM⁻¹, nM⁻¹, pM⁻¹, etc. As used herein, the term “avidity” refers to the strength of the antigen-antibody bond after formation of reversible complexes. Anti-RAGE antibodies may be characterized in terms of the Kd for their binding to a RAGE protein, as binding “with a dissociation constant (Kd) in the range of from about (lower Kd value) to about (upper Kd value).” In this context, the term “about” is intended to mean the indicated Kd value ±20%; i.e., Kd of about 1=Kd in the range of from 0.8 to 1.2.

As used herein, the term “monoclonal antibody” refers to an antibody derived from a clonal population of antibody-producing cells (e.g., B lymphocytes or B cells) which is homogeneous in structure and antigen specificity. The term “polyclonal antibody” refers to a plurality of antibodies originating from different clonal populations of antibody-producing cells which are heterogeneous in their structure and epitope specificity but which recognize a common antigen. Monoclonal and polyclonal antibodies may exist within bodily fluids, as crude preparations, or may be purified, as described herein.

The term “binding portion” of an antibody (or “antibody portion”) includes one or more complete domains, e.g., a pair of complete domains, as well as fragments of an antibody that retain the ability to specifically bind to RAGE. It has been shown that the binding function of an antibody can be performed by fragments of a full-length antibody. Binding fragments are produced by recombinant DNA techniques, or by enzymatic or chemical cleavage of intact immunoglobulins. Binding fragments include Fab, Fab′, F(ab′)₂, Fabc, Fd, dAb, Fv, single chains, single-chain antibodies, e.g., scFv, and single domain antibodies (Muyldermans et al., 2001, 26:230-5), and an isolated complementarity determining region (CDR). Fab fragment is a monovalent fragment consisting of the VL, VH, CL and CH1 domains. F(ab′)₂ fragment is a bivalent fragment comprising two Fab fragments linked by a disulfide bridge at the hinge region. Fd fragment consists of the VH and CH1 domains, and Fv fragment consists of the VL and VH domains of a single arm of an antibody. A dAb fragment consists of a VH domain (Ward et al., (1989) Nature 341:544-546). While the two domains of the Fv fragment, VL and VH, are coded for by separate genes, they can be joined, using recombinant methods, by a synthetic linker that enables them to be made as a single protein chain in which the VL and VH regions pair to form monovalent molecules (known as single chain Fv (scFv) (Bird et al., 1988, Science 242:423-426). Such single chain antibodies are also intended to be encompassed within the term “binding portion” of an antibody. Other forms of single chain antibodies, such as diabodies are also encompassed. Diabodies are bivalent, bispecific antibodies in which VH and VL domains are expressed on a single polypeptide chain, but using a linker that is too short to allow for pairing between the two domains on the same chain, thereby forcing the domains to pair with complementary domains of another chain and creating two antigen binding sites (see e.g., Holliger, et al., 1993, Proc. Natl. Acad. Sci. USA 90:6444-6448). An antibody or binding portion thereof also may be part of a larger immunoadhesion molecules formed by covalent or non-covalent association of the antibody or antibody portion with one or more other proteins or peptides. Examples of such immunoadhesion molecules include use of the streptavidin core region to make a tetrameric scFv molecule (Kipriyanov, S. M., et al. (1995) Human Antibodies and Hybridomas 6:93-101) and use of a cysteine residue, a marker peptide and a C-terminal polyhistidine tag to make bivalent and biotinylated scFv molecules (Kipriyanov, S. M., et al. (1994) Mol. Immunol. 31:1047-1058). Binding fragments such as Fab and F(ab′)₂ fragments, can be prepared from whole antibodies using conventional techniques, such as papain or pepsin digestion, respectively, of whole antibodies. Moreover, antibodies, antibody portions and immunoadhesion molecules can be obtained using standard recombinant DNA techniques, as described herein and as known in the art. Other than “bispecific” or “bifunctional” antibodies, an antibody is understood to have each of its binding sites identical. A “bispecific” or “bifunctional antibody” is an artificial hybrid antibody having two different heavy/light chain pairs and two different binding sites. A bispecific antibody can also include two antigen binding regions with an intervening constant region. Bispecific antibodies can be produced by a variety of methods including fusion of hybridomas or linking of Fab′ fragments. See, e.g., Songsivilai et al., Clin. Exp. Immunol. 79:315-321, 1990.; Kostelny et al., 1992, J. Immunol. 148, 1547-1553.

The term “backmutation” refers to a process in which some or all of the somatically mutated amino acids of a human antibody are replaced with the corresponding germline residues from a homologous germine antibody sequence. The heavy and light chain sequences of the human antibody of the invention are aligned separately with the germline sequences in the VBASE database to identify the sequences with the highest homology. Differences in the human antibody of the invention are returned to the germline sequence by mutating defined nucleotide positions encoding such different amino acid. The role of each amino acid thus identified as candidate for backmutation should be investigated for a direct or indirect role in antigen binding and any amino acid found after mutation to affect any desirable characteristic of the human antibody should not be included in the final human antibody; as an example, activity enhancing amino acids identified by the selective mutagenesis approach will not be subject to backmutation. To minimize the number of amino acids subject to backmutation those amino acid positions found to be different from the closest germline sequence but identical to the corresponding amino acid in a second germline sequence can remain, provided that the second germline sequence is identical and colinear to the sequence of the human antibody of the invention for at least 10, preferably 12 amino acids, on both sides of the amino acid in question. Backmutation may occur at any stage of antibody optimization; preferably, backmutation occurs directly before or after the selective mutagenesis approach. More preferably, backmutation occurs directly before the selective mutagenesis approach.

Intact antibodies, also known as immunoglobulins, are typically tetrameric glycosylated proteins composed of two light (L) chains of approximately 25 kDa each and two heavy (H) chains of approximately 50 kDa each. Two types of light chain, termed lambda and kappa, are found in antibodies. Depending on the amino acid sequence of the constant domain of heavy chains, immunoglobulins can be assigned to five major classes: A, D, E, G, and M, and several of these may be further divided into subclasses (isotypes), e.g., IgG1, IgG2, IgG3, IgG4, IgA1, and IgA2. Each light chain is composed of an N terminal variable (V) domain (VL) and a constant (C) domain (CL). Each heavy chain is composed of an N terminal V domain (VH), three or four C domains (CHs), and a hinge region. The CH domain most proximal to VH is designated as CH1. The VH and VL domains consist of four regions of relatively conserved sequences called framework regions (FR1, FR2, FR3, and FR4), which form a scaffold for three regions of hypervariable sequences (complementarity determining regions, CDRs). The CDRs contain most of the residues responsible for specific interactions of the antibody with the antigen. CDRs are referred to as CDR1, CDR2, and CDR3. Accordingly, CDR constituents on the heavy chain are referred to as H1, H2, and H3, while CDR constituents on the light chain are referred to as L1, L2, and L3. CDR3 is the greatest source of molecular diversity within the antibody-binding site. H3, for example, can be as short as two amino acid residues or greater than 26 amino acids. The subunit structures and three-dimensional configurations of different classes of immunoglobulins are well known in the art. For a review of the antibody structure, see Antibodies: A Laboratory Manual, Cold Spring Harbor Laboratory, eds. Harlow et al., 1988. One of skill in the art will recognize that each subunit structure, e.g., a CH, VH, CL, VL, CDR, FR structure, comprises active fragments, e.g., the portion of the VH, VL, or CDR subunit that binds to the antigen, i.e., the binding fragment, or, e.g., the portion of the CH subunit that binds to and/or activates, e.g., an Fc receptor and/or complement.

Antibody diversity is created by the use of multiple germline genes encoding variable regions and a variety of somatic events. The somatic events include recombination of variable gene segments with diversity (D) and joining (J) gene segments to make a complete VH region, and the recombination of variable and joining gene segments to make a complete VL region. The recombination process itself is imprecise, resulting in the loss or addition of amino acids at the V(D)J junctions. These mechanisms of diversity occur in the developing B-cell prior to antigen exposure. After antigenic stimulation, the expressed antibody genes in B-cells undergo somatic mutation. Based on the estimated number of germline gene segments, the random recombination of these segments, and random VH-VL pairing, up to 1.6×107 different antibodies could be produced (Fundamental Immunology, 3rd ed. (1993), ed. Paul, Raven Press, New York, N.Y.). When other processes that contribute to antibody diversity (such as somatic mutation) are taken into account, it is thought that upwards of 1×1010 different antibodies could be generated (Immunoglobulin Genes, 2nd ed. (1995), eds. Jonio et al., Academic Press, San Diego, Calif.). Because of the many processes involved in generating antibody diversity, it is unlikely that independently derived monoclonal antibodies with the same antigen specificity will have identical amino acid sequences.

The term “dimerizing polypeptide” or “dimerizing domain” includes any polypeptide that forms a diner (or higher order complex, such as a trimer, tetramer, etc.) with another polypeptide. Optionally, the dimerizing polypeptide associates with other, identical dimerizing polypeptides, thereby forming homomultimers. An IgG Fc element is an example of a dimerizing domain that tends to form homomultimers. Optionally, the dimerizing polypeptide associates with other different dimerizing polypeptides, thereby forming heteromultimers. The Jun leucine zipper domain forms a dimer with the Fos leucine zipper domain, and is therefore an example of a dimerizing domain that tends to form heteromultimers. Dimerizing domains may form 25 both hetero- and homomultimers.

The term “human antibody” includes antibodies having variable and constant regions corresponding to human germline immunoglobulin sequences as described by Kabat et al. (See Kabat, et al. (1991) Sequences of proteins of Immunological Interest, Fifth Edition, U.S. Department of Health and Human Services, NIH Publication No. 91-3242). The human antibodies of the invention may include amino acid residues not encoded by human germline immunoglobulin sequences (e.g., mutations introduced by random or site-specific mutagenesis in vitro or by somatic mutation in vivo), for example in the CDRs and in particular CDR3. The mutations preferably are introduced using the “selective mutagenesis approach” described herein. The human antibody can have at least one position replaced with an amino acid residue, e.g., an activity enhancing amino acid residue, which is not encoded by the human germline immunoglobulin sequence. The human antibody can have up to twenty positions replaced with amino acid residues that are not part of the human germline immunoglobulin sequence. Further, up to ten, up to five, up to three or up to two positions are replaced. These replacements may fall within the CDR regions as described in detail below. However, the term “human antibody”, as used herein, is not intended to include antibodies in which CDR sequences derived from the germline of another mammalian species, such as a mouse, have been grafted onto human framework sequences.

The phrase “recombinant human antibody” includes human antibodies that are prepared, expressed, created or isolated by recombinant means, such as antibodies expressed using a recombinant expression vector transfected into a host cell (described further in Section II, below), antibodies isolated from a recombinant, combinatorial human antibody library (described further in Section III, below), antibodies isolated from an animal (e.g., a mouse) that is transgenic for human immunoglobulin genes (see e.g., Taylor, L. D., et al. (1992) Nucl. Acids Res. 20:6287-6295) or antibodies prepared, expressed, created or isolated by any other means that involves splicing of human immunoglobulin gene sequences to other DNA sequences. Such recombinant human antibodies have variable and constant regions derived from human germline immunoglobulin sequences (See Kabat, E. A., et al. (1991) Sequences of Proteins of Immunological Interest, Fifth Edition, U.S. Department of Health and Human Services, NIH Publication No. 91-3242). However, such recombinant human antibodies may be subjected to in vitro mutagenesis (or, when an animal transgenic for human Ig sequences is used, in vivo somatic mutagenesis) and thus the amino acid sequences of the VH and VL regions of the recombinant antibodies are sequences that, while derived from and related to human germline VH and VL sequences, may not naturally exist within the human antibody germline repertoire in vivo. In certain embodiments, however, such recombinant antibodies may be the result of selective mutagenesis approach or backmutation or both.

An “isolated antibody” includes an antibody that is substantially free of other antibodies having different antigenic specificities (e.g., an isolated antibody that specifically binds RAGE is substantially free of antibodies that specifically bind RAGE other than hRAGE). An isolated antibody that specifically binds RAGE may bind RAGE molecules from other species. Moreover, an isolated antibody may be substantially free of other cellular material and/or chemicals.

A “neutralizing antibody” (or an “antibody that neutralized RAGE activity”) includes an antibody whose binding to hRAGE results in modulation of the biological activity of hRAGE. This modulation of the biological activity of hRAGE can be assessed by measuring one or more indicators of hRAGE biological activity, such as inhibition of receptor binding in a human RAGE receptor binding assay (see, e.g., Examples 6 and 7). These indicators of hRAGE biological activity can be assessed by one or more of several standard in vitro or in vivo assays known in the art (see, e.g., Examples 6 and 7).

“Humanized” forms of non-human (e.g., murine) antibodies are chimeric antibodies that contain minimal sequence derived from non-human immunoglobulin. For the most part, humanized antibodies are human immunoglobulins (recipient antibody) in which residues from a hypervariable region of the recipient are replaced by residues from a hypervariable region of a non-human species (donor antibody) such as mouse, rat, rabbit or nonhuman primate having the desired specificity, affinity, and capacity. In some instances, FR residues of the human immunoglobulin are replaced by corresponding non-human residues. Furthermore, humanized antibodies may comprise residues that are not found in the recipient antibody or in the donor antibody. These modifications are made to further refine antibody performance. In general, the humanized antibody will comprise substantially all of at least one, and typically two, variable domains, in which all or substantially all of the hypervariable regions correspond to those of a non-human immunoglobulin and all or substantially all of the FR regions are those of a human immunoglobulin sequence. The humanized antibody optionally also will comprise at least a portion of an immunoglobulin constant region (Fc), typically that of a human immunoglobulin. For further details, see Jones et al., Nature 321:522-525 (1986); Riechmann et al., Nature 332:323-329 (1988); and Presta, Curr. Op. Struct. Biol. 2:593-596 (1992).

The term “activity” includes activities such as the binding specificity/affinity of an antibody for an antigen, for example, an anti-hRAGE antibody that binds to RAGE and/or the neutralizing potency of an antibody, for example, an anti-hRAGE antibody whose binding to hRAGE inhibits the biological activity of RAGE, e.g., inhibition of receptor binding in a human RAGE receptor binding assay.

An “expression construct” is any recombinant nucleic acid that includes an expressible nucleic acid and regulatory elements sufficient to mediate expression of the expressible nucleic acid protein or polypeptide in a suitable host cell.

The terms “fusion protein” and “chimeric protein” are interchangeable and refer to a protein or polypeptide that has an amino acid sequence having portions corresponding to amino acid sequences from two or more proteins. The sequences from two or more proteins may be full or partial (i.e., fragments) of the proteins. Fusion proteins may also have linking regions of amino acids between the portions corresponding to those of the proteins. Such fusion proteins may be prepared by recombinant methods, wherein the corresponding nucleic acids are joined through treatment with nucleases and ligases and incorporated into an expression vector. Preparation of Fusion Proteins is Generally Understood by Those Having Ordinary Skill in the art.

The term “nucleic acid” refers to polynucleotides such as deoxyribonucleic acid (DNA), and, where appropriate, ribonucleic acid (RNA). The term should also be understood to include, as equivalents, analogs of either RNA or DNA made from nucleotide analogs, and, as applicable to the embodiment being described, single (sense or antisense) and double-stranded polynucleotides.

The term “percent identical” or “percent identity” refers to sequence identity between two amino acid sequences or between two nucleotide sequences. Percent identity can be determined by comparing a position in each sequence that may be aligned for purposes of comparison. Expression as a percentage of identity refers to a function of the number of identical amino acids or nucleic acids at positions shared by the compared sequences. Various alignment algorithms and/or programs may be used, including FASTA, BLAST, or ENTREZ. FASTA and BLAST are available as a part of the GCG sequence analysis package (University of Wisconsin, Madison, Wis.), and can be used with, e.g. default settings. ENTREZ is available through the National Center for Biotechnology Information, National Library of Medicine, National Institutes of Health, Bethesda, Md. The percent identity of two sequences may be determined by the GCG program with a gap weight of 1, e.g. each amino acid gap is weighted as if it were a single amino acid or nucleotide mismatch between the two sequences.

Other techniques for alignment are described in Methods in Enzymology, vol. 266: Computer Methods for Macromolecular Sequence Analysis (1996), ed. Doolittle, Academic Press, Inc., a division of Harcourt Brace & Co., San Diego, Calif., USA. Preferably, an alignment program that permits gaps in the sequence is utilized to align the sequences. The Smith-Waterman is one type of algorithm that permits gaps in sequence alignments. See Meth. Mol. Viols. 70: 173-187 (1997). Also, the GAP I program using the Needlenan and Wunsch alignment method can be utilized to align sequences. An alternative search strategy uses MPSRCH software, which runs on a MASPAR computer. MPSRCH uses a Smith-Waterman algorithm to score sequences 5 on a massively parallel computer. This approach improves the ability to pick up distantly related matches, and is especially tolerant of small gaps and nucleotide sequence errors. Nucleic acid-encoded amino acid sequences can be used to search both protein: and DNA databases.

The terms “polypeptide” and “protein” are used interchangeably herein.

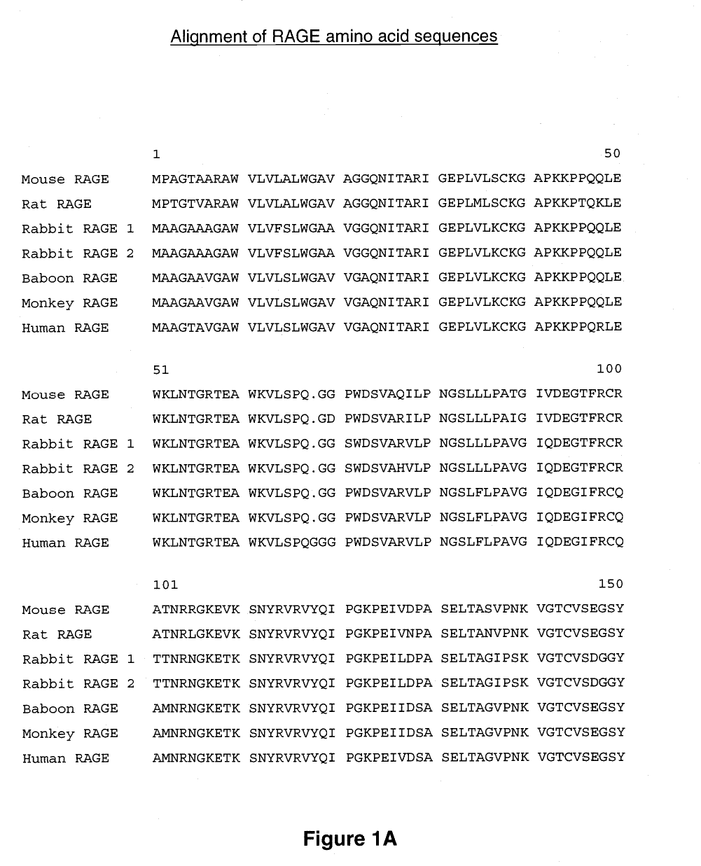

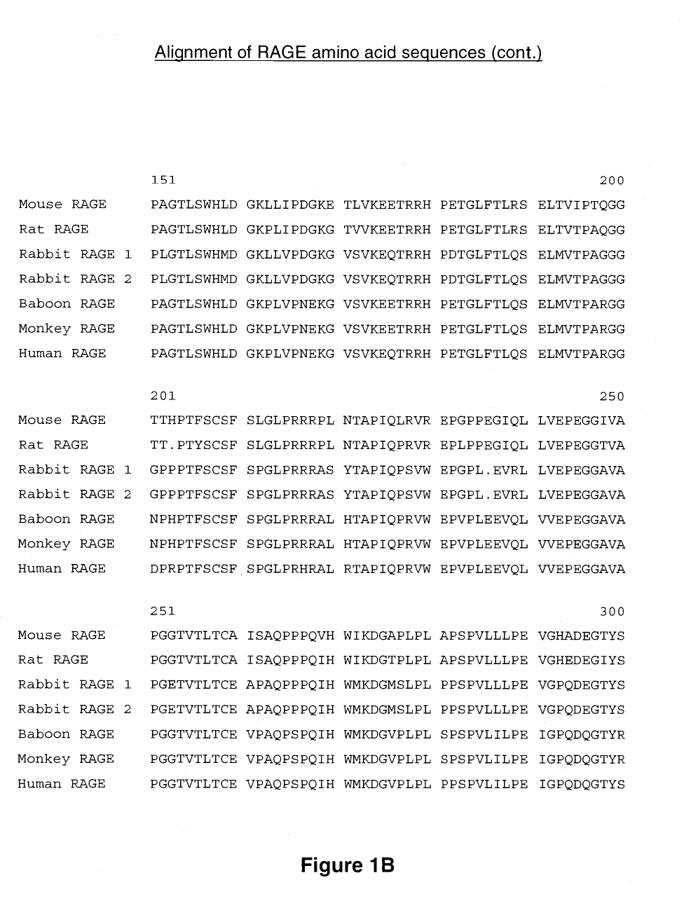

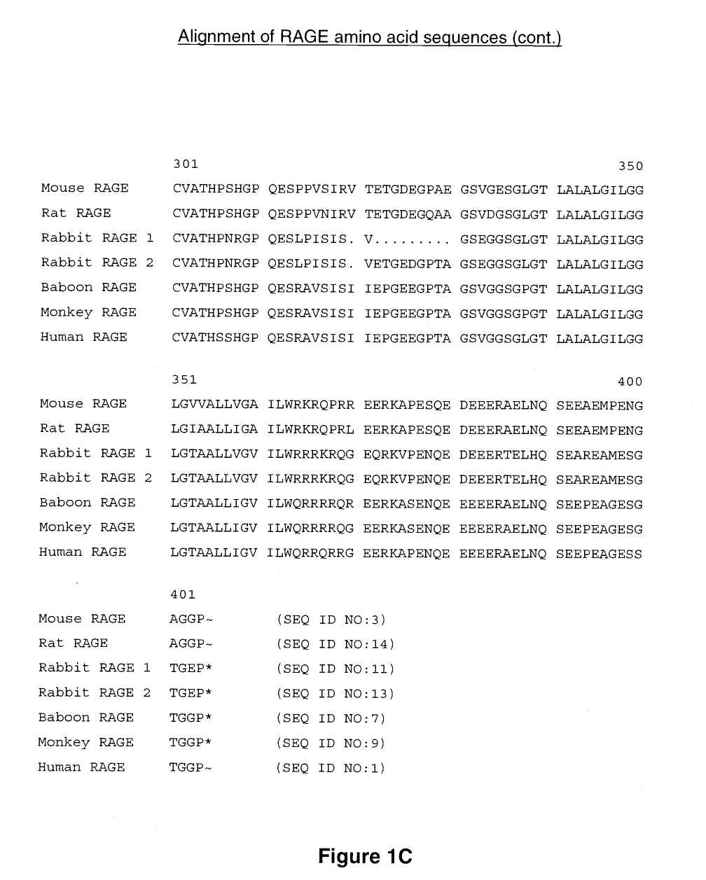

A “RAGE” protein is a “Receptor for Advanded Glycation End Products,” as known in the art. Representative RAGE proteins are set forth in FIGS. 1A-1C. RAGE proteins include soluble RAGE (sRAGE) and endogenous secretory RAGE (esRAGE). Endogenous secretory RAGE is a RAGE splice variant that is released outside of the cells, where it is capable of binding AGE ligands and neutralizing AGE actions. See e.g., Koyama et al., ATVE, 2005; 25:2587-2593. Inverse association has been observed between human plasma esRAGE and several components of metabolic syndrome (BMI, insulin resistance, BP, hypertriglyceridemia and IGT). Plasma esRAGE levels have also been inversely associated with carotid and femoral atherosclerosis (quantitated by ultrasound) in subjects with or without diabetes. Moreover, plasma esRAGE levels are significantly lower in nondiabetic patients with angiographically proved coronary artery disease than age-matched healthy control.

A “Receptor for Advanced Glycation End Products Ligand Binding Element” or “RAGE-LBE” (also referred to herein as “RAGE-Fc” and “RAGE-strep”) includes any extracellular portion of a transmembrane RAGE polypeptide and fragments thereof that retain the ability to bind a RAGE ligand. This term also encompasses polypeptides having at least 85% identity, preferably at least 90% identity or more preferably at least 95% identity with a RAGE polypeptide, for example, the human or murine polypeptide to which a RAGE ligand or RAGE-BP will bind.

A “Receptor for Advanced Glycation End Products Binding Partner” or “RAGE-BP” includes any substance (e.g., polypeptide, small molecule, carbohydrate structure, etc.) that binds in a physiological setting to an extracellular portion of a RAGE protein (a receptor polypeptide such as, e.g., RAGE or RAGE-LBE).

“RAGE-related disorders” or “RAGE-associated disorders” include any disorder in which an affected cell or tissue exhibits an increase or decrease in the expression and/or activity of RAGE or one or more RAGE ligands. RAGE-related disorders also include any disorder that is treatable (i.e., one or more symptom may be eliminated or ameliorated) by a decrease in RAGE function (including, for example, administration of an agent that disrupts RAGE:RAGE-BP interactions).

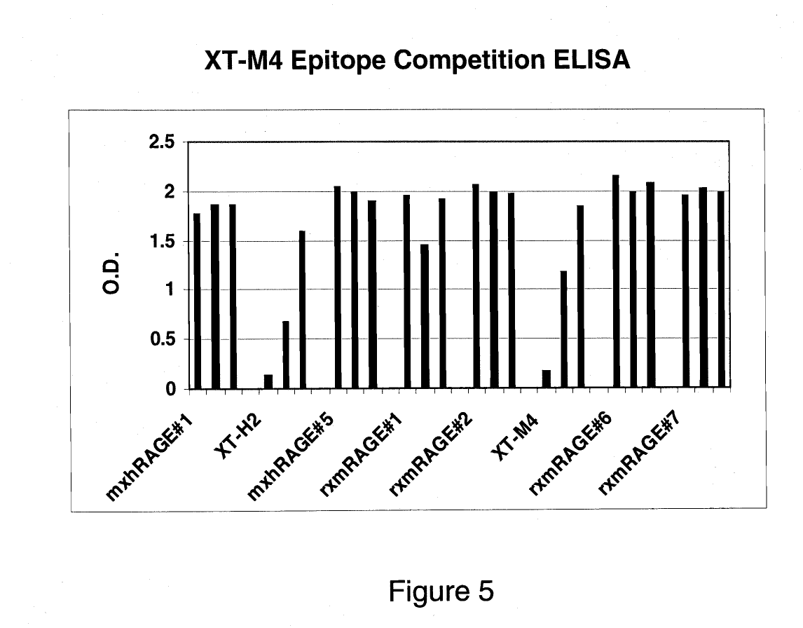

“V-domain of RAGE” refers to the immunoglobulin-like variable domain as shown in FIG. 5 of Neeper, et al, “Cloning and expression of RAGE: a cell surface receptor for advanced glycosylation end products of proteins,” J. Biol. Chem. 267:14998-15004 (1992), the contents of which are hereby incorporated by reference. The V-domain includes amino acids from position 1 to position 120 as shown in SEQ ID NO:1 and SEQ ID NO:3.

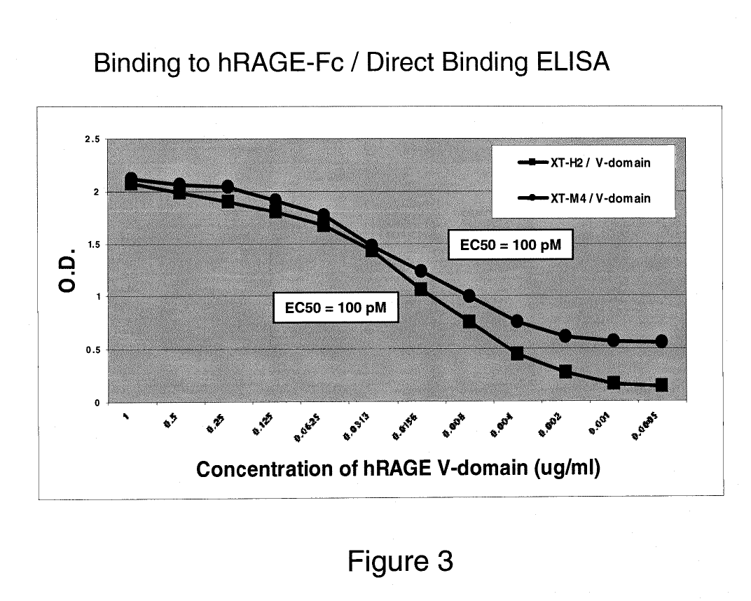

The human cDNA of RAGE is 1406 base pairs and encodes a mature protein of 404 amino acids. See FIG. 3 of Neeper et al. 1992.

The term “recombinant nucleic acid” includes any nucleic acid comprising at least two sequences that are not present together in nature. A recombinant nucleic acid may be generated in vitro, for example by using the methods of molecular biology, or in vivo, for example by insertion of a nucleic acid at a novel chromosomal location by homologous or non-homologous recombination.

The term “treating” with regard to a subject, refers to improving at least one symptom of the subject's disease or disorder. Treating can be curing the disease or condition or improving it.

The term “vector” refers to a nucleic acid molecule capable of transporting another nucleic acid to which it has been linked. One type of vector is an episome, i.e., a nucleic acid capable of extra-chromosomal replication. Another type of vector is an integrative vector that is designed to recombine with the genetic material of a host cell. Vectors may be both autonomously replicating and integrative, and the properties of a vector may differ depending on the cellular context (i.e., a vector may be autonomously replicating in one host cell type and purely integrative in another host cell type). Vectors capable of directing the expression of expressible nucleic acids to which they are operatively linked are referred to herein as “expression vectors.”

“Specifically immunoreactive” refers to the preferential binding of compounds [an antibody] to a particular peptide sequence, when an antibody interacts with a specific peptide sequence.

The phrase “effective amount” as used herein means that amount of one or more agent, material, or composition comprising one or more agents of the present invention that is effective for producing some desired effect in an animal. It is recognized that when an agent is being used to achieve a therapeutic effect, the actual dose which comprises the “effective amount” will vary depending on a number of conditions including the particular condition being treated, the severity of the disease, the size and health of the patient, the route of administration, etc. A skilled medical practitioner can readily determine the appropriate dose using methods well known in the medical arts.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The phrase “pharmaceutically acceptable carrier” as used herein means a pharmaceutically acceptable material, composition or vehicle, such as a liquid or solid filler, diluent, excipient, solvent or encapsulating material, involved in carrying or transporting the subject agents from one organ, or portion of the body, to another organ, or portion of the body. Each carrier must be “acceptable” in the sense of being compatible with the other ingredients of the formulation. Some examples of materials which can serve as pharmaceutically acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline, (18) Ringer's solution, (19) ethyl alcohol; (20) phosphate buffer solutions; and (21) other non-toxic compatible substances employed in pharmaceutical formulations.

Preparation of Monoclonal Antibodies

A mammal, such as a mouse, a rat, a hamster or rabbit can be immunized with the full length protein or fragments thereof, or the cDNA encoding the full length protein or a fragment thereof an immunogenic form of the peptide. Techniques for conferring immunogenicity on a protein or peptide include conjugation to carriers or other techniques well known in the art. An immunogenic portion of a polypeptide can be administered in the presence of adjuvant. The progress of immunization can be monitored by detection of antibody titers in plasma or serum. Standard ELISA or other immunoassays can be used with the immunogen as antigen to assess the levels of antibodies.

Following immunization of an animal with an antigenic preparation of the subject polypeptides, antisera can be obtained and, if desired, polyclonal antibodies isolated from the serum. To produce monoclonal antibodies, antibody-producing cells (lymphocytes) can be harvested from an immunized animal and fused by standard somatic cell fusion procedures with immortalizing cells such as myeloma cells to yield hybridoma cells. Such techniques are well known in the art, and include, for example, the hybridoma technique (originally developed by Kohler and Milstein, (1975) Nature, 256: 495-497), the human B cell hybridoma technique (Kozbar et al. (1983) Immunology Today, 4: 72), and the EBV-hybridoma technique to produce human monoclonal antibodies (Cole et al., (1985) Monoclonal Antibodies and Cancer Therapy, Alan R. Liss, Inc. pp. 77-96). Hybridoma cells can be screened immunochemically for production of antibodies specifically reactive with an epitope of the RAGE polypeptide and monoclonal antibodies isolated from a culture comprising such hybridoma cells.

Humanization

Chimeric antibodies comprise sequences from at least two different species. As one example, recombinant cloning techniques may be used to include variable regions, which contain the antigen-binding sites, from a non-human antibody (i.e., an antibody prepared in a non-human species immunized with the antigen) and constant regions derived from a human immunoglobulin.

Humanized antibodies are a type of chimeric antibody wherein variable region residues responsible for antigen binding (i.e., residues of a complementarity determining region, abbreviated complementarity determining region, or any other residues that participate in antigen binding) are derived from a non-human species, while the remaining variable region residues (i.e., residues of the framework regions) and constant regions are derived, at least in part, from human antibody sequences. A subset of framework region residues and constant region residues of a humanized antibody may be derived from non-human sources. Variable regions of a humanized antibody are also described as humanized (i.e., a humanized light or heavy chain variable region). The non-human species is typically that used for immunization with antigen, such as mouse, rat, rabbit, non-human primate, or other non-human mammalian species. Humanized antibodies are typically less immunogenic than traditional chimeric antibodies and show improved stability following administration to humans. See e.g., Benincosa et al. (2000) J. Pharmacol. Exp. Ther. 292:810-6; Kalofonos et al. (1994) Eur. J. Cancer 30A:1842-50; Subramanian et al. (1998) Pediatr. Infect. Dis. J. 17:110-5.

Complementarity determining regions (CDRs) are residues of antibody variable regions that participate in antigen binding. Several numbering systems for identifying CDRs are in common use. The Kabat definition is based on sequence variability, and the Chothia definition is based on the location of the structural loop regions. The AbM definition is a compromise between the Kabat and Chothia approaches. The CDRs of the light chain variable region are bounded by the residues at positions 24 and 34 (CDR1-L), 50 and 56 (CDR2-L), and 89 and 97 (CDR3-L) according to the Kabat, Chothia, or AbM algorithm. According to the Kabat definition, the CDRs of the heavy chain variable region are bounded by the residues at positions 31 and 35B (CDR1-H), 50 and 65 (CDR2-H), and 95 and 102 (CDR3-H) (numbering according to Kabat). According to the Chothia definition, the CDRs of the heavy chain variable region are bounded by the residues at positions 26 and 32 (CDR1-H), 52 and 56 (CDR2-H), and 95 and 102 (CDR3-H) (numbering according to Chothia). According to the AbM definition, the CDRs of the heavy chain variable region are bounded by the residues at positions 26 and 35B (CDR1-H), 50 and 58 (CDR2-H), and 95 and 102 (CDR3-H) (numbering according to Kabat). See Martin et al. (1989) Proc. Natl. Acad. Sci. USA 86: 9268-9272; Martin et al. (1991) Methods Enzymol. 203: 121-153; Pedersen et al. (1992) Immunomethods 1: 126; and Rees et al. (1996) In Sternberg M. J. E. (ed.), Protein Structure Prediction, Oxford University Press, Oxford, pp. 141-172.

As used herein, the term “CDR” refer to CDRs as defined either by Kabat or by Chothia; moreover, a humanized antibody variable of the invention may be constructed to comprise one or more CDRs as defined by Kabat, and to also comprise one or more CDRs as defined by Chothia.

Specificity determining regions (SDRs) are residues within CDRs that directly interact with antigen. The SDRs correspond to hypervariable residues. See (Padlan et al. (1995) FASEB J. 9: 133-139).

Framework residues are those residues of antibody variable regions other than hypervariable or CDR residues. Framework residues may be derived from a naturally occurring human antibody, such as a human framework that is substantially similar to a framework region of the an anti-RAGE antibody of the invention. Artificial framework sequences that represent a consensus among individual sequences may also be used. When selecting a framework region for humanization, sequences that are widely represented in humans may be preferred over less populous sequences. Additional mutations of the human framework acceptor sequences may be made to restore murine residues believed to be involved in antigen contacts and/or residues involved in the structural integrity of the antigen-binding site, or to improve antibody expression. A peptide structure prediction may be used to analyze the humanized variable heavy and light region sequences to identify and avoid post-translational protein modification sites introduced by the humanization design.

Humanized antibodies may be prepared using any one of a variety of methods including veneering, grafting of complementarity determining regions (CDRs), grafting of abbreviated CDRs, grafting of specificity determining regions (SDRs), and Frankenstein assembly, as described below. Humanized antibodies also include superhumanized antibodies, in which one or more changes have been introduced in the CDRs. For example, human residues may be substituted for non-human residues in the CDRs. These general approaches may be combined with standard mutagenesis and synthesis techniques to produce an anti-RAGE antibody of any desired sequence.

Veneering is based on the concept of reducing potentially immunogenic amino acid sequences in a rodent or other non-human antibody by resurfacing the solvent accessible exterior of the antibody with human amino acid sequences. Thus, veneered antibodies appear less foreign to human cells than the unmodified non-human antibody. See Padlan (1991) Mol. Immunol. 28:489-98. A non-human antibody is veneered by identifying exposed exterior framework region residues in the non-human antibody, which are different from those at the same positions in framework regions of a human antibody, and replacement of the identified residues with amino acids that typically occupy these same positions in human antibodies.

Grafting of CDRs is performed by replacing one or more CDRs of an acceptor antibody (e.g., a human antibody or other antibody comprising desired framework residues) with CDRs of a donor antibody (e.g., a non-human antibody). Acceptor antibodies may be selected based on similarity of framework residues between a candidate acceptor antibody and a donor antibody. For example, according to the Frankenstein approach, human framework regions are identified as having substantial sequence homology to each framework region of the relevant non-human antibody, and CDRs of the non-human antibody are grafted onto the composite of the different human framework regions. A related method also useful for preparation of antibodies of the invention is described in U.S. Patent Application Publication No. 2003/0040606.

Grafting of abbreviated CDRs is a related approach. Abbreviated CDRs include the specificity-determining residues and adjacent amino acids, including those at positions 27d-34, 50-55 and 89-96 in the light chain, and at positions 31-35b, 50-58, and 95-101 in the heavy chain (numbering convention of (Kabat et al. (1987)). See (Padlan et al. (1995) FASEB J. 9: 133-9). Grafting of specificity-determining residues (SDRs) is premised on the understanding that the binding specificity and affinity of an antibody combining site is determined by the most highly variable residues within each of the complementarity determining regions (CDRs). Analysis of the three-dimensional structures of antibody-antigen complexes, combined with analysis of the available amino acid sequence data may be used to model sequence variability based on structural dissimilarity of amino acid residues that occur at each position within the CDR. SDRs are identified as minimally immunogenic polypeptide sequences consisting of contact residues. See Padlan et al. (1995) FASEB J. 9: 133-139.

Acceptor frameworks for grafting of CDRs or abbreviated CDRs may be further modified to introduce desired residues. For example, acceptor frameworks may comprise a heavy chain variable region of a human sub-group I consensus sequence, optionally with non-human donor residues at one or more of positions 1, 28, 48, 67, 69, 71, and 93. As another example, a human acceptor framework may comprise a light chain variable region of a human sub-group I consensus sequence, optionally with non-human donor residues at one or more of positions 2, 3, 4, 37, 38, 45 and 60. Following grafting, additional changes may be made in the donor and/or acceptor sequences to optimize antibody binding and functionality. See e.g., PCT International Publication No. WO 91/09967.

Human frameworks of a heavy chain variable region that may be used to prepare humanized anti-RAGE antibodies include framework residues of DP-75, DP54, DP-54 FW VH 3 JH4, DP-54 VH3 3-07, DP-8 (VH1-2), DP-25, VI-2b and VI-3 (VH1-03), DP-15 and V1-8 (VH1-08), DP-14 and V1-18 (VH1-18), DP-5 and V1-24P (VH1-24), DP-4 (VH1-45), DP-7 (VH1-46), DP-10, DA-6 and YAC-7 (VH1-69), DP-88 (VH1-e), DP-3, and DA-8 (VH1-f).

Human frameworks of a light chain variable region that may be used to prepare humanized anti-RAGE antibodies include framework residues of human germ line clone DPK24, DPK-12, DPK-9 Vk1, DPK-9 Jk4, DPK9 Vk1 02, and germ line clone subgroups VκIII and VκI. The following mutations of a DPK24 germ line may increase antibody expression: F10S, T45K, 163S, Y67S, F73L, and T77S.

Representative humanized anti-RAGE antibodies of the invention include antibodies having one or more CDRs of a variable region amino acid sequence selected from SEQ ID NOs:16-27. For example, humanized anti-RAGE antibodies may comprise two or more CDRs selected from CDRs of a heavy chain variable region of any one of SEQ ID NOs:16, 18, 21, 24, 20, and 26, or a light chain variable region of any one of SEQ ID NOs:17, 19, 22, 25, 23, and 27. Humanized anti-RAGE antibodies may also comprise a heavy chain comprising a variable region having two or three CDRs of any one of SEQ ID NOs:16, 18, 21, 24, 20, and 26, and a light chain comprising a variable region having two or three CDRs of any one of SEQ ID NOs: 17, 19, 22, 25, 23, and 27.

Humanized anti-RAGE antibodies of the invention may be constructed wherein the variable region of a first chain (i.e., the light chain variable region or the heavy chain variable region) is humanized, and wherein the variable region of the second chain is not humanized (i.e., a variable region of an antibody produced in a non-human species). These antibodies are a type of humanized antibody referred to as semi-humanized antibodies.

The constant regions of chimeric and humanized anti-RAGE antibodies may be derived from constant regions of any one of IgA, IgD, IgE, IgG, IgM, and any isotypes thereof (e.g., IgG1, IgG2, IgG3, or IgG4 isotypes of IgG). The amino acid sequences of many antibody constant regions are known. The choice of a human isotype and modification of particular amino acids in the isotype may enhance or eliminate activation of host defense mechanisms and alter antibody biodistribution. See (Reff et al. (2002) Cancer Control 9: 152-66). For cloning of sequences encoding immunoglobulin constant regions, intronic sequences may be deleted.

Chimeric and humanized anti-RAGE antibodies may be constructed using standard techniques known in the art. For example, variable regions may be prepared by annealing together overlapping oligonucleotides encoding the variable regions and ligating them into an expression vector containing a human antibody constant region. See e.g., Harlow & Lane (1988) Antibodies: A Laboratory Manual, Cold Spring Harbor Laboratory Press, Cold Spring Harbor, N.Y. and U.S. Pat. Nos. 4,196,265; 4,946,778; 5,091,513; 5,132,405; 5,260,203; 5,677,427; 5,892,019; 5,985,279; 6,054,561. Tetravalent antibodies (H₄L₄) comprising two intact tetrameric antibodies, including homodimers and heterodimers, may be prepared, for example, as described in PCT International Publication No. WO 02/096948. Antibody dimers may also be prepared via introduction of cysteine residue(s) in the antibody constant region, which promote interchain disulfide bond formation, by use of heterobifunctional cross-linkers (Wolff et al. (1993) Cancer Res. 53: 2560-5), or by recombinant production to include a dual constant region (Stevenson et al. (1989) Anticancer Drug Des. 3: 219-30). Antigen-binding fragments of antibodies of the invention may be prepared, for example, by expression of truncated antibody sequences, or by post-translation digestion of full-length antibodies.

Variants of anti-RAGE antibodies of the invention may be readily prepared to include various changes, substitutions, insertions, and deletions. For example, antibody sequences may be optimized for codon usage in the cell type used for antibody expression. To increase the serum half life of the antibody, a salvage receptor binding epitope may be incorporated, if not present already, into the antibody heavy chain sequence. See U.S. Pat. No. 5,739,277. Additional modifications to enhance antibody stability include modification of IgG4 to replace the serine at residue 241 with proline. See Angal et al. (1993) Mol. Immunol. 30: 105-108. Other useful changes include substitutions as required to optimize efficiency in conjugating the antibody with a drug. For example, an antibody may be modified at its carboxyl terminus to include amino acids for drug attachment, for example one or more cysteine residues may be added. The constant regions may be modified to introduce sites for binding of carbohydrates or other moieties.

Additional antibody variants include glycosylation isoforms that result in improved functional properties. For example, modification of Fc glycosylation can result in altered effector functions, e.g., increased binding to Fc gamma receptors and improved ADCC and/or could decreased C1q binding and CDC (e.g., changing of Fc oligosaccharides from complex form to high-mannose or hybrid type may decrease C1q binding and CDC (see, e.g., Kanda et al., Glycobiology, 2007:17:104-118)). Modification can be done by bioengineering bacteria, yeast, plant cells, insect cells, and mammalian cells; it can also be done by manipulating protein or natural product glycosylation pathways in genetically engineered organisms. Glycosylation can also be altered by exploiting the liberality with which sugar-attaching enzymes (glycosyltransferases) tolerate a wide range of different substrates. Finally, one of skill in the art can glycosylate proteins and natural products through a variety of chemical approaches: with small molecules, enzymes, protein ligation, metabolic bioengineering, or total synthesis. Examples of suitable small molecule inhibitors of N-glycan processing include, Castanospermine (CS), Kifunensine (KF), Deoxymannojirimycin (DMJ), Swainsonine (Sw), Monensin (Mn).

Variants of anti-RAGE antibodies of the invention may be produced using standard recombinant techniques, including site-directed mutagenesis, or recombination cloning. A diversified repertoire of anti-RAGE antibodies may be prepared via gene arrangement and gene conversion methods in transgenic non-human animals (U.S. Patent Publication No. 2003/0017534), which are then tested for relevant activities using functional assays. In particular embodiments of the invention, variants are obtained using an affinity maturation protocol for mutating CDRs (Yang et al. (1995) J. Mol. Biol. 254: 392-403), chain shuffling (Marks et al. (1992) Biotechnology (NY) 10: 779-783), use of mutator strains of E. coli (Low et al. (1996) J. Mol. Biol. 260: 359-368), DNA shuffling (Patten et al. (1997) Curr. Opin. Biotechnol. 8: 724-733), phage display (Thompson et al. (1996) J. Mol. Biol. 256: 77-88), and sexual PCR (Crameri et al. (1998) Nature 391: 288-291). For immunotherapy applications, relevant functional assays include specific binding to human RAGE antigen, antibody internalization, and targeting to a tumor site(s) when administered to a tumor-bearing animal, as described herein below.

The present invention further provides cells and cell lines expressing anti-RAGE antibodies of the invention. Representative host cells include mammalian and human cells, such as CHO cells, HEK-293 cells, HeLa cells, CV-1 cells, and COS cells. Methods for generating a stable cell line following transformation of a heterologous construct into a host cell are known in the art. Representative non-mammalian host cells include insect cells (Potter et al. (1993) Int. Rev. Immunol. 10(2-3):103-112). Antibodies may also be produced in transgenic animals (Houdebine (2002) Curr. Opin. Biotechnol. 13(6):625-629) and transgenic plants (Schillberg et al. (2003) Cell Mol. Life Sci. 60(3):433-45).

As discussed above, monoclonal, chimeric and humanized antibodies, which have been modified by, e.g., deleting, adding, or substituting other portions of the antibody, e.g., the constant region, are also within the scope of the invention. For example, an antibody can be modified as follows: (i) by deleting the constant region; (ii) by replacing the constant region with another constant region, e.g., a constant region meant to increase half-life, stability or affinity of the antibody, or a constant region from another species or antibody class; or (iii) by modifying one or more amino acids in the constant region to alter, for example, the number of glycosylation sites, effector cell function, Fc receptor (FcR) binding, complement fixation, among others.

Methods for altering an antibody constant region are known in the art. Antibodies with altered function, e.g. altered affinity for an effector ligand, such as FcR on a cell, or the C1 component of complement can be produced by replacing at least one amino acid residue in the constant portion of the antibody with a different residue (see e.g., EP 388,151 A1, U.S. Pat. No. 5,624,821 and U.S. Pat. No. 5,648,260, the contents of all of which are hereby incorporated by reference). Similar type of alterations could be described which if applied to the murine, or other species immunoglobulin would reduce or eliminate these functions.

For example, it is possible to alter the affinity of an Fc region of an antibody (e.g., an IgG, such as a human IgG) for an FcR (e.g., FcγR1), or for C1q binding by replacing the specified residue(s) with a residue(s) having an appropriate functionality on its side chain, or by introducing a charged functional group, such as glutamate or aspartate, or perhaps an aromatic non-polar residue such as phenylalanine, tyrosine, tryptophan or alanine (see e.g., U.S. Pat. No. 5,624,821).

The antibody or binding fragment thereof may be conjugated with a cytotoxin, a therapeutic agent, or a radioactive metal ion. In one embodiment, the protein that is conjugated is an antibody or fragment thereof. A cytotoxin or cytotoxic agent includes any agent that is detrimental to cells. Non-limiting examples include, calicheamicin, taxol, cytochalasin B, gramicidin D, ethidium bromide, emetine, mitomycin, etoposide, tenoposide, vincristine, vinblastine, colchicin, doxorubicin, daunorubicin, dihydroxy anthracin dione, mitoxantrone, mithramycin, actinomycin D, 1-dehydrotestosterone, glucocorticoids, procaine, tetracaine, lidocaine, propranolol, puromycin, and analogs, or homologs thereof. Therapeutic agents include, but are not limited to, antimetabolites (e.g., methotrexate, 6-mercaptopurine, 6-thioguanine, cytarabine, and 5-fluorouracil decarbazine), alkylating agents (e.g., mechlorethamine, thioepa chlorambucil, melphalan, carmustine (BSNU) and lomustine (CCNU), cyclothosphamide, busulfan, dibromomannitol, streptozotocin, mitomycin C, and cis-dichlorodiamine platinum (II) (DDP), cisplatin), anthracyclines (e.g., daunorubicin and doxorubicin), antibiotics (e.g., dactinomycin, bleomycin, mithramycin, and anthramycin), and anti-mitotic agents (e.g., vincristine and vinblastine). Techniques for conjugating such moieties to proteins are well known in the art.

Alternatively, it is now possible to produce transgenic animals (e.g., mice) that are capable, upon immunization, of producing a full repertoire of human antibodies in the absence of endogenous immunoglobulin production. For example, it has been described that the homogeneous deletion of the antibody heavy-chain joining region (JM) gene in chimeric and germ-line mutant mice results in complete inhibition of endogenous antibody production. Transfer of the human germ-line immunoglobulin gene array in such germ-line mutant mice will result in the production of human antibodies upon antigen challenge. See, e.g., Jackobovits et al., Proc. Natl. Acad. Sci. USA, 90:2551 (1993); Jakobovits et al., Nature, 362:255-258 (1993); Bruggermann et al., Year in Immune, 7:33 (1983); and Duchosal et al. Nature 355:258 (1992). Human antibodies can also be derived from phage-display libraries (Hoogenboom et al., J. Mol. Biol. 227:381 (1991); Marks et al., J. Mol. Biol., 222:581-597 (1991); Vaughan et al. Nature Biotech 14:309 (1996)).

In certain embodiments, antibodies of the present invention can be administered in combination with other agents as part of a combinatorial therapy. For example, in the case of inflammatory conditions, the subject antibodies can be administered in combination with one or more other agents useful in the treatment of inflammatory diseases or conditions. In the case of cardiovascular disease conditions, and particularly those arising from atherosclerotic plaques, which are thought to have a substantial inflammatory component, the subject antibodies can be administered in combination with one or more other agents useful in the treatment of cardiovascular diseases. In the case of cancer, the subject antibodies can be administered in combination with one or more anti-angiogenic factors, chemotherapeutics, or as an adjuvant to radiotherapy. It is further envisioned that the administration of the subject antibodies will serve as part of a cancer treatment regimen that may combine many different cancer therapeutic agents. In the case of IBD, the subject antibodies can be administered with one or more anti-inflammatory agents, and may additionally be combined with a modified dietary regimen.

Methods for Inhibiting an Interaction Between a RAGE-LBE and a RAGE-BP

The invention includes methods for inhibiting the interaction between RAGE and a RAGE-BP, or modulating RAGE activity. Preferably, such methods are used for treating RAGE-associated disorders.

Such methods may comprise administering an antibody raised to RAGE as disclosed herein. Such methods comprise administering an antibody that binds specifically to one or more epitopes of a RAGE protein having an amino acid sequence as set forth in SEQ ID NO:1, SEQ ID NO:3, SEQ ID NO:7, SEQ ID NO:9, SEQ ID NO:11, or SEQ ID NO:13. In yet another embodiment, such methods comprise administering a compound that inhibits the binding of RAGE to one or more RAGE-BPs. Exemplary methods of identifying such compounds are discussed below.

In certain embodiments, the interaction is inhibited in vitro, such as in a reaction mixture comprising purified proteins, cells, biological samples, tissues, artificial tissues, etc. In certain embodiments, the interaction is inhibited in vivo, for example, by administering an antibody that binds to RAGE or a RAGE-binding fragment thereof. The antibody or fragment thereof bind to RAGE and inhibit binding of a RAGE-BP.

The invention includes methods for preventing or treating a RAGE related disorder by inhibiting the interaction between RAGE and a RAGE-BP, or modulating RAGE activity. Such methods include administering an antibody to RAGE in an amount effective to inhibit the interaction and for a time sufficient to prevent or treat said disorder.

Nucleic Acids

Nucleic acids are deoxyribonucleotides or ribonucleotides and polymers thereof in single-stranded, double-stranded, or triplexed form. Unless specifically limited, nucleic acids may contain known analogues of natural nucleotides that have similar properties as the reference natural nucleic acid. Nucleic acids include genes, cDNAs, mRNAs, and cRNAs. Nucleic acids may be synthesized, or may be derived from any biological source, including any organism.

Representative nucleic acids of the invention comprise a nucleotide sequence encoding RAGE shown in any one of SEQ ID NOs: 6, 8, 10, 12, corresponding to disclosed cDNAs encoding RAGE of baboon, cynomologus monkey, and rabbit, or shown in SEQ ID NO: 15, corresponding to a genomic DNA sequence encoding baboon RAGE. Nucleic acids of the invention also comprise a nucleotide sequence encoding any of the antibody variable region amino acid sequences shown in SEQ ID NOs: 16-49.

Nucleic acids of the invention may also comprise a nucleotide sequence that is substantially identical to any one of SEQ ID NOs: 6, 8, 10, 12, and 15, including nucleotide sequences that are at least 90%, 91%, 92%, 93%, 94%, 95%, 96%, 97%, 98%, 99%, 99.5%, or 99.9% identical to any one of SEQ ID NOs: 6, 8, 10, 12, and 15.

Nucleic acids of the invention may also comprise a nucleotide sequence encoding a RAGE protein having an amino acid sequence that is substantially identical to any of the amino acid sequences shown in SEQ ID NOs: 7, 9, 11, and 13, including nucleotide sequences that are at least 90%, 91%, 92%, 93%, 94%, 95%, 96%, 97%, 98%, 99%, 99.5%, or 99.9% identical to any one of in SEQ ID NOs: 7, 9, 11, and 13.

Nucleic acids of the invention may also comprise a nucleotide sequence encoding an anti-RAGE antibody variable region having an amino acid sequence that is substantially identical to any of the amino acid sequences shown in SEQ ID NOs: 16-49, including a nucleotide sequence encoding an amino acid sequence that is at least 85%, 86%, 87%, 88%, 89%, 90%, 91%, 92%, 93%, 94%, 95%, 96%, 97%, 98%, 99%, 99.5%, or 99.9% identical to any of SEQ ID NOs: 16-49.

Sequences are compared for maximum correspondence using a sequence comparison algorithm using the full-length variable region encoding sequence of any one of SEQ ID NOs: 16-49, a nucleotide sequence encoding a full length variable region having any one of the sequences shown in SEQ ID NO: 16-49 as the query sequence, as described herein below, or by visual inspection.

Substantially identical sequences may be polymorphic sequences, i.e., alternative sequences or alleles in a population. An allelic difference may be as small as one base pair. Substantially identical sequences may also comprise mutagenized sequences, including sequences comprising silent mutations. A mutation may comprise one or more residue changes, a deletion of one or more residues, or an insertion of one or more additional residues.

Substantially identical nucleic acids are also identified as nucleic acids that hybridize specifically to or hybridize substantially to the full length of any one of SEQ ID NOs: 6, 8, 10, 12, or 15, or to the full length of any nucleotide sequence encoding a RAGE amino acid sequence shown in SEQ ID NOs: 7, 9, 11, and 13, or encoding an antibody variable region amino acid sequence shown in SEQ ID NOs: 16-49, under stringent conditions. In the context of nucleic acid hybridization, two nucleic acid sequences being compared may be designated a probe and a target. A probe is a reference nucleic acid molecule, and a target is a test nucleic acid molecule, often found within a heterogeneous population of nucleic acid molecules. A target sequence is synonymous with a test sequence.

For hybridization studies, useful probes are complementary to or mimic at least about 14 to 40 nucleotide sequence of a nucleic acid molecule of the present invention. Preferably, probes comprise 14 to 20 nucleotides, or even longer where desired, such as 30, 40, 50, 60, 100, 200, 300, or 500 nucleotides or up to the full length of any one of SEQ ID NOs: 6, 8, 10, 12, or 15, or to the full length of any nucleotide sequence encoding a RAGE amino acid sequence shown in SEQ ID NOs: 7, 9, 11, and 13, or encoding an antibody variable region amino acid sequence shown in SEQ ID NOs: 16-49. Such fragments may be readily prepared, for example, by chemical synthesis of the fragment, by application of nucleic acid amplification technology, or by introducing selected sequences into recombinant vectors for recombinant production.

Specific hybridization refers to the binding, duplexing, or hybridizing of a molecule only to a particular nucleotide sequence under stringent conditions when that sequence is present in a complex nucleic acid mixture (e.g., total cellular DNA or RNA). Specific hybridization may accommodate mismatches between the probe and the target sequence depending on the stringency of the hybridization conditions.

Stringent hybridization conditions and stringent hybridization wash conditions in the context of nucleic acid hybridization experiments such as Southern and Northern blot analysis are both sequence- and environment-dependent. Longer sequences hybridize specifically at higher temperatures. An extensive guide to the hybridization of nucleic acids is found in Tijssen (1993) Laboratory Techniques in Biochemistry and Molecular Biology-Hybridization with Nucleic Acid Probes, part I chapter 2, Elsevier, New York, N.Y. Generally, highly stringent hybridization and wash conditions are selected to be about 5° C. lower than the thermal melting point (Tₘ) for the specific sequence at a defined ionic strength and pH. Typically, under stringent conditions a probe will hybridize specifically to its target subsequence, but to no other sequences.

The Tₘ is the temperature (under defined ionic strength and pH) at which 50% of the target sequence hybridizes to a perfectly matched probe. Very stringent conditions are selected to be equal to the Tₘ for a particular probe. An example of stringent hybridization conditions for Southern or Northern Blot analysis of complementary nucleic acids having more than about 100 complementary residues is overnight hybridization in 50% formamide with 1 mg of heparin at 42° C. An example of highly stringent wash conditions is 15 minutes in 0.1×SSC at 65° C. An example of stringent wash conditions is 15 minutes in 0.2×SSC buffer at 65° C. See Sambrook et al., eds (1989) Molecular Cloning: A Laboratory Manual, Cold Spring Harbor Laboratory Press, Cold Spring Harbor, N.Y., for a description of SSC buffer. Often, a high stringency wash is preceded by a low stringency wash to remove background probe signal. An example of medium stringency wash conditions for a duplex of more than about 100 nucleotides, is 15 minutes in 1×SSC at 45° C. An example of low stringency wash for a duplex of more than about 100 nucleotides, is 15 minutes in 4× to 6×SSC at 40° C. For short probes (e.g., about 10 to 50 nucleotides), stringent conditions typically involve salt concentrations of less than about 1M Na⁺ ion, typically about 0.01 to 1M Na⁺ ion concentration (or other salts) at pH 7.0-8.3, and the temperature is typically at least about 30° C. Stringent conditions may also be achieved with the addition of destabilizing agents such as formamide. In general, a signal to noise ratio of 2-fold (or higher) than that observed for an unrelated probe in the particular hybridization assay indicates detection of a specific hybridization.

The following are examples of hybridization and wash conditions that may be used to identify nucleotide sequences that are substantially identical to reference nucleotide sequences of the present invention: a probe nucleotide sequence preferably hybridizes to a target nucleotide sequence in 7% sodium dodecyl sulphate (SDS), 0.5M NaPO₄, 1 mM EDTA at 50° C. followed by washing in 2×SSC, 0.1% SDS at 50° C.; more preferably, a probe and target sequence hybridize in 7% sodium dodecyl sulphate (SDS), 0.5M NaPO₄, 1 mM EDTA at 50° C. followed by washing in 1×SSC, 0.1% SDS at 50° C.; more preferably, a probe and target sequence hybridize in 7% sodium dodecyl sulphate (SDS), 0.5M NaPO₄, 1 mM EDTA at 50° C. followed by washing in 0.5×SSC, 0.1% SDS at 50° C.; more preferably, a probe and target sequence hybridize in 7% sodium dodecyl sulphate (SDS), 0.5M NaPO₄, 1 mM EDTA at 50° C. followed by washing in 0.1×SSC, 0.1% SDS at 50° C.; more preferably, a probe and target sequence hybridize in 7% sodium dodecyl sulphate (SDS), 0.5M NaPO₄, 1 mM EDTA at 50° C. followed by washing in 0.1×SSC, 0.1% SDS at 65° C.

A further indication that two nucleic acid sequences are substantially identical is that proteins encoded by the nucleic acids are substantially identical, share an overall three-dimensional structure, or are biologically functional equivalents. These terms are defined further herein below. Nucleic acid molecules that do not hybridize to each other under stringent conditions are still substantially identical if the corresponding proteins are substantially identical. This may occur, for example, when two nucleotide sequences comprise conservatively substituted variants as permitted by the genetic code.

Conservatively substituted variants are nucleic acid sequences having degenerate codon substitutions wherein the third position of one or more selected (or all) codons is substituted with mixed-base and/or deoxyinosine residues. See Batzer et al. (1991) Nucleic Acids Res. 19:5081; Ohtsuka et al. (1985) J. Biol. Chem. 260:2605-2608; and Rossolini et al. (1994) Mol. Cell Probes 8:91-98.

Nucleic acids of the invention also comprise nucleic acids complementary to any one of SEQ ID NOs: 6, 8, 10, 12, or 15, or nucleotide sequences encoding a RAGE amino acid sequence shown in SEQ ID NOs: 7, 9, 11, and 13, or encoding an antibody variable region amino acid sequence shown in SEQ ID NOs: 16-49, and complementary sequences thereof. Complementary sequences are two nucleotide sequences that comprise antiparallel nucleotide sequences capable of pairing with one another upon formation of hydrogen bonds between base pairs. As used herein, the term complementary sequences means nucleotide sequences which are substantially complementary, as may be assessed by the same nucleotide comparison methods set forth below, or is defined as being capable of hybridizing to the nucleic acid segment in question under relatively stringent conditions such as those described herein. A particular example of a complementary nucleic acid segment is an antisense oligonucleotide.

A subsequence is a sequence of nucleic acids that comprises a part of a longer nucleic acid sequence. An exemplary subsequence is a probe, described herein above, or a primer. The term primer as used herein refers to a contiguous sequence comprising about 8 or more deoxyribonucleotides or ribonucleotides, preferably 10-20 nucleotides, and more preferably 20-30 nucleotides of a selected nucleic acid molecule. The primers of the invention encompass oligonucleotides of sufficient length and appropriate sequence so as to provide initiation of polymerization on a nucleic acid molecule of the present invention.

An elongated sequence comprises additional nucleotides (or other analogous molecules) incorporated into the nucleic acid. For example, a polymerase (e.g., a DNA polymerase) may add sequences at the 3′ terminus of the nucleic acid molecule. In addition, the nucleotide sequence may be combined with other DNA sequences, such as promoters, promoter regions, enhancers, polyadenylation signals, intronic sequences, additional restriction enzyme sites, multiple cloning sites, and other coding segments. Thus, the invention also provides vectors comprising the disclosed nucleic acids, including vectors for recombinant expression, wherein a nucleic acid of the invention is operatively linked to a functional promoter. When operatively linked to a nucleic acid, a promoter is in functional combination with the nucleic acid such that the transcription of the nucleic acid is controlled and regulated by the promoter region. Vectors refer to nucleic acids capable of replication in a host cell, such as plasmids, cosmids, and viral vectors.

Nucleic acids of the present invention may be cloned, synthesized, altered, mutagenized, or combinations thereof. Standard recombinant DNA and molecular cloning techniques used to isolate nucleic acids are known in the art. Site-specific mutagenesis to create base pair changes, deletions, or small insertions is also known in the art. See e.g., Sambrook et al. (eds.) (1989) Molecular Cloning: A Laboratory Manual. Cold Spring Harbor Laboratory Press, Cold Spring Harbor, N.Y.; Silhavy et al. (1984) Experiments with Gene Fusions. Cold Spring Harbor Laboratory Press, Cold Spring Harbor, N.Y.; Glover & Hames (1995) DNA Cloning: A Practical Approach, 2nd ed. IRL Press at Oxford University Press, Oxford/New York; Ausubel (ed.) (1995) Short Protocols in Molecular Biology, 3rd ed. Wiley, New York.

Methods of Treatment

The invention relates to and includes methods of treating RAGE-related or RAGE-associated disorders. RAGE-related disorders may be characterized generally as including any disorder in which an affected cell exhibits elevated expression of RAGE or one or more RAGE ligands. RAGE-related disorders may also be characterized as any disorder that is treatable (i.e., one or more symptoms may be eliminated or ameliorated) by a decrease in RAGE function. For example, RAGE function can be decreased by administration of an agent that disrupts the interaction between RAGE and a RAGE-BP, such as an antibody to RAGE.

The increased expression of RAGE is associated with several pathological states, such as diabetic vasculopathy, nephropathy, retinopathy, neuropathy, and other disorders, including immune/inflammatory reactions of blood vessel walls and sepsis. RAGE ligands are produced in tissue affected with many inflammatory disorders, including arthritis (such as rheumatoid arthritis). In diabetic tissues, the production of RAGE is thought to be caused by the overproduction of advanced glycation endproducts. This results in oxidative stress and endothelial cell dysfunction that leads to vascular disease in diabetics.

The invention includes a method of treating inflammation and diseases or conditions characterized by activation of the inflammatory cytokine cascade in a subject, comprising administering an effective amount of an anti-RAGE antibody or a RAGE-binding fragment thereof and/or a composition (e.g., pharmaceutical composition) comprising an anti-RAGE antibody or a RAGE-binding fragment thereof. For example, the S100/calgranulins have been shown to comprise a family of closely related calcium-binding polypeptides characterized by two EF-hand regions linked by a connecting peptide (e.g., see Schafer et al., 1996, TIBS, 21:134-140; Zimmer et al., 1995, Brain Res. Bull., 37:417-429; Rammes et al., 1997, J. Biol. Chem., 272:9496-9502; Lugering et al., 1995, Eur. J. Clin. Invest., 25:659-664). Although they lack signal peptides, it has long been known that S100/calgranulins gain access to the extracellular space, especially at sites of chronic immune/inflammatory responses, as in cystic fibrosis and rheumatoid arthritis. RAGE is a receptor for many members of the S100/calgranulin family, mediating their proinflammatory effects on cells such as lymphocytes and mononuclear phagocytes. Also, studies on delayed-type hypersensitivity response, colitis in IL-10 null mice, collagen-induced arthritis, and experimental autoimmune encephalitis models suggest that RAGE-ligand interaction (presumably with S-100/calgranulins) has a proximal role in the inflammatory cascade. An inflammatory condition that is suitable for the methods of treatment described herein can be one in which the inflammatory cytokine cascade is activated.

The inflammatory cytokine cascade may cause a systemic reaction, as occurs with septic shock. The anti-RAGE antibodies and RAGE-binding fragments thereof of the invention can be used to treat sepsis, septic shock, and systemic listeriosis. Sepsis is a systemic inflammatory response to infection, and is associated with organ dysfunction, hypoperfusion, or hypotension. In septic shock, a severe form of sepsis, hypotension is induced despite adequate fluid resuscitation. Listeriosis is a serious infection caused by eating food contaminated with the bacterium Listeria monocytogenes. RAGE has been shown to mediate the lethal effects of septic shock (Liliensek et al., 2004, 113:11641-50). Sepsis has a complex physiology, defined by systemic inflammation and organ dysfunction, including abnormalities in body temperature; cardiovascular parameters and leukocyte count; elevated liver enzymes and altered cerebral function. The response in sepsis is to an infection or stimulus that becomes amplified and dysregulated. The murine CLP model of sepsis results in a polymicrobial infection, with abdominal abscess and bacteremia, and recreates the hemodynamic and metabolic phases observed in human disease. Experimental results obtained with the murine CLP model of sepsis described herein show that RAGE plays an important role in the pathogenesis of sepsis. The data also demonstrates that administration of an anti-RAGE antibody that binds specifically to RAGE at the time of surgery, as well as up to 36 hours after the surgery, provides significant therapeutic protection to the mice, as evidenced by increased survival and improved pathology scores. Antibodies used for the treatment of sepsis, listeriosis, and other RAGE-related diseases can be antibodies that bind to the V domain of RAGE and prevent a RAGE ligand or binding partner from binding to the RAGE protein.

The inflammatory condition that is treated or prevented by the antibodies and methods of the invention may be mediated by a localized inflammatory cytokine cascade, as in rheumatoid arthritis. Nonlimiting examples of inflammatory conditions that can be usefully treated using anti-RAGE antibodies and RAGE-binding fragments thereof and/or compositions of the present invention include, e.g., diseases involving the gastrointestinal tract and associated tissues (such as ileus, appendicitis, peptic, gastric and duodenal ulcers, peritonitis, pancreatitis, ulcerative, pseudomembranous, acute and ischemic colitis, diverticulitis, epiglottitis, achalasia, cholangitis, cholecystitis, coeliac disease, hepatitis, Crohn's disease, enteritis, and Whipple's disease); systemic or local inflammatory diseases and conditions (such as asthma, allergy, anaphylactic shock, immune complex disease, organ ischemia, reperfusion injury, organ necrosis, hay fever, sepsis, septicemia, endotoxic shock, cachexia, hyperpyrexia, eosinophilic granuloma, granulomatosis, and sarcoidosis); diseases involving the urogenital system and associated tissues (such as septic abortion, epididymitis, vaginitis, prostatitis, and urethritis); diseases involving the respiratory system and associated tissues (such as bronchitis, emphysema, rhinitis, cystic fibrosis, pneumonitis, adult respiratory distress syndrome, pneumoultramicroscopicsilicovolcanoconiosis, alvealitis, bronchiolitis, pharyngitis, pleurisy, and sinusitis); diseases arising from infection by various viruses (such as influenza, respiratory syncytial virus, HIV, hepatitis B virus, hepatitis C virus and herpes), bacteria (such as disseminated bacteremia, Dengue fever), fingi (such as candidiasis) and protozoal and multicellular parasites (such as malaria, filariasis, amebiasis, and hydatid cysts); dermatological diseases and conditions of the skin (such as burns, dermatitis, dermatomyositis, sunburn, urticaria warts, and wheals); diseases involving the cardiovascular system and associated tissues (such as stenosis, restenosis, vasulitis, angiitis, endocarditis, arteritis, atherosclerosis, thrombophlebitis, pericarditis, congestive heart failure, myocarditis, myocardial ischemia, periarteritis nodosa, and rheumatic fever); diseases involving the central or peripheral nervous system and associated tissues (such as meningitis, encephalitis, multiple sclerosis, cerebral infarction, cerebral embolism, Guillame-Barre syndrome, neuritis, neuralgia, spinal cord injury, paralysis, and uveitis); diseases of the bones, joints, muscles and connective tissues (such as the various arthritides and arthralgias, osteomyelitis, fasciitis, Paget's disease, gout, periodontal disease, rheumatoid arthritis, and synovitis); other autoimmune and inflammatory disorders (such as myasthenia gravis, thryoiditis, systemic lupus erythematosus, Goodpasture's syndrome, Behcets's syndrome, allograft rejection, graft-versus-host disease, Type I diabetes, ankylosing spondylitis, Berger's disease, and Retier's syndrome); as well as various cancers, tumors and proliferative disorders (such as Hodgkins disease); and, in any case the inflammatory or immune host response to any primary disease.

Anti-RAGE antibodies and RAGE-binding fragments thereof of the invention can be used to treat cancer. Tumor cells evince an increased expression of a RAGE ligand, particularly amphoterin, a high mobility group I nonhistone chromosomal DNA binding protein (Rauvala et al., J. Biol. Chem., 262:16625-16635 (1987); Parkikinen et al., J. Biol. Chem., 268:19726-19738 (1993)) which has been shown to interact with RAGE. Amphoterin promotes neurite outgrowth, as well as serving as a surface for assembly of protease complexes in the fibrinolytic system (also known to contribute to cell mobility). indicating that cancers are also a RAGE-related disorder. The oxidative effects and other aspects of chronic inflammation also have a contributory effect to the genesis of certain tumors. For example, In addition, a local tumor growth inhibitory effect of blocking RAGE has been observed in a primary tumor model (C6 glioma), the Lewis lung metastasis model (Taguchi et al., 2000, Nature 405:354-360), and spontaneously arising papillomas in mice expressing the v-Ha-ras transgene (Leder et al., 1990, Proc. Natl. Acad. Sci., 87:9178-9182).

Antibodies or binding fragments thereof of the invention can be used to treat or prevent diabetes, complications of diabetes, and pathological conditions associated with diabetes. It has been shown that nonenzymatic glycoxidation of macromolecules ultimately resulting in the formation of advanced glycation endproducts (AGEs) is enhanced at sites of inflammation, in renal failure, in the presence of hyperglycemia and other conditions associated with systemic or local oxidant stress (Dyer et al., J. Clin. Invest., 91:2463-2469 (1993); Reddy et al., Biochem., 34:10872-10878 (1995); Dyer et al., J. Biol. Chem., 266:11654-11660 (1991); Degenhardt et al., Cell Mol. Biol., 44:1139-1145 (1998)). Accumulation of AGEs in the vasculature can occur focally, as in the joint amyloid composed of AGE-β2-microglobulin found in patients with dialysis-related amyloidosis (Miyata et al., J. Clin. Invest., 92:1243-1252 (1993); Miyata et al., J. Clin. Invest., 98:1088-1094 (1996)), or generally, as exemplified by the vasculature and tissues of patients with diabetes (Schmidt et al., Nature Med., 1:1002-1004 (1995)). The progressive accumulation of AGEs over time in patients with diabetes suggests that endogenous clearance mechanisms are not able to function effectively at sites of AGE deposition. Such accumulated AGEs have the capacity to alter cellular properties by a number of mechanisms. Although RAGE is expressed at low levels in normal tissues and vasculature, in an environment where the receptor's ligands accumulate, it has been shown that RAGE becomes upregulated (Li et al., J. Biol. Chem., 272:16498-16506 (1997); Li et al., J. Biol. Chem., 273:30870-30878 (1998); Tanaka et al., J. Biol. Chem., 275:25781-25790 (2000)). RAGE expression is increased in endothelium, smooth muscle cells and infiltrating mononuclear phagocytes in diabetic vasculature. Also, studies in cell culture have demonstrated that AGE-RAGE interaction caused changes in cellular properties important in vascular homeostasis.

Anti-RAGE antibodies or binding fragments thereof can also be used to treat erectile dysfunction. RAGE activation produces oxidants via an NADH oxidase-like enzyme, therefore suppressing the circulation of nitric oxide, which is the principle stimulator of cavernosal smooth muscle relaxation that results in penile erection. By inhibiting the activation of RAGE signaling pathways, generation of oxidants is attenuated.

Antibodies or binding fragments thereof of the invention can be used to treat or prevent atherosclerosis. It has been shown that ischemic heart disease is particularly high in patients with diabetes (Robertson, et al., Lab Invest, 18:538-551 (1968); Kannel et al., J. Am. Med. Assoc., 241:2035-2038 (1979); Kannel et al., Diab. Care, 2:120-126 (1979)). In addition, studies have shown that atherosclerosis in patients with diabetes is more accelerated and extensive than in patients not suffering from diabetes (see e.g. Wailer et at., Am. J. Med. 69:498-506 (1980); Crall et. al., Am. J. Med. 64:221-230 (1978); Hamby et. al., Chest. 2:251-257 (1976); and Pyorala et al., Diaib. Metab. Rev., 3:463-524 (1987)). Although the reasons for accelerated atherosclerosis in the setting of diabetes are many, it his been shown that reduction of AGEs can reduce plaque formation.

Accordingly, the list of RAGE-related disorders that may be treated or prevented with an inventive composition include: acute inflammatory diseases (such as sepsis), shock (e.g., septic shock, hemorrhagic shock), chronic inflammatory diseases (such as rheumatoid and psoriatic arthritis, osteoarthritis, ulcerative colitis, irritable bowel disease, multiple sclerosis, psoriasis, lupus, systemic lupus nephritis, and inflammatory lupus nephritis, and other autoimmune diseases), cardiovascular diseases (e.g., atherosclerosis, stroke, fragile plaque disorder, angina and restenosis), diabetes (and particularly cardiovascular diseases in diabetics), complications of diabetes, erectile dysfunction, cancers (e.g., lung cancer, squamous cell carcinoma, prostate cancer, human pancreatic cancer, renal cell carcinoma melanoma), vasculitis and other vasculitis syndromes such as necrotizing vasculitides, nephropathies, retinopathies, and neuropathies.

The invention provides for the administration of anti-RAGE antibodies and RAGE-binding fragments in vivo. The subject antibodies may be administered as pharmaceutical compositions, and may also be administered with one or more additional agents. The administration of the subject antibodies can be part of a therapeutic regimen to treat a particular condition. Conditions that can be treated by administration of either the antibodies alone, or by administration of the subject antibodies in combination with other agents, include RAGE-associated disorders. By way of example, RAGE-associated disorders include, but are not limited to, rheumatoid arthritis, osteoarthritis, inflammatory bowel disease, atherosclerosis, vasculitis and other vasculitis syndromes such as necrotizing vasculitides, Alzheimer's disease, cancer, complications of diabetes such as diabetic retinopathy, autoimmune diseases such as psoriasis and lupus. RAGE-associated disorders further include acute inflammatory diseases (e.g., sepsis), chronic inflammatory diseases, and other conditions that are aggravated by inflammation (i.e., the symptoms of which may be ameliorated by decreasing inflammation).

Methods of administration of the antibody based compositions can be by any of a number of methods well known in the art. These methods include local or systemic administration and further include intradermal, intramuscular, intraperitoneal, intravenous, subcutaneous, oral, and intranasal routes of administration, including use of a nebulizer and inhalation. In addition, it may be desirable to introduce the pharmaceutical compositions of the invention into the central nervous system by any suitable route, including intraventricular and intrathecal injection. Intraventricular injection may be facilitated by an intraventricular catheter, for example, attached to a reservoir, such as an Ommaya reservoir. Methods of introduction may also be provided by rechargeable or biodegradable devices, e.g., depots. Furthermore, it is contemplated that administration may occur by coating a device, implant, stent, or prosthetic.

For example, cartilage severely damaged by conditions of the joints such as rheumatoid arthritis and osteoarthritis can be replaced, in whole or in part, by various prosthetics. A variety of suitable transplantable materials exist including those based on collagen-glycosaminoglycan templates (Stone et al. (1990) Clin. Orthop. Relat. Red. 252: 129), isolated chondrocytes (Grande et al. (1989) J Orthop Res 7: 208; and Taligawa et al. (1987) Bone Miner 2: 449), and chondrocytes attached to natural or synthetic polymers (Walitani et al. (1989) J Bone Jt Surg 71B: 74; Vacanti et al. (1991) Plast Reconstr Surg 88: 753; von Schroeder et al. (1991) J Biomed Mater Res 25:329; Freed et al. (1993) J Biomed Mater Res 27: 11; and the Vacanti et al. U.S. Pat. No. 5,041,138). For example, chondrocytes can be grown in culture on biodegradable, biocompatible highly porous scaffolds formed from polymers such as polyglycolic acid, polylactic acid, agarose gel, or other polymers that degrade over time as a function of hydrolysis of the polymer backbone into innocuous monomers. The matrices are designed to allow adequate nutrient and gas exchange to the cells until engraftment occurs. The cells can be cultured in vitro until adequate cell volume and density has developed for the cells to be implanted. One advantage of the matrices is that they can be cast or molded into a desired shape on an individual basis, so that the final product closely resembles the patient's own ear or nose (by way of example), or flexible matrices can be used which allow for manipulation at the time of implantation, as in a joint.

These and other implants and prosthetics can be treated with and used to administer the subject antibodies or binding fragments thereof. For example, a composition including the antibody or binding fragment can be applied to or coated on the implant or prosthetic. In this way, the antibodies or fragments thereof can be administered directly to the specific affected tissue (e.g., to the damaged joint).

The subject antibodies can be administered as part of a combinatorial therapy with other agents. Combination therapy refers to any form of administration in combination of two or more different therapeutic compounds such that the second compound is administered while the previously administered therapeutic compound is still effective in the body (e.g., the two compounds are simultaneously effective in the patient, which may include synergistic effects of the two compounds). For example, the different therapeutic compounds can be administered either in the same formulation or in a separate formulation, either concomitantly or sequentially. Thus, an individual who receives such treatment can have a combined (conjoint) effect of different therapeutic compounds.

For example, in the case of inflammatory conditions, the subject antibodies can be administered in combination with one or more other agents useful in the treatment of inflammatory diseases or conditions. Agents useful in the treatment of inflammatory diseases or conditions include, but are not limited to, anti-inflammatory agents, or antiphlogistics. Antiphlogistics include, for example, glucocorticoids, such as cortisone, hydrocortisone, prednisone, prednisolone, fluorcortolone, triamcinolone, methylprednisolone, prednylidene, paramethasone, dexamethasone, betamethasone, beclomethasone, fluprednylidene, desoxymethasone, fluocinolone, flunethasone, diflucortolone, clocortolone, clobetasol and fluocortin butyl ester; immunosuppressive agents such as anti-TNF agents (e.g., etanercept, infliximab) and IL-1 inhibitors; penicillamine; non-steroidal anti-inflammatory drugs (NSAIDs) which encompass anti-inflammatory, analgesic, and antipyretic drugs such as salicyclic acid, celecoxib, difunisal and from substituted phenylacetic acid salts or 2phenylpropionic acid salts, such as alclofenac, ibutenac, ibuprofen, clindanac, fenclorac, ketoprofen, fenoprofen, indoprofen, fenclofenac, diclofenac, flurbiprofen, piprofen, naproxen, benoxaprofen, carprofen and cicloprofen; oxican derivatives, such as piroxican; anthranilic acid derivatives, such as mefenamic acid, flufenamic acid, tolfenamic acid and meclofenamic acid, anilino-substituted nicotinic acid derivatives, such as the fenamates miflumic acid, clonixin and flunixin; heteroarylacetic acids wherein heteroaryl is a 2-indol-3-yl or pyrrol-2-yl group, such as indomethacin, oxmetacin, intrazol, acemetazin, cinmetacin, zomepirac, tolmetin, colpirac and tiaprofenic acid; idenylacetic acid of the sulindac type; analgesically active heteroaryloxyacetic acids, such as benzadac; phenylbutazone; etodolac; nabunetone; and disease modifying antirheumatic drugs (DMARDs) such as methotrexate, gold salts, hydroxychloroquine, sulfasalazine, ciclosporin, azathioprine, and leflunomide.

Other therapeutics useful in the treatment of inflammatory diseases or conditions include antioxidants. Antioxidants may be natural or synthetic. Antioxidants are, for example, superoxide dismutase (SOD), 21-aminosteroids/aminochromans, vitamin C or E, etc. Many other antioxidants are well known to those of skill in the art.

The subject antibodies may serve as part of a treatment regimen for an inflammatory condition, which may combine many different anti-inflammatory agents. For example, the subject antibodies may be administered in combination with one or more of an NSAID, DMARD, or immunosuppressant. In one embodiment of the application, the subject antibodies or fragments thereof may be administered in combination with methotrexate. In another embodiment, the subject subject antibodies may be administered in combination with a TNF-α inhibitor.

In the case of cardiovascular disease conditions, and particularly those arising from atherosclerotic plaques, which are thought to have a substantial inflammatory component, the subject antibodies can be administered in combination with one or more other agents useful in the treatment of cardiovascular diseases. Agents useful in the treatment of cardiovascular diseases include, but are not limited to, β-blockers such as carvedilol, metoprolol, bucindolol, bisoprolol, atenolol, propranolol, nadolol, timolol, pindolol, and labetalol; antiplatelet agents such as aspirin and ticlopidine; inhibitors of angiotensin-converting enzyme (ACE) such as captopril, enalapril, lisinopril, benazopril, fosinopril, quinapril, ramipril, spirapril, and moexipril; and lipid-lowering agents such as mevastatin, lovastatin, simvastatin, pravastatin, fluvastatin, atorvastatin, and rosuvastatin.

In the case of cancer, the subject antibodies can be administered in combination with one or more anti-angiogenic factors, chemotherapeutics, or as an adjuvant to radiotherapy. It is further envisioned that the administration of the subject antibodies will serve as part of a cancer treatment regimen, which may combine many different cancer therapeutic agents. Antibodies or binding fragments thereof may be linked or coupled to a cytotoxin or radiotherapeutics to kill cancer cells expressing RAGE. Such antibodies or fragments thereof may be administered to a patient such that the antibody will bind to cancer cells expressing RAGE. In the case of IBD, the subject antibodies can be administered with one or more anti-inflammatory agents, and may additionally be combined with a modified dietary regimen.

For the treatment of sepsis and sepsis-related disorders or conditions such as septic shock, as well as for the treatment of systemic listeriosis, anti-RAGE antibodies of the invention can be administered in combination with other agents and therapeutic regimens to treat sepsis and sepsis-related disorders or conditions, or to treat systemic listeriosis. For example, sepsis or listeriosis can be treated by administering the subject antibodies in combination with antibiotics and/or other pharmaceutical compositions that are the standard of care for the particular symptoms and state of the patient.

In one aspect, the present invention also provides a method for inhibiting the interaction of an AGE with RAGE in a subject which comprises administering to the subject a therapeutically effective amount of a compound identified by the methods of the invention. A therapeutically effective amount is an amount that is capable of preventing interaction of AGE/RAGE in a subject. Accordingly, the amount will vary with the subject being treated. Administration of the compound may be hourly, daily, weekly, monthly, yearly or a single event. For example, the effective amount of the compound may comprise from about 1 μg/kg body weight to about 100 mg/kg body weight. In one embodiment, the effective amount of the compound comprises from about 1 μg/kg body weight to about 50 mg/kg body weight. In a further embodiment, the effective amount of the compound comprises from about 10 μg/kg body weight to about 10 mg/kg body weight. The actual effective amount will be established by dose/response assays using methods standard in the art (Johnson et al., Diabetes. 42:1179, (1993)). Thus, as is known to those in the art, the effective amount will depend on bioavailability, bioactivity, and biodegradability of the compound.

For example, the anti-RAGE antibodies and compositions of the invention are administered to a patient in need thereof in an amount sufficient to inhibit release of proinflammatory cytokine from a cell and/or to treat an inflammatory condition. The invention includes inhibiting release of a proinflammatory cytokine by at least 10%, 20%, 25%, 50%, 75%, 80%, 90%, or 95%, as assessed using methods described herein or other methods known in the art.

In an embodiment, the subject is an animal. In an embodiment, the subject is a human. In an embodiment, the subject is suffering from an AGE-related disease such as diabetes, amyloidoses, renal failure, aging, or inflammation. In another embodiment, the subject comprises an individual with Alzheimer's disease. In an alternative embodiment, the subject comprises an individual with cancer. In yet another embodiment, the subject comprises an individual with systemic lupus erythmetosis, or inflammatory lupus nephritis.

The subject antibodies or binding fragments thereof can be administered in a dose of from about 1 μg/kg body weight to about 100 mg/kg body weight. In one embodiment, the effective amount of the compound comprises from about 1 μg/kg body weight to about 50 mg/kg body weight. The length frequency of treatment will depend upon inter alia the particular disease state as well as the state of the patient.

Biomarkers

Biomarkers that measure sepsis disease activity, such as CRP, IL-6, pro-calcitonin, pro-adrenomedullin, and coagulation parameters (D-dimer, PAI-1 levels, protein-C, fibrinogen) can be monitored to characterize subjects with regard to disease state and potential and actual response to treatment with ant-RAGE antibodies of the invention.

In addition, soluble RAGE (sRAGE) is found in plasma as either a secreted form or a cleaved form from the cell membrane. An assay for measuring plasma levels of sRAGE has been developed and can also be used to characterize the subjects. Since the antibodies of the invention binds to sRAGE, the presence of sRAGE in the patient's plasma may influence the pharmacodynamics of treatment with antibodies of the invention, if the sRAGE is present in concentrations close to the concentrations of the antibody.

Drug Screening Assays

In certain embodiments, the present invention provides assays for identifying test antibodies that inhibit the binding of a RAGE-BP (e.g., HMGB1, AGE, Aβ, SAA, S100, amphoterin, S100P, S100A, S100A4, A100A8, S100A9, CRP, β2-integrin, Mac-1, and p150,95) to a receptor polypeptide (e.g., RAGE or RAGE-LBE, as described above).

In certain embodiments, the assays detect test antibodies that modulate the signaling activities of the RAGE receptor induced by a RAGE-BP selected from the group consisting of HMGB1, AGE, Aβ, SAA, S100, amphoterin, S100P, S100A, S100A4, A100A8, S100A9, CRP, β2-integrin, Mac-1, and p150,95. Such signaling activities include, but are not limited to, binding to other cellular components, activating enzymes such as mitogen-activated protein kineses (MAPKs), activating NF-κB transcriptional activity, and the like.

The above-noted RAGE binding proteins are relevant to signaling pathways involved in cell growth and proliferation, including cancerous cell growth. For example, S100P is a member of the S100 family of calcium binding proteins (>20 members) and is a 95 amino acid protein first isolated from placenta. S100P is expressed and secreted by >90% of all pancreatic tumors and expression increases with progression of pancreatic cancer. S100P is also expressed in lung, breast, prostate and colon cancer, expression in colon cell lines is correlated with resistance to chemotherapy and in lung cancer, high expression of S100P indicates poor prognosis. Gene transfer or extra-cellular addition of S100P increases tumor cell proliferation, motility, invasion and survival of cells in vitro and tumor growth and metastasis in vivo, while silencing of S100P expression results in a decrease of proliferation and metastasis. The only known receptor for S100P is RAGE, expression of which has been correlated with the invasion and metastasis of gastric carcinoma and glioma. Inhibitors of RAGE abrogate the effects of S100P-RAGE interaction on cell signaling, proliferation and survival and an inhibitory protein derived from amphoterin acts as an antagonist for the S100P-RAGE interaction. Anti-RAGE antibodies and the expression of dominant negative RAGE inhibit the effects of S100P.

A variety of assay formats will suffice and, in light of the present disclosure, those not expressly described herein will nevertheless be comprehended by one of ordinary skill in the art. Assay formats which approximate such conditions as formation of protein complexes, enzymatic activity, may be generated in many different forms, and include assays based on cell-free systems, e.g., purified proteins or cell lysates, as well as cell-based assays which utilize intact cells. Simple binding assays can be used to detect compounds that inhibit the interaction between a RAGE BP (e.g., HMGB1, AGE, Aβ, SAA, S100, amphoterin, S100P, S100A, S100A4, A100A8, S100A9, CRP, β2-integrin, Mac-1, and p150,95) and a receptor polypeptide (e.g., RAGE or RAGE-LBE). Compounds to be tested can be produced, for example, by bacteria, yeast or other organisms (e.g., natural products), produced chemically (e.g., small molecules, including peptidonimetics), or produced recombinantly.

In many embodiments, a cell is manipulated after incubation with a candidate compound and assayed for signaling activities of the RAGE receptor induced by a RAGE-BP (e.g., HMGB1, AGE, Aβ, SAM, S100, amphoterin, S100P, S100A, S100A4, A100A8, S100A9, CRP, β2-integrin, Mac-1, and p150,95). In certain embodiments, bioassays for such activities may include NF-κB activity assays (e.g., NF-κB luciferase or GFP reporter gene assays).

Exemplary NF-κB luciferase or GFP reporter gene assays may be carried out as described by Shona et al. (2002) FEBS Letters. 515: 119-126. Briefly, cells expressing RAGE receptor or a variant thereof are transfected with an NF-κB-luciferase reporter gene. The transfected cells are then incubated with a candidate compound. Subsequently, NF-κB-stimulated luciferase activity is measured in cells treated with the compound or without the compound. Alternatively, cells can be transfected with an NF-κB-GFP reporter gene (Stratagene). The transfected cells are then incubated with a candidate compound. Subsequently, NF-κB-stimulated gene activity are monitored by measuring GFP expression with a fluorescence/visible light microscope set-up or by FACS analysis.

In certain embodiments, the present invention provides reconstituted protein so preparations including a receptor polypeptide (e.g., RAGE or RAGE-LBE), and one or more RAGE-BPs (e.g., HMGB1, AGE, Aβ, SAM, S100, amphoterin, S100P, S100A, S100A4, A100A8, S100A9, CRP, β2-integrin, Mac-1, and p150,95). Assays of the present invention include labeled in vitro protein-protein binding assays, immunoassays for protein binding, and the like. The purified protein may also be used for determination of three-dimensional crystal structure, which can be used for modeling intermolecular interactions. The purified antibody may also be used for determination of three-dimensional crystal structure, which can be used for modeling intermolecular interactions.

In certain embodiments of the present assays, a RAGE-BP polypeptide (e.g., HMGB1, AGE, Aβ, SAA, S100, amphoterin, S100P, S100A, S100A4, A100A8, S100A9, CRP, β2-integrin, Mac-1, and p150,95) or a receptor polypeptide (e.g., RAGE) can be endogenous to the cell selected to support the assays. Alternatively, a RAGE-BP polypeptide or a receptor polypeptide (e.g., RAGE or RAGE-LBE) can be derived from exogenous sources. For instance, polypeptides can be introduced into the cell by recombinant techniques (such as through the use of an expression vector), as well as by microinjecting the polypeptide itself or mRNA encoding the polypeptide.

In further embodiments of the assays, a complex between a RAGE-BP and a receptor polypeptide can be generated in whole cells, taking advantage of cell culture techniques to support the subject assays. For example, as described below, a complex can be constituted in a eukaryotic cell culture system, including mammalian and yeast cells. Advantages to generating the subject assays in an intact cell include the ability to detect compounds that are functional in an environment more closely analogous to that for therapeutic use of the compounds. Furthermore, certain of the in vivo embodiments of the assay, such as examples given below, are amenable to high through-put analysis of candidate compounds.

In certain in vitro embodiments of the present assay, a reconstituted complex comprises a reconstituted mixture of at least semi-purified proteins. By semi-purified, it is meant that the proteins utilized in the reconstituted mixture have been previously separated from other cellular proteins. For instance, in contrast to cell lysates, proteins involved in the complex formation are present in the mixture to at least 50% purity relative to all other proteins in the mixture, in one embodiment are present at 90-95% purity, and in a further embodiment are present at 95-99% purity. In certain embodiments of the subject method, the reconstituted protein mixture is derived by mixing highly purified proteins such that the reconstituted mixture substantially lacks other proteins (such as of cellular origin) that might interfere with or otherwise alter the ability to measure the complex assembly and/or disassembly.

In certain embodiments, assaying in the presence and absence of a candidate compound, can be accomplished in any vessel suitable for containing the reactants. Examples include microtitre plates, test tubes and micro-centrifuge tubes.

In certain embodiments, drug screening assays can be generated which detect test antibodies on the basis of their ability to interfere with assembly, stability or function of a complex between a RAGE-BP (e.g., HMGB1, AGE, Aβ, SAA, S100, amphoterin, S100P, S100A, S100A4, A100A8, S100A9, CRP, β2-integrin, Mac-1, and p150,95) and a receptor polypeptide (e.g., RAGE or RAGE-LBE). In an exemplary binding assay, the compound of interest is contacted with a mixture comprising a RAGE-LBE polypeptide and a RAGE-BP such as HMGB1, AGE, Aβ, SAA, S100, amphoterin, S100P, S100A, S100A4, A100A8, S100A9, CRP, β2-integrin, Mac-1, and p150,95. Detection and quantification of the complex provide a means for determining the compound's efficacy at inhibiting interaction between the two components of the complex. The efficacy of the compound can be assessed by generating dose response curves from data obtained using various concentrations of the test antibody. Moreover, a control assay can also be performed to provide a baseline for comparison. In the control assay, the formation of complexes is quantitated in the absence of the test antibody.

In certain embodiments, association between the two polypeptides in a complex (e.g., a RAGE-BP and a receptor polypeptide), may be detected by a variety of techniques, many of which are effectively described above. For instance, modulation in the formation of complexes can be quantitated using, for example, detectably labeled proteins (e.g., radiolabeled, fluorescently labeled, or enzymatically labeled), by immunoassay, by two-hybrid assay, or by chromatographic detection. Surface plasmon resonance systems, such as those available from Biacore International AB (Uppsala, Sweden), may also be used to detect protein-protein interaction.

In certain embodiments, one polypeptide in a complex comprising a RAGE BP and a receptor polypeptide, can be immobilized to facilitate separation of the complex from uncomplexed forms of the other polypeptide, as well as to accommodate automation of the assay. In an illustrative embodiment, an antibody can be provided which adds a domain that permits the antibody to be bound to an insoluble matrix. For example, an antibody can be absorbed onto glutathione sepharose beads (Sigma Chemical, St. Louis, Mo.) or glutathione derivatized microtitre plates, or directly or indirectly attached to magnetic beads, which are then combined with a potential interacting protein (e.g., an ³⁵S-labeled S100 polypeptide, or other labeled RAGE-BP), and the test antibody are incubated under conditions conducive to complex formation. Following incubation, the beads are washed to remove any unbound interacting antibody, and the matrix bead-bound radiolabel determined directly (e.g., beads placed in scintillant), or in the supernatant after the complexes are dissociated, e.g., when microtitre plate is used. Alternatively, after washing away unbound antibody, the complexes can be dissociated frown the matrix, separated by SDS-PAGE gel, and the level of interacting polypeptide found in the matrix-bound fraction quantitated from the gel using standard electrophoretic techniques.

In another embodiment, a two-hybrid assay (also referred to as an interaction trap assay) can be used for detecting the interaction of two polypeptides in the complex of RAGE-LBE and RAGE-BP (see also, U.S. Pat. No. 5,283,317; Zervos et al. (1993) Cell 72: 223-232; Madura et al. (1993) J Biol Chem 268: 12046-12054; Bartel et al. (1993) Biotechniques 14: 920-924; and Iwabuchi et al. (1993) Oncogene 8: 1693-1696), and for subsequently detecting test antibodies which inhibit binding between a RAGE-LBE and a RAGE-BP polypeptide. This assay includes providing a host cell, for example, a yeast cell (preferred), a mammalian cell or a bacterial cell type. The host cell contains a reporter gene having a binding site for the DNA-binding domain of a transcriptional activator used in the bait protein, such that the reporter gene expresses a detectable gene product when the gene is transcriptionally activated. A first chimeric gene is provided which is capable of being expressed in the host cell, and encodes a “bait” polypeptide. A second chimeric gene is also provided which is capable of being expressed in the host cell, and encodes the “fish” polypeptide. In one embodiment, both the first and the second chimeric genes are introduced into the host cell in the form of plasmids. Preferably, however, the first chimeric gene is present in a chromosome of the host cell and the second chimeric gene is introduced into the host cell as part of a plasmid.

In certain embodiments, the invention provides a two-hybrid assay to identify test antibodies that inhibit the binding of a RAGE-BP polypeptide (e.g., HMGB1, AGE, Aβ, SAA, S100, amphoterin, S100P, S100A, S100A4, A100A8, S100A9, CRP, β2-integrin, Mac-1, and p150,95) and a receptor polypeptide (e.g., RAGE or RAGE-LBE). To illustrate, a “bait” polypeptide comprising a receptor polypeptide and a “fish” polypeptide comprising a RAGE-BP polypeptide (such as HMGB1, AGE, Aβ, SAM, S100, amphoterin, S100P, S100A, S100A4, A100A8, S100A9, CRP, β2-integrin, Mac-1, and p150,95), are introduced in the host cell. In one embodiment, the bait comprises the V-domain of human or murine RAGE, or a sequence with 80 to 99% identity to the V-domain of human or murine RAGE that can still bind RAGE-BP. Cells are subjected to conditions under which the bait and fish polypeptides are expressed in sufficient quantity for the reporter gene to be activated.

The interaction of the two fusion polypeptides results in a detectable signal produced by the expression of the reporter gene. Accordingly, the level of interaction between the two polypeptides in the presence of a test antibody and in the absence of the test antibody can be evaluated by detecting the level of expression of the reporter gene in each case. Various reporter constructs may be used in accord with the methods of the invention and include, for example, reporter genes which produce such detectable signals as selected front the group consisting of an enzymatic signal, a fluorescent signal, a phosphorescent signal and drug resistance.

In many drug screening programs that test libraries of compounds and natural extracts, high throughput assays are desirable in order to maximize the number of compounds surveyed in a given period of time. Assays of the present invention which are performed in cell-free systems, such as may be developed with purified or semi-purified proteins or with lysates, are often preferred as “primary” screens in that they can be generated to permit rapid development and relatively easy detection of an alteration in a molecular target which is mediated by a test antibody. Moreover, the effects of cellular toxicity and/or bioavailability of the test antibody can be generally ignored in the in vitro system, the assay instead being focused primarily on the effect of the drug on the molecular target as may be manifest in an alteration of binding affinity with other proteins or changes in enzymatic properties of the molecular target.

In certain embodiments, a complex formation between a RAGE-BP and a receptor may be assessed by immunoprecipitation and analysis of co-immunoprecipitated proteins or affinity purification and analysis of co-purified proteins. Fluorescence Resonance Energy Transfer (FRET)-based assays may also be used to determine such complex formation. Fluorescent molecules having the proper emission and excitation spectra that are brought into close proximity with one another can exhibit FRET. The fluorescent molecules are chosen such that the emission spectrum of one of the molecules (the donor molecule) overlaps with the excitation spectrum of the other molecule (the acceptor molecule). The donor molecule is excited by light of appropriate intensity within the donor's excitation spectrum. The donor then emits the absorbed energy as fluorescent light. The fluorescent energy it produces is quenched by the acceptor molecule. FRET can be manifested as a reduction in the intensity of the fluorescent signal from the donor, reduction in the lifetime of its excited state, and/or re-emission of fluorescent light at the longer wavelengths (lower energies) characteristic of the acceptor. When the fluorescent proteins physically separate, FRET effects are diminished or eliminated (see, for example, U.S. Pat. No. 5,981,200).

The occurrence of FRET also causes the fluorescence lifetime of the donor fluorescent moiety to decrease. This change in fluorescence lifetime can be measured using a technique termed fluorescence lifetime imaging technology (FLIM) (Verveer et al. (2000) Science 290: 1567-1570, Squire et al. (1999) J: Microsc. 193: 36; Verveer et al. (2000) Biophys. J. 78: 2127). Global analysis techniques for analyzing FLIM data have been developed. These algorithms use the understanding that the donor fluorescent moiety exists in only a limited number of states each with a distinct fluorescence lifetime. Quantitative maps of each state can be generated on a pixel-by-pixel basis.

To perform FRET-based assays, a RAGE-BP polypeptide (e.g., HMGB1, AGE, Aβ, SAA, S100, amphoterin, SLOOP, S100A, S100A4, A100A8, S100A9, CRP, β2-integrin, Mac-1, and p150,95) and a receptor polypeptide (e.g., RAGE or RAGE-LBE) are both fluorescently labeled. Suitable fluorescent labels are well known in the art. Examples are provided below, but suitable fluorescent labels not specifically discussed are also available to those of skill in the art and may be used. Fluorescent labeling may be accomplished by expressing a polypeptide as a polypeptide with a fluorescent protein, for example fluorescent proteins isolated from jellyfish, corals and other coelenterates. Exemplary fluorescent proteins include the many variants of the green fluorescent protein (GFP) of Aequoria victoria. Variants may be brighter, dimmer, or have different excitation and/or emission spectra. Certain variants are altered such that they no longer appear green, and may appear blue, cyan, yellow or red (termed BFP, CFP, YFP, and REP, respectively). Fluorescent proteins may be stably attached to polypeptides through a variety of covalent and noncovalent linkages, including, for example, peptide bonds (e.g., expression as a fusion protein), chemical cross-linking and biotin-streptavidin coupling. For examples of fluorescent proteins, see U.S. Pat. Nos. 5,625,048, 5,777,079, 6,066,476, and 6,124,128, Prasher et al. (1992) Gene, 111: 229-233; Reign et al. (1994) Proc. Natl. Acad. Sci., USA, 91: 12501-04; Ward et al. (1982) Photochem. Photobiol., 35: 803-808; Levine et al. (1982) Comp. Biochem. Physiol., 72B: 77-g5; Tersikh et al. (2000) Science 290: 1585-88.

FRET-based assays may be used in cell-based assays and in cell-free assays. FRET-based assays are amenable to high-throughput screening methods including Fluorescence Activated Cell Sorting and fluorescent scanning of microtiter arrays.

In general, where a screening assay is a binding assay (whether protein-protein binding, compound-protein binding, etc.), one or more of the molecules may be coupled or linked to a label, where the label can directly or indirectly provide a detectable signal. Various labels include radioisotopes, fluorescers, chemiluminescers, enzymes, specific binding molecules, particles, e.g., magnetic particles, and the like. Specific binding molecules include pairs, such as biotin and streptavidin, digoxin and antidigoxin, etc. For the specific binding members, the complementary member would normally be labeled with a molecule that provides for detection, in accordance with known procedures.

A variety of other reagents may be included in the screening assay. These include reagents like salts, neutral proteins, e.g., albumin, detergents, etc that are used to facilitate optimal protein-protein binding and/or reduce nonspecific or battleground interactions. Reagents that improve the efficiency of the assay, such as protease inhibitors, nuclease inhibitors, anti-microbial compounds, etc. may be used. The mixture of components are added in any order that provides for the requisite binding. Incubations are performed at any suitable temperature, typically between 4° C. and 40° C. Incubation periods are selected for optimum activity, but may also be optimized to facilitate rapid high-throughput screening.

In certain embodiments, the invention provides complex-independent assays. Such assays comprise identifying a test antibody that is a candidate inhibitor of the binding of a RAGE-BP to a receptor polypeptide (e.g., RAGE or RAGE-LBE).

In an exemplary embodiment, a compound that binds to a receptor polypeptide may be identified by using an receptor RAGE-LBE polypeptide. In an illustrative embodiment, a RAGE-LBE can be provided which adds an additional domain that permits the protein to be bound to an insoluble matrix. For example, a RAGE-LBE fused with a GST protein can be adsorbed onto glutathione sepharose beads (Sigma Chemical, St. Louis, Mo.) or glutathione derivatized microtitre plates, which are then combined with a potential labeled binding compound and incubated under conditions conducive to binding. Following incubation, the beads are washed to remove any unbound compound, and the matrix bead-bound label determined directly, or in the supernatant after the bound compound is dissociated.

In certain embodiments, a label can directly or indirectly provide a detectable signal. Various labels include radioisotopes, fluorescers, chemiluminescers, enzymes, specific binding molecules, particles, e.g., magnetic particles, and the like. Specific binding molecules include pairs, such as biotin and streptavidin, digoxin and antidigoxin etc. For the specific binding members, the complementary member would normally be labeled with a molecule that provides for detection, in accordance with known procedures. In certain embodiments, such methods comprise forming the mixture in vitro. In certain embodiments, such methods comprise cell-based assays by forming the mixture in vivo. In certain embodiments, the methods comprise contacting a cell that expresses a receptor polypeptide (e.g., RAGE or RAGE-LBE) or a variant thereof with the test antibody.

In certain embodiments, assays are based on cell-free systems, e.g., purified proteins or cell lysates, as well as cell-based assays that utilize intact cells. Simple binding assays can be used to detect compounds that interact with the receptor polypeptide. Compounds to be tested can be produced, for example, by bacteria, yeast or other organisms (e.g., natural products), produced chemically (e.g., small molecules, including peptidomimetics), or produced recombinantly.

Optionally, test antibodies identified from these assays may be used to treat RAGE-associated disorders.

Pharmaceutical Preparations

The subject proteins or nucleic acids of the present invention are most preferably administered in the form of appropriate compositions. As appropriate compositions there may be cited all compositions usually employed for systemically or locally administering drugs. The pharmaceutically acceptable carrier should be substantially inert, so as not to act with the active component. Suitable inert carriers include water, alcohol, polyethylene glycol, mineral oil or petroleum gel, propylene glycol, phosphate buffer saline (PBS), baceriostatic water for injection (BWFI), sterile water for injection (SWFI), and the like. Said pharmaceutical preparations (including the subject antibodies or nucleic acids encoding the subject antibodies) may be formulated for administration in any convenient way for use in human or veterinary medicine.

Thus, another aspect of the present invention provides pharmaceutically acceptable compositions comprising an effective amount of an antibody, formulated together with one or more pharmaceutically acceptable carriers (additives) and/or diluents. As described in detail below, the pharmaceutical compositions of the present invention may be specially formulated for administration in solid or liquid form, including those adapted for the following: (1) oral administration, for example, drenches (aqueous or non-aqueous solutions or suspensions), tablets, boluses, powders, granules, pastes for application to the tongue; (2) parenteral administration, for example, by subcutaneous, intramuscular or intravenous injection as, for example, a sterile solution or suspension; (3) topical application, for example, as a cream, ointment or spray applied to the skin; or (4) intravaginally or intrarectally, for example, as a pessary, cream or foam. However, in certain embodiments the subject agents may be simply dissolved or suspended in sterile water. In certain embodiments, the pharmaceutical preparation is non-pyrogenic, i.e., does not elevate the body temperature of a patient. Parenteral administration, in particular subcutaneous and intravenous injection, is the preferred route of administration.

In certain embodiments, one or more agents may contain a basic functional group, such as amino or alkylamino, and are, therefore, capable of forming pharmaceutically acceptable salts with pharmaceutically acceptable acids. The term “pharmaceutically acceptable salts” in this respect, refers to the relatively non-toxic, inorganic and organic acid addition salts of compounds of the present invention. These salts can be prepared in situ during the final isolation and purification of the compounds of the invention, or by separately reacting a purified compound of the invention in its free base form with a suitable organic or inorganic acid, and isolating the salt thus formed. Representative salts include the hydrobromide, hydrochloride, sulfate, bisulfate, phosphate, nitrate, acetate, valerate, oleate, palmitate, stearate, laurate, benzoate, lactate, phosphate, tosylate, citrate, maleate, fumarate, succinate, tartrate, napthylate, mesylate, glucoheptonate, lactobionate, and laurylsulphonate salts and the like. (See, for example, Berge et al. (1977) “Pharmaceutical Salts,” J. Pharm. Sci. 66: 1-19).

The pharmaceutically acceptable salts of the agents include the conventional nontoxic salts or quaternary ammonium salts of the compounds, e.g., from non-toxic organic or inorganic acids. For example, such conventional nontoxic salts include those derived from inorganic acids such as hydrochloride, hydrobromic, sulfuric, sulfamic, phosphoric, nitric, and the like; and the salts prepared from organic acids such as acetic, propionic, succinic, glycolic, stearic, lactic, malic, tartaric, citric, ascorbic, palmitic, maleic, hydroxymaleic, phenylacetic, glutamic, benzoic, salicyclic, sulfanilic, 2-acetoxybenzoic, fumaric, toluenesulfonic, methanesulfonic, ethane disulfonic, oxalic, isothionic, and the like.

In other cases, the one or more agents may contain one or more acidic functional groups and, thus, are capable of forming pharmaceutically acceptable salts with pharmaceutically acceptable bases. These salts can likewise be prepared in situ during the final isolation and purification of the compounds, or by separately reacting the purified compound in its free acid form with a suitable base, such as the hydroxide, carbonate or bicarbonate of a pharmaceutically acceptable metal cation, with ammonia, or with a pharmaceutically acceptable organic primary, secondary or tertiary amine. Representative alkali or alkaline earth salts include the lithium, sodium, potassium, calcium, magnesium, and aluminum salts and the like. Representative organic amines useful for the formation of base addition salts include ethylamine, diethylamine, ethylenediamine, ethanolamine, diethanolamine, piperazine and the like. (see, for example, Berge et al., supra)

Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

Examples of pharmaceutically acceptable antioxidants include: (1) water soluble antioxidants, such as ascorbic acid, cysteine hydrochloride, sodium bisulfate, sodium metabisulfite, sodium sulfite and the like, (2) oil-soluble antioxidants, such as ascorbyl palpitate, butylated hydroxyanisole (BHA), butylated hydroxytoluene (BHT), lecithin, propyl gallate, alpha-tocopherol, and the like, and (3) metal chelating agents, such as citric acid, ethylenediamine tetraacetic acid (EDTA), sorbitol, tartaric s acid, phosphoric acid, and the like.

Formulations of the present invention include those suitable for oral, nasal, topical (including buccal and sublingual), rectal, vaginal and/or parenteral administration. The formulations may conveniently be presented in unit dosage form and may be prepared by any methods well known in the art of pharmacy. The amount of active ingredient which can be combined with a carrier material to produce a single dosage form will vary depending upon the host being treated, the particular mode of administration, etc. The amount of active ingredient that can be combined with a carrier material to produce a single dosage form will generally be that amount of the compound that produces a therapeutic effect. Generally, out of one hundred percent, this amount will range frown about 1 percent to about ninety-nine percent of active ingredient, preferably from about 5 percent to about 70 percent, most preferably from about 10 percent to about 30 percent.

Methods of preparing these formulations or compositions include the step of bringing into association an agent with the carrier and, optionally, one or more accessory ingredients. In general, the formulations are prepared by uniformly and intimately bringing into association an agent of the present invention with liquid carriers, or timely divided solid carriers, or both, and then, if necessary, shaping the product.

Formulations of the invention suitable for oral administration may be in the form of capsules, cachets, pills, tablets, lozenges (using a flavored basis, usually sucrose and acacia or tragacanth), powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouth washes and the like, each containing a predetermined amount of a compound of the present invention as an active ingredient. A compound of the present invention may also be administered as a bolus, electuary or paste.

In solid dosage forms of the invention for oral administration (capsules, tablets, pills, dragees, powders, granules and the like), the active ingredient is mixed with one or more pharmaceutically acceptable carriers, such as sodium citrate or dicalcium phosphate, and/or any of the following: (1) fillers or extenders, such as starches, lactose, sucrose, glucose, mannitol, and/or silicic acid; (2) binders, such as, for example, carboxymethylcellulose, alginates, gelatin, polyvinyl pyrrolidone, sucrose and/or acacia; (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium compounds; (7) wetting agents, such as, for example, cetyl alcohol and glycerol monostearate; (8) absorbents, such as kaolin and bentonite clay; (9) lubricants, such as talc, calcium stearate, magnesium stearate, solid polyethylene glycols, sodium lauryl sulfate, and mixtures thereof; and (10) coloring agents. In the case of capsules, tablets and pills, the pharmaceutical compositions may also comprise buffering agents. Solid compositions of a similar type may also be employed as fillers in soft and hard-filled gelatin capsules using such excipients as lactose or milk sugars, as well as high molecular weight polyethylene glycols and the like.

A tablet may be made by compression or molding, optionally with one or more accessory ingredients. Compressed tablets may be prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets may be made by molding in a suitable machine a mixture of the powdered compound moistened with an inert liquid diluent.

The tablets, and other solid dosage forms of the pharmaceutical compositions of the present invention, such as dragees, capsules, pills and granules, may optionally be scored or prepared with coatings and shells, such as enteric coatings and other coatings well known in the pharmaceutical-formulating art. They may also be formulated so as to provide slow or controlled release of the active ingredient therein using, for example, hydroxypropylmethyl cellulose in varying proportions to provide the desired release profile, other polymer matrices, liposomes and/or microspheres. They may be sterilized by, for example, filtration through a bacteria-retaining filter, or by incorporating sterilizing agents in the form of sterile solid compositions that can be dissolved in sterile water, or some other sterile injectable medium immediately before use. These compositions may also optionally contain opacifying agents and may be of a composition that they release the active ingredient(s) only, or preferentially, in a certain portion of the gastrointestinal tract, optionally, in a delayed manner. Examples of embedding compositions that can be used include polymeric substances and waxes. The active ingredient can also be in micro-encapsulated form, if appropriate, with one or more of the above-described excipients.

Liquid dosage forms for oral administration of the compounds of the invention include pharmaceutically acceptable emulsions, microemulsions, solutions, suspensions, syrups and elixirs. In addition to the active ingredient, the liquid dosage forms may contain inert diluents commonly used in the art, such as, for example, water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof.

Besides inert diluents, the oral compositions can also include adjuvants such as wetting agents, emulsifying and suspending agents, sweetening, flavoring, coloring, perfuming and preservative agents.

Suspensions, in addition to the active compounds, may contain suspending agents as, for example, ethoxylated isostearyl alcohols, polyoxyethylene sorbitol and sorbitan esters, microcrystalline cellulose, aluminum metahydroxide, bentonite, agar and tragacanth, and mixtures thereof.

Formulations of the pharmaceutical compositions of the invention for rectal or vaginal administration may be presented as a suppository, which may be prepared by mixing one or more compounds of the invention with one or more suitable nonirritating excipients or carriers comprising, for example, cocoa butter, polyethylene glycol, a suppository wax or a salicylate, and which is solid at room temperature, but liquid at body temperature and, therefore, will melt in the rectum or vaginal cavity and release the agents.

Formulations of the present invention which are suitable for vaginal administration also include pessaries, tampons, creams, gels, pastes, foams or spray formulations containing such carriers as are known in the art to be appropriate.

Dosage forms for the topical or transdermal administration of a compound of this invention include powders, sprays, ointments, pastes, creams, lotions, gels, solutions, patches and inhalants. The active compound may be mixed under sterile conditions with a pharmaceutically acceptable carrier, and with any preservatives, buffers, or propellants that may be required.

The ointments, pastes, creams and gels may contain, in addition to an active compound of this invention, excipients, such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays can contain, in addition to a compound of this invention, excipients such as lactose, talc, silicic acid, aluminum hydroxide, calcium silicates and polyamide powder, or mixtures of these substances. Sprays can additionally contain customary propellants, such as chlorofluorohydrocarbons and volatile unsubstituted hydrocarbons, such as butane and propane.

Transdermal patches have the added advantage of providing controlled delivery of a compound of the present invention to the body. Such dosage forms can be made by dissolving or dispersing the agents in the proper medium. Absorption enhancers can also be used to increase the flux of the agents across the slain. The rate of such flux can be controlled by either providing a rate controlling membrane or dispersing the compound in a polymer matrix or gel.

Ophthalmic formulations, eye ointments, powders, solutions and the like, are also contemplated as being within the scope of this invention.

Pharmaceutical compositions of this invention suitable for parenteral administration comprise one or more compounds of the invention in combination with one or more pharmaceutically acceptable sterile isotonic aqueous or nonaqueous solutions, dispersions, suspensions or emulsions, or sterile powders which may be reconstituted into sterile injectable solutions or dispersions just prior to use, which may contain antioxidants, buffers, bacteriostats, solutes which render the formulation isotonic with the blood of the intended recipient or suspending or thickening agents.

Examples of suitable aqueous and nonaqueous carriers that may be employed in the pharmaceutical compositions of the invention include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions may also contain adjuvants such as preservatives, wetting agents, emulsifying agents and dispersing agents. Prevention of the action of microorganisms may be ensured by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents that delay absorption such as aluminum monostearate and gelatin.

In some cases, in order to prolong the effect of an agent, it is desirable to slow the absorption of the agent from subcutaneous or intramuscular injection. This may be accomplished by the use of a liquid suspension of crystalline or amorphous material having poor water solubility. The rate of absorption of the agent then depends upon its rate of dissolution, which, in turn, may depend upon crystal size and crystalline form. Alternatively, delayed absorption of a parenterally administered agent is accomplished by dissolving or suspending the agent in an oil vehicle.

Injectable depot forms are made by forming microencapsule matrices of the subject compounds in biodegradable polymers such as polylactide-polyglycolide. Depending on the ratio of agent to polymer, and the nature of the particular polymer employed, the rate of agent release can be controlled. Examples of other biodegradable polymers include poly(orthoesters) and poly(anhydrides). Depot injectable formulations are also prepared by entrapping the agent in liposomes or microemulsions that are compatible with body tissue.

When the compounds of the present invention are administered as pharmaceuticals, to humans and animals, they can be given per se or as a pharmaceutical composition containing, for example, 0.1 to 99.5% (more preferably, 0.5 to 90%) of active ingredient in combination with a pharmaceutically acceptable carrier.

Apart from the above-described compositions, use may be made of covers, e.g., plasters, bandages, dressings, gauze pads and the like, containing an appropriate amount of a therapeutic. As described in detail above, therapeutic compositions may be administered/delivered on stems, devices, prosthetics, and implants.

The tissue sample for analysis is typically blood, plasma, serum, mucous fluid or cerebrospinal fluid from the patient. The sample is analyzed, for example, for levels or profiles of antibodies to RAGE peptide, e.g., levels or profiles of humanized antibodies. ELISA methods of detecting antibodies specific to RAGE are described in the Examples.

The antibody profile following passive immunization typically shows an immediate peak in antibody concentration followed by an exponential decay. Without a further dosage, the decay approaches pretreatment levels within a period of days to months depending on the half-life of the antibody administered.

In some methods, a baseline measurement of antibody to RAGE in the patient is made before administration, a second measurement is made soon thereafter to determine the peak antibody level, and one or more further measurements are made at intervals to monitor decay of antibody levels. When the level of antibody has declined to baseline or a predetermined percentage of the peak less baseline (e.g., 50%, 25% or 10%), administration of a further dosage of antibody is administered. In some methods, peak or subsequent measured levels less background are compared with reference levels previously determined to constitute a beneficial prophylactic or therapeutic treatment regime in other patients. If the measured antibody level is significantly less than a reference level (e.g., less than the mean minus one standard deviation of the reference value in population of patients benefiting from treatment) administration of an additional dosage of antibody is indicated.

### EXAMPLES

The invention now being generally described, it will be more readily understood by reference to the following examples, which are included merely for purposes of illustration of certain aspects and embodiments of the present invention, and are not intended to limit the invention.

### Example 1

### Preparation of RAGE Constructs

The amino acid sequences of murine RAGE (mRAGE, Genbank accession no. NP_{—}031451; SEQ ID NO: 3) and human RAGE (hRAGE, Genbank accession no. NP_{—}00127.1; SEQ ID NO: 1) are shown in FIG. 1A-1C. Full length cDNAs encoding mRAGE (accession no. NM_{—}007425.1; SEQ ID NO: 4) and hRAGE (accession no. NM_{—}001136; SEQ ID NO: 2) were inserted into the Adori1-2 expression vector, which comprises a cytomegalovirus (CMV) promoter driving expression of the cDNA sequences, and contains adenovirus elements for virus generation. A human RAGE-Fc fusion protein formed by appending amino acids 1-344 of human RAGE to the Fc domain of human IgG was prepared by expressing a DNA construct encoding the fusion protein in cultured cells using the Adori expression vector. A human RAGE V-region-Fc fusion protein formed by appending amino acids 1-118 of human RAGE to the Fc domain of human IgG was similarly prepared. Human and murine RAGE-strep tag fusion proteins formed by appending a streptavidin (strep) tag sequence (WSHPQFEK) (SEQ ID NO: 5) to amino acids 1-344 of human or murine RAGE, respectively, were prepared by expressing DNA constructs encoding the RAGE-strep tag fusion proteins, also using Adori expression vectors. All constructs were verified by extensive restriction digest analyses and by sequence analyses of cDNA inserts within the plasmids

Recombinant adenovirus (Ad5 E1a/E3 deleted) expressing the full-length RAGE, hRAGE-Fc, and hRAGE V-domain-Fc were generated by homologous recombination in a human embryonic kidney cell line 293 (HEK293) (ATCC, Rockland Md.). Recombinant adenovirus virus was isolated and subsequently amplified in HEK293 cells. The virus was released from infected HEK293 cells by three cycles of freeze thawing. The virus was further purified by two cesium chloride centrifugation gradients and dialyzed against phosphate buffered saline (PBS) pH 7.2 at 4° C. Following dialysis, glycerol was added to a concentration of 10% and the virus was stored at −80° C. until use. Viral constructs were characterized for infectivity (plaque forming units on 293 cells), PCR analysis of the virus, sequence analysis of the coding region, expression of the transgene, and endotoxin measurements.

Adori expression vectors containing DNA encoding human RAGE-Fc, human RAGE-V region-Fc, and human and murine RAGE-strep tag fusion proteins were stably transfected into Chinese Hamster Ovary (CHO) cells using lipofectin (Invitrogen). Stable transfectants were selected in 20 nM and 50 nM methotrexate. Conditioned media were harvested from individual clones and analyzed with the use of sodium dodecyl sulfate-polyacrylamide gel electrophoresis (SDS-PAGE) and Western blotting to confirm RAGE expression. (Kaufman, R. J., 1990, Methods in Enzymology, 185:537-66; Kaufman, R. J., 1990, Methods in Enzymology, 185:487-511; Pittman, D. D. et al., 1993, Methods in Enzymology, 222: 236-237).

CHO or transduced HEK 293 cells expressing soluble RAGE fusion proteins were cultured to harvest conditioned medium for protein purification. Proteins were purified with the use of indicated affinity-tag methods. Purified proteins were subjected to reducing and non-reducing SDS-PAGE, visualized by Coomassie Blue staining (Current Protocols in Protein Sciences, Wiley Interscience), and shown to be of the expected molecular weights.

### Example 2

### Generation of Murine Anti-RAGE Monoclonal Antibodies

6-8 week old female BALB/c mice (Charles River, Andover, Mass.) were immunized subcutaneously with the use of a GeneGun device (BioRad, Hercules, Calif.). The pAdori expression vector containing cDNA encoding full-length human RAGE was pre-absorbed onto colloidal gold particles (BioRad, Hercules, Calif.) before subcutaneous administration. Mice were immunized with 3 ug of vector twice per week, for two weeks. Mice were bled one week after the last immunization and antibody titers were evaluated. The mouse with highest RAGE-antibody titer received one additional injection of 10 μg of recombinant human RAGE-strep protein three days before cell fusion.

Splenocytes were fused with mouse myeloma cells P3X63Ag8.653 (ATCC, Rockville, Md.) at a 4:1 ratio using 50% polyethylene glycol (MW 1500) (Roche Diagnostics Corp, Mannheim, Germany). After fusion, cells were seeded and cultured in 96-well plates at 1×10⁵ cells/well in the RPMI1640 selection medium, containing 20% FBS, 5% Origen (IGEN International Inc. Gaithersburg, Md.), 2 mM L-glutamine, 100 U/ml penicillin, 100 μg/ml streptomycin, 10 mM HEPES and 1× hypoxanthine-aminopterin-thymidine (Sigma, St. Louis, Mo.).

### Example 3

### Generation of Rat Anti-RAGE Monoclonal Antibodies

LOU rats (Harlan, Harlan, Mass.) rats were immunized subcutaneously with the use of a GeneGun (BioRad, Hercules, Calif.). The pAdori expression vector containing cDNA encoding full-length murine RAGE was pre-absorbed onto colloidal gold particles (BioRad, Hercules, Calif.) before subcutaneous administration. Rats were immunized with 3 ug of vector once every two weeks for four times. Rats were bled one week after the last immunization and antibody titers were evaluated. The rat with highest RAGE-antibody titer received one additional injection of 10 μg of recombinant murine RAGE-strep protein three days before cell fusion.

Splenocytes were fused with mouse myeloma cells P3X63Ag8.653 (ATCC, Rockville, Md.) at a 4:1 ratio using 50% polyethylene glycol (MW 1500) (Roche Diagnostics Corp, Mannheim, Germany). After fusion, cells were seeded and cultured in 96-well plates at 1×10⁵ cells/well in the RPMI1640 selection medium, containing 20% FBS, 5% Origen (IGEN International Inc. Gaithersburg Md.), 2 mM L-glutamine, 100 U/ml penicillin, 100 μg/ml streptomycin, 10 mM HEPES and 1× hypoxanthine-aminopterin-thymidine (Sigma, St. Louis, Mo.).

### Example 4

### Hybridoma Screening

Panels of rat anti-murine RAGE and murine anti-human RAGE mAbs were generated by cDNA immunization using the GeneGun, and the Adori expression plasmids expressing the full-length coding region of murine or human RAGE. Hybridoma supernatants were screened for binding to recombinant human or murine RAGE-Fc by ELISA and by FACS analysis on human embryonic kidney cells (HEK-293) transiently expressing RAGE. Positive supernatants were further tested for their ability to neutralize RAGE binding to the ligand HMGB1. Seven rat monoclonal antibodies (XT-M series) and seven mouse monoclonal antibodies (XT-H series) were identified. Selected hybridomas were subcloned four times by serial dilution and once by FACS sorting. Conditioned media were harvested from the stable hybridoma cultures and immunoglobulins were purified using Protein A antibody purification columns (Millipore Billerica, Mass.). The Ig class of each mAb was determined with a mouse mAb isotyping kit or rat mAb isotyping kit as indicated (IsoStrip; Boehringer Mannheim Corp.). The isotypes of the selected rat and mouse monoclonal antibodies are set forth in Table 1 (below).

TABLE 1

| Rat monoclonal | | | Murine monoclonal | | |
|---|---|---|---|---|---|
| anti-muRAGE antibodies | | | anti-huRAGE antibodies | | |
| Hybridoma | | | Hybridoma | | |
| clones | Mabs | Ig isotypes | clones | Mabs | Ig isotypes |
| 1mRAGEP3/1* | XT-M1 | Rat IgG2a, k | 1hRAGEP3/6* | XT-H1 | Mouse IgG1, k |
| 1mRAGEP3/7 | XT-M2 | Rat IgG2b, k | 1hRAGEP3/16* | XT-H2 | Mouse IgG1, k |
| 1mRAGEP3/8 | XT-M3 | Rat IgG2a, k | 1hRAGEP3/18 | XT-H3 | Mouse IgG1, k |
| 1mRAGEP3/10* | XT-M4 | Rat IgG2b, k | 1hRAGEP3/48 | XT-H4 | Mouse IgG1, k |
| 1mRAGEP3/15 | XT-M5 | Rat IgG2a, k | 1hRAGEP3/55* | XT-H5 | Mouse IgG1, k |
| 1mRAGEP3/16 | XT-M6 | Rat IgG2b, k | 1hRAGEP3/65 | XT-H6 | Mouse IgG1, k |
| 1mRAGEP3/18* | XT-M7 | Rat IgG2b, k | 1hRAGEP3/66 | XT-H7 | Mouse IgG1, k |

### Example 5

### FACS Analysis

Human 293 cells were infected with the human and murine RAGE adenovirus. Infected cells were suspended in PBS containing 1% BSA at a density of 4×10⁴ cells/ml. Cells were incubated with 100 ul of sample (diluted immune sera, hybridoma supernatants or purified antibodies) for 30 min at 4° C. After washing, cells were incubated with PE-labeled goat, anti-mouse, IgG, F(ab′)₂ (DAKO Corporation GlostrupDenmark) for 30 min at 4° C. in the dark. Cell-associated fluorescence signals were measured by a FACScan flow cytofluorometer (Becton Dickinson) using 5000 cells per treatment. Propidium iodide was used to identify dead cells, which were excluded from the analysis. The seven murine monoclonal antibodies XT-H1 to XT-H7 and the seven rat monoclonal antibodies XT-M1 to XT-M7 were shown by FACS analysis to bind to cell-surface hRAGE (Table 2).

### Example 6

### ELISA Binding Assay

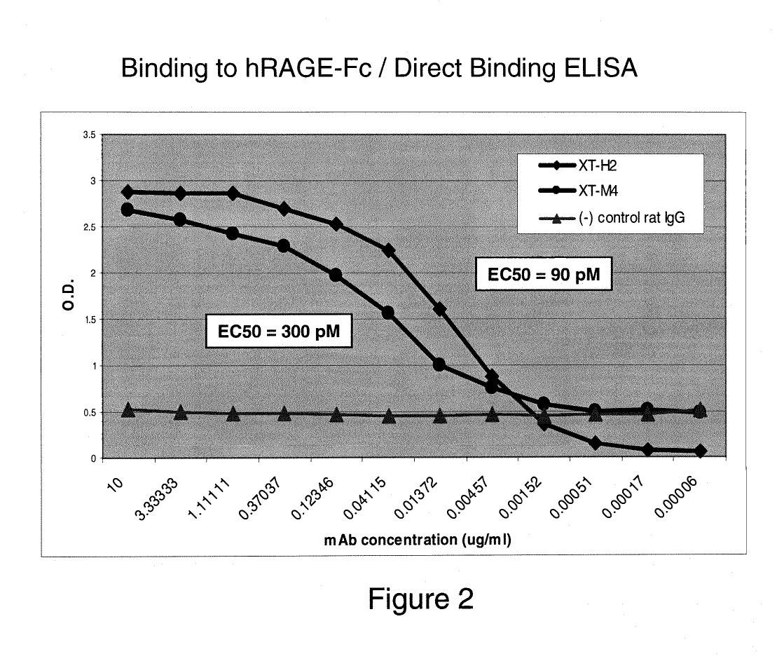

Antibodies were purified from hybridoma supernatants using standard procedures. Purified antibodies were evaluated for binding to soluble forms of RAGE with the use of ELISA. Ninety-six well plates (Corning, Corning, N.Y.) were coated with 100 ul of recombinant human RAGE-Fc or recombinant human RAGE V-region-Fc (1 μg/ml) and incubated overnight at 4° C. After washing and blocking with PBS containing 1% BSA and 0.05% Tween-20, 100 ul of sample (samples were in several forms: diluted immune serum, hybridoma supernatants, or purified antibodies, as indicated) was added and incubated for 1 hour at room temperature. The plates were washed with PBS, pH 7.2 and bound anti-RAGE antibodies were detected with the use of peroxidase-conjugated goat, anti-mouse IgG (H+L) (IgG) (Pierce, Rockford, Ill.) followed by incubation with the substrate TMB (BioFX Laboratories Owings Mills, Md. Laboratories). Absorbance values were determined at 450 nm in a spectrophotometer. The concentrations of monoclonal antibodies were determined with the use of peroxidase-labeled goat, anti-mouse IgG (Fcγ) (Pierce Rockford, Ill.) and a standard curve was generated by a purified, isotype-matched mouse IgG. ELISA results for the abilities of the seven murine antibodies XT-H1 to XT-H7 and the seven rat antibodies XT-M1 to XT-M7 to bind to hRAGE-Fc, hRAGE V-region-Fc, mRAGE-Fc, and mRAGE-strep, are summarized in Table 2. As shown in FIGS. 2 and 3, rat antibody XT-M4 and murine antibody XT-H2 both bind to human RAGE-Fc and to the V-domain of hRAGE. The EC50 values for binding of XT-M4 to human RAGE and to human RAGE V-domain were 300 pM and 100 pM, respectively. The EC50 values for binding of XT-H2 to human RAGE and human RAGE V-domain were 90 pM and 100 pM, respectively.

TABLE 2

| | | | | | ELISA | ELISA |
|---|---|---|---|---|---|---|
| | FACS | FACS | ELISA | ELISA | mRAGE- | hRAGE- |
| Mabs | hRAGE-Fc | mRAGE-Fc | hRAGE-Fc | mRAGE-Fc | strep | V-Fc (CM) |
| XT-H1 | + | + | +++ | − | + | − |
| XT-H2 | + | − | +++ | − | − | ++ |
| XT-H3 | + | − | +++ | | − | |
| XT-H4 | + | − | +++ | | − | |
| XT-H5 | + | − | +++ | − | − | ++ |
| XT-H6 | + | − | +++ | − | − | |
| XT-H7 | + | − | +++ | − | +/− | |
| XT-M1 | − | + | − | +++ | +++ | +++ |
| XT-M2 | + | + | ++ | + | + | + |
| XT-M3 | + | + | − | | | |
| XT-M4 | + | + | ++ | + | + | + |
| XT-M5 | − | + | − | | | |
| XT-M6 | + | + | ++ | + | + | + |
| XT-M7 | + | + | ++ | +++ | +++ | +++ |

### Example 7

### RAGE Ligand and Antibody Competition ELISA Binding Assays

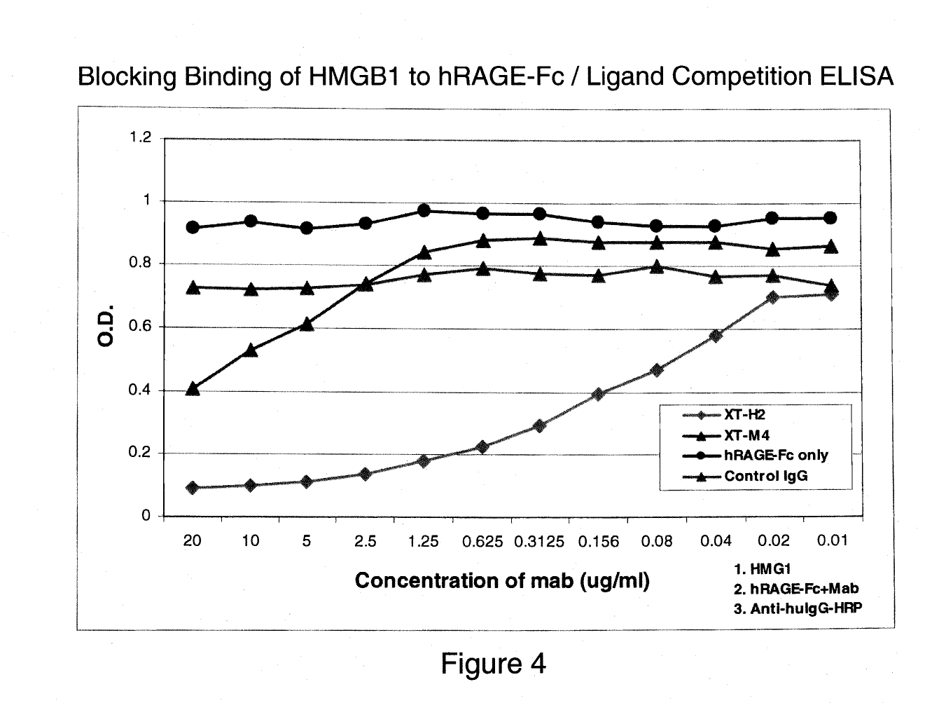

To determine whether RAGE monoclonal antibodies affect the binding of a RAGE ligand (HMGB1; Sigma, St. Louis, Mo.) to RAGE, competition ELISA binding assays were performed. Ninety-six well plates were coated with 1 μg/ml of HMGB1 overnight at 4° C. Wells were washed and blocked as described above and exposed to 100 μl of pre-incubated mixtures of RAGE-Fc or TrkB-Fc (a non-specific Fc control), at 0.1 μg/ml, plus various forms of the indicated antibody preparation (dilutions of immune sera, hybridoma supernatants or purified antibodies) for 1 hour at room temperature. Plates were washed with PBS, pH 7.2 and ligand-bound recombinant human RAGE-Fc was detected with the use of peroxidase-conjugated goat, anti-human IgG (Fcγ) (Pierce, Rockford, Ill.), followed by incubation with the substrate TMB (BioFX Laboratories Owings Mills, Md. Laboratories Owings Mills, Md.). Binding of recombinant human RAGE-Fc to ligand without any antibodies or with diluted pre-immune serum was used as a control and defined as 100% binding. The abilities of the seven murine antibodies XT-H1 to XT-H7 and the seven rat antibodies XT-M1 to XT-M7 to block the binding of HMGB1 to hRAGE-Fc as determined by the competition ELISA binding assay are shown in Table 3. Table 3 also summarizes the abilities of murine antibodies XT-H1, XT-H2, and XT-H5 to block the binding to RAGE of a different ligand of hRAGE, amyloid β 1-42 peptide, and the abilities of rat antibodies XT-M1 to XT-M7 to block the binding of HMGB1 to murine RAGE-Fc, as determined by similar competition ELISA binding assays. As shown in FIG. 4, rat antibody XT-M4 and murine antibody XT-H2 both block the binding of HMGB1 to human RAGE.

TABLE 3

| | RAGE ligand competition ELISA | | | Antibody compe- |
|---|---|---|---|---|
| | binding assays | | | tition ELISA |
| | | hRAGE-Fc + | | binding assays |
| | hRAGE-Fc + | Aβ 1-42 | mRAGE-Fc + | ELISA hRAGE- |
| Mabs | HMGB1 | peptide | HMGB1 | V-Fc (CM) |
| XT-H1 | − | + | | |
| XT-H2 | + | +++ | | |
| XT-H3 | − | | | |
| XT-H4 | +/− | | | |
| XT-H5 | + | +++ | | |
| XT-H6 | − | | | |
| XT-H7 | +/− | | | |
| XT-M1 | − | | − | − |
| XT-M2 | + | | + | XT-H3 & XT-H7 |
| | | | | compete |
| XT-M3 | − | | − | |
| XT-M4 | ++ | | + | XT-H2 & XT-H7 |
| | | | | compete |
| XT-M5 | − | | − | |
| XT-M6 | + | | + | − |
| XT-M7 | + | | + | − |

A similar competition approach was used to determine the relative binding epitopes between pairs of antibodies. First, 1 μg/ml of recombinant human RAGE-Fc was coated on ninety six-well plates over night at 4° C. After washing and blocking (see above) wells were exposed to 100 μl of pre-incubated mixtures of biotinylated target antibody and dilutions of a competing antibody for 1 hour at room temperature. Bound biotinylated antibody was detected using peroxidase-conjugated streptavidin (Pierce, A similar competition approach was used to determine the relative binding epitopes between pairs of antibodies. First, 1 μg/ml of recombinant human RAGE-Fc was coated on ninety six-well plates over night at 4° C. After washing and blocking (see above) wells were exposed to 100 μl of pre-incubated mixtures of biotinylated target antibody and dilutions of a competing antibody for 1 hour at room temperature. Bound biotinylated antibody was detected using peroxidase-conjugated streptavidin (Pierce, Rockford, Ill.) followed by incubation with the substrate TMB (BioFX Laboratories Owings Mills, Md. Laboratories). Binding of biotinylated antibody to recombinant human RAGE-Fc without any competing antibodies was used as a control and defined as 100%. Results of competition ELISA binding assays analyzing the competition between rat and murine antibodies for binding to hRAGE are shown in Table 3. FIG. 5 present a graph of data from competition ELISA binding assays analyzing the competition between rat XT-M4 and antibodies XT-H1, XT-H2, XT-H5, XT-M2, XT-M4, XT-M6, and XT-M7 for binding to hRAGE. The competition ELISA binding data shown in FIG. 5 demostrate that XT-M4 and XT-H2 bind to overlapping sites on human RAGE.

### Example 8

### BIACORE™ Binding Assays of Binding of Murine and Rat Anti-RAGE Antibodies to Human and Murine RAGE-Fc

A. Binding to Human and Murine RAGE

The binding of selected murine and rat anti-RAGE antibodies to human and murine RAGE and to the V domains of human and murine RAGE was analyzed by BIACORE® direct binding assay. Assays were performed using human or murine RAGE-Fc coated on a CM5 chip at high density (2000 RU) using standard amine coupling. Solution of the anti-RAGE antibodies at two concentrations, 50 and 100 nm, were run over the immobilized RAGE-Fc proteins in duplicate. BIACORE™ technology utilizes changes in the refractive index at the surface layer upon binding of the anti-RAGE antibodies to the immobilized RAGE antigen. Binding is detected by surface plasmon resonance (SPR) of laser light refracting from the surface. Results of the BIACORE™ direct binding assays are summarized in Table 4.

TABLE 4

| Rat anti-muRAGE | | | Murine anti-huRAGE | | |
|---|---|---|---|---|---|
| antibodies | | | antibodies | | |
| | huRAGE- | muRAGE- | | huRAGE- | muRAGE- |
| Mabs | Fc | Fc | Mabs | Fc | Fc |
| XT-M1 | | +++ | XT-H1 | +++ | +/− |
| XT-M2 | + | ++ | XT-H2 | +++ | − |
| XT-M3 | | | XT-H3 | + | + |
| XT-M4 | +++ | +++ | XT-H4 | + | + |
| XT-M5 | | | XT-H5 | ++ | − |
| XT-M6 | ++ | +++ | XT-H6 | +++ | − |
| XT-M7 | ++ | +++ | XT-H7 | +++ | − |

The kinetic rate constants (kₐ and k_{d}) and association and dissociation constants (Kₐ and K_{d}) for the binding of murine and rat anti-RAGE antibodies to human and murine RAGE were determined by BIACORE™ direct binding assay. Analysis of the signal kinetics data for on-rate and off-rate allows the discrimination between non-specific and specific interactions. Kinetic rate constants and equilibrium constants determined by the BIACORE™ direct binding assay for the binding of murine XT-H2 antibody and rat XT-M4 antibody to hRAGE-Fc are shown in Table 5.

TABLE 5
Kinetic rate constants and equilibrium constants for binding to hRAGE-Fc

| | kₐ (1/Ms) | k_{d} (1/s) | Kₐ (1/M) | K_{d} (M) | R_{Max} | X² |
|---|---|---|---|---|---|---|
| XT-H2 | 5.76 × 10⁶ | 5.04 × 10⁻⁴ | 1.14 × 10¹⁰ | 8.76 × 10⁻¹¹ | 55.7 | 2.68 |
| XT-M4 | 1.16 × 10⁶ | 1.16 × 10⁻³ | 1.00 × 10⁹ | 9.95 × 10⁻¹⁰ | 89.9 | 14.3 |

B. Binding to the Human RAGE V-Domain

The kinetic rate constants and association and dissociation constants for the binding of murine and rat anti-RAGE antibodies to the human RAGE V-domain were also determined by BIACORE™ direct binding assay. Human RAGE V-domain-Fc was captured by anti-human Fc antibodies coated on a CM5 chip, and BIACORE™ direct binding assays of the binding of murine and rat anti-RAGE antibodies to the immobilized hRAGE V domain-Fc were performed as described above for assays of binding to full-length RAGE-Fc.

### Example 9

### Amino Acid Sequences of Anti-RAGE Antibody Variable Regions

DNA sequences encoding the light and heavy chain variable regions of murine anti-RAGE antibodies XT-H1, XT-H2, XT-H3, XT-H5 and XT-H7, and of rat anti-RAGE antibody XT-M4 were cloned and sequenced, and the amino acid sequences of the variable regions were determined. The aligned amino acid sequences of the heavy chain variable regions of these six antibodies are shown in FIG. 6, and the aligned amino acid sequences of the light chain variable regions are shown in FIG. 7.

### Example 10

### Isolation of Rabbit, Baboon, and Cynomologus Monkey cDNA Sequences Encoding RAGE

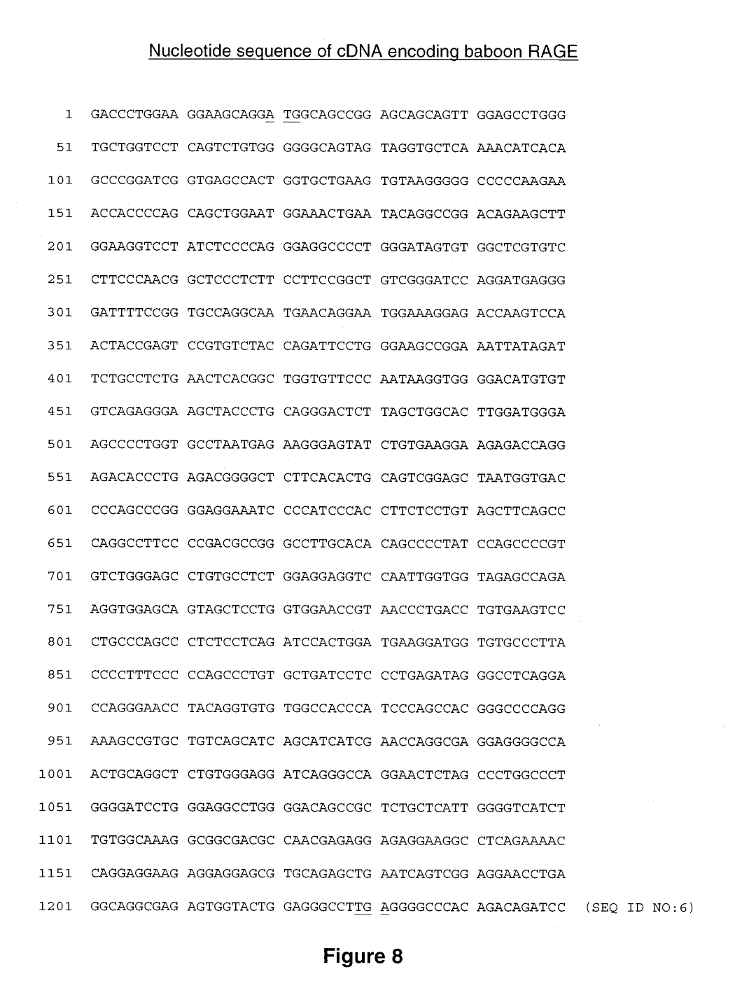

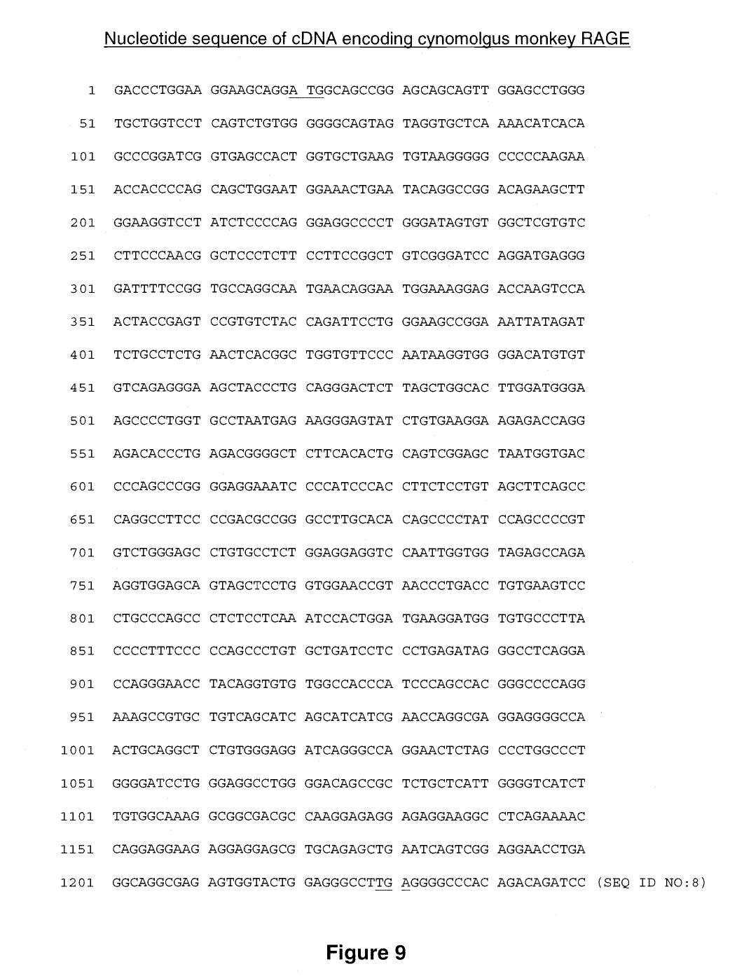

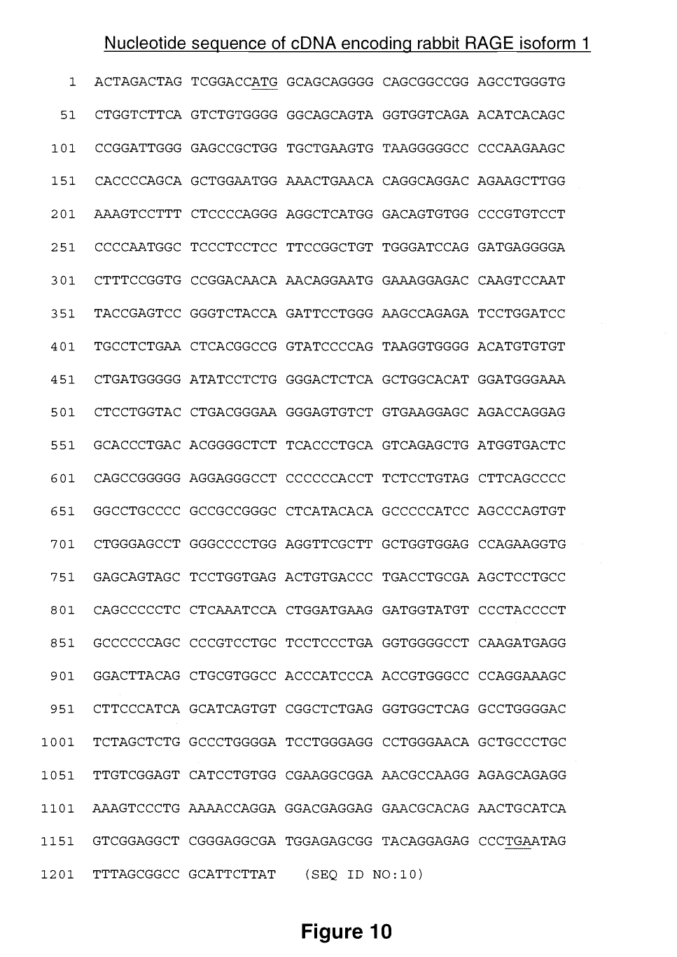

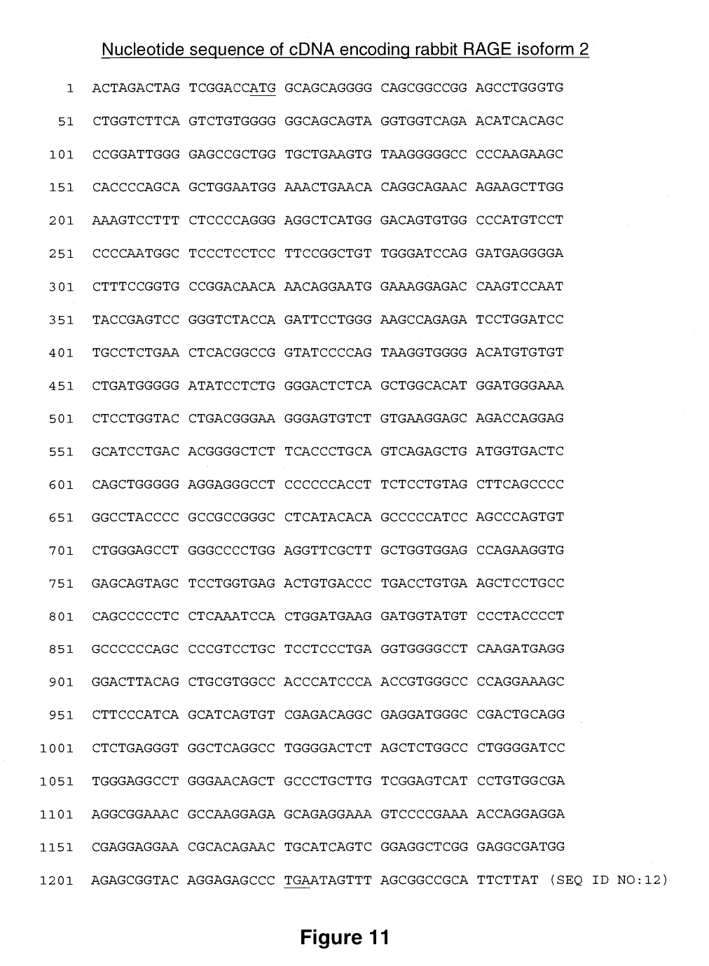

cDNA sequences encoding RAGE were isolated and cloned using standard reverse transcription-polymerase chain reaction (RT-PCR) methods. RNA was extracted and purified from lung tissue using Trizol (Gibco Invitrogen, Carlsbad, Calif.) via the manufacturer's protocol. mRNA was reverse transcribed to generate cDNA using TaqMan Reverse Transcription Reagent (Roche Applied Science Indianapolis, Ind.) and manufacturer's protocol. Cynomologus monkey (Macaca fascicularis) and baboon (Papio cyanocephalus) RAGE sequences were amplified from cDNA using Invitrogen Taq DNA polymerase (Invitrogen, Carlsbad Calif.) and protocol and oligonucleotides (5′-GACCCTGGAAGGAAGCAGGATG (SEQ ID NO: 59) and 5′-GGATCTGTCTGTGGGCCCCTCAAGGCC) (SEQ ID NO: 60) that add SpeI and EcoRV restriction sites. PCR amplification products were digested with SpeI/EcoRV and cloned into the corresponding sites in the plasmid pAdori1-3. Rabbit RAGE was cloned using RT-PCR as described above using the oligonucleotides: 5′-ACTAGACTAGTCGGACCATGGCAGCAGGGGCAGCGGCCGGA (SEQ ID NO: 61) and 5′-ATAAGAATGCGGCCGCTAAACTATTCAGGGCTCTCCTGTACCGCTCTC (SEQ ID NO: 62) that add SpeI and NotI sites, and cloned into the corresponding sites in pAdori1-3. The nucleotide sequences of the cloned cDNA sequences encoding baboon, monkey, and two isoforms of rabbit RAGE in the resultant plasmids were determined. The nucleotide sequence encoding baboon RAGE is shown in FIG. 8 (SEQ ID NO: 6), and the nucleotide sequence encoding cynomologus monkey RAGE is shown in FIG. 9 (SEQ ID NO: 8). The nucleotide sequences encoding two isoforms of rabbit RAGE are shown in FIG. 10 (SEQ ID NO:10) and FIG. 11 (SEQ ID NO:12).

### Example 11

### Isolation of a Genomic DNA Sequence Encoding Baboon RAGE

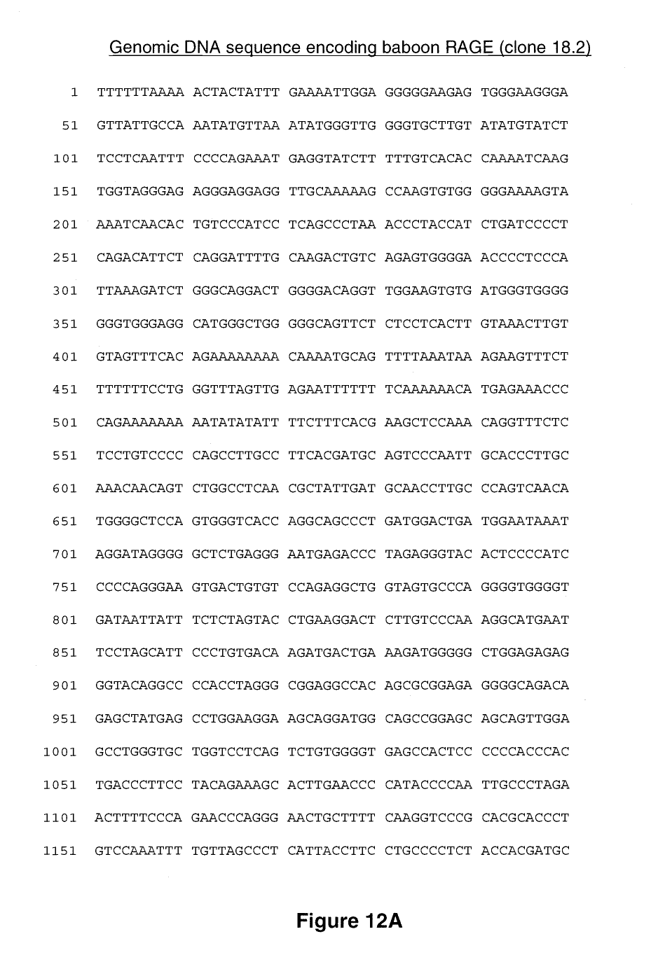

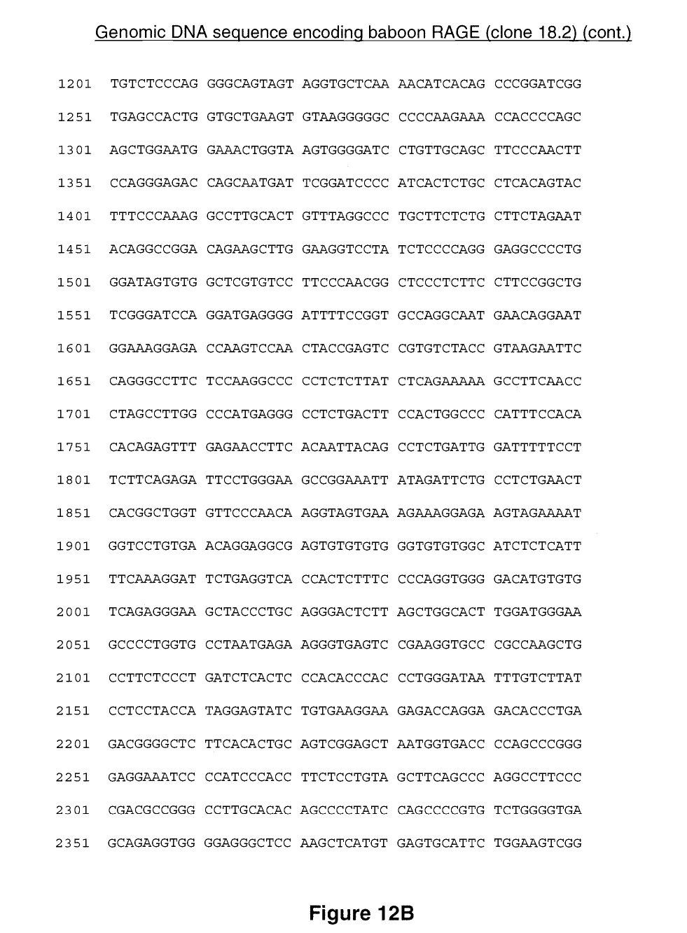

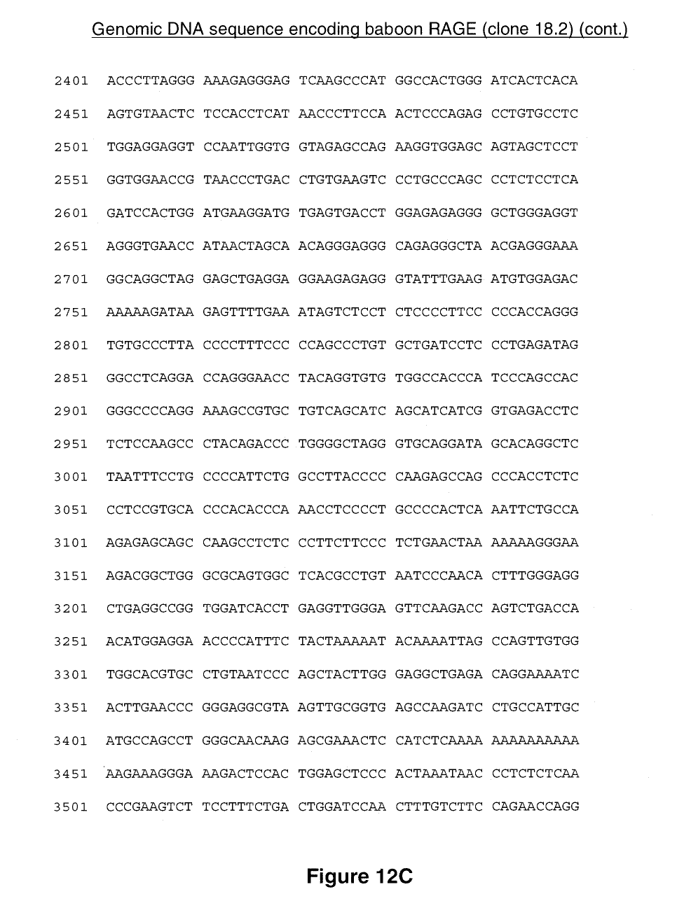

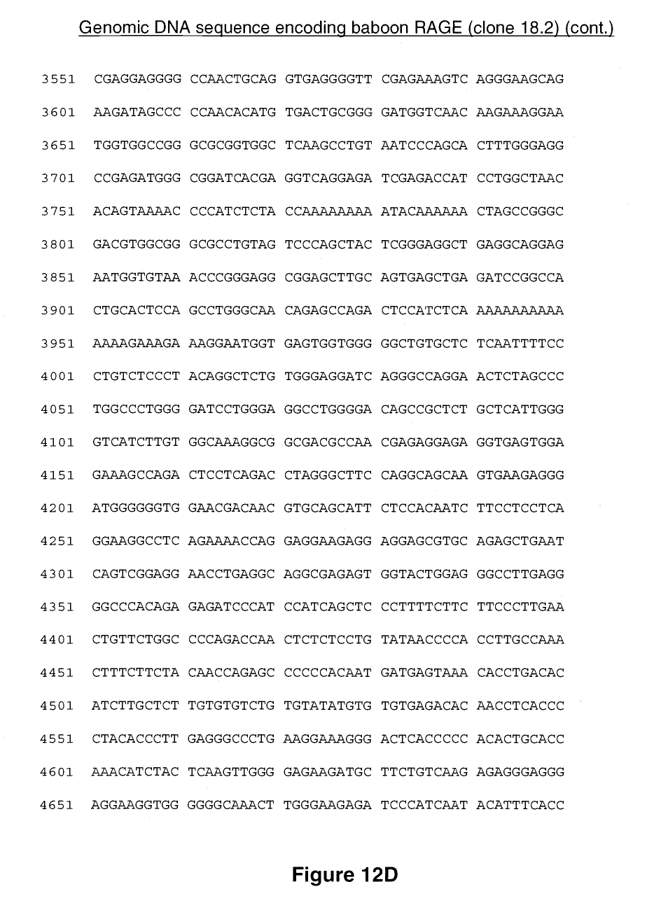

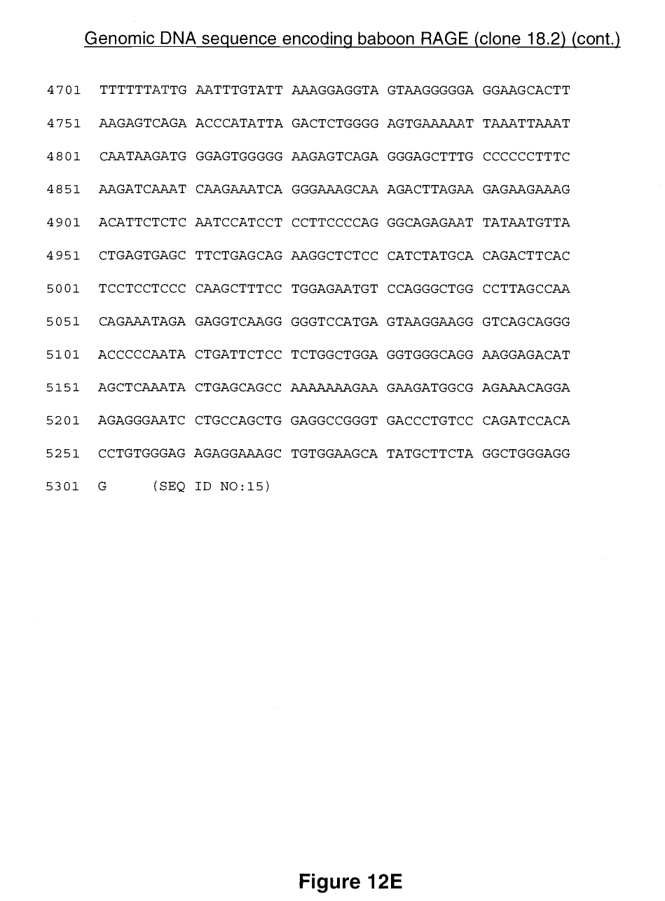

A baboon genomic DNA sequence encoding RAGE was isolated using standard genomic cloning techniques (e.g., see Sambrook, J. et al., Molecular Cloning: A Laboratory Manual, 2^{nd} Ed., 1989, Cold Spring Harbor Laboratory Press, Cold Spring Harbor, N.Y.). A baboon (Papio cyanocephalus) Lambda genomic library (Stratagene, La Jolla, C) in the Lambda DASH II vector was screened using ³²P random primed human RAGE cDNA. Positive phage plaques were isolated and subjected to two additional rounds of screening to obtain single isolates. Lambda DNA was prepared, digested with NotI, and size fractionated to separate insert DNA from Lambda genomic arms, using common procedure. The NotI fragments were ligated into NotI-digested pBluescript SK+, and the insert was sequenced using RAGE specific primers. The clone that was obtained was designated clone 18.2. The nucleotide sequence of the cloned baboon genomic DNA encoding a baboon RAGE is shown in FIGS. 12A-12-E (SEQ ID NO: 15).

### Example 12

### Chimeric XT-M4 Antibody

A chimeric XT-M4 was generated by fusing the light and heavy chain variable regions of rat anti-murine RAGE antibody XT-M4 to human kappa light chain and IgG1 heavy chain constant regions, respectively. To reduce the potential Fc-mediated effector activity of the antibody, chimeric mutations L234A and G237A were introduced into XT-M4 in the human IgG1 Fc region. The chimeric antibody is given molecule number XT-M4-A-1. The chimeric XT-M4 antibody contains 93.83% human amino acid sequence, and 6.18% rat amino acid sequence.

### Example 13

### Assessing the Binding of Chimeric XT-M4 to RAGE

The abilities of chimeric antibody XT-M4 and selected rat and murine anti-RAGE antibodies to bind to human RAGE and RAGE of other species, and to block the binding of RAGE ligands was measured by ELISA and BIACORE™ binding assays.

A. Binding to Soluble Human RAGE Measured by BIACORE™ Binding Assay

The binding of chimeric antibody XT-M4, the parental rat antibody XT-M4, and murine antibodies XT-H2 and XT-H5 to soluble human RAGE (hRAGE-SA) was measured by BIACORE™ capture binding assay. The assays were performed by coating antibodies onto a CM5 BIA chip with 5000-7000 RU. Solutions of a purified soluble human streptavidin-tagged RAGE (hRAGE-SA) at concentrations of 100 nM, 50 nM, 25 nM, 12.5 nM, 6.25 nM, 3.12 nM, 1.56 nM and 0 nM were flowed over the immobilized antibodies in triplicate, and kinetic rate constants (kₐ and k_{d}) and association and dissociation constants (Kₐ and K_{d}) for binding to hRAGE-SA were determined. The results are shown in Table 6.

TABLE 6
Kinetic rate constants and equilibrium constants for binding to hRAGE-SA

| | kₐ (1/Ms) | k_{d} (1/s) | Kₐ (1/M) | K_{d} (M) | R_{Max} | X² |
|---|---|---|---|---|---|---|
| XT-M4 | 3.78 × 10⁶ | 1.86 × 10⁻² | 2.03 × 10⁸ | 4.92 × 10⁻⁹ | 61.5 | 0.563 |
| chimeric | 4.39 × 10⁶ | 2.48 × 10⁻² | 1.77 × 10⁸ | 5.66 × 10⁻⁹ | 33.1 | 0.436 |
| antibody XT-M4 | | | | | | |
| XT-H2 | 1.10 × 10⁶ | 1.16 × 10⁻³ | 9.48 × 10⁸ | 1.06 × 10⁻⁹ | 48.1 | 2.7 |
| XT-H5 | 1.66 × 10⁶ | 4.51 × 10⁻³ | 3.69 × 10⁸ | 2.71 × 10⁻⁹ | 24.5 | 0.996 |

The XT-M4 antibody and chimeric antibody XT-M4 bind to monomeric soluble human RAGE with similar kinetics. The affinity of chimeric XT-M4 for human soluble monomeric RAGE is approximately 5.5 nM.
B. RAGE Ligand Competition ELISA Binding Assay

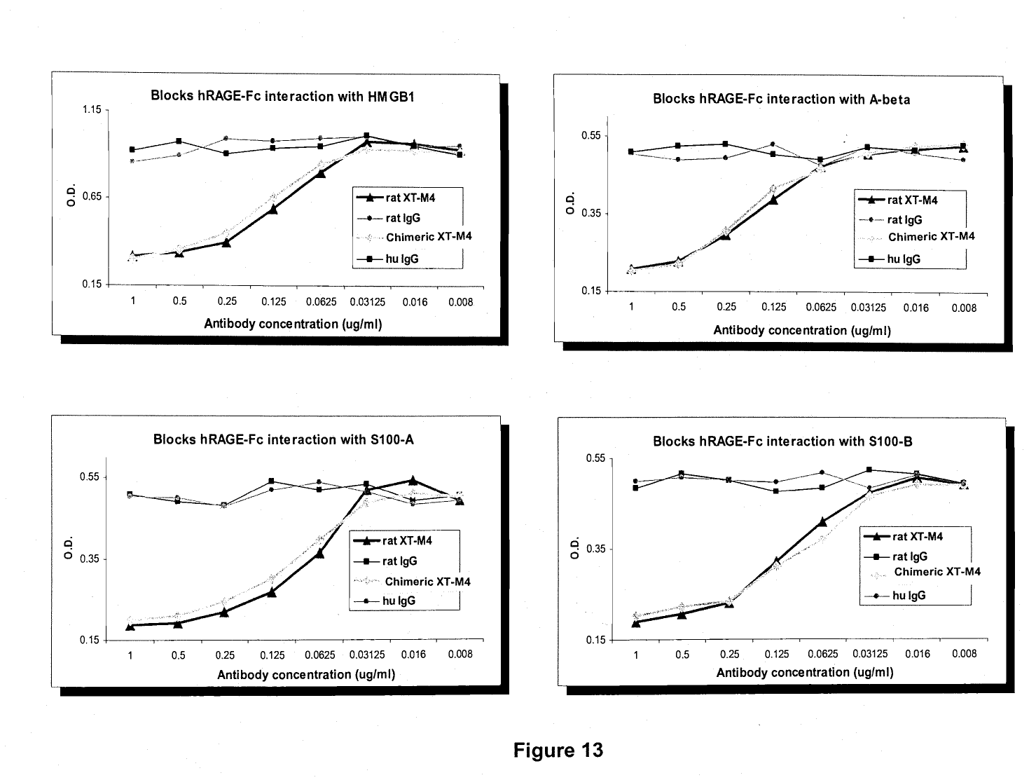

The abilities of chimeric antibody XT-M4 antibody and rat antibody XT-M4 to block the binding of RAGE ligands HMGB1, amyloid β 1-42 peptide, S100-A, and S100-B to hRAGE-Fc were determined by ligand competition ELISA binding assay as described in Example 7. As shown in FIG. 13, chimeric antibody XT-M4 and XT-M4 are nearly identical in their abilities to block the binding of HMGB1, amyloid β 1-42 peptide, S100-A, and S100-B to human RAGE.

C. Antibody competition ELISA binding assay

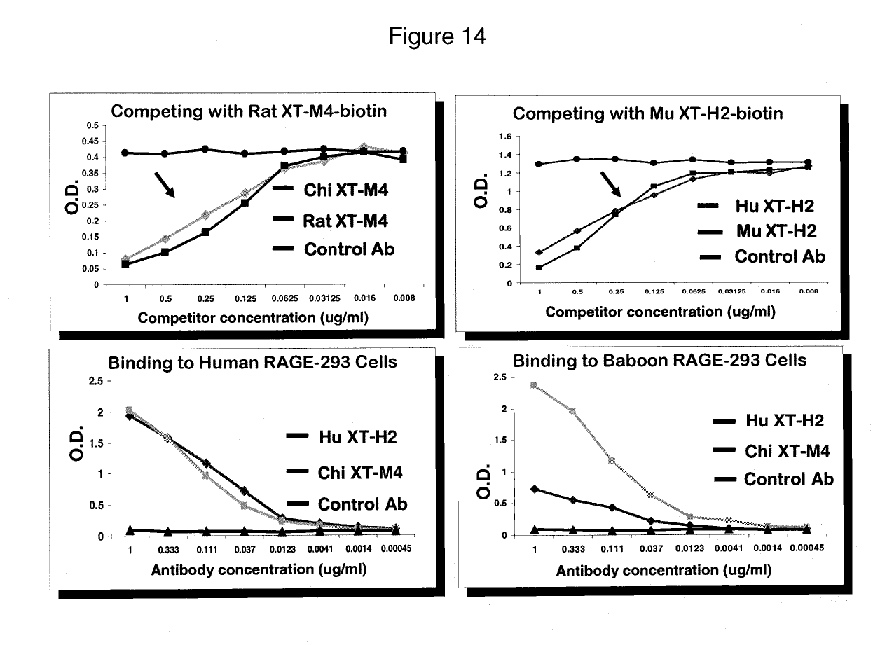

The ability of chimeric antibody XT-M4 antibody to compete with rat antibody XT-M4 and murine antibody XT-H2 in binding to hRAGE-Fc was determined by antibody competition ELISA binding assay, using biotin-linked XT-M4 and XT-H2 antibodies, in the manner described in Example 7. As shown in FIG. 14, chimeric antibody XT-M4 competes with rat antibody XT-M4 and with murine antibody XT-H2 in binding to hRAGE-Fc.

### Example 14

### Antibody Binding to RAGE of Different Species was Measured by Cell-Based ELISA

Cell Transfection

Human embryonic kidney 293 cells (American Tissue Type Culture, Manassas, Va.) cells were plated at 5×10⁶ cells per 10 cm² tissue culture plate and cultured overnight at 37° C. The next day cells were transfected with RAGE expression plasmids (pAdori1-3 vector encoding mouse, human, baboon, cynomologus monkey or rabbit RAGE) using LF2000 reagent (Invitrogen, Carlsbad Calif.) at a 4:1 ratio of reagent to plasmid DNA using the manufacturers protocol. Cells were harvested 48 hrs post-transfection using trypsin, washed once with phosphate buffered saline (PBS), then suspended in growth media without serum at a concentration of 2×10⁶ cells/ml.

Cell-Based ELISA

Primary antibodies at 1 μg/ml were serially diluted at 1:2 or 1:3 in PBS containing 1% bovine serum albumin (BSA) in a 96-well plate. RAGE-transfected 293 cells or control parental 293 cells (50 μl) at 2×10⁶ cells/ml in serum-free growth medium were added to U-bottom 96 well plate for a final concentration of 1×10⁵ cells/well. The cells were centrifuged at 1600 rpm for 2 minutes. The supernatants were gently discarded by hand with a one-time swing and the plate was patted gently to loose the cell pellet. The diluted primary anti-RAGE antibodies or isotype-matching control antibodies (100 μl) in cold PBS containing 10% fetal calf serum (FCS) were added to the cells and incubated on ice for 1 hour. The cells were stained with 100 μl of diluted secondary anti-IgG antibody HRP conjugates (Pierce Biotechnology, Rockford, Ill.) on ice for 1 hour. Following each step of primary antibody and secondary antibody incubations, the cells were washed 3 times with ice-cold PBS. 100 μl of substrate TMB1 component (BIO FX, TMBW-0100-01) was added to the plate and incubated for 5-30 minutes at room temperature. The color development was stopped by adding 100 μl of 0.18M H₂SO₄. The cells were centrifuged and the supernatants are transferred to a fresh plate and read at 450 nm (Soft MAX pro 4.0, Molecular Devices Corporation, Sunnyvale, Calif.).

The abilities of antibodies chimeric XT-M4 and XT-M4 to bind to human & baboon RAGE as determined by cell-based ELISA are shown in FIG. 14. The EC50 values for the binding of chimeric antibody XT-M4 and XT-M4 to cell surface human, baboon, monkey, mouse & rabbit RAGE expressed by 293 cells, as determined by cell-based ELISA, are shown in Table 7.

TABLE 7
EC50 values for binding to RAGE
determined by cell-based ELISA

| | chimeric XT-M4 | rat XT-M4 |
|---|---|---|
| 293-murine RAGE | ˜1.5 nM | ˜2.2 nM |
| 293-human RAGE | ˜0.8 nM | ˜0.84 nM |
| 293-cyno monkey RAGE | ˜1.66 nM | ˜2.33 nM |
| 293-baboon RAGE | ˜1.25 nM | ˜1.33 nM |

### Example 15

### Binding to RAGE of Different Species—Determined by Immunohistochemical Staining

The abilities of the chimeric antibody XT-M4, the rat XT-M4 antibody, and murine antibodies XT-H1, XT-H2, and XT-H5 to bind to endogenous cell surface RAGE in lung tissue of human, cynomologus monkey, baboon, and rabbit were determined by immunohistochemical (IHC) staining of lung tissue sections.

Stably transfected Chinese Hamster Ovary (CHO) cells were engineered to express murine and human RAGE full length proteins. The murine and human RAGE cDNAs were cloned into the mammalian expression vector, linearized and transfected into CHO cells using lipofectin (methods (Kaufman, R. J., 1990, Methods in Enzymology 185:537-66; Kaufman, R. J., 1990, Methods in Enzymology 185:487-511; Pittman, D. D. et al., 1993, Methods in Enzymology 222: 236). Cells were further selected in 20 nM methotrexate and cell extracts were harvested from individual clones and analyzed by SDS sodium dodecyl sulfate-polyacrylamide gel electrophoresis (SDS-PAGE) and Western blotting to confirm expression.

Immunohistochemistry for RAGE lung tissues isolated from baboon, cynomolgus monkey, rabbit or Chinese Hamster Ovary cells over-expressing human RAGE or control CHO cells were performed using standard techniques. RAGE antibodies and rat IgG2b isotype control or mouse isotype control were used at 1-15 mg. Chimeric XT-M4, XT-M4-hVH-V2.0-2 m/hVL-V2.10, XT-M4-hVH-V2.0-2 m/hVL-V2.11, XT-M4-hVH-V2.0-2 m/hVL-V2.14 were biotinalyted and Sigma IgG1 biotinalyted control antibody at 0.2, 1, 5 and 10 μg/ml was used. Following detection with HRP and Alexa Fluor 594, Alexa Fluor 488 or anti-biotin conjugated with FITC, sections were also stained with 4′-6-Diamidino-2-phenylindole (DAPI).

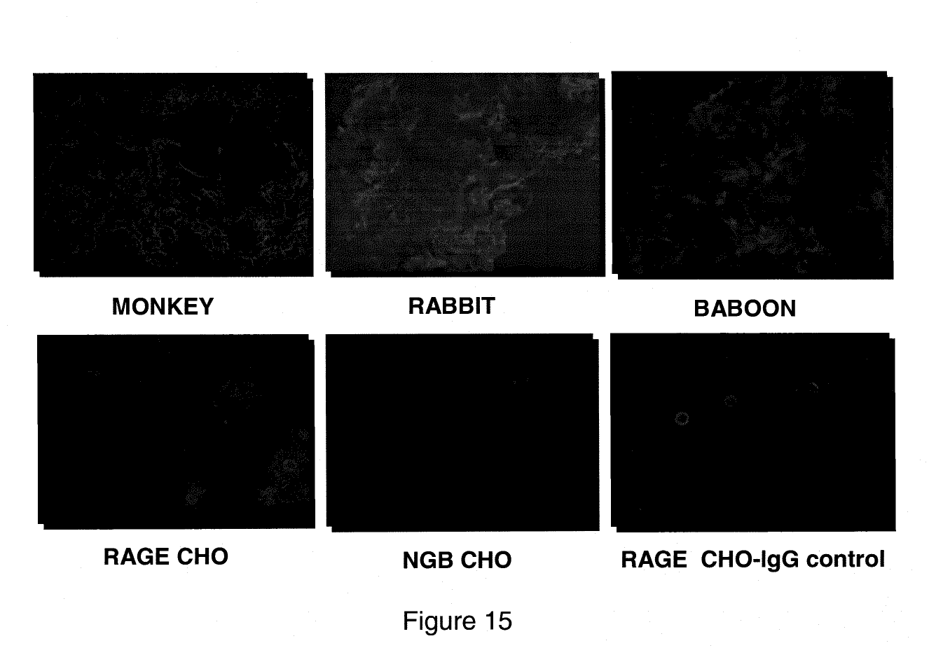

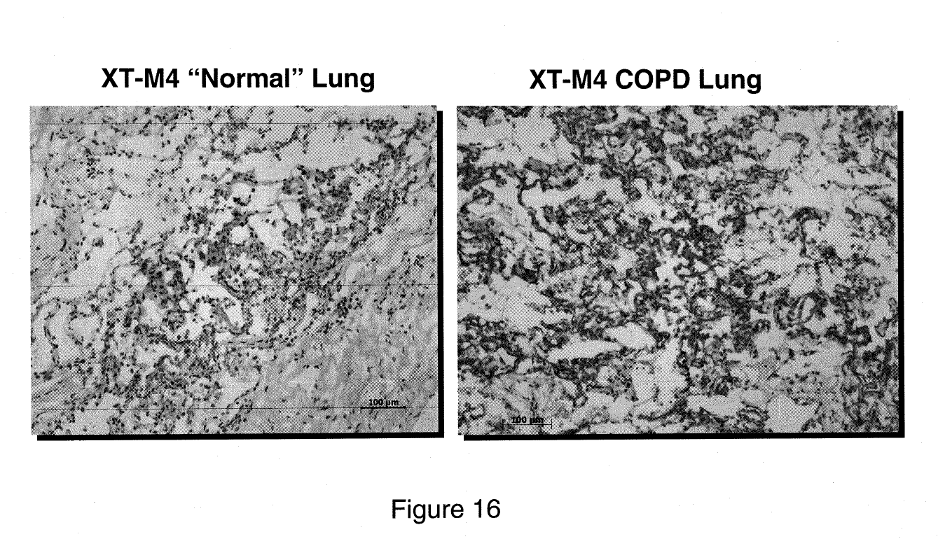

FIG. 15 shows that the chimeric antibody XT-M4 binds to RAGE in lung tissues of cynomologus monkey, rabbit, and baboon. Positive IHC-staining patterns are visible in the samples in which RAGE-producing cells are contacted with chimeric XT-M4, but not in samples in which either RAGE or a RAGE-binding antibody are absent. FIG. 16 shows that the rat antibody XT-M4 binds to RAGE in normal human lung and lung of a human with chronic obstructive pulmonary disease (COPD). The binding of rat XT-M4 antibody and murine antibodies XT-H1, XT-H2, and XT-H5 to endogenous cell surface RAGE in septic baboon lung and normal cynomologus monkey lung, as determined by IHC staining of lung tissue sections, is summarized in Table 8. CHO cells stable transfected with an expression vector that expresses DNA encoding hRAGE is used as a positive control.

TABLE 8
Binding to RAGE in non-human primate lung - assayed by IHC

| | | | | | | | | hRAGE | |
|---|---|---|---|---|---|---|---|---|---|
| | Baboon lung (septic) | | | | Monkey lung (normal) | | | CHO | CHO |
| μg/ml | 1 | 5 | 10 | 15 | 5 | 10 | 15 | 1 | 1 |
| XT-M4 | +++ | +++ | ++++ | ++++ | + | ++ | | +++ | − |
| XT-H1 | ++++ | ++++ | ++++ | ++++ | +++ | +++ | | +++ | − |
| XT-H2 | − | − | + | ++ | − | − | | +++ | − |
| XT-H5 | ++++ | ++++ | ++++ | ++++ | − | − | | +++ | − |
| mRA109 | | | − | − | | − | − | − | − |
| control | | | | | | | | | |
| rSFR | | | − | − | | − | − | − | − |
| control | | | | | | | | | |

### Example 16

### Molecular Modeling for Humanizing Murine Anti-Human RAGE Antibody XT-H2

Molecular Modeling of Murine Anti-Human RAGE Antibody XT-H2 HV Domain

Antibody structure templates for modeling murine XT-H2 heavy chain were selected based BLASTP search against Protein Data Bank (PDB) sequence database. Molecular model of murine XT-H2 was built based on 6 template structures, 1SY6 (anti-CD3 antibody), 1MRF (anti-RNA antibody), and 1RIH (anti-tumor antibody) using the Homology module of Insightil (Accelrys, San Diego). The structurally conserved regions (SCRs) of the templates were determined based on the Cα distance matrix for each molecule and the templates structures were superposed based on minimum RMS deviation of corresponding atoms in SCRs. Sequence of the target protein rat XT-H2 VH was aligned to the sequences of the superposed template proteins and the atomic coordinates of the SCRs were assigned to the corresponding residues of the target protein. Based on the degree of sequence similarity between the target and the templates in each of the SCRs, coordinates from different templates were used for different SCRs. Coordinates for loops and variable regions not included in the SCRs were generated by Search Loop or Generate Loop methods as implemented in the Homology module.

Briefly, the Search Loop method scans protein structures that would mimic the region between 2 SCRs by comparing the Cα distance matrix of flanking SCR residues with a pre-calculated matrix derived from protein structures that have the same number of flanking residues and an intervening peptide segment of a given length. The output of the Search Loop method was evaluated to first find a match having minimal RMS deviations and maximum sequence identity in the flanking SCR residues. Then an evaluation of sequence similarity between the potential matches and the sequence of the target loop was undertaken. The Generate Loop method generates atom coordinates de novo was used in those cases where Search Loops did not find optimal matches. Conformation of amino acid side chains was kept the same as that in the template if the amino acid residue was identical in the template and the target. However, a conformational search of rotamers was performed and the energetically most favorable conformation was retained for those residues that are not identical in the template and target. To optimize the splice junctions between two adjacent SCRs, whose coordinates were adapted from different templates, and those between SCRs and loops, the Splice Repair function of the Homology module was used. The Splice Repair sets up a molecular mechanics simulation to derive optimal bond lengths and bond angles at junctions between 2 SCRs or between SCR and a variable region. Finally the model was subjected to energy minimization using Steepest Descents algorithm until a maximum derivative of 5 kcal/(mol A) or 500 cycles and Conjugate Gradients algorithm until a maximum derivative of 5 kcal/(mol A) or 2000 cycles. Quality of the model was evaluated using ProStat/Struct_Check utility of the Homology module.

Molecular Modeling of Humanized Anti-RAGE XT-H2 HV Domain

A molecular model of the humanized (CDR grafted) anti-RAGE antibody XT-H2 heavy chain was built with Insight II following the same procedure as described for the modeling of the mouse XT H2 antibody heavy chain, except that the templates used were different. The structure templates used in this case were 1L7I (anti-Erb B2 antibody), 1FGV (anti-CD18 antibody), 1JPS (anti-tissue factor antibody) and 1N8Z (anti-Her2 antibody).

Model Analysis and Prediction of Frame Work Back Mutations-Humanization

The parental mouse antibody model was compared to the model of the CDR-grafted humanized version with respect to similarities and differences in one or more of the following features: CDR-framework contacts, potential hydrogen bonds influencing CDR conformation, and RMS deviations in various regions such as framework 1, framework 2, framework 3, framework 4 and the 3 CDRs.

The following back mutations singly and in combinations were predicted to be important for successful humanization by CDR grafting: E46Y, R72A, N77S, N74K, R67K, K76S, A23K, F68A, R38K, A40R.

### Example 17

### Molecular Modeling for Humanizing Rat Anti-RAGE Antibody XT-M4

Molecular Modeling of Rat Anti-Murine RAGE Antibody XT-M4 HV Domain

Antibody structure templates for modeling rat XT-M4 heavy chain were selected based upon BLASTP search against Protein Data Bank (PDB) sequence database. Molecular models of rat XT-M4 were built based on 6 template structures, 1QKZ (anti-peptide antibody), 1IGT (anti-canine lymphoma monoclonal antibody), 8FAB (anti-p-azophenyl arsonate antibody), 1 MQK (anti-cytochrome C oxidase antibody), 1 HOD (anti-angiogenin antibody), and 1 MHP (anti-alpha1beta1 antibody) using the Homology module of Insightil (Accelrys, San Diego). The structurally conserved regions (SCRs) of the templates were determined based on the Cα distance matrix for each molecule and the templates structures were superposed based on minimum RMS deviation of corresponding atoms in SCRs. The sequence of the target protein rat XT-M4 VH was aligned to the sequences of the superposed template proteins and the atomic coordinates of the SCRs were assigned to the corresponding residues of the target protein. Based on the degree of sequence similarity between the target and the templates in each of the SCRs, coordinates from different templates were used for different SCRs. Coordinates for loops and variable regions not included in the SCRs were generated by Search Loop or Generate Loop methods as implemented in the Homology module.

Briefly, the Search Loop method scans protein structures that would mimic the region between 2 SCRs by comparing the Cα distance matrix of flanking SCR residues with a pre-calculated matrix derived from protein structures that have the same number of flanking residues and an intervening peptide segment of a given length. The output of the Search Loop method was evaluated to first find a match having minimal RMS deviations and maximum sequence identity in the flanking SCR residues. Then an evaluation of sequence similarity between the potential matches and the sequence of the target loop was undertaken. The Generate Loop method generates atom coordinates de novo was used in those cases where Search Loops did not find optimal matches. Conformation of amino acid side chains was kept the same as that in the template if the amino acid residue was identical in the template and the target. However, a conformational search of rotamers was performed and the energetically most favorable conformation was retained for those residues that are not identical in the template and target. To optimize the splice junctions between two adjacent SCRs, whose coordinates were adapted from different templates, and those between SCRs and loops, the Splice Repair function of the Homology module was used. The Splice Repair sets up a molecular mechanics simulation to derive optimal bond lengths and bond angles at junctions between 2 SCRs or between SCR and a variable region. Finally the model was subjected to energy minimization using Steepest Descents algorithm until a maximum derivative of 5 kcal/(mol A) or 500 cycles and Conjugate Gradients algorithm until a maximum derivative of 5 kcal/(mol A) or 2000 cycles. Quality of the model was evaluated using ProStat/Struct_Check utility of the Homology module.

XT-M4 Light Chain Variable Domain

Structural models for XT M4 light chain variable domain were generated with Modeler 8v2 using 1K6Q (anti-tissue factor antibody), 1WTL, 1D5B (antibody AZ-28) and 1 BOG (anti-p24 antibody) as the templates. For each target, out of the 100 initial models, one model with the lowest restraint violations, as defined by the molecular probability density function, was chosen for further optimization. For model optimization an energy minimization cascade consisting of Steepest Descent, Conjugate Gradient and Adopted Basis Newton Raphson methods was performed until an RMS gradient of 0.01 was satisfied using Charmm 27 force field (Accelrys Software Inc.) and Generalized Born implicit solvation as implemented in Discovery Studio 1.6 (Accelrys Software Inc.). During energy minimization, movement of backbone atoms was restrained using a harmonic constraint of 10 mass force.

Molecular Modeling of Humanized Anti-RAGE XT-M4 VH Domain

A molecular model of the humanized (CDR grafted) anti-RAGE XT M4 antibody heavy chain was built with Insight II following the same procedure as described for the modeling of the rat XT M4 antibody heavy chain, except that the templates used were different. The structure templates used in this case were 1 MHP (anti-alpha1beta1 antibody), 1IGT (anti-canine lymphoma monoclonal antibody), 8FAB (anti-p-azophenyl arsonate antibody), 1 MQK (anti-cytochrome C oxidase antibody) and 1 HOD (anti-angiogenin antibody).

Humanized XT-M4 Light Chain Variable Domain

A molecular model of the humanized (CDR grafted) anti-RAGE XT M4 antibody light chain was built using Modeler 8v2 following the same procedure as described for the modeling of the rat XT M4 antibody light chain, except that the templates used were different. Structure templates used in this case were 1B6D,1FGV (anti-CD18 antibody), 1UJ3 (anti-tissue factor antibody) and 1 WTL as the templates.

### Model Analysis and Prediction of Frame Work Back Mutations-Humanization

The parental rat antibody model was compared to the model of the CDR-grafted humanized version with respect to similarities and differences in one or more of the following features: CDR-framework contacts, potential hydrogen bonds influencing CDR conformation, RMS deviations in various regions such as framework 1, framework 2, framework 3, framework 4 and the 3 CDRs, and calculated energies of residue-residue interactions. The potential back mutation(s) identified were incorporated, singly or in combinations, into another round of models built using either Insight II or Modeler 8v2 and the models of the mutants were compared to the parental rat antibody model to evaluate the suitability of mutants in silico.

The following back mutations singly and in combinations were predicted to be important for successful humanization by CDR grafting:

Heavy chain: L114M, T113V and A88S;

Light chain: K₄₅R, L46R, L47M, D701, G66R, T85D, Y87H, T69S, Y36F, F71Y.

### Example 18

### Humanized Variable Regions with the CDRs of Murine XT-H2 and Rat XT-M4 Antibodies

Humanized heavy chain variable regions were prepared by grafting the CDRs of the murine XT-H2 and rat XT-M4 antibodies onto human germline framework sequences shown in Table 9, and introducing selected back mutations.

TABLE 9

| Antibody | Isotype | Human Germline | Identity |
|---|---|---|---|
| XT-H2_VH | mG1/K | DP-75 VH1; 1-46 | 77.50% |
| XT-M4_VH | rG2b/K | DP-54 VH3; 3-07 | 77.50% |
| XT-H2_VL | mG1/K | DPK-12 VK2; A2 | 80.00% |
| XT-M4_VL | rG2b/K | DPK-9 VK1; 02 | 64.50% |

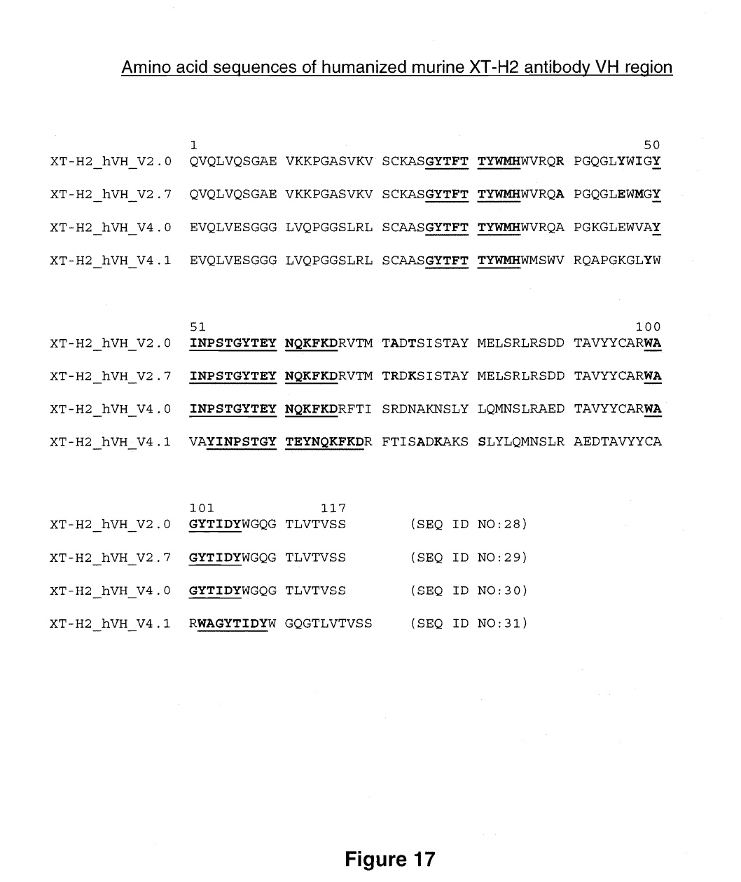

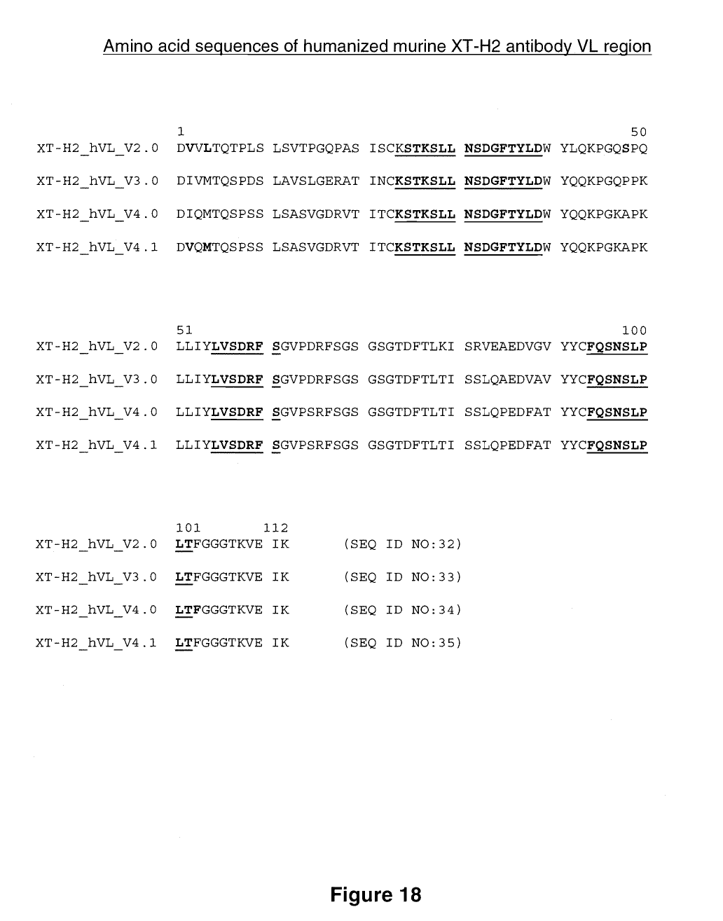

The amino acid sequences of humanized murine XT-H2 heavy and light chain variable regions are shown in FIG. 17 (SEQ ID NOs: 28-31) and FIG. 18 (SEQ ID NOs: 32-35), respectively.

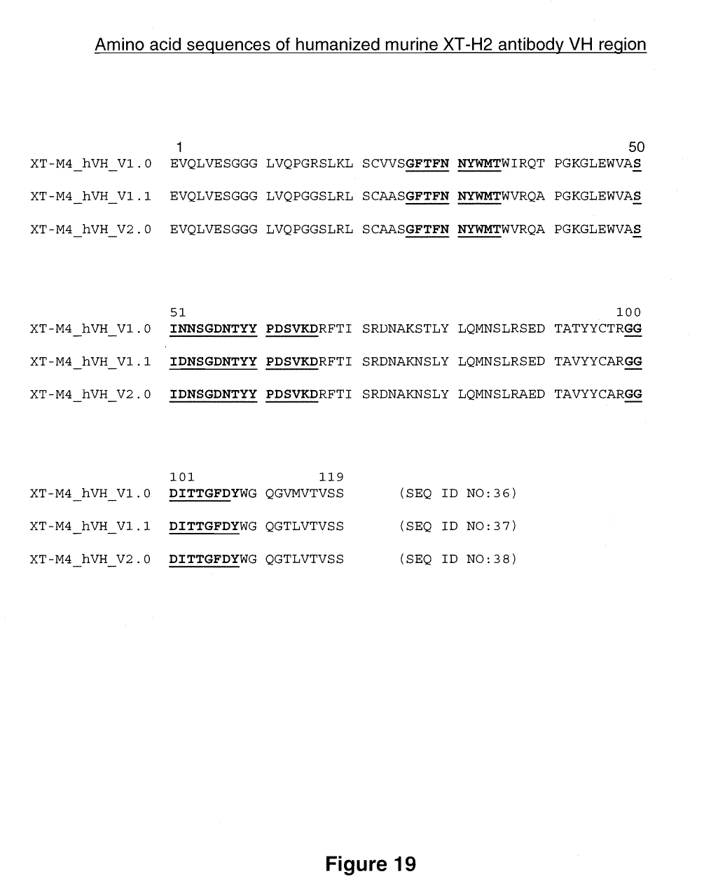

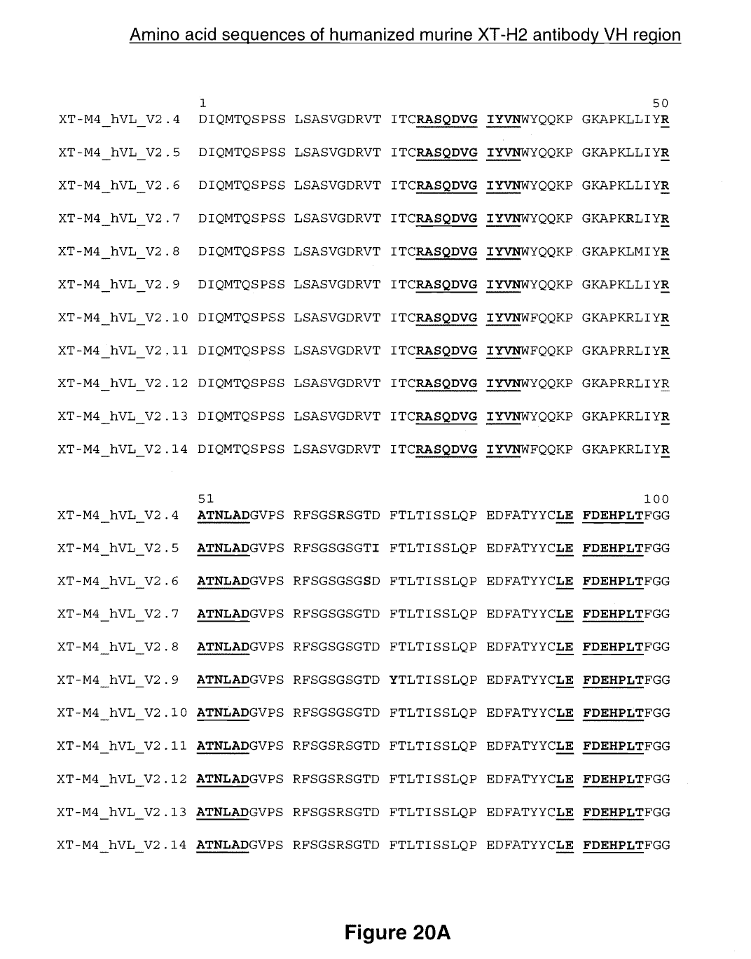

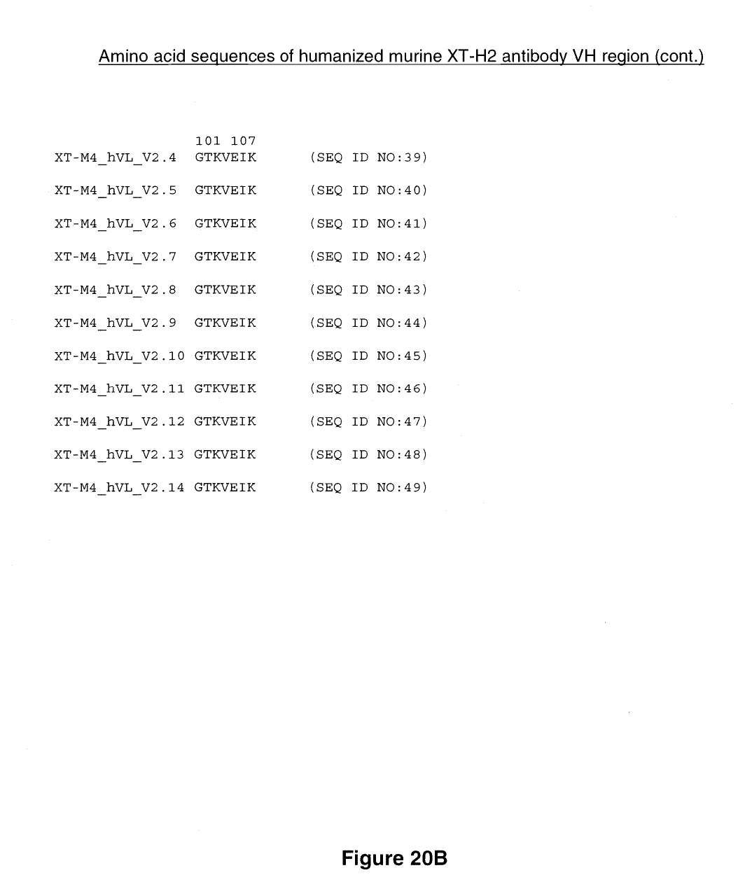

The amino acid sequences of humanized rat XT-M4 heavy and light chain variable regions are shown in FIG. 19 (SEQ ID NOs: 36-38) and FIGS. 20A-20B (SEQ ID NOs: 39-49), respectively.

Germline sequences from which the framework sequences were derived and specific backmutations in the humanized variable regions are identified in Table 10.

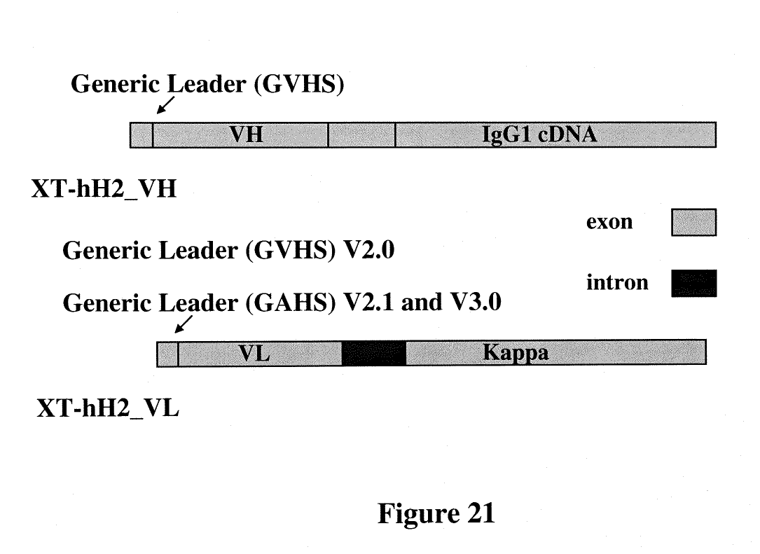

DNA sequences encoding the humanized variable regions were subcloned into expression vectors containing sequences encoding human immunoglobulin constant regions, and DNA sequences encoding the full-length light and heavy chains were expressed in COS cells, using standard procedures. DNAs encoding heavy chain variable regions were subcloned into a pSMED2hIgG1 m_(L234, L237)cDNA vector, producing humanized IgG1 antibody heavy chains. DNAs encoding light chain variable regions were subcloned into a pSMEN2 hkappa vector, producing humanized kappa antibody light chains. See FIG. 21.

TABLE 10

| Humanized V domain | Germline | Backmutations |
|---|---|---|
| XT-H2_hVH_V2.0 | DP-75 | A40R, E46Y, M48I, |
| | | R71A, and T73K |
| XT-H2_hVH_V2.7 | DP-75 | |
| XT-H2_hVH_V4.0 | DP-54 FW, VH 3, JH4 | |
| XT-H2_hVH_V4.1 | DP-54 FW, VH 3, JH4 | |
| XT-H2_hVL_V2.0 | DPK-12 | I2V, M4L and P48S |
| XT-H2_hVL_V3.0 | DPK-24 | |
| XT-H2_hVL_V4.0 | DPK-9 Vk1 | |
| XT-H2_hVL_V4.1 | DPK-9 Vk1, Jk 4 | |
| XT-M4_hVH_V1.0 | DP-54, VH3; 3-07 | |
| XT-M4_hVH_V1.1 | DP-54, VH3; 3-07 | |
| XT-M4_hVH_V1.0 | DP-54, VH3; 3-07 | |
| XT-M4_hVL_V2.4 | DPK-9 Vk1; 02 | G66R |
| XT-M4_hVL_V2.5 | DPK-9 Vk1; 02 | D70I |
| XT-M4_hVL_V2.6 | DPK-9 Vk1; 02 | T69S |
| XT-M4_hVL_V2.7 | DPK-9 Vk1; 02 | L46R |
| XT-M4_hVL_V2.8 | DPK-9 Vk1; 02 | |
| XT-M4_hVL_V2.9 | DPK-9 Vk1; 02 | F71Y |
| XT-M4_hVL_V2.10 | DPK-9 Vk1; 02 | |
| XT-M4_hVL_V2.11 | DPK-9 Vk1; 02 | |
| XT-M4_hVL_V2.12 | DPK-9 Vk1; 02 | |
| XT-M4_hVL_V2.13 | DPK-9 Vk1; 02 | |
| XT-M4_hVL_V2.14 | DPK-9 Vk1; 02 | |

### Example 19

### Competition ELISA Protocol

The binding of humanized XT-H2 and XT-M4 antibodies and of chimeric XT-M4 to human RAGE-Fc was characterized by competition enzyme-linked immunosorbent assay (ELISA). To generate a competitor, parental rat XT-M4 antibody was biotinylated. ELISA plates were coated overnight with 1 ug/ml human RAGE-Fc. Varying concentrations of the biotinylated XT-M4 were added in duplicate to wells (0.11-250 ng/ml), incubated, washed and detected with streptavidin-HRP. The calculated ED50 of biotinylated parental rat XT-M4 was 5 ng/ml. The IC50 of chimeric and each humanized XT-M4 antibody was calculated when competed with 12.5 ng/ml biotinylated parental XT-M4 antibody. Briefly, plates were coated overnight with 1 ug/ml human RAGE-Fc. Varying concentrations of chimeric or humanized antibodies mixed with 12.5 ng/ml biotinylated parental rat XT-M4 were added in duplicate to wells (in the range of 9 ng/ml to 20 ug/ml). Biotinylated parental rat XT-M4 antibodies were detected with streptavidin-HRP and IC50 values were calculated. The IC50 values determined for the humanized antibodies by competition ELISA analysis are shown in Table 11.

TABLE 11
IC50 Values for Humanized XT-M4 Antibodies

| | | IC 50 in competition |
|---|---|---|
| Heavy Chain | Light Chain | ELISA with rat XT-M4, ug/m |
| hVH-V1.0 | hVL-V1.0 | 1.5-2.5 |
| hVH-V1.0 | hVL-V2.0 | 7.5>8.6 |
| hVH-V1.0 | hVL-V2.1 | 1.5-2 |
| hVH-V1.0 | hVL-V2.2 | 1.5-2 |
| hVH-V1.0 | hVL-V2.3 | 4.5-8 |
| hVH-V1.0 | hVL-V2.4 | 4.5-8.5 |
| hVH-V1.0 | hVL-V2.5 | 6.5>20 |
| hVH-V1.0 | hVL-V2.6 | >10.9 |
| hVH-V1.0 | hVL-V2.7 | 4-9.5 |
| hVH-V1.0 | hVL-V2.8 | >17 |
| hVH-V1.0 | hVL-V2.9 | >6.8 |
| hVH-V2.0 | hVL-V1.0 | >9.5 |
| hVH-V2.0 | hVL-V2.0 | 10.4 |
| hVH-V2.0 | hVL-V2.1 | 1.1 |
| hVH-V2.0 | hVL-V2.2 | >1.8 |
| hVH-V2.0 | hVL-V2.3 | 3.3 |
| hVH-V2.0 | hVL-V2.4 | 2.9 |
| hVH-V2.0 | hVL-V2.7 | 8.5 |
| hVH-V2.0 | hVL-V2.10 | 0.95 |
| hVH-V2.0 | hVL-V2.11 | 0.15-1.05 |
| hVH-V2.0 | hVL-V2.12 | 2.7 |
| hVH-V2.0 | hVL-V2.13 | 1.5 |
| hVH-V2.0 | hVL-V2.14 | 0.2 |
| hVH-V2.0 | hVL-V2.10 | 0.3-0.4 |
| hVH-V2.0 | hVL-V2.11 | 0.1-0.45 |
| hVH-V2.0 | hVL-V2.14 | 0.2 |

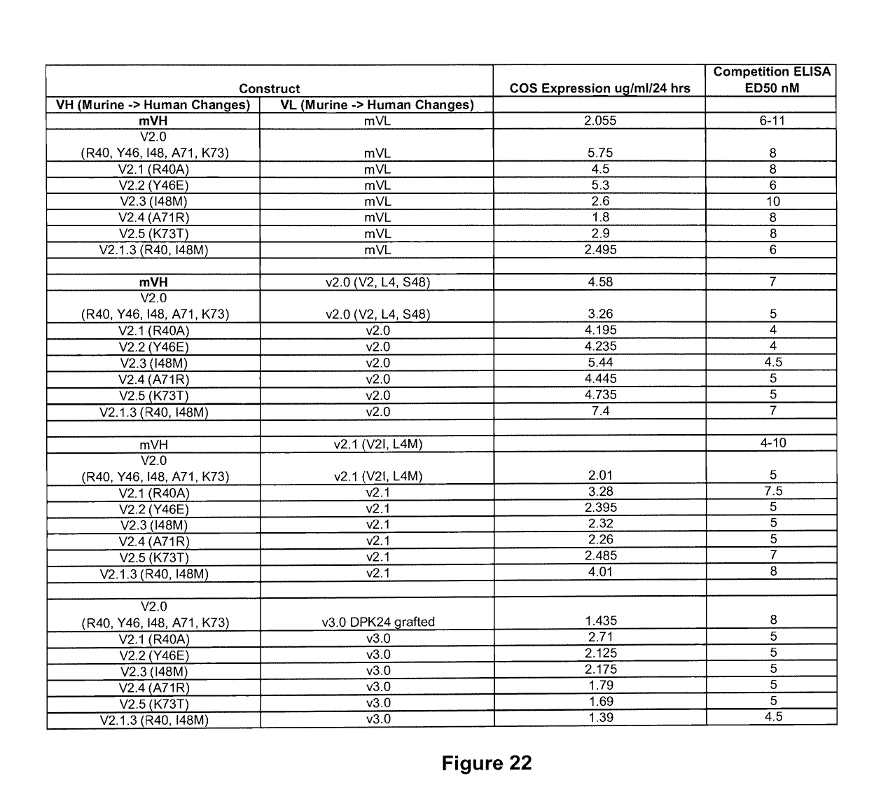

ED50 values for the binding of humanized XT-H2 antibodies to human RAGE-Fc were similarly determined by competition ELISA, and are shown in FIG. 22.

### Example 20

### Cross-Reactivity of Chimeric and Humanized XT-M4 Antibody to Other Cell Surface Receptors

Humanized XT-M4 antibodies XT-M4-hVH-V2.0-2 m/hVL-V2.10 and XT-M4-hVH-V2.0-2 m/hVL-V2.11, were tested along with chimeric XT-M4 for cross-reactivity with other RAGE-like receptors. These receptors were chosen because they are cell-surface expressed, like RAGE, and their interaction with ligand is similarly dependent on charge. Tested receptors were rhVCAM-1, rhICAM-1-Fc, rhTLR4 (C-terminal His tag), rhNCAM-1, rhB7-H1-Fc mLoxl-Fc, hLoxl-Fc and hRAGE-Fc (as a positive control). ELISA plates were coated overnight with 1 μg/ml of the listed receptor proteins. Varying concentrations of the above listed humanized and chimeric XT-M4 antibodies were added in duplicate to wells (0.03 to 20 μg/ml), incubated, washed and detected with anti-human IgG HRP. Table 12 shows the results of direct binding ELISA analysis of the binding of chimeric and humanized XT-M4 antibodies to human and mouse cell surface proteins. The data shown are OD450 values for binding detected between receptor and antibody at 20 μg/ml (highest concentration tested).

TABLE 12

| | XT-M4-hVH- | XT-M4-hVH- | |
|---|---|---|---|
| | 2.0-2m/ | V2.0-2m/ | Chimeric |
| | hVL-V2.10 | hVL-V2.11 | XT-M4 |
| rhVCAM-1 | 0.010 | 0.012 | 0.004 |
| rhlCAM-1-Fc | 0.007 | 0.004 | 0.004 |
| rhTLR4 | 0.001 | 0.003 | 0.000 |
| rhNCAM-1 | 0.004 | 0.011 | 0.006 |
| rhB7-H1-Fc | 0.010 | 0.009 | 0.003 |
| mLox1-Fc | 0.016 | 0.010 | 0.010 |
| hLox1-Fc | 0.007 | 0.022 | 0.017 |
| hRAGE-Fc | 3.808 | 3.832 | 3.797 |

### Example 21

### BIACORE™ Binding Assay of Binding to Soluble Human RAGE

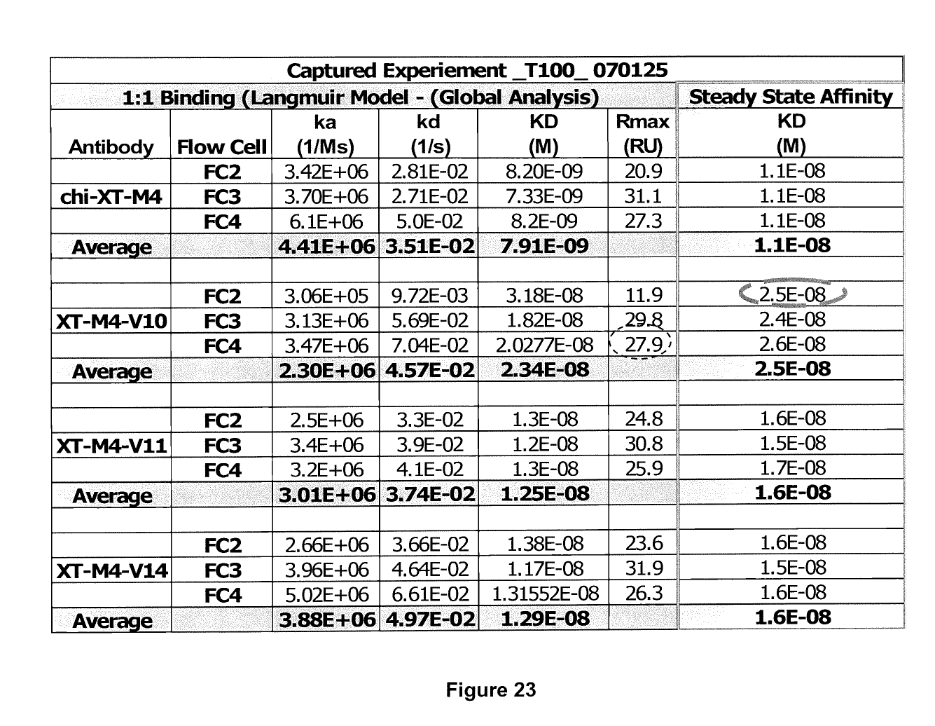

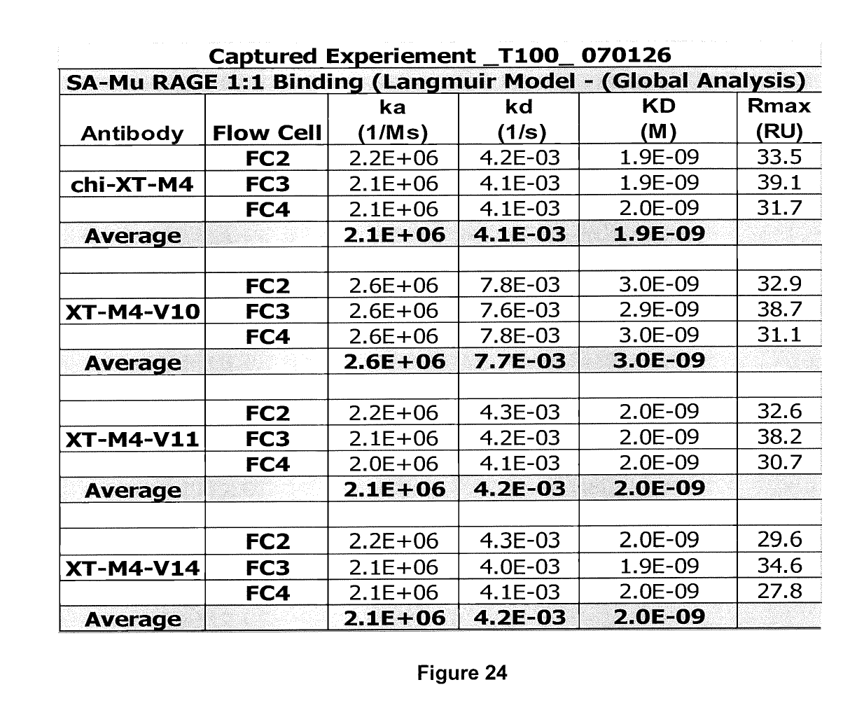

The binding of chimeric antibody XT-M4 and of humanized XT-M4 antibodies to soluble human RAGE (hRAGE-SA) and soluble murine RAGE (mRAGE-SA) was measured by BIACORE™ capture binding assay. The assays were performed by coating anti-human Fc antibodies onto a CM5 BIA chip with 5000 RU (pH 5.0, 7 min.) in flow cells 1-4. Each antibody was captured by flowing at 2.0 μg/ml over the anti-Fc antibodies in flow cells 2-4 (flow cell 1 was used as a reference). Solutions of a purified soluble human streptavidin-tagged RAGE (hRAGE-SA) at concentrations of 100 nM, 50 nM, 25 nM, 12.5 nM, 6.25 nM, 3.125 nM, 1.25 nM and 0 nM were flowed over the immobilized antibodies in duplicate, with dissociation for 5 minutes, and kinetic rate constants (kₐ and k_{d}) and association and dissociation constants (Kₐ and K_{d}) for binding to hRAGE-SA were determined. The results for binding of chimeric XT-M4 and humanized antibodies XT-M4-V10, XT-M4-V11, and XT-M4-V14 for binding to hRAGE-SA and mRAGE-SA are shown in FIGS. 23 and 24, respectively.

### Example 22

### Optimization of Species Cross Reactivity of Lead Antibody XT-H2

Species cross reactivity is engineered by a process of randomly mutating the XT-H2 antibody, generating a library of protein variants and selectively enriching those molecule that have acquired mutations that result in mouse-human RAGE cross reactivity. Ribosome display (Hanes et al., 2000, Methods Enzymol., 328:404-30) and phage display (McAfferty et al., 1989, Nature, 348: 552-4) technologies are used.

Preparing ScFv Antibodies Based on Antibodies XT-H2 and HT-M4

A. ScFv Antibodies Based on XT-H2

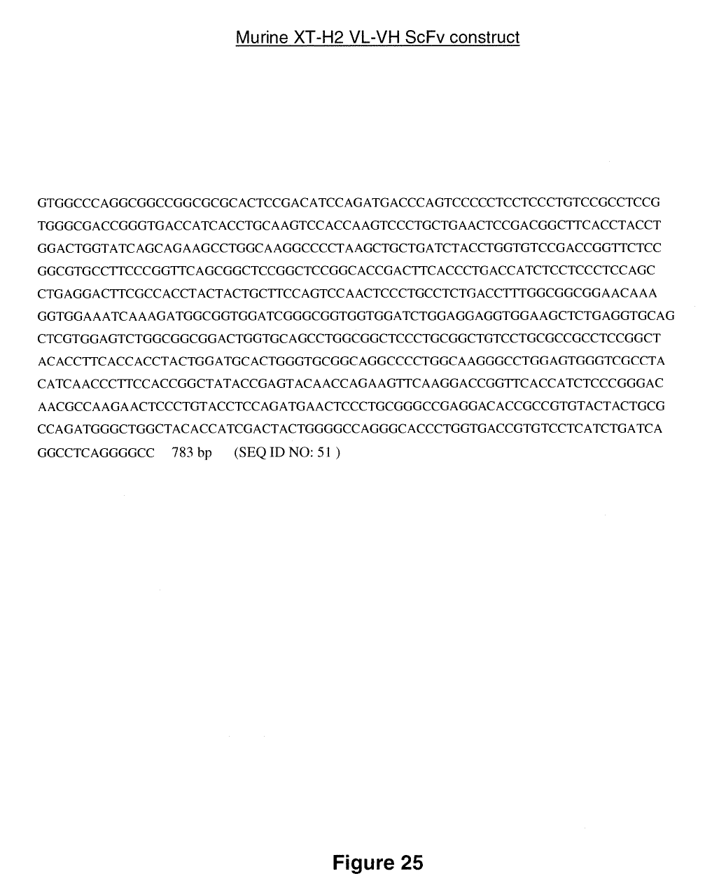

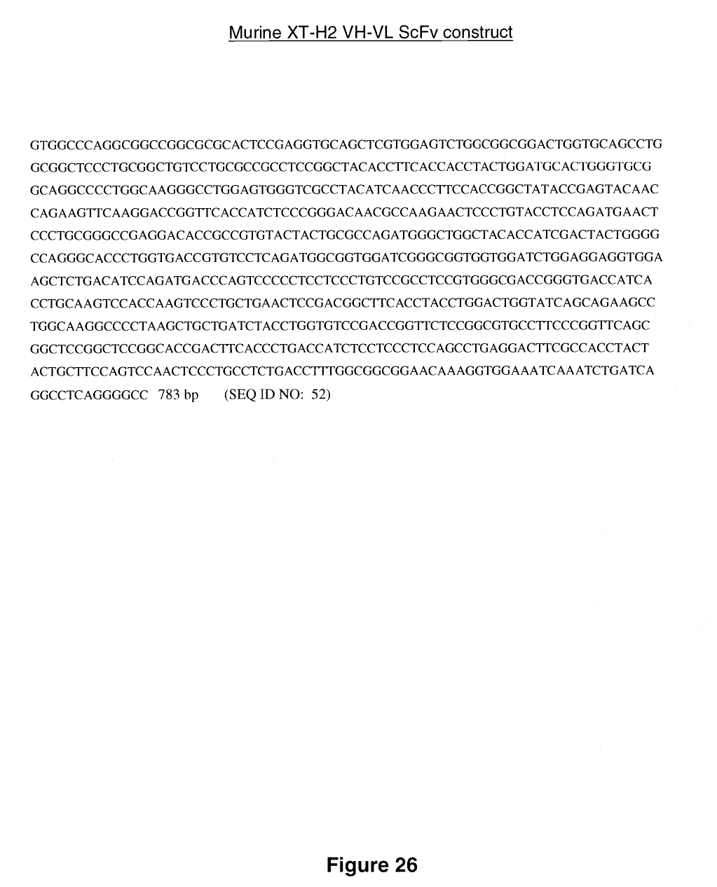

Two ScFv constructs comprising the V regions of XT-H2 were synthesized in either the VHNL format or the VLNH format connected by means of a flexible linker of DGGGSGGGGSGGGGSS (SEQ ID NO: 50). The sequences of the ScFv constructs configured as VL-VH and VH-VL are shown in FIG. 25 (SEQ ID NO:51) and FIG. 26 (SEQ ID NO:52), respectively.

B. ScFv Antibodies Based on XT-M4

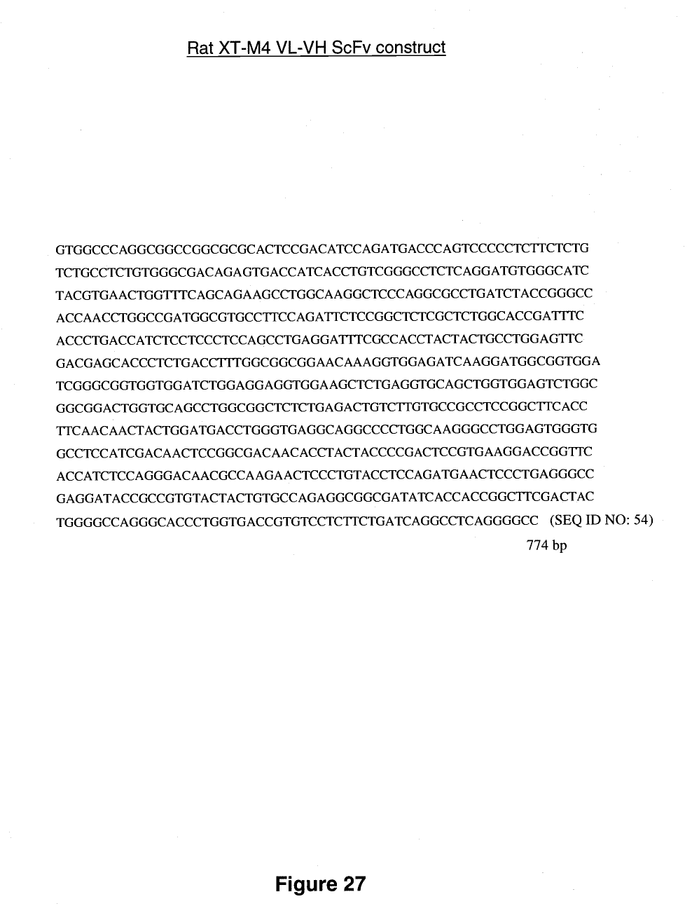

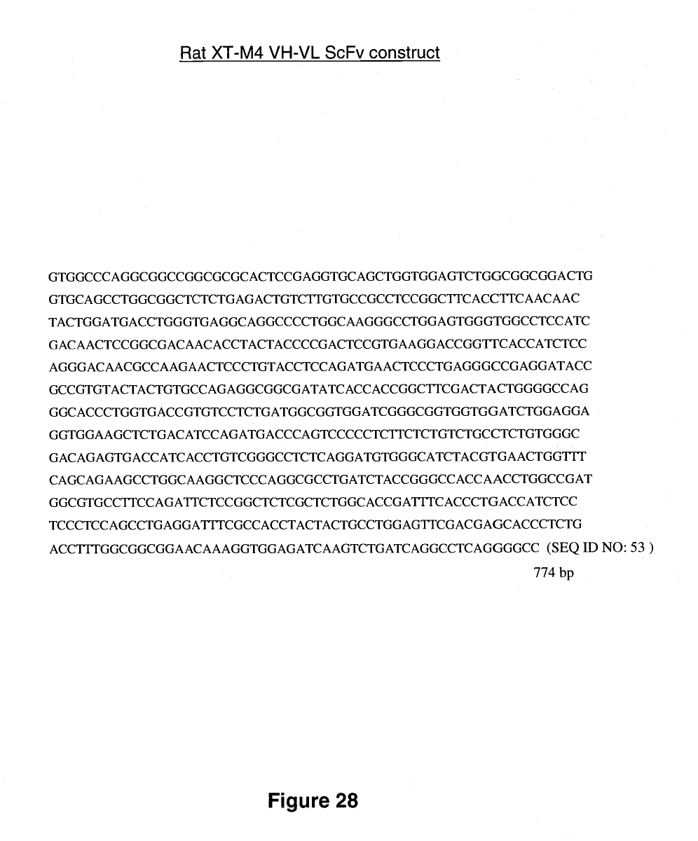

Two ScFv constructs comprising the V regions of XT-M4 were synthesized in either the VHNL format or the VLNH format connected by means of a flexible linker of DGGGSGGGGSGGGGSS (SEQ ID NO: 50). The sequences of the ScFv constructs configured as VL-VH and VH-VL are shown in FIG. 27 (SEQ ID NO:54) and FIG. 28 (SEQ ID NO: 53), respectively.

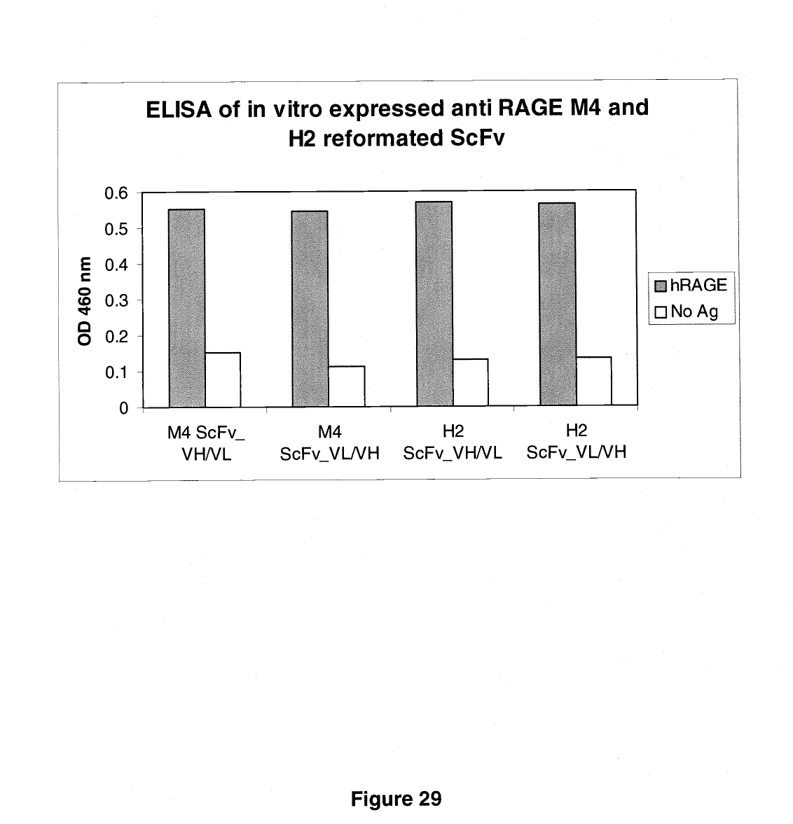

FIG. 29 shows ELISA data of in vitro transcribed and translated M4 and H2 constructs. ELISA plates coated with human RAGE-Fc (5 ug/ml) or BSA (200 ug/ml) in bicarbonate buffer overnight at 4° C., washed with PBS+tween 0.05% and blocked for 1 hour at room temperature with 2% milk powder PBS. Plates were incubated with in vitro translated ScFv for 2 hours at room temp. Plates were blocked and detection was with anti-Flag antibody (1/1000 dilution) followed by rabbit anti-mouse HRP (1/1000 dilution). The data shows that ScFv constructs of the variable regions of the XT-H2 and XT-M4 anti-RAGE antibodies in either the VLNH or VHNL configurations can produce functional folded protein that binds specifically to human RAGE. Values for Kd of the ScFv in both formats as determined by BIACORE™ are used to determine the optimum antigen concentrations for selection experiments.

C. Selection and Screening Strategy to Recovery Variants with Improved Mouse/Human RAGE Cross Reactivity

A library of variants is created by error-prone PCR (Gram et al., 1992, PNAS 89:3576-80). This mutagenesis strategy introduces random mutations over the whole length of the ScFv gene. The library is then transcribed and translated in vitro using established procedures (e.g., Hanes et al., 2000, Methods Enzymol., 328:404-30). This library is subjected to round 1 of selection on human-RAGE-Fc, the non-bound ribosomal complexes are washed away, and the antigen-bound ribosomal complexes are eluted. The RNA is recovered, converted to cDNA by RT-PCR and subjected to round 2 of selection on mouse RAGE-Fc. This alternating selection strategy preferentially enriches clones which bind to both human and mouse RAGE-Fc. The output from this selection is then put through a second 2 of error-prone PCR. The library generated is then subjected to round 3 and round selections on human-RAGE-Fc and mouse RAGE-Fc, respectively. This process is repeated as required. The output pools of RNA from each selection step are converted to cDNA and cloned into a protein expression vector pWRIL-1 to evaluate species cross reactivity of variant ScFvs. The pools of diversity are also sequenced to evaluate diversity to determine if selections are moving towards dominant clones that have species cross reactivity.

### Example 23

Affinity Maturation of Lead Antibody XT-M4

Improved affinity translates into a potential benefit of reduced dose or frequency of dose and/or increased potency. The affinity for hRAGE is improved by affinity maturation, using a combined process of targeted mutagenesis to the VH-CDR3 coupled to random error-prone PCR mutagenesis (Gram et al., 1992, PNAS 89:3576-80). This generates a library of antibody variants from which molecules are recovered that have an improved affinity for human-RAGE whilst maintaining species cross reactivity for mouse-RAGE-Fc. Ribosome display technology (Hanes et al, 1997, supra) and phage display technology (McAfferty et al., 1989, supra) are used.

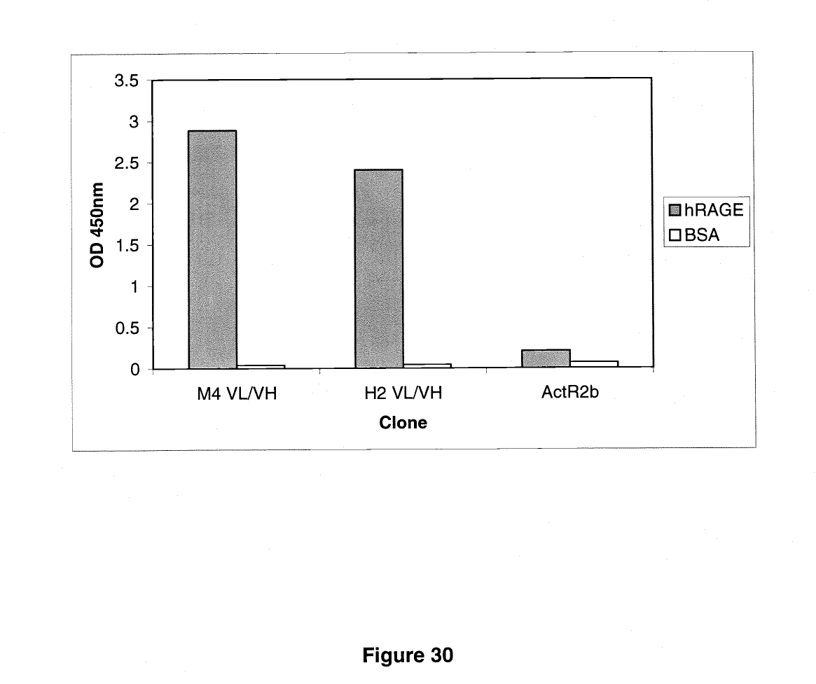

FIG. 30 shows ELISA binding data of XT-M4 and XT-H2 ScFv constructs in pWRIL-1 in the VL-VH format, expressed as soluble protein in Escherichia coli and tested for binding on human RAGE-Fc and BSA. ActRIIb represents a non-binding protein expressed from the same vector as a negative control. ELISA plates were coated with human RAGE-Fc (5 ug/ml) or BSA (200 ug/ml) in bicarbonate buffer overnight at 4oC, washed with PBS+tween 0.05% and blocked for 1 hour at room temperature with 2% milk powder PBS. Periplasmic preparations of 20 ml E. coli cultures were performed using standard procedures. The final volume of periplasmic preparations of unpurified ScFv antibodies was 1 ml of which 50 ul was pre-incubated with anti-His antibody at 1/1000 dilution for 1 hour at room temperature in a total volume of 100 ul with 2% milk powder PBS. The cross linked periplasmic preparations were added to the ELISA plate and incubated for a further 2 hours at room temperature. The plates were washed 2 times with PBS+0.05% tween and 2 times with PBS and incubated with rabbit anti-mouse HRP at 1/1000 dilution in 2% milk powder PBS. The plates were washed as before and binding was detected using standard TMB reagents. The data shows that ScFv constructs of XT-M4 and XT-H2 antibodies in the VLNH configuration can produce functional folded soluble protein in E. coli that binds specifically to human RAGE. Starting Kd values of the ScFv in both formats as determined by BIACORE™ are used to determine the optimum antigen concentrations for affinity selections.

### Example 24

### Selection and Screening Strategy to Recovery Variants with Improved Affinity for hRAGE-Fc while Maintaining Species Cross Reactivity

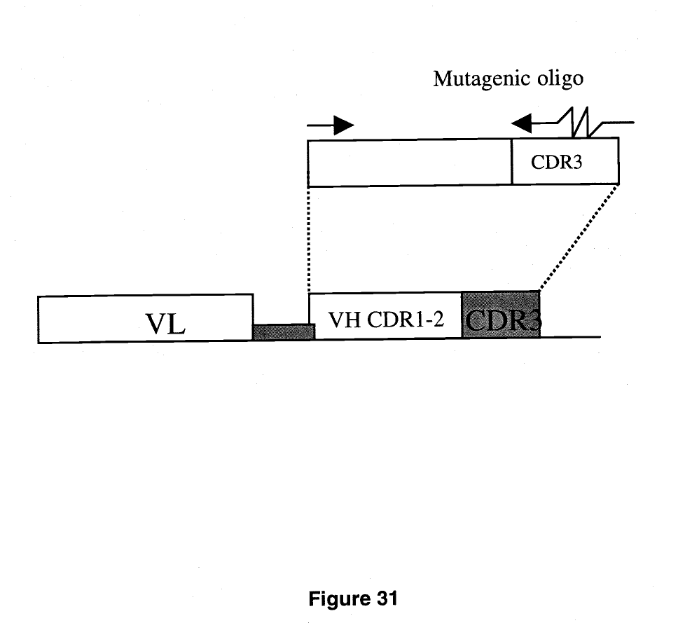

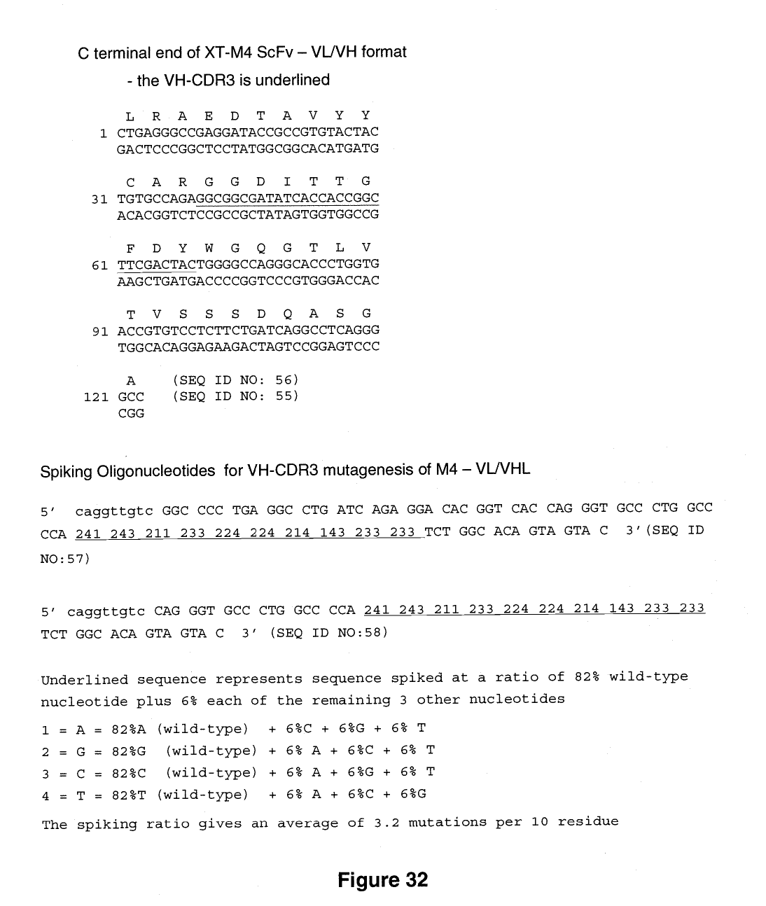

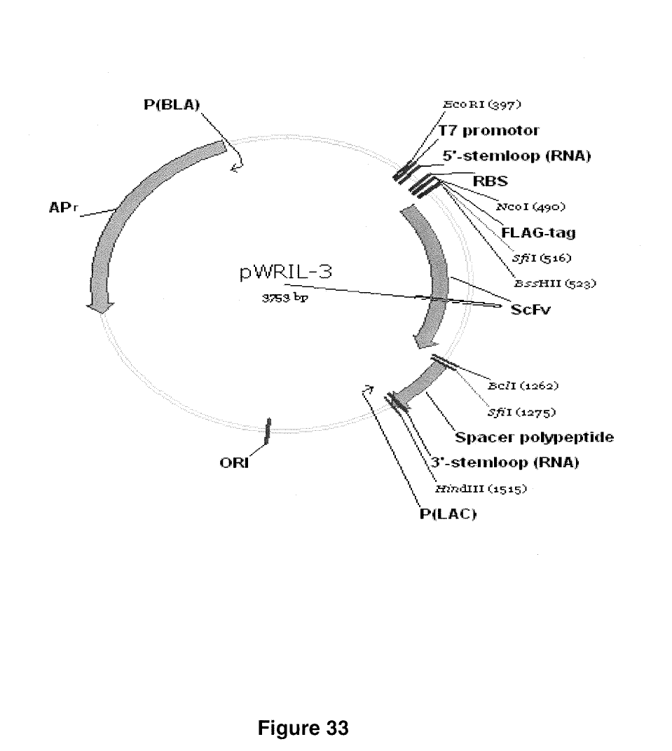

A library of variants is created by spiked mutagenesis of the VH-CDR3 of XT-M4 using PCR. FIG. 31 schematically represents how PCR is used to introduce spiked mutations into a CDR of XT-M4. (1) A spiked oligonucleotide is designed carrying a region of diversity over the length of the CDR loop and bracketed by regions of homology with the target V gene in the FR3 and FR4. (2) The oligonucleotide is used in a PCR reaction with a specific primer that anneals to the 5′ end of target V gene and is homologous to the FR1 region. FIG. 32 shows the nucleotide sequence of the C terminal end of the XT-M4 VL-VH ScFv construct (SEQ ID NO: 56). VH-CDR3 is underlined. Also shown are two spiking oligonucleotides (SEQ ID NOs:57-58) with a number at each mutation site that identifies the spiking ratio used for mutation at that site. The nucleotide compositions of the spiking ratios corresponding to the numbers are also identified.

The XT-M4-VHCDR3 spiked PCR product is cloned into the ribosome display vector pWRIL-3 as a Sfi1 fragment to generate a library. This library is subjected to selection on human biotinylated RAGE using ribosome display (Hanes and Pluckthun., 2000). Biotin labelled antigen is used as this allows for solution based selection which allows for more kinetic control over the process and increases the likelihood of preferentially enriching the higher affinity clones. Selections are performed either in an equilibrium mode at a decreasing antigen concentration relative to starting affinity or in a kinetic mode where improved off rate is specifically selected for using competition with unlabelled antigen over a empirically determined time frame. The non-bound ribosomal complexes are washed away, the antigen bound ribosomal complexes are eluted, the RNA is recovered, converted to cDNA by RT-PCR and a second round of selection on biotinylated mouse-RAGE-Fc is performed to maintain species cross reactivity. The output from this selection step containing ScFv variants with mutations in the VH-CDR3 is then subjected to a cycle 2 step of mutagenesis. This mutagenesis step involves the generation of random mutations using error prone PCR. The library generated is then subjected to round 3 selections on biotinylated human-RAGE-Fc at a 10 fold lower antigen concentration. This process is repeated as required. The output pools of RNA from each selection step are converted to cDNA and cloned into a protein expression vector pWRIL-1 to rank affinity and species cross reactivity of variant ScFv's. The pools of diversity are also sequenced to evaluate diversity to determine if selections are moving towards dominant clones.

### Example 25

Affinity Maturation of XT-M4 Using Phage Display

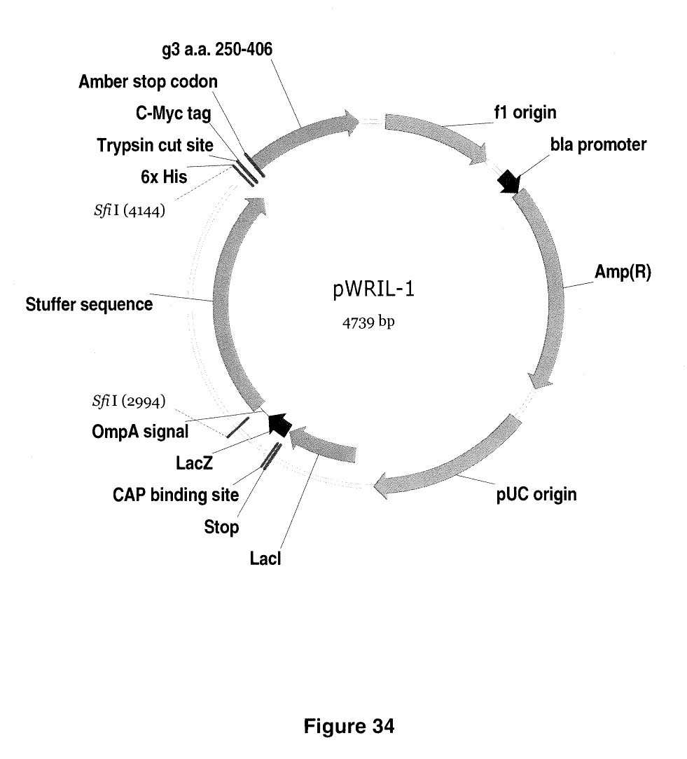

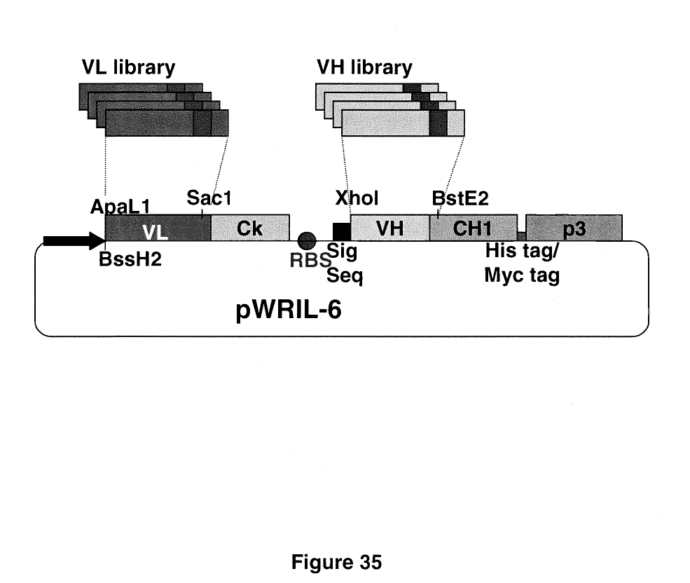

The VH-CDR3 spiked library is cloned into the phage display vector pWRIL-1 shown in FIG. 34 for selection on biotinylated hRAGE. Biotin labelled antigen will be used as this format is more compatible with affinity driven selections in solution. Selections are performed either in an equilibrium mode at a decreasing antigen concentration relative to starting affinity or in a kinetic mode where improved off rate is specifically selected for using competition with unlabelled antigen over an empirically determined time frame. Standard procedures for phage display are used.

ScFv can dimerize, which complicates selection and screening procedures. Dimerized ScFv potentially shows avidity-based binding and this increased binding activity can dominate selections. Such improvements in the ability of ScFv to dimerize rather than in any intrinsic improvement in affinity have little relevance in the final therapeutic antibody, which is generally an IgG. To avoid artifacts resulting from changes in ability to dimerize, Fab antibody formats are used, as they generally do not dimerize. XT-M4 has been reformatted as a Fab antibody and cloned into a new phage display vector pWRIL-6. This vector has restriction sites that span both the VH and VL regions and do not cut frequently in human germline V genes. These restriction sites can be used for shuffling and combinatorial assembly of VL and VH repertoires. In one strategy, VH-CDR3 and VL-CDR3 spiked libraries are both combinatorially assembled in the Fab display vector as shown in FIG. 34, and are selected for improved affinity.

### Example 26

### Physical Characterization of Chimeric Antibody XT-M4

Preliminary characterization by high-performance liquid chromatography (HPLC)/mass spectrometry (MS) peptide mapping and subunit analysis with on-line MS detection have confirmed that the amino acid sequence is as predicted from the chimeric XT-M4 DNA sequence. These MS data also indicated that the expected N-linked oligosaccharide sequence consensus site at Asn²⁹⁹SerThr is occupied and the two major species are complex N-linked biantennary core fucosylated glycans that contain zero or one terminal galactose residues, respectively. In addition to the expected N-linked oligosaccharide located in the Fc region of the molecule, an N-linked oligosaccharide was observed at a sequence consensus site (Asn⁵²AsnSer) in the CDR2 region of the heavy chain of chimeric XT-M4. The extra N-linked oligosaccharide is found primarily on only one of the heavy chains and comprises approximately 38% of the molecules as determined by CEX-HPLC analysis (there may be other acidic species that cannot be differentiated by primary structure, which may contribute to the total percent acidic species). The predominant species is a core fucosylated biantennary structure with two sialic acids. The absorptivity is used to calculate the concentration by measuring A₂₈₀. The theoretical absorptivity of chimeric XT-M4 was calculated to be 1.35 mL mg⁻¹ cm⁻¹.

The apparent molecular weight of chimeric XT-M4 as determined by non-reducing SDS-PAGE is approximately 200 kDa. The antibody migrates more slowly than expected from its sequence. This phenomenon has been observed for all recombinant antibodies analyzed to date. Under reducing conditions, chimeric XT-M4 has a single heavy chain band migrating at approximately 50 kDa and a single light chain migrating at approximately 25 kDa. There is also has an additional band that migrates just above the heavy chain band. This band was characterized by automated Edman degradation and was determined to have an NH₂-terminal that corresponds to the heavy chain of chimeric XT-M4. These results, along with the increase in molecular weight observed by SDS-PAGE, indicate that the additional band is consistent with a heavy chain that has the extra N-linked oligosaccharide in the CDR2 region.

The predicted isoelectric point (pI) of chimeric XT-M4 based on the amino acid sequence is 7.2 (without COOH-terminal Lys in the heavy chain). IEF resolved chimeric XT-M4 into approximately ten bands migrating within a pI range of approximately 7.4-8.3 with one dominant band that migrates with a pI of approximately 7.8. The pI determined by capillary electrophoresis isoelectric focusing was approximately 7.7.

Analysis of development material by cation exchange high performance liquid chromatography (CEX-HPLC) provides further resolution for chimeric XT-M4 species that differ in molecular charge. The majority of the observed charge heterogeneity is most likely due to the contributions from the sialic acids that are found on the additional N-linked oligosaccharide located in CDR2 region of the heavy chain. A minor portion of the charge heterogeneity observed may be attributed to the heterogeneity of COOH-terminal lysine.

### Example 27

### Removal of the Glycosylation Site

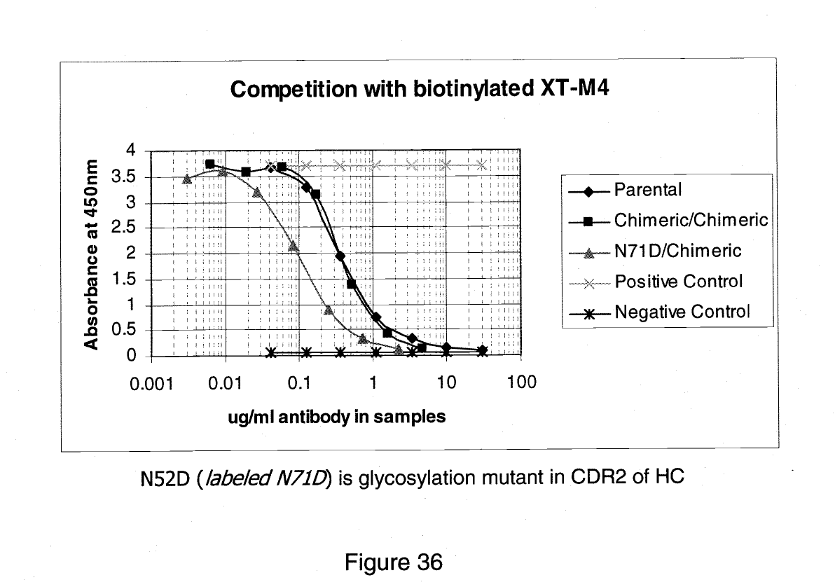

Mutation that converts asparagine (N) to aspartic acid (D) at position 52 (by Kabat numbering) in the heavy chain variable region of antibody XT-M4 improves the binding of the XT-M4 antibody to human RAGE as determined by ELISA analysis of direct binding to hRAGE-Fc, as shown in FIG. 36. The N52D mutation is in CDR2 of the heavy chain variable region of antibody XT-M4.

### Example 28

### Treatment of Sepsis and Listeriosis

Anti-RAGE antibodies were shown to provide significant therapeutic benefit in a standard murine model of polymicrobial, intra-abdominal sepsis. The results also showed that RAGE expression is highly detrimental to animals challenged systemically with Listeria monocytogenes as evidenced by the marked survival benefits observed in homozygous RAGE knock-outs and heterozygotes compared with wild-type animals.

A. Materials and Methods

All reagents and chemicals were purchased from Sigma (St. Louis, Mo.) unless otherwise stated. Rat monoclonal antibody XT-M4 IgG, with an affinity constant of 0.3 nM for murine dimeric RAGE, is described above. The anti-tumor necrosis factor alpha (TNF) monoclonal antibody TN3.1912 is a neutralizing IgG antibody derived from hamsters with high affinity binding to murine TNF. The challenge strain of Listeria monocytogenes was purchased from American Type Cell Cultures (ATCC # 19115, Manassas, Va.). All mouse strains used in these experiments were 2-6 months old and were specific-pathogen free animals maintained under Biosafety Level 2 conditions. BALB/c (Charles River Laboratories, Inc, Wilmington, Mass.) wild-type male mice, homozygous RAGE^{-/-} 129SvEvBrd male mice, heterozygous RAGE^{+/-} 129SvEvBrd male mice, and wild-type 129SvEvBrd male mice (breed in house at Wyeth). The RAGE knockout mouse was designed at Wyeth Research as a gene targeted conditional knockout in 129SvEv-Brd mice in which Cre recombinase excises exons 2, 3 and 4 (Lexicon Genetics, Inc, The Woodlands, Tex.). The resulting deletion results in frame shift truncation of the RAGE protein and protein is not produced. RAGE is not essential for viability in mice. RAGE null mice have no obvious phenotype and breed normally. Mice were assessed for survival up to seven days after CLP or L. monocytogenes challenge.

Quantitative microbiology was performed from organ samples obtained at necropsy from mice following both the CLP and listeriosis experiments. Blood samples were obtained from surviving animals at the time of sacrifice, and serum was collected and immediately placed on ice for cytokine determination. Serum cytokines were measured by an enzyme-linked immunosorbent assay multiplex assay using the custom-made plates and protocol provided by Meso Scale Delivery (Gaithersburg, Md.). The cytokines assayed were MCP-1, IL-1 beta, TNF alpha, Interferon γ and IL-6. Tissue samples were collected from the lung, liver, and spleen. Peritoneal fluid was obtained by ravaging the peritoneal cavity with 5 ml of sterile saline and withdrawing the fluid. The organ tissues were weighed and then pulverized to generate a suspension of tissue in TSB. Specimens were serially diluted and cultured at 37C aerobically on TSB (for gram-negative and gram-positive bacteria) and MacConkey agar (for gram-negative bacteria) to obtain quantitative bacterial counts standardized per gram of organ weight or CFU/ml peritoneal lavage fluid.

Animal tissues (lung, distal ileum) were also analyzed histologically by a pathologist blinded to the treatment assignment of each animal and scored on a defined pathology score graded from 0 (normal) to 4 (diffuse and extensive necrosis of tissue). Total lung water as a measure of pulmonary edema fluid was calculated from wet-to-dry ratios of lung tissue.

Statistical Design and Data Analysis. The primary endpoint in each experiment was survival. The animal experiments were performed using a numeric code system that blinded the investigators to the animal genotype or antibody treatment (versus serum control) until completion of the study. Numeric data are presented as mean (+/−SEM). Differences in survival were analyzed by a Kaplan-Meier survival plot and the log-rank statistic. The non-parametric one way ANOVA statistic Kruskal-Wallis (for multiple groups) or the Mann-Whitney U test (for two groups) was used to analyze differences between groups. Dunn's multiple comparisons post-test was utilized to confirm differences when analyzing comparisons involving multiple groups. A two-tailed P value of <0.05 was considered significant.

B. Cecal Ligation and Puncture Model

The CLP procedure has been described in detail previously [Echtenacher et al., 1990, J. Immunol., 145:3762-6]. Briefly, animals were anesthetized with an intraperitoneal injection of 200 microliters of a combination of ketamine (Bedford Co. Bedford, Ohio) (9 mg/ml) and xylazine (Phoenix, St. Josephs, Mo.) (1 mg/ml). The cecum was exteriorized through a midline abdominal incision approximately 1 cm in length. The cecum was then ligated with 5.0 monofilament at a level just distal to the ileocecal junction (>90% of the cecum ligated). The ante-mesenteric side of the cecum was punctured through and through with a 23 gauge needle. A scant amount of luminal contents was then expressed through both puncture sites to assure patency. The cecum was returned to the abdominal cavity, and the fascia and skin incisions were closed with 6.0 monofilament and surgical staples, respectively. Topical 1% lidocaine and bacitracin were applied to the surgical site post-operatively. All animals received a single intramuscular injection of trovafloxacin (Pfizer, New York) at a dose of 20 mg/kg immediately post-operatively, and a standard fluid resuscitation was administered with 1.0 ml subcutaneous injection of normal saline. Test animals were then returned to their individual cages and rewarmed using heat lamps until they regained normal posture and mobility.

Anti-RAGE mAb XT-M4 at doses of 7.5 mg/kg or 15 mg/kg (or serum control) was given once intravenously to wild-type mice 30-60 minutes before CLP or at the following time intervals post-CLP: 6, 12, 24, or 36 hours. As an additional control, five animals underwent sham surgery (laparotomy with mobilization and exteriorization of the cecum but without ligation or puncture).

Results

A. Survival of Homozygous RAGE Knock-Outs, RAGE Heterozygotes, and Wild-Type Animals after CLP.

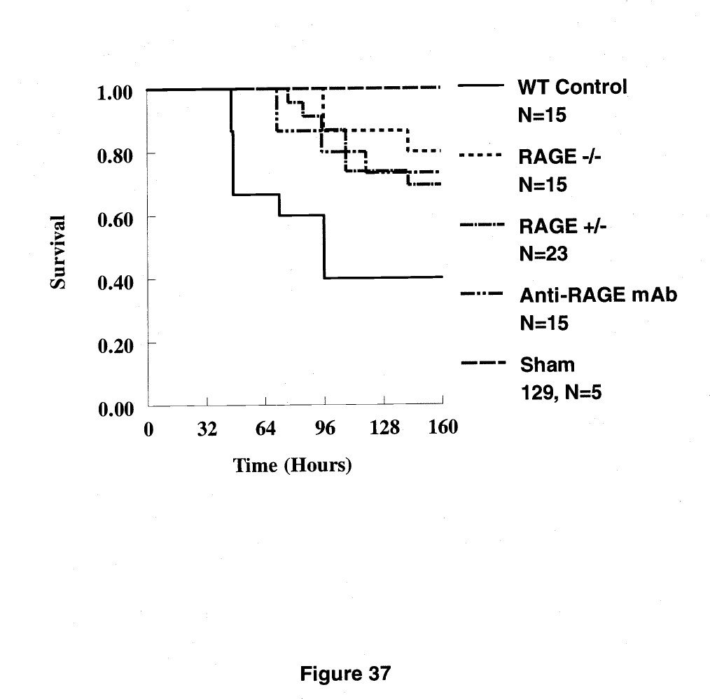

FIG. 37 shows that there was a significant survival advantage for both homozygous RAGE knockouts (n=15) and RAGE heterozygotes (n=23) compared to wild-type control animals (n=15) (P<0.001). RAGE heterozygotes were protected from lethal polymicrobial sepsis nearly as well as the homozygous RAGE knock-outs (RAGE^{-/-} vs. RAGE^{+/-}, P=ns). As expected sham surgery animals (n=5) all survived. An additional group of 15 wild-type 129SvEvBrd animals were given anti-RAGE mAb 30 minutes before CLP and had a similar survival advantage as the RAGE knock-outs when compared to the wild-type, serum-treated, control animals.

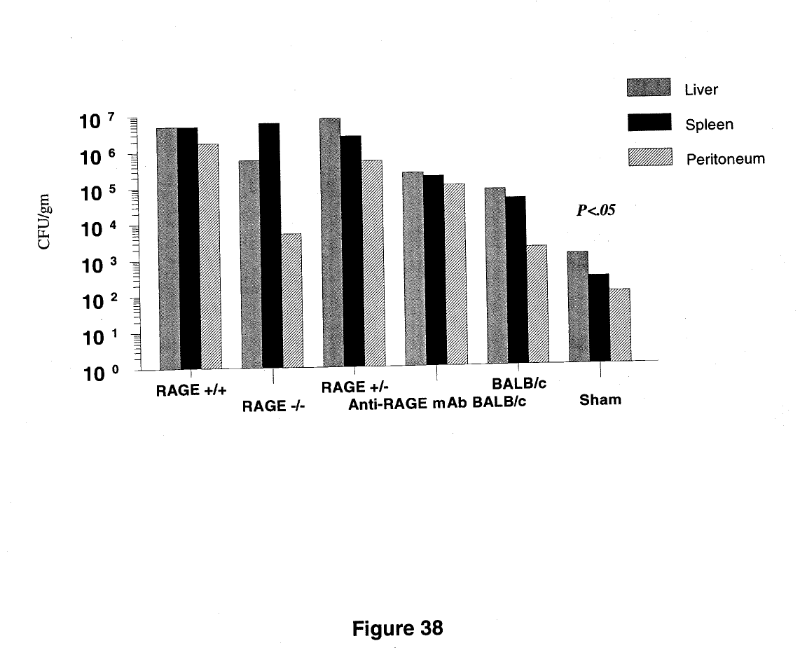

FIG. 38 shows tissue colony counts for aerobic gram-positive and gram-negative enteric bacterial organisms following CLP. The tissue concentrations in liver and splenic tissues and peritoneal fluid were similar in all three groups (P=ns) but were all significantly higher than sham-operated animals (P<0.05). The homozygous RAGE knock-outs had the lowest amount of lung water compared to other groups, although this did not reach significance (wet to dry ratio: 4.8±0.2-RAGE^{-/-} vs. 5.0±0.4-RAGE^{+/-} vs. 5.3±0.3-wt; P=ns).

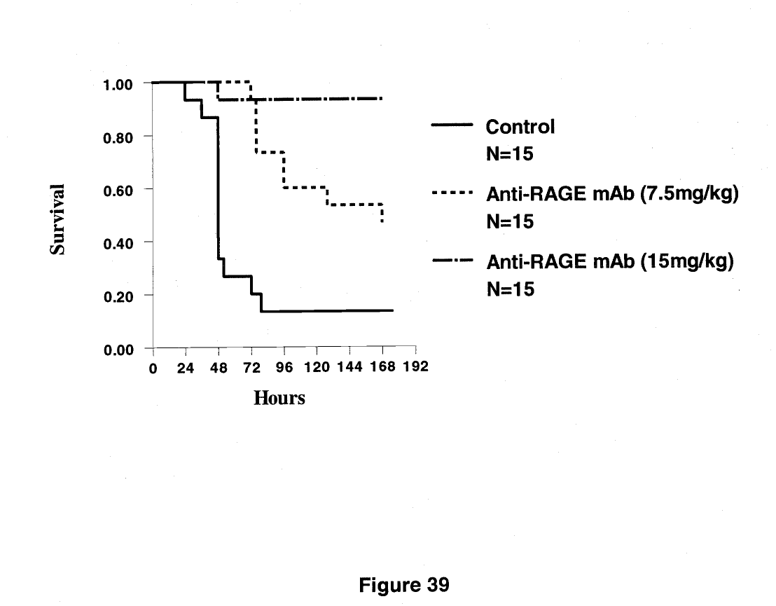

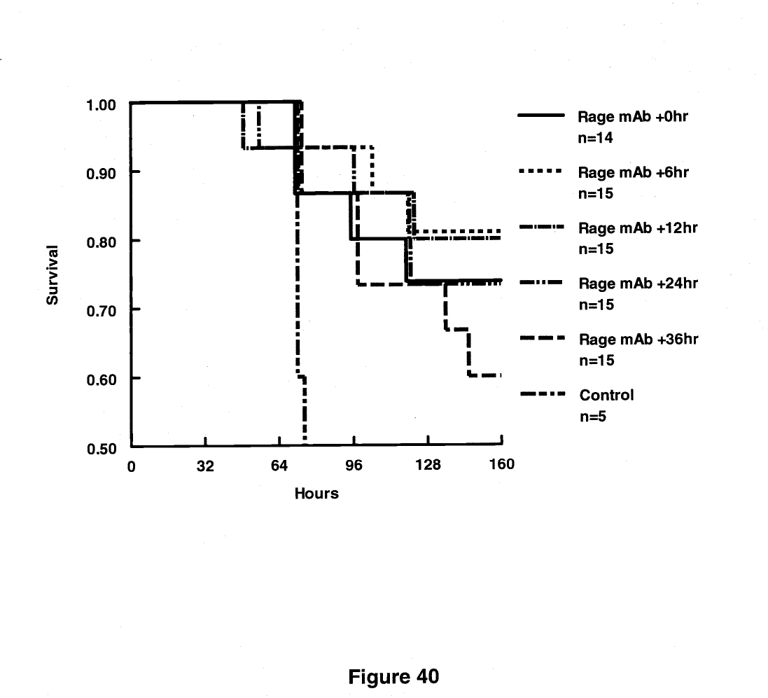

FIG. 39 shows that there was a significant difference in survival in BALB/c animals given control serum (n=15) and animals given anti-RAGE antibody (7.5 mg/kg group [n=15] or 15 mg/kg group [n=15]) 30-60 minutes before CLP. Optimal protective effects were achieved at 15 mg/kg of anti-RAGE mAb (P<0.05 vs. 7.5 mg/kg group; P<0.001 vs. serum control) and therefore this dose was employed in subsequent experiments with delayed mAb treatment following CLP. Animals given anti-RAGE antibody did not have significantly increased organ bacterial loads compared to control animals, but both groups had significantly more colony forming units (CFU)/gm of spleen and liver tissue than sham-treated control (n=5) animals. See Table 1. Histopathology of lung tissue and small bowel mucosa at necropsy examination was markedly abnormal in the serum control group while the pathological findings were significantly reduced in the anti-RAGE mAb group and the sham surgery group (Table 13).

TABLE 13
MICROBIOLOGIC AND PATHOLOGIC FINDINGS
FOLLOWING ANTI-RAGE mAb THERAPY IN CLP

| | | | Anti-RAGE mAb |
|---|---|---|---|
| Parameter | Sham | Serum Control | (15 mg/kg) |
| N | 5 | 15 | 15 |
| Aerobic Gram-negative | 0.6 ± 1.5* | 5643 ± 1281 | 4910 ± 395 |
| Bacteria (CFU/gm) | | | |
| Aerobic Gram-positive | 601 ± 548* | 15,616 ± 6800 | 11,222 ± 1873 |
| Bacteria (CFU/gm) | | | |
| Pathology score | 0.6 ± 0.5 | 3.0 ± 0.9** | 1.8 ± 1.1 |
| (lung, small bowel) | | | |
| Wet-to-dry ratio | 4.6 ± 0.6 | 5.3 ± 0.5 | 5.1 ± 0.6 |
| (lung tissue) | | | |

*P < .05 sham vs. other groups
**P < .005 control vs. sham or anti-RAGE mAb

FIG. 40 shows the effects of delayed administration of a single 15 mg/kg dose of anti-RAGE antibody at time intervals extended out to as long as 36 hours after CLP. The delayed monoclonal antibody treatment provided significant protection against lethality up to 24 hours after CLP (P<0.01). Delayed mAb administration up to 36 hours after CLP showed a favorable survival trend, but the differences were no longer significant compared the serum-treated control group (P=0.12). The tissue concentrations of aerobic enteric gram-negative and gram-positive bacteria did not differ between treatment groups (P=ns). The finding of a survival benefit after delayed administration of anti-RAGE antibody has important clinical implications since an intervention such as anti-RAGE antibody treatment typically cannot be given immediately after the inciting event in septic patients. These data provide support for the use of anti-RAGE mAb as a salvage therapy for patients with established severe sepsis.

C. Murine Listeriosis Challenge Model

BALB/c wild-type male mice, wild-type males, heterozygous RAGE^{+/-}-129SvEvBrd males, and homozygous RAGE^{-/-}-129SvEvBrd males were used in these experiments. A standard inoculum of L. monocytogenes was prepared from cultures grown 18 hours at 37° C. in trypticase soy broth (TSB) (BBL, Cockseyville, Md.). Bacteria were centrifuged at 10,000 g for 15 min at 4C and resuspended in phosphate buffered saline (PBS). Bacterial concentrations were adjusted spectrophotometrically and checked by quantitative dilutional plate counts on trypticase soy agar plates with 5% sheep RBCs (BBL, Cockseyville, Md.). Serial dilutions ranging from 10³-10⁶ colony forming units (CFU) L. monocytogenes were administered intravenously to determine the LD₅₀ for wild-type mice, homozygous RAGE^{-/-} knock-outs, RAGE^{+/-} heterozygotes, and wild-type mice given 15 mg/kg anti-RAGE mAb iv one hour before bacterial challenge. Animals were followed for 7 days after the administration of the intravenous challenge with L. monocytogenes and survivors were euthanized for tissue analysis and microbiologic study.

For the detailed differential quantitative microbiology and cytokine determinations, a standard inoculum of 10⁴ CFU was given intraperitoneally one hour after an intravenous infusion of the anti-RAGE mAb (15 mg/kg), anti-TNF mAb (20 mg/kg), or equal volume of 1% autologous murine serum as a control. Wild-type, RAGE^{+/-} and RAGE^{-/-} were also studied after 48 hours from this standard inoculum (n=5/group). Animals were euthanized 48 hours after L. monocytogenes challenge and quantitative microbiology was performed from liver and spleen tissues by mincing the tissue samples and serial dilution on blood agar plates.

Results

The LD₅₀ for wild-type mice was (logio) 3.31±0.2 CFU, while the LD₅₀ for heterozygous RAGE knock-outs was 5.98±0.39, and 5.10±0.47 for homozygous RAGE knock-outs. This difference of more than two orders of magnitude in LD₅₀ from systemic listeriosis was statistically significant (P<0.01) for both the RAGE heterozygotes and homozygotes compared to wild-type mice. The single dose of XY-M4 anti-RAGE antibody also provided wild-type mice significant protection from lethal systemic listeriosis with a LD₅₀ 4.69±0.55 (P<0.05 vs. wild-type control). The level of protection against listeriosis provided by the anti-RAGE mAb was similar to that observed in RAGE^{-/-} animals, but was not as great as that afforded RAGE^{+/-} animals (P<0.05).

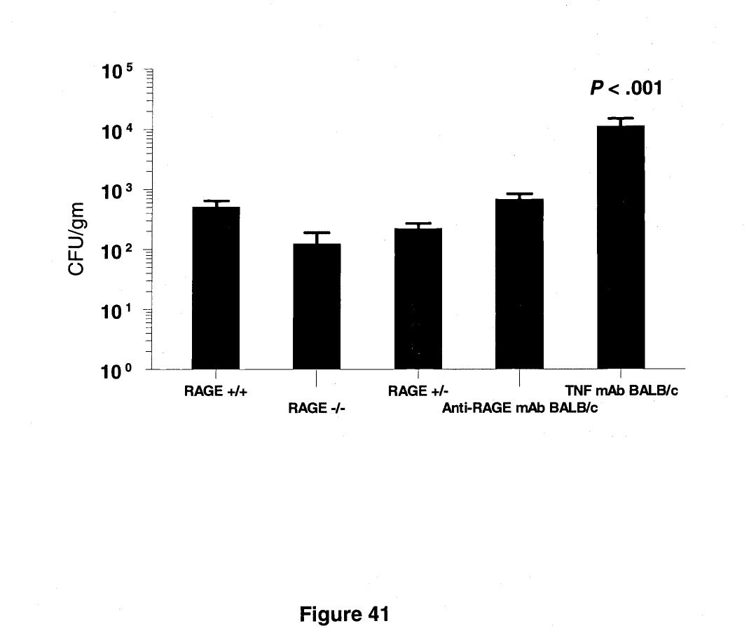

There was no statistically significant difference in quantitative level of L. monocytogenes isolated in liver and spleen tissues following a standard systemic challenge of 10⁴ CFU among groups (n=10/group) of wild-type control animals, animals given anti-RAGE antibodies, homozygous RAGE knock-outs, or RAGE heterozygotes. See FIG. 41. However, there was a highly statistically significant increase in organ bacterial concentrations in animals given the same inoculum of L. monocytogenes following the administration of an anti-TNF antibody (P<0.001).

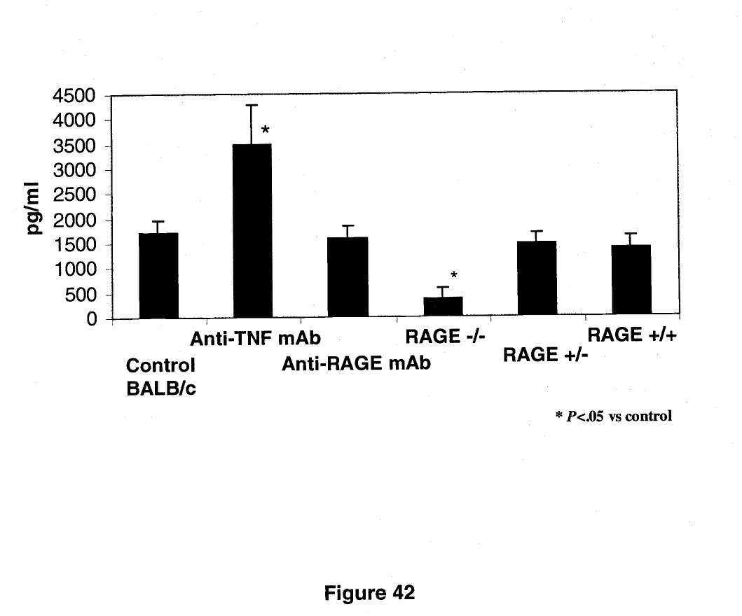

FIG. 42 shows serum levels of interferon γ following treatment. Cytokine determinations after Listeria challenge showed a significantly lower level of interferon γ in the homozygous RAGE knock-outs compared to control BALB/c animals. The BALB/c animals given anti-TNF mAb had a significantly higher level of interferon γ compared to BALB/c controls, whereas the animals given anti-RAGE mAb had interferon γ levels that were not statistically different than those of control animals. Similar results were observed with IL-6 (anti-TNF mAb group-459±121 pg/ml vs. control group-38±14 pg/ml; P<0.01) and MCP-1 (anti-TNF mAb-1363±480 pg/ml vs. control group 566±70 pg/ml; P<0.05). No significant differences were found in IL-6 or MCP-1 levels in RAGE deficient animals or in the anti-RAGE antibody treated group compared with the control group. Other cytokine determinations showed no significant differences.

Systemic Listeria monocytogenes challenge is a classic model for study of the innate and acquired immune response in mice. The Listeria challenge experiments show that homozygous RAGE knock-out animals and heterozygotes tolerate this infection remarkably better than do wild-type animals, indicating that the deleterious effects of RAGE are seen in an inflammatory state other than that accompanying polymicrobial sepsis. Wild-type animals given anti-RAGE mAb and RAGE knock-out animals appear to clear L. monocytogenes as well as wild-type animals. This is in contrast to animals given anti-TNF antibody in which the L. monocytogenes colony counts in tissue samples were markedly increased. Similarly, cytokine levels were increased after Listeria challenge in animals given anti-TNF mAb, but the levels were similar to those of controls in animals given anti-RAGE mAb.

These findings demonstrate that RAGE plays an important role in the pathogenesis of sepsis. In two separate CLP studies, a single dose (7.5 mg/kg at 1-6 hours post-CLP) of XT-M4 showed significant protection (65% survival) at day seven when compared to mice injected with 1.0% autologous mouse serum (20% survival). Two doses of XT-M4 (7 mg/kg at 6 and 12 hours post-CLP) protected about 85% of mice at day seven, compared to about 25% survival among mice that received diluted BALB/c serum. Administration of a single dose of anti-RAGE XT-M4 24 hours post CLP was also protective compared to control animals. The foregoing experiments demonstrate that RAGE plays an important role in the pathogenesis of sepsis and suggests that anti-RAGE antibodies may be useful therapeutic agents for the treatment of sepsis.

### Example 29

### Further Evaluation of Anti-RAGE Antibodies in the Murine CLP Model

The murine CLP model of sepsis results in a polymicrobial infection, with abdominal abscess and bacteremia, and recreates the hemodynamic and metabolic phases observed in human disease. In this model, the cecum is exteriorized through a midline abdominal incision approximately one centimeter in length, then ligated, and the anti-mesenteric side of the cecum is punctured through with a 23 gauge needle. The cecum is returned to the abdominal cavity, and the fascia and skin incisions are closed. The animals receive one intramuscular injection of trovafloxacin (20 mg/kg), and standard fluid resuscitation with 1.0 ml of normal saline subcutaneously. Animals were observed for 7 days after CLP, with deaths recorded as they were noted on interval checks throughout the day. As an additional control, animals underwent sham surgery consisting of a laparotomy with mobilization and exteriorization of the cecum, but without ligation or puncture. Survival outcomes are compared by Kaplan-Meier survival plots and analyzed with a non-parametric ANOVA test. The efficacy of the RAGE antibodies in prophylactic and therapeutic dosings and RAGE genetically modified mice were evaluated in the murine CLP model.

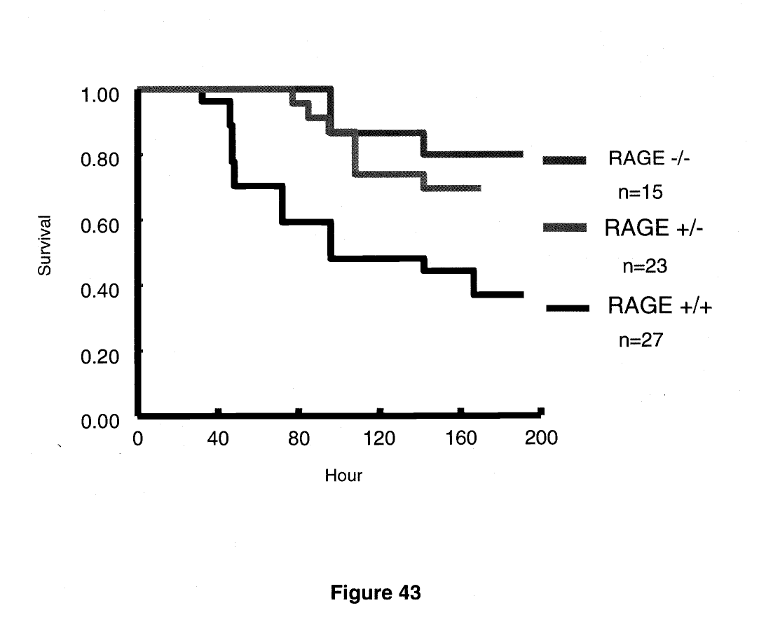

Homozygous RAGE null mice (RAGE−/−) mice showed a significant degree of protection from the lethal effects of cecal ligation and puncture, when compared to parental, wild-type mice, as shown in FIG. 43. By eight days post CLP, 80% of the RAGE−/− mice survived CLP, compared to 35% of the wild-type mice. RAGE−/+ animals behave similarly to RAGE−/− animals. As seen in the survival time analysis, the RAGE−/− animals had a significant survival advantage over the wild-type animals following CLP. These findings demonstrate that RAGE plays an important role in the pathogenesis of sepsis. RAGE is not essential for viability in mice.

Homozygous RAGE deleted mice have no obvious phenotype. The RAGE−/−, RAGE+/− and RAGE+/+ are on the 129SvEvBrd background strain.

The pharmacokinetic analysis of intraperitoneally (IP) administered, radiolabeled, XT-M4 (4 mg/kg) showed a T_{1/2} of 73 h, and a Tₘₐₓ of 6 h. XT-M4 also exhibited favorable pharmacokinetics in several mouse strains. Intravenous administration of 5 mg/kg XT-M4 to male BALB/c mice exhibited a very low serum clearance and T_{1/2} of 4˜5 days. Intraperitioneal administration of 5 mg/kg XT-M4 to male db/db mice also showed similar pharmacokinetics.

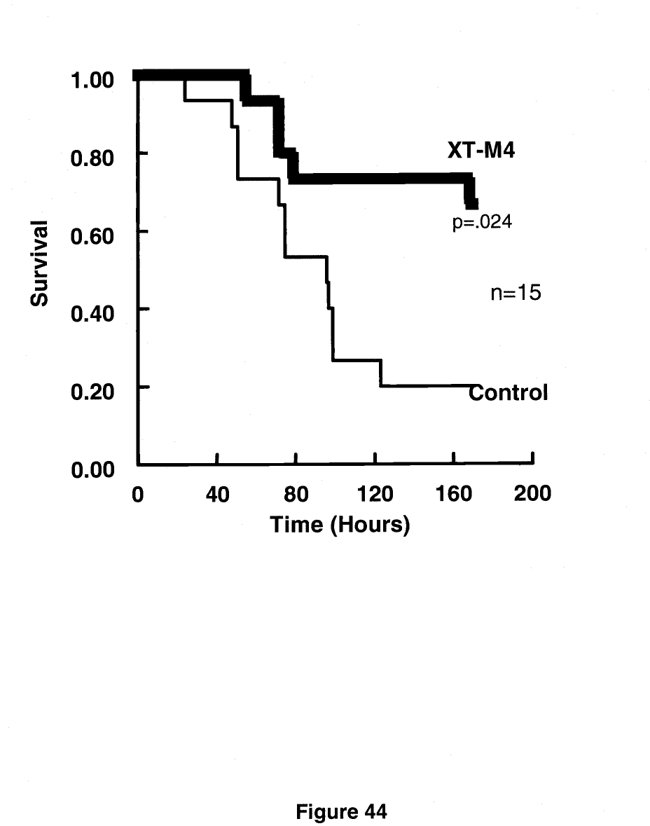

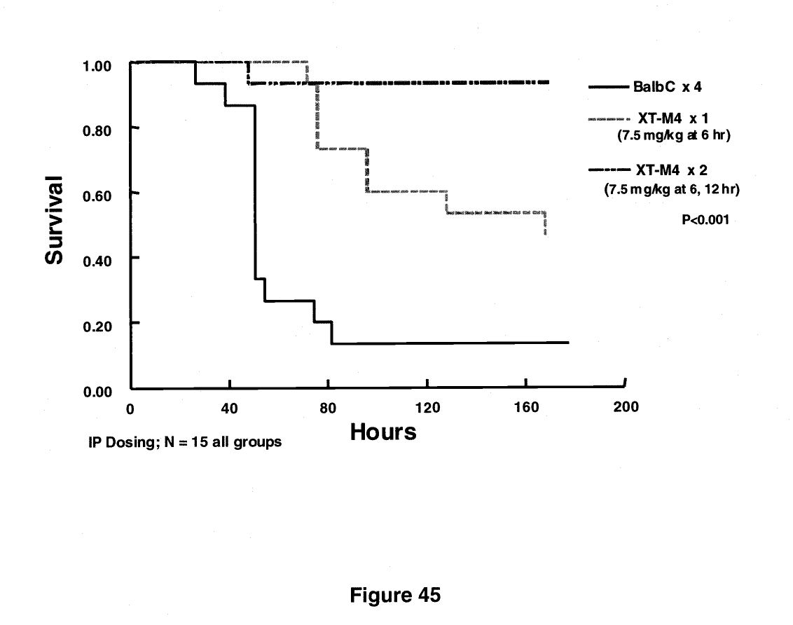

In two separate CLP studies of male BALB/c mice, a single dose (7.5 mg/kg at 0-6 hours post-CLP of XT-M4 showed significant protection (>50% survival) from the effects of CLP, when compared to mice injected with 1.0% autologous mouse serum (15%-20% survival), at day seven. See FIGS. 44 and 45. Two doses of XT-M4 (7.5 mg/kg at 6 and 12 hours post-CLP, final dose of 15 mg/kg, (FIG. 45) protected 90% of mice at day seven post-CLP compared to 15% survival in the control group. Optimal protection was observed with 15 mg/kg of XT-M4.

Pathological Scores from Mice with a CLP are Reduced in Anti-RAGE Antibody Treated Animals

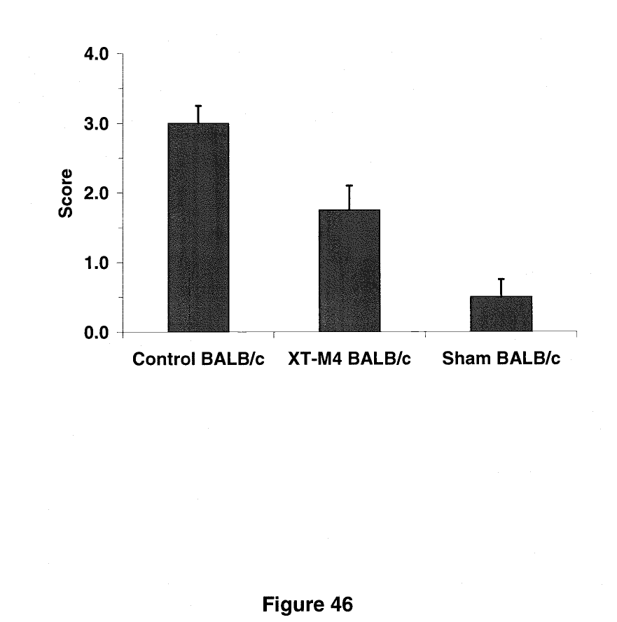

All animals surviving to day 8 were killed and underwent necropsy examination for histological evidence of organ injury, as well as pathology scoring of lung and small bowel. A defined pathology score graded from 0 (normal) to 4 (diffuse and extensive necrosis of tissue) was applied. Histopathology of lung tissue and small bowel mucosa at necropsy examination was markedly abnormal in the serum control group while the pathological findings were significantly reduced in the anti-RAGE XT-M4 treated group (15 mg/kg) and the sham surgery group. See FIG. 46. The reduction in the histopathology is consistent with the increased survival.

The tissue concentrations of aerobic enteric gram-negative and gram-positive bacteria did not differ between treatment groups. Quantitative microbiology was performed from organ samples obtained at necropsy from mice that survived following CLP. Tissue samples were collected from lung, liver, and spleen. Peritoneal fluid was obtained by lavaging the peritoneal cavity. Quantitative bacterial counts were standardized per gram of organ weight or colony forming units (CFU)/ml of peritoneal lavage fluid. Animals given XT-M4 antibody or RAGE−/− did not have significantly increased organ bacterial loads compared to control animals (p=ns) but both groups had significantly more colony forming units (CFU)/gm of spleen and liver tissue than sham-treated control (n=5) animals (p<0.05).

Anti-RAGE Antibodies are Protective in a Murine CLP Model with Antibiotics

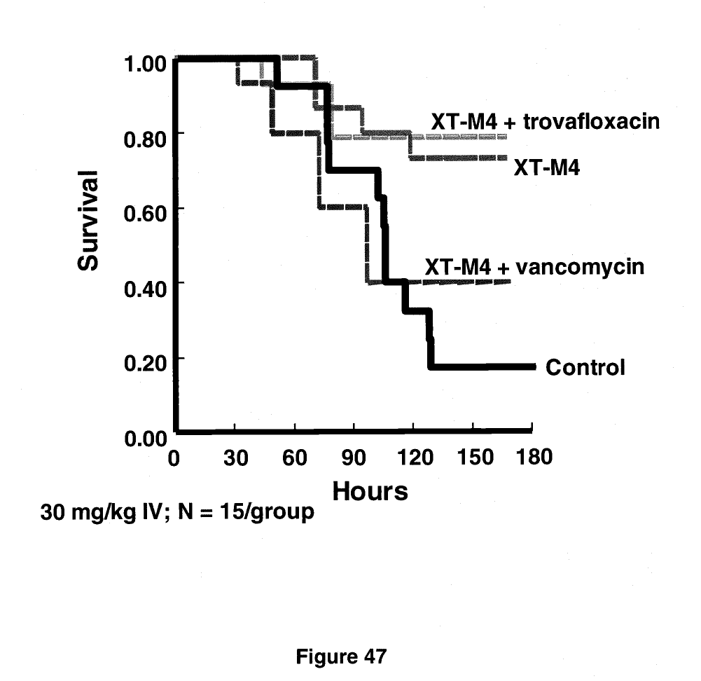

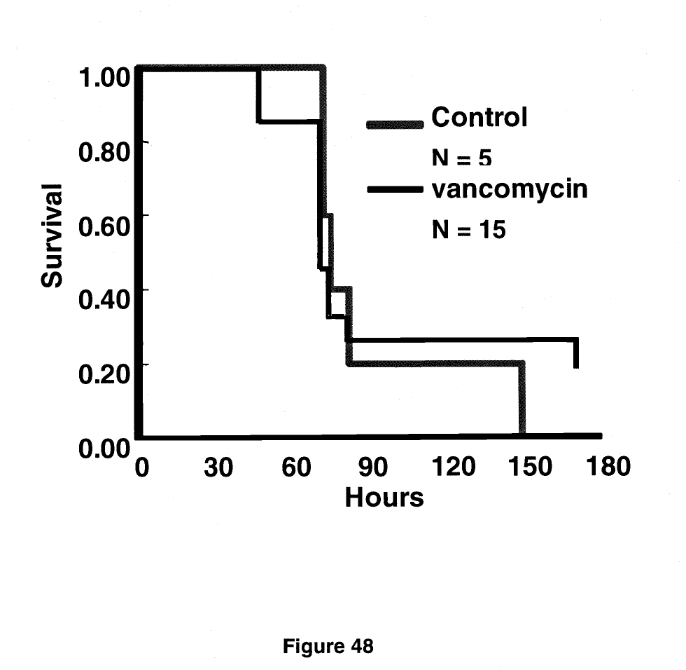

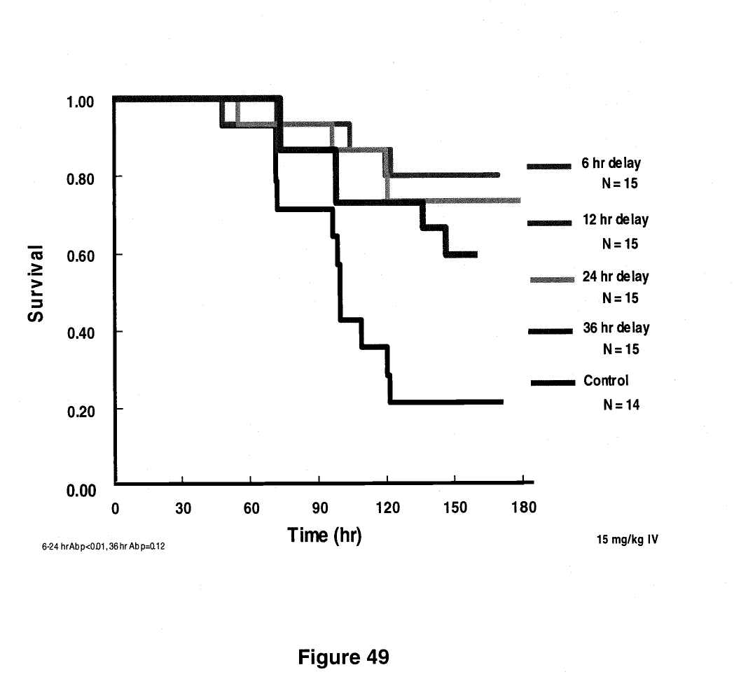

The intravenous administration of 30 mg/kg XT-M4 in the presence or absence of antibiotics protected the animals from the lethal effects of CLP. See FIG. 47. Mice were subjected to CLP at 0 h. Mice received an intravenous injection of 30 mg/kg XT-M4 or an equal volume of 1% autologous mouse serum. All groups received a dose of trovafloxacin (20 mg/kg IM) at time 0. In addition, trovafloxacin (20 mg/kg intramuscular) given at times of 24 and 48 h, or vancomycin (20 mg/kg IP) were administered at times of 0, 12, 24, 36, and 24 h post-CLP. Injection of vanocmycin alone resulted in a decrease in survival. See FIG. 48. No additive effects were observed when vancomycin or trovafloxacin were administered.

Anti-RAGE Antibodies are Protective in a Murine CLP Model with a Delayed Administration

Kaplan-Meier survival analysis following cecal ligation and puncture in animals with delayed treatment with anti-RAGE mAb versus serum control treatment given at various time intervals after CLP (FIG. 49). A delayed intravenous administration of the XT-M4 to male BALB/c mice at a dose of 15 mg/kg at 6, 12, or 24 hours post-CLP also resulted in significant survival of the animals (N=15, Control; n=14). The delayed monoclonal antibody treatment provided significant protection against lethality up to 24 hours after CLP (p<0.01). Delayed administration up to 36 hours after CLP showed a favorable survival trend (9/15 animals surviving), but the differences were no longer significant compared the serum-treated control group (p=0.12). The tissue concentration of aerobic enteric gram negative and gram-positive bacteria did not differ between treatment groups (p=ns).

### Example 30

### RAGE Modulation Does not Exacerbate Systemic Listeria Monocytogenes Infection

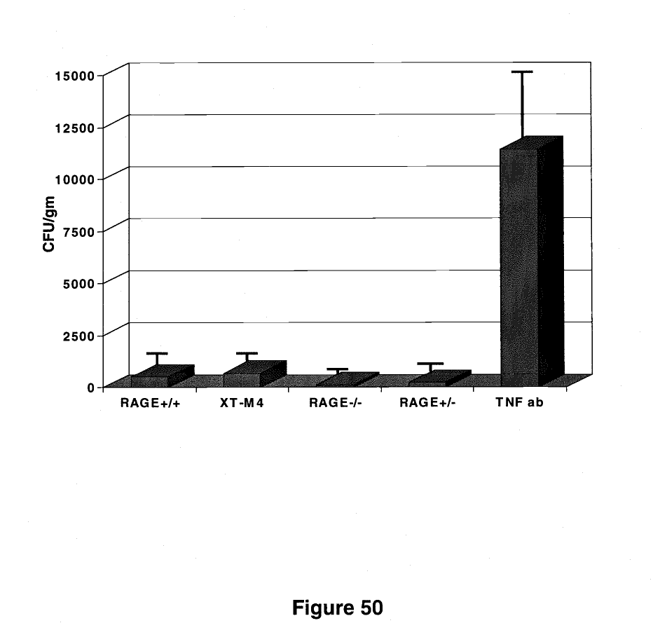

Inhibition or deletion of RAGE does not disrupt the host mechanism or clearance of microbial pathogens. The Listeria monocytogenes challenge is a well-known model for study of the innate and acquired immune response in mice. The LD₅₀ for wild-type mice was (log 10) 3.31±0.2 CFU, while the LD₅₀ for heterozygous RAGE+/−was 5.98±0.39, and 5.10±0.47 for homozygous RAGE−/−. This difference of more than two orders of magnitude in LD₅₀ from systemic listeriosis was statistically significant (p<0.01) for both the RAGE heterozygotes and homozygotes compared to wild-type mice. Mice were challenged with a systemic administration of Listeria monocytogenes (10⁴ colony forming units (CFU)) one hour after administration of antibody or control serum. Wild-type animals given anti-RAGE XT-M4 and RAGE−/− animals appear to clear L. monocytogenes as well as wild-type animals. Compared with the control group, the quantitative level (CFU/gm) of L. monocytogenes in hepatic and splenic tissue was unchanged by administration of the XT-M4 antibody (15 mg/kg) or in the RAGE null and RAGE heterozygous animals. In contrast, levels were increased with the administration of anti-TNF-α antibody (monoclonal antibody TN3.1912, 20 mg/kg). See FIG. 50. As expected, the anti-TNF monoclonal antibody significantly increased susceptibility of mice to listeriosis. Deletion or inhibition of RAGE did not exacerbate infection in this model.

### Example 31

### In Vivo Pre-Clinical Assay of Efficacy of Chimeric Anti-RAGE Antibody

A. Pharmacokinetics (PK)

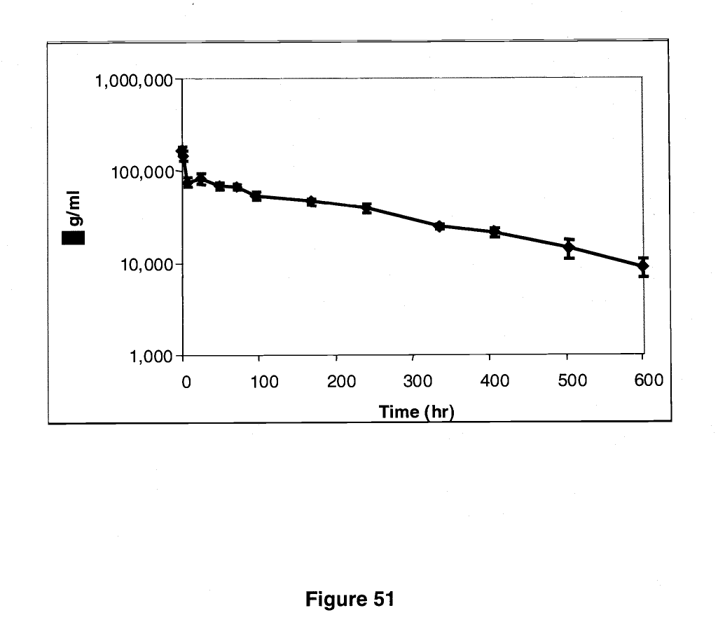

Serum concentration of chimeric antibody chimeric XT-M4 following a single IV dose of 5 mg/kg to male BALB/c mice (n=3) were evaluated for chimeric XT-M4 Serum concentration of antibody over time was measured with an IgG ELISA. The average serum exposure of the chimeric XT-M4 was (23,235 g•hr/mL) and the half-life is approximately one week (152 hours). See FIG. 51.

B. Evaluation of Protective Effect of Different Doses of Chimeric XT-M4 after CLP

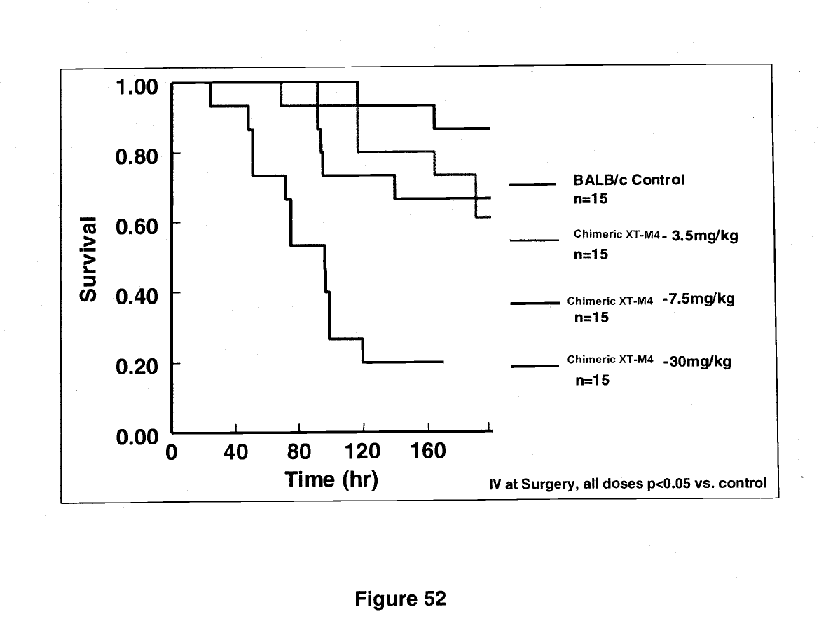

Abilities of chimeric antibody XT-M4 and the parental rat XT-M4 antibody to prolong survival of male BALB/c mice following CLP were determined following dosing at 3.5 mg/kg, 7.5 mg/kg and 30 mg/kg intravenously at the time of surgery, in comparison with serum control animals. The survival plot is shown in FIG. 52. A single intravenous dose (7.5 mg/kg at 0 hours post-CLP) of chimeric XT-M4 protected about 90% of mice at day seven post-CLP, when compared to mice injected with 1.0% autologous mouse serum (20% survival), at day seven (p<0.05). Doses of 3.5 mg/kg and 30 mg/kg of chimeric XT-M4 also provided significant protection (about 70% compared to control, p<0.05) of the mice at day seven post-CLP.

C. Evaluation of Protection Provided by Chimeric XT-M4 Given 24 Hours after CLP

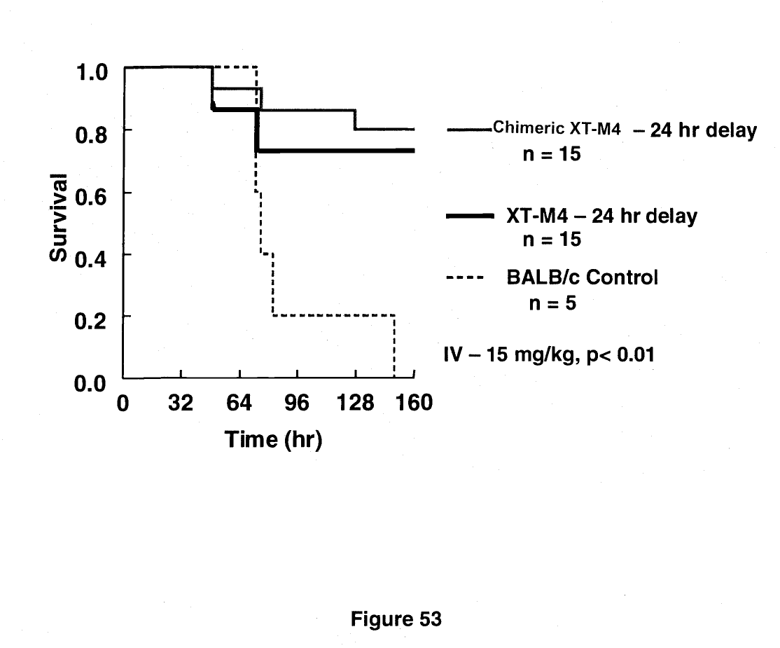

Differences in survival were analyzed by Kaplan-Meier survival plot following cecal ligation and puncture in animals with delayed treatment (p<0.01 for both antibody-treated groups compared to the serum control group). The comparability of chimeric to the rat anti-RAGE XT-M4 when administered at a dose of 15 mg/kg intravenously 24 hours after CLP model is depicted in FIG. 53. The level of protection provided by chimeric XT-M4 in the CLP model is similar to that provided by the parental rat XT-M4 antibody when administered therapeutically 24 hours post-CLP.

Summary of Results

The absence of RAGE protects mice from the lethal effects of CLP-induced sepsis. A single dose of XT-M4 protects mice from the lethal effects of CLP. No significant difference in tissue concentration of Listeria monocytogenes 48 hours post-systemic Listeria challenge in RAGE−/− or antibody treated mice, suggests no gross immunosuppression. The data show that replacement of the constant regions of rat antibody XT-M4 with human constant regions did not affect the binding activity of the antibody. In addition, the efficacy in the CLP model dosed prophylactically with chimeric XT-M4 showed that 90% of the animals were protected at a dose of 7.5 mg/kg. Chimeric XT-M4 and the parental XT-M4 antibody provide similar levels of protection in the CLP model when administered therapeutically 24 hours post-CLP.

All publications and patents mentioned herein are hereby incorporated by reference in their entirety as if each individual publication or patent was specifically and individually indicated to be incorporated by reference. In case of conflict, the present application, including any definitions herein, will control.

While specific embodiments of the subject invention have been discussed, the above specification is illustrative and not restrictive. Many variations of the invention will become apparent to those skilled in the art upon review of this specification and the claims below. The full scope of the invention should be determined by reference to the claims, along with their full scope of equivalents, and the specification, along with such variations.

We claim:

1. An antibody that binds specifically to RAGE and:
(a) competes for binding to RAGE with an antibody selected from the group consisting of XT-H1, XT-H2, XT-H3, XT-H5, XT-H7, and XT-M4;
(b) binds to an epitope of RAGE that is bound by an antibody selected from the group consisting of XT-H1, XT-H2, XT-H3, XT-H5, XT-H7, and XT-M4;
(c) comprises one or more complementarity determining regions (CDRs) of a light chain or heavy chain of an antibody selected from the group consisting of XT-H1, XT-H2, XT-H3, XT-H5, XT-H7, and XT-M4; or
(d) is a RAGE-binding fragment of an antibody according to (a), (b) or (c).

2. The antibody of claim 1, comprising a light chain variable region comprising at least two of the CDRs of a light chain variable region of an antibody selected from the group consisting of XT-H1, XT-H2, XT-H3, XT-H5, XT-H7, and XT-M4.

3. The antibody of claim 2, comprising a light chain variable region comprising three CDRs of a light chain variable region of an antibody selected from the group consisting of XT-H1, XT-H2, XT-H3, XT-H5, XT-H7, and XT-M4.

4. The antibody of claim 1, comprising a heavy chain variable region comprising at least two of the CDRs of a heavy chain variable region of an antibody selected from the group consisting of XT-H1, XT-H2, XT-H3, XT-H5, XT-H7, and XT-M4.

5. The antibody of claim 4, comprising a heavy chain variable region comprising three CDRs of a light chain variable region of an antibody selected from the group consisting of XT-H1, XT-H2, XT-H3, XT-H5, XT-H7, and XT-M4.

6. The antibody of claim 1, comprising
a light chain variable region comprising three CDRs of a light chain variable region of an antibody selected from the group consisting of XT-H1, XT-H2, XT-H3, XT-H5, XT-H7, and XT-M4; and
a heavy chain variable region comprising three CDRs of a heavy chain variable region of an antibody selected from the group consisting of XT-H1, XT-H2, XT-H3, XT-H5, XT-H7, and XT-M4.

7. The antibody of claim 1, comprising light and heavy chain variable regions which comprise three CDRs of a light chain variable region and three CDRs of a heavy chain variable region, respectively, of an antibody selected from the group consisting of XT-H1, XT-H2, XT-H3, XT-H5, XT-H7, and XT-M4.

8. The antibody of claim 1, wherein the antibody binds to human RAGE with a dissociation constant (Kd) in the range of from at least about 1×10⁻⁷ M to about 1×10⁻¹⁰ M.

9. The antibody of claim 1, wherein the antibody binds to the V domain of human RAGE.

10. The antibody of claim 1, wherein the antibody binds specifically to RAGE-expressing cells in vitro.

11. The antibody of claim 1, wherein the antibody binds specifically to RAGE-expressing cells in vivo.

12. The antibody of claim 1, wherein the antibody binds to RAGE and inhibits the binding of a RAGE binding partner (RAGE-BP) to the RAGE.

13. An antibody that binds specifically to RAGE, and
(a) comprises a light chain variable region selected from the group consisting of: XT-H1_VL (SEQ ID NO: 19), XT-H2_VL (SEQ ID NO: 22), XT-H3_VL (SEQ ID NO: 25), XT-H5_VL (SEQ ID NO: 23), XT-H7_VL (SEQ ID NO: 27), and XT-M4_VL (SEQ ID NO: 17), or
(b) comprises a light chain variable region having an amino acid sequence that is at least 90% identical to an amino acid sequence selected from SEQ ID NO: 19, SEQ ID NO: 22, SEQ ID NO: 25, SEQ ID NO: 23, SEQ ID NO: 27, and SEQ ID NO: 17; or
(c) is a RAGE-binding fragment of an antibody according to (a) or (b).

14. An antibody that binds specifically to RAGE and,
(a) comprises a heavy chain variable region selected from the group consisting of: IXT-H1_VH (SEQ ID NO: 18), XT-H2_VH (SEQ ID NO: 21), XT-H3_VH (SEQ ID NO: 24), XT-H5_VH (SEQ ID NO: 20), XT-H7_VH (SEQ ID NO: 26), and XT-M4_VH (SEQ ID NO: 16), or
(b) comprises a heavy chain variable region having an amino acid sequence that is at least 90% identical to an amino acid sequence selected from SEQ ID NO: 18, SEQ ID NO: 21, SEQ ID NO: 24, SEQ ID NO: 20, SEQ ID NO: 26, and SEQ ID NO: 16; or
(c) is a RAGE-binding fragment of an antibody according to (a) or (b).

15. The antibody of claim 13, which
(a) further comprises a heavy chain variable region selected from the group consisting of: XT-H1_VH (SEQ ID NO: 18), XT-H2_VH (SEQ ID NO: 21), XT-H3_VH (SEQ ID NO: 24), XT-H5_VH (SEQ ID NO: 20), XT-H7_VH (SEQ ID NO: 26), and XT-M4_VH (SEQ ID NO: 16), or
(b) further comprises a heavy chain variable region having an amino acid sequence that is at least 90% identical to an amino acid sequence selected from SEQ ID NO: 18, SEQ ID NO: 21, SEQ ID NO: 24, SEQ ID NO: 20, SEQ ID NO: 26, and SEQ ID NO: 16; or
(c) is a RAGE-binding fragment of an antibody according to (a) or (b).

16. The antibody of claim 1, comprising light and heavy chain variable regions having amino acid sequences of the light and heavy chain variable regions, respectively, of an antibody selected from the group consisting of XT-H1, XT-H2, XT-H3, XT-H5, XT-H7, and XT-M4.

17. The antibody of claim 1, which is selected from the group consisting of a chimeric antibody, a humanized antibody, a human antibody, a single chain antibody, a tetrameric antibody, a tetravalent antibody, a multispecific antibody, a domain-specific antibody, a domain-deleted antibody, a fusion protein, an Fab fragment, an Fab′ fragment, an F(ab′)₂ fragment, an Fv fragment, an ScFv fragment, an Fd fragment, a single domain antibody, and a dAb fragment.

18. The antibody of claim 1, comprising at least one mutation of an amino acid in a light or heavy chain variable region that removes a glycosylation site.

19. A chimeric antibody, or a RAGE-binding fragment thereof, comprising a light chain variable region amino acid sequence that is at least 90% identical to the XT-M4 light chain variable region amino acid sequence (SEQ ID NO: 17), and a heavy chain variable region amino acid sequence that is at least 90% identical to the XT-M4 heavy chain variable region amino acid sequence (SEQ ID NO: 16), and further comprising constant regions derived from human constant regions.

20. A chimeric antibody, or a RAGE-binding fragment thereof, comprising
a light chain variable region having the amino acid sequence of the XT-M4 light chain variable region (SEQ ID NO: 17),
a heavy chain variable region having the amino acid sequence of the XT-M4 heavy chain variable region sequence (SEQ ID NO: 16),
a human kappa light chain constant region and a human IgG1 heavy chain constant region.

21. A humanized antibody, or a RAGE-binding fragment thereof, that comprises at least one humanized light chain variable region that is at least 90% identical to an amino acid sequence of a humanized light chain variable region selected from the group consisting of:
XT-H2_hVL_V2.0 (SEQ ID NO:32), XT-H2_hVL_V3.0 (SEQ ID NO: 33), XT-H2_hVL_V4.0 (SEQ ID NO: 34), XT-H2_hVL_V4.1 (SEQ ID NO: 35), XT-M4_hVL_V2.4 (SEQ ID NO:39), XT-M4_hVL_V2.5 (SEQ ID NO: 40), XT-M4_hVL_V2.6 (SEQ ID NO: 41), XT-M4_hVL_V2.7 (SEQ ID NO: 42), XT-M4_hVL_V2.8 (SEQ ID NO: 43), XT-M4_hVL_V2.9 (SEQ ID NO: 44), XT-M4_hVL_V2.10 (SEQ ID NO: 45), XT-M4_hVL_V2.11 (SEQ ID NO: 46), XT-M4_hVL_V2.12 (SEQ ID NO: 47), XT-M4_hVL_V2.13 (SEQ ID NO: 48), and XT-M4_hVL_V2.14 (SEQ ID NO: 49).

22. A humanized antibody, or a RAGE-binding fragment thereof, comprising humanized heavy chain variable region that is at least 90% identical to an amino acid sequence of a humanized heavy chain variable region selected from the group consisting of:
XT-H2_hVH_V2.0 (SEQ ID NO: 28), XT-H2_hVH_V2.7 (SEQ ID NO: 29), XT-H2_hVH_V4 (SEQ ID NO: 30), XT-H2_hVH_V4.1 (SEQ ID NO: 31), XT-M4_hVH_V1.0 (SEQ ID NO: 36), XT-M4_hVH_V1.1 (SEQ ID NO: 37), and XT-M4_hVH_V2.0 (SEQ ID NO: 38).

23. The humanized antibody or fragment thereof of claim 21, further comprising a humanized heavy chain variable region that is at least 90% identical to an amino acid sequence of a humanized heavy chain variable region selected from the group consisting of:
XT-H2_hVH_V2.0 (SEQ ID NO: 28), XT-H2_hVH_V2.7 (SEQ ID NO: 29), XT-H2_hVH_V4 (SEQ ID NO: 30), XT-H2_hVH_V4.1 (SEQ ID NO: 31), XT-M4_hVH_V1.0 (SEQ ID NO: 36), XT-M4_hVH_V1.1 (SEQ ID NO: 37), and XT-M4_hVH_V2.0 (SEQ ID NO: 38).

24. A humanized antibody that binds specifically to RAGE, or a RAGE-binding fragment thereof, which antibody is a humanized XT-M4 antibody.

25. A humanized antibody that binds specifically to RAGE, or a RAGE-binding fragment thereof, which antibody is a humanized XT-H2 antibody.

26. An antibody that binds specifically to RAGE and blocks the binding of a RAGE body partner, which antibody has CDRs having at least 8 of the following characteristics;
a. amino acid sequence Y-X-M (Y32; X33; M34) in VH CDR1, where X is preferentially W or N;
b. amino acid sequence I-N-X-S (I51; N52; X53 and S54) in VH CDR2,
where X is P or N;
c. amino acid at position 58 in CDR2 of VH is Threonine;
d. amino acid at position 60 in CDR2 of VH is Tyrosine;
e. amino acid at position 103 in CDR3 of VH is Threonine;
f. one or more Tyrosine residues in CDR3 of VH;
g. positively charged residue (Arg or Lys) at position 24 in CDR1 of VL;
h. hydrophilic residue (Thr or Ser) at position 26 in CDR1 of VL;
i. small residue Ser or Ala at the position 25 in CDR1 of VL;
j. negatively charged residue (Asp or Glu) at position 33 in CDR1 of VL;
k. aromatic residue (Phe or Tyr or Trp) at position 37 in CDR1 of VL;
l. hydrophilic residue (Ser or Thr) at position 57 in CDR2 of VL;
m. P-X-T sequence at the end of CDR3 of VL where X could be hydrophobic residue Leu or Trp;
wherein amino acid position is as shown in the light and heavy chain amino acid sequences in SEQ ID NO:22 and SEQ ID NO:16, respectively.

27. An isolated nucleic acid comprising a nucleotide sequence encoding an anti-RAGE antibody variable region selected from the group consisting of:
XT-H1_VL (SEQ ID NO: 19), XT-H2_VL (SEQ ID NO: 22), XT-H3_VL (SEQ ID NO: 25), XT-H5_VL (SEQ ID NO: 23), XT-H7_VL (SEQ ID NO: 27), XT-M4_VL (SEQ ID NO: 17), XT-H1_VH (SEQ ID NO: 18), XT-H2_VH (SEQ ID NO: 21), XT-H3_VH (SEQ ID NO: 24), XT-H5_VH (SEQ ID NO: 20), XT-H7_VH (SEQ ID NO: 26), and XT-M4_VH (SEQ ID NO: 16).

28. An isolated nucleic acid that specifically hybridizes to a nucleic acid having a nucleotide sequence that is the complement of a nucleotide sequence encoding an anti-RAGE antibody variable region selected from the group consisting of:
XT-H1_VL (SEQ ID NO: 19), XT-H2_VL (SEQ ID NO: 22), XT-H3_VL (SEQ ID NO: 25), XT-H5_VL (SEQ ID NO: 23), XT-H7_VL (SEQ ID NO: 27), XT-M4_VL (SEQ ID NO: 17), XT-H1_VH (SEQ ID NO: 18), XT-H2_VH (SEQ ID NO: 21), XT-H3_VH (SEQ ID NO: 24), XT-H5_VH (SEQ ID NO: 20), XT-H7_VH (SEQ ID NO: 26), and XT-M4_VH (SEQ ID NO: 16), under stringent hybridization conditions.

29. An isolated nucleic acid comprising a nucleotide sequence encoding an anti-RAGE antibody variable region selected from the group consisting of:
XT-H2_hVL_V2.0 (SEQ ID NO:32), XT-H2_hVL_V3.0 (SEQ ID NO: 33), XT-H2_hVL_V4.0 (SEQ ID NO: 34), XT-H2_hVL_V4.1 (SEQ ID NO: 35), XT-M4_hVL_V2.4 (SEQ ID NO:39), XT-M4_hVL_V2.5 (SEQ ID NO: 40), XT-M4_hVL_V2.6 (SEQ ID NO: 41), XT-M4_hVL_V2.7 (SEQ ID NO: 42), XT-M4_hVL_V2.8 (SEQ ID NO: 43), XT-M4_hVL_V2.9 (SEQ ID NO: 44), XT-M4_hVL_V2.10 (SEQ ID NO: 45), XT-M4_hVL_V2.11 (SEQ ID NO: 46), XT-M4_hVL_V2.12 (SEQ ID NO: 47), XT-M4_hVL_V2.13 (SEQ ID NO: 48), and XT-M4_hVL_V2.14 (SEQ ID NO: 49), XT-H2_hVH_V2.0 (SEQ ID NO: 28), XT-H2_hVH_V2.7 (SEQ ID NO: 29), XT-H2_hVH_V4 (SEQ ID NO: 30), XT-H2_hVH_V4.1 (SEQ ID NO: 31), XT-M4_hVH_V1.0 (SEQ ID NO: 36), XT-M4_hVH_V1.1 (SEQ ID NO: 37), and XT-M4_hVH_V2.0 (SEQ ID NO: 38).

30. An isolated nucleic acid that specifically hybridizes to a nucleic acid having a nucleotide sequence that is the complement of a nucleotide sequence encoding an anti-RAGE antibody variable region selected from the group consisting of:
XT-H2_hVL_V2.0 (SEQ ID NO:32), XT-H2_hVL_V3.0 (SEQ ID NO: 33), XT-H2_hVL_V4.0 (SEQ ID NO: 34), XT-H2_hVL_V4.1 (SEQ ID NO: 35), XT-M4_hVL_V2.4 (SEQ ID NO:39), XT-M4_hVL_V2.5 (SEQ ID NO: 40), XT-M4_hVL_V2.6 (SEQ ID NO: 41), XT-M4_hVL_V2.7 (SEQ ID NO: 42), XT-M4_hVL_V2.8 (SEQ ID NO: 43), XT-M4_hVL_V2.9 (SEQ ID NO: 44), XT-M4_hVL_V2.10 (SEQ ID NO: 45), XT-M4_hVL_V2.11 (SEQ ID NO: 46), XT-M4_hVL_V2.12 (SEQ ID NO: 47), XT-M4_hVL_V2.13 (SEQ ID NO: 48), and XT-M4_hVL_V2.14 (SEQ ID NO: 49), XT-H2_hVH_V2.0 (SEQ ID NO: 28), XT-H2_hVH_V2.7 (SEQ ID NO: 29), XT-H2_hVH_V4 (SEQ ID NO: 30), XT-H2_hVH_V4.1 (SEQ ID NO: 31), XT-M4_hVH_V1.0 (SEQ ID NO: 36), XT-M4_hVH_V1.1 (SEQ ID NO: 37), and XT-M4_hVH_V2.0 (SEQ ID NO: 38), under stringent hybridization conditions.

31. An isolated nucleic acid comprising
(a) a nucleotide sequence encoding RAGE of baboon, monkey or rabbit having an amino acid sequence selected from the group consisting of SEQ ID NO: 7, SEQ ID NO: 9, SEQ ID NO: 11, and SEQ ID NO: 13;
(b) a nucleic acid that specifically hybridizes to the complement of (a): or
(c) a nucleotide sequence that is 95% identical to a nucleotide sequence encoding RAGE of baboon, monkey or rabbit selected from the group consisting of SEQ ID NO: 6, SEQ ID NO: 8, SEQ ID NO: 10, and SEQ ID NO: 12, when the query coverage is 100%;

32. A method of treating a subject having a RAGE-related disease or disorder comprising administering to the subject a therapeutically effective amount of antibody that:
(a) competes for binding to RAGE with an antibody selected from the group consisting of XT-H1, XT-H2, XT-H3, XT-H5, XT-H7, and XT-M4;
(b) binds to an epitope of RAGE that is bound by an antibody selected from the group consisting of XT-H1, XT-H2, XT-H3, XT-H5, XT-H7, and XT-M4;
(c) comprises one or more complementarity determining regions (CDRs) of a light chain or heavy chain of an antibody selected from the group consisting of XT-H1, XT-H2, XT-H3, XT-H5, XT-H7, and XT-M4; or
(d) is a RAGE-binding fragment of an antibody according to (a), (b) or (c).

33. The method of claim 32, wherein the RAGE-related disease or disorder is selected from the group consisting of sepsis, septic shock, listeriosis, inflammatory diseases, cancers, arthritis, Crohn's disease, chronic acute inflammatory diseases, cardiovascular diseases, erectile dysfunction, diabetes, complications of diabetes, vasculitis, nephropathies, retinopathies, and neuropathies.

34. The method of claim 32, comprising administering the antibody or RAGE-binding fragment thereof in combination with one or more agents useful in the treatment of the RAGE-related disease or disorder that is to be treated.

35. The method of claim 34, wherein the agent is selected from the group consisting of: anti-inflammatory agents, antioxidants, β-blockers, antiplatelet agents, ACE inhibitors, lipid-lowering agents, anti-angiogenic agents, and chemotherapeutics.

36. A method of treating sepsis or septic shock in a human subject comprising administering to the subject a therapeutically effective amount of a chimeric or humanized anti-RAGE antibody that comprises constant regions derived from human constant regions, and:
(a) competes for binding to RAGE with an antibody selected from the group consisting of XT-H1, XT-H2, XT-H3, XT-H5, XT-H7, and XT-M4;
(b) binds to an epitope of RAGE that is bound by an antibody selected from the group consisting of XT-H1, XT-H2, XT-H3, XT-H5, XT-H7, and XT-M4;
(c) comprises one or more complementarity determining regions (CDRS) of a light chain or heavy chain of an antibody selected from the group consisting of XT-H1, XT-H2, XT-H3, XT-H5, XT-H7, and XT-M4; or
(d) is a RAGE-binding fragment of an antibody according to (a), (b) or (c).

37. A method of treating sepsis or septic shock in a human subject comprising administering to the subject a therapeutically effective amount of a chimeric anti-RAGE antibody, or a RAGE-binding fragment thereof that comprises:
a light chain variable region having the amino acid sequence of the XT-M4 light chain variable region (SEQ ID NO: 17),
a heavy chain variable region having the amino acid sequence of the XT-M4 heavy chain variable region sequence (SEQ ID NO: 16),
a human kappa light chain constant region and a human IgG1 heavy chain constant region.

38. A method of treating systemic listeriosis in a human subject comprising administering to the subject a therapeutically effective amount of a chimeric or humanized anti-RAGE antibody that comprises constant regions derived from human constant regions, and:
(a) competes for binding to RAGE with an antibody selected from the group consisting of XT-H1, XT-H2, XT-H3, XT-H5, XT-H7, and XT-M4;
(b) binds to an epitope of RAGE that is bound by an antibody selected from the group consisting of XT-H1, XT-H2, XT-H3, XT-H5, XT-H7, and XT-M4;
(c) comprises one or more complementarity determining regions (CDRs) of a light chain or heavy chain of an antibody selected from the group consisting of XT-H1, XT-H2, XT-H3, XT-H5, XT-H7, and XT-M4; or
(d) is a RAGE-binding fragment of an antibody according to (a), (b) or (c).

39. A method of treating listeriosis in a human subject comprising administering to the subject a therapeutically effective amount of a chimeric anti-RAGE antibody, or a RAGE-binding fragment thereof, comprising
a light chain variable region having the amino acid sequence of the XT-M4 light chain variable region (SEQ ID NO: 17),
a heavy chain variable region having the amino acid sequence of the XT-M4 heavy chain variable region sequence (SEQ ID NO: 16),
a human kappa light chain constant region and a human IgG1 heavy chain constant region.

40. A method of inhibiting the binding of a RAGE binding partner (RAGE-BP) the RAGE in a mammalian subject, administering to the subject an inhibitory amount of a chimeric or humanized anti-RAGE antibody that comprises constant regions derived from human constant regions, and:
(a) competes for binding to RAGE with an antibody selected from the group consisting of XT-H1, XT-H2, XT-H3, XT-H5, XT-H7, and XT-M4;
(b) binds to an epitope of RAGE that is bound by an antibody selected from the group consisting of XT-H1, XT-H2, XT-H3, XT-H5, XT-H7, and XT-M4;
(c) comprises one or more complementarity determining regions (CDRs) of a light chain or heavy chain of an antibody selected from the group consisting of XT-H1, XT-H2, XT-H3, XT-H5, XT-H7, and XT-M4; or
(d) is a RAGE-binding fragment of an antibody according to (a), (b) or (c).

41. The antibody of claim 1, which antibody binds specifically to soluble RAGE (sRAGE).

42. The antibody of claim 41, which antibody binds specifically to sRAGE selected from the group consisting of murine sRAGE and human sRAGE.

43. The antibody of claim 42 which antibody binds specifically to sRAGE with a dissociation constant (Kd) in the range of from about 1×10⁻⁹ M to about 5×10⁻⁹ M.
